(19) United States
(12) Patent Application Publication
(10) Pub. No.: US 2023/0115907 A1
(43) Pub. Date: Apr. 13, 2023

(54) HETEROCYCLIC COMPOUND AND PHARMACEUTICAL COMPOSITION, PREPARATION METHOD, INTERMEDIATE AND USE THEREOF

(71) Applicant: EVOPOINT BIOSCIENCES CO., LTD., Suzhou, Jiangsu (CN)

(72) Inventors: Yonghan HU, Suzhou (CN); Dongdong WU, Suzhou (CN); Wei PENG, Suzhou (CN); Xiuchun ZHANG, Suzhou (CN); Yuchuan WU, Suzhou (CN)

(21) Appl. No.: 17/786,280

(22) PCT Filed: Dec. 18, 2020
(86) PCT No.: PCT/CN2020/137618

§ 371 Date: Jun. 16, 2022

(30) Foreign Application Priority Data
Dec. 20, 2019 (CN) 201911329611.X
Apr. 20, 2020 (CN) 202010312893.9
Aug. 28, 2020 (CN) 202010882490.8

(51) Int. Cl. C07D 487/04; C07D 471/04; A61P 35/00

(52) U.S. Cl. CPC C07D 487/04; C07D 471/04; A61P 35/00

(57) ABSTRACT

A heterocyclic compound, and a pharmaceutical composition thereof, a preparation method therefor, an intermediate thereof and an application thereof. The structure of the heterocyclic compound is as shown in formula (I) below. The compound has CDK7 inhibitory activity, and can be used to treat tumors and other diseases.

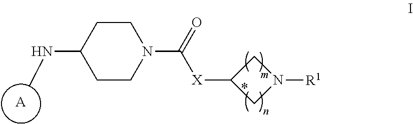

### CROSS-REFERENCE TO RELATED APPLICATIONS

The present application claims priority to the Chinese Patent Application 201911329611.X filed on Dec. 20, 2019, the Chinese Patent Application 202010312893.9 filed on Apr. 20, 2020, and the Chinese Patent Application 202010882490.8 filed on Aug. 28, 2020, which are incorporated herein by reference in entirety.

### TECHNICAL FIELD

The present application relates to a heterocyclic compound and a pharmaceutical composition, a preparation method, an intermediate and use thereof.

### BACKGROUND

Cyclin-dependent kinases (CDKs) belong to the serine/threonine kinase family, and the monomers thereof are not active. They must be bound to corresponding cyclins to form active heterodimer complexes to exert a regulatory effect, and thus phosphorylation of corresponding substrates can be catalyzed, and cells can be directly or indirectly regulated to complete the cell cycle, thereby causing cell growth and proliferation. It has now been found that the human genome encodes 21 CDKs and more than 15 cyclins. CDKs can be divided into two major classes according to their functions: CDKs that control cell cycle and CDKs that control cell transcription. Among these, CDK 1/2/4/6 are primarily associated with the cell cycle, while CDK7/8/9/10 are mainly associated with the transcriptional mechanism of the genetic information within the cells (Asghar U, Witkiewicz A K, Turner N C, et al., The history and future of targeting cyclin-dependent kinases in cancer therapy. Nat Rev Drug Discov, 2015 (2): 130-146).

CDK7 is an important member of the CDK family, and it regulates the cell cycle mainly in two indirect ways. One is that CDK7, together with cyclin H and Mat1, constitutes CAK (CDKs activating kinase), and further phosphorylates CDK1/2, thereby activating their function in the cell cycle (Yee A, Nichols M A, Wu L, Hall F L, Kobayashi R, Xiong Y. Molecular cloning of CDK7-associated human MAT1, a cyclin-dependent kinase-activating kinase (CAK) assembly factor. Cancer Res. 1995; 55: 6058-6062). Another way is that CDK7, as a subunit component of the universal transcription factor TFIIH, phosphorylates the carboxy-terminal domain (CTD) of the large subunit of RNA polymerase II (RNAP II), and regulates the gene transcription process in cells (Kelso T W, Baumgart K, Eickhoff J, Albert T, Antrecht C, Lemcke S et al. Cyclin-dependent kinase 7 controls mRNA synthesis by affecting stability of preinitiation complexes, leading to altered gene expression, cell cycle progression, and survival of tumor cells. Mol Cell Biol. 2014; 34: 3675-3688). Because of its dual function of CAK and CTD phosphorylation, CDK7 plays an important role in cell proliferation, cell cycle and transcription.

In recent years, inhibition of CDK7 has gradually become a promising therapeutic strategy for a variety of cancers. Inhibition of CDK7 can inhibit expression of key oncogenes such as c-Myc (Chipumuro E, Marco E, Christensen C L, Kwiatkowski N, Zhang T, Hatheway C M, et al. CDK7 inhibition suppresses super-enhancer-linked oncogenic transcription in MYCN-driven cancer. Cell. 2014, 159: 1126-39). Preclinical research data show that small molecule inhibitors that inhibit CDK7 have good anti-cancer effect in hormone receptor positive and triple negative breast cancers (Wang Y, Zhang T, Kwiatkowski N, Abraham B J, Lee T I, Xie S, et al. CDK7dependent transcriptional addiction in triple-negative breast cancer. Cell. 2015; 163: 174-86), and in cancers driven by transcription factors, such as small cell lung cancer (SCLC) (Christensen C L, Kwiatkowski N, Abraham B J, Carretero J, Al-Shahrour F, Zhang T, et al. Targeting transcriptional addictions in small cell lung cancer with a covalent CDK7 inhibitor. Cancer Cell 2014; 26: 909-22). There is currently a lack of effective treatment approaches for these cancers, and therefore there are significant unmet medical needs. Also, CDK7 inhibitors may be effective against cancers that have become resistant to current therapies because they have a different mechanism of action. Therefore, the development of CDK7 inhibitors would likely be an effective means for treating these malignant tumors.

CDK7 inhibitors that have been reported include THZ1, SY-1365 (a compound in clinic trials from Syros), CT7001 (a compound in clinic trials from Carrick), and the like. Their structures are as follows:

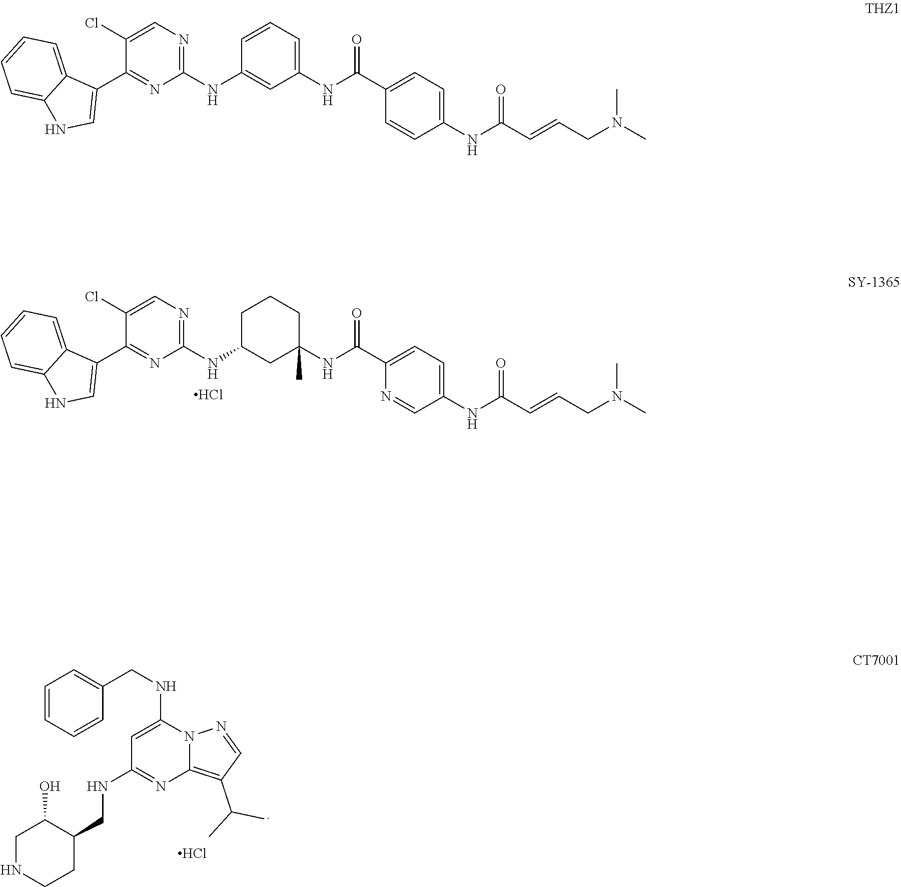 [SMILES: CN(C)C/C=C/C(=O)Nc1ccc(C(=O)Nc2cccc(Nc3ncc(Cl)c(-c4c[nH]c5ccccc45)n3)c2)cc1 ; CN(C)C/C=C/C(=O)Nc1ccc(C(=O)N[C@@]2(C)CCC[C@@H](Nc3ncc(Cl)c(-c4c[nH]c5ccccc45)n3)C2)nc1 ; Cl ; CC(C)c1cnn2c(NCc3ccccc3)cc(NC[C@H]3CCNC[C@@H]3O)nc12 ; Cl]

Patent application WO2019099298A1 of the Lilly Company discloses two CDK7 inhibitors in Example 1 and Example 3, and the structures of the compounds are as follows:

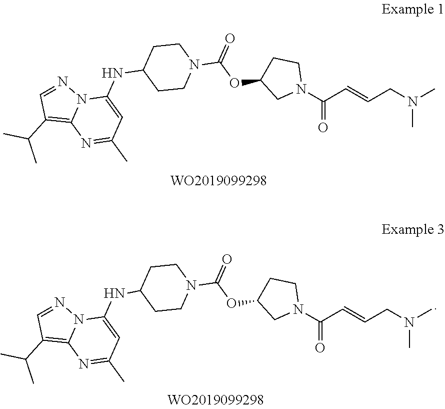

### SUMMARY

The technical problem to be solved by the present invention is that the existing CDK7 inhibitors are relatively homogeneous in structure, and thus the present application provides a heterocyclic compound with a new structure and a pharmaceutical composition, a preparation method, an intermediate and use thereof. The heterocyclic compound disclosed herein has good inhibition effect on CDK7, and can be used for treating diseases such as tumors.

The present invention provides a compound of formula I:

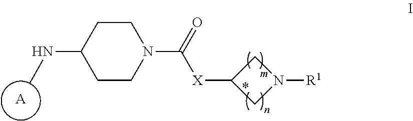

or a tautomer, a stereoisomer or an isotopic derivative thereof, or a pharmaceutically acceptable salt of any of the foregoing (namely the aforementioned compound of formula I, tautomer, stereoisomer or isotopic derivative), or a crystalline form or a solvate of any of the foregoing (namely the aforementioned compound of formula I, tautomer, stereoisomer, isotopic derivative or pharmaceutically acceptable salt);

wherein, ring A is

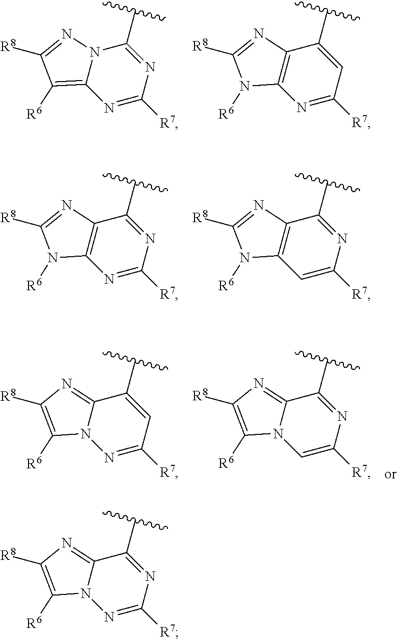

X is O or NR^{a};

R^{a} is hydrogen, methyl or ethyl;

m is 1 or 2;

n is 1, 2 or 3;

R¹ is

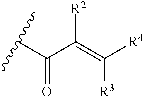

(e.g.,

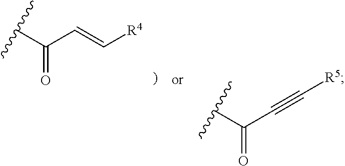

R² is hydrogen or C₁₋₆ alkyl;
R³ is hydrogen or C₁₋₆ alkyl;
R⁴ is hydrogen, halogen, C₁₋₆ alkyl or —CH₂—NR^{4a}R^{4b} (e.g., —CH₂—N(CH₃)₂);
R^{4a} and R^{4b} are each independently hydrogen or C₁₋₆ alkyl (e.g., methyl); or, R^{4a} and R^{4b}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{b};
each R^{b} is independently halogen, hydroxy or C₁₋₄ alkyl;
R⁵ is hydrogen or C₁₋₆ alkyl (e.g., methyl);
each R⁶ is independently hydrogen, cyclopropyl or C₁₋₆ alkyl (e.g., methyl, ethyl, n-propyl or isopropyl, preferably isopropyl);
each R⁷ is independently hydrogen, a substituted or unsubstituted C₁₋₆ alkyl (the C₁₋₆ alkyl is, e.g., methyl, ethyl or isopropyl; further, the substituted or unsubstituted C₁₋₆ alkyl is, e.g., methyl, ethyl, isopropyl or —CH₂OH), a substituted or unsubstituted phenyl, a substituted or unsubstituted 5-6 membered heteroaryl (e.g., the 5-6 membered heteroaryl is, e.g., pyrazolyl, such as

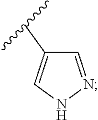 [SMILES: *c1cn[nH]c1]

further, the substituted or unsubstituted 5-6 membered heteroaryl is, e.g.,

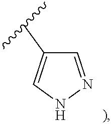 [SMILES: *c1cn[nH]c1]

a substituted or unsubstituted C₃₋₆ cycloalkyl (e.g., cyclopropyl), —OR^{7a} (e.g.,

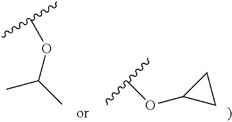

or —NR^{7b}R^{7c} (e.g.,

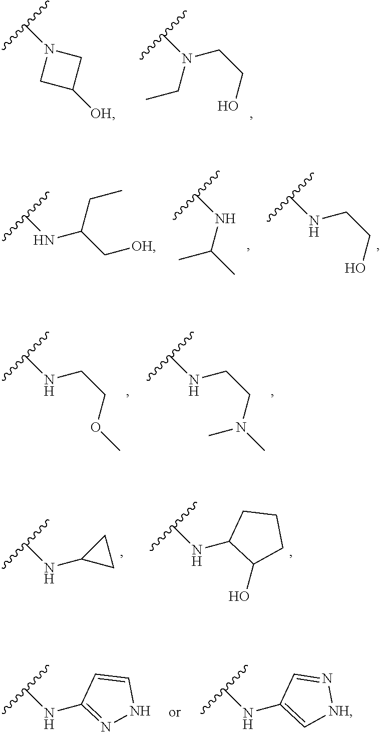

wherein

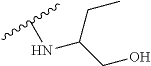 [SMILES: *NC(CC)CO]

is, e.g.,

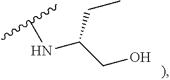 [SMILES: *N[C@H](CC)CO]

wherein the substituted C₁₋₆ alkyl, the substituted phenyl, the substituted C₃₋₆ cycloalkyl and the substituted 5-6 membered heteroaryl mean that the C₁₋₆ alkyl, the phenyl, the C₃₋₆ cycloalkyl and the 5-6 membered heteroaryl are each independently substituted with 1, 2, 3 or 4 R^{7d} (e.g., substituted with 1 R^{7d});
each R^{7d} is independently hydroxy, halogen, C₁₋₄ alkyl, —NR^{a1}R^{a2} (e.g., —N(CH₃)₂) or C₁₋₄ alkoxy (e.g., methoxy);

R^{7a} is hydrogen, a substituted or unsubstituted C₁₋₆ alkyl (the C₁₋₆ alkyl is, e.g., isopropyl; further, the substituted or unsubstituted C₁₋₆ alkyl is isopropyl), a substituted or unsubstituted C₃₋₆ cycloalkyl (the C₃₋₆ cycloalkyl is, e.g., cyclopropyl; further, the substituted or unsubstituted C₃₋₆ cycloalkyl is, e.g., cyclopropyl), a substituted or unsubstituted 4-6 membered heterocycloalkyl or a substituted or unsubstituted 5-6 membered heteroaryl, wherein the substituted C₁₋₆ alkyl, the substituted C₃₋₆ cycloalkyl, the substituted 4-6 membered heterocycloalkyl and the substituted 5-6 membered heteroaryl mean that the C₁₋₆ alkyl, the C₃₋₆ cycloalkyl, the 4-6 membered heterocycloalkyl and the 5-6 membered heteroaryl are each independently substituted with 1, 2. 3 or 4 R^{c} (e.g., substituted with 1 R^{c});

each R^{c} is independently hydroxy, halogen, C₁₋₄ alkyl, —NR^{c1}R^{c2} (e.g., —N(CH₃)₂) or C₁₋₄ alkoxy (e.g., methoxy);

R^{7b} and R^{7c} are each independently hydrogen, a substituted or unsubstituted C₁₋₄ alkyl (the C₁₋₄ alkyl is, e.g., ethyl, isopropyl or sec-butyl; further, the substituted or unsubstituted C₁₋₄ alkyl is, e.g., ethyl, isopropyl,

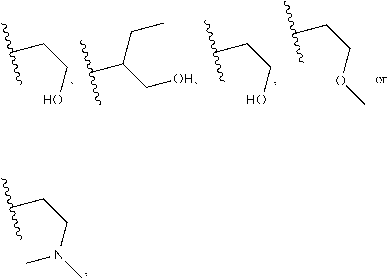

wherein

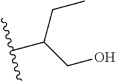 [SMILES: *C(CC)CO]

is, e.g.

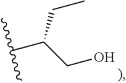 [SMILES: *[C@H](CC)CO]

a substituted or unsubstituted C₃₋₆ cycloalkyl (the C₃₋₆ cycloalkyl is, e.g., cyclopropyl or cyclopentyl; further, the substituted or unsubstituted C₃₋₆ cycloalkyl is, e.g., cyclopropyl or

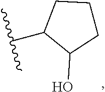

wherein

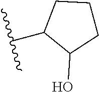 [SMILES: *C1CCCC1O]

is, e.g.,

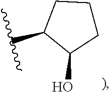 [SMILES: *[C@H]1CCC[C@H]1O]

a substituted or unsubstituted 4-6 membered heterocycloalkyl, or a substituted or unsubstituted 5-6 membered heteroaryl (the 5-6 membered heteroaryl is, e.g., pyrazolyl, such as

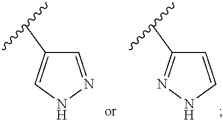 [SMILES: *c1cn[nH]c1 ; *c1cc[nH]n1]

further, the substituted or unsubstituted 5-6 membered heteroaryl is, e.g.,

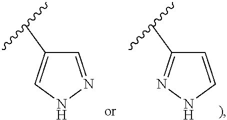 [SMILES: *c1cn[nH]c1 ; *c1cc[nH]n1]

wherein the substituted C₁₋₄ alkyl, the substituted C₃₋₆ cycloalkyl, the substituted 4-6 membered heterocycloalkyl and the substituted 5-6 membered heteroaryl mean that the C₁₋₄ alkyl, the C₃₋₆ cycloalkyl, the 4-6 membered heterocycloalkyl and the 5-6 membered heteroaryl are each independently substituted with 1, 2, 3, or 4 R^{d} (e.g., substituted with 1 R^{d});

or, R^{7b} and R^{7c}, together with a nitrogen atom attached thereto, form a substituted or unsubstituted 4-6 membered heterocycloalkyl (the 4-6 membered heterocycloalkyl is, e.g., azetidinyl; further, the substituted or unsubstituted 4-6 membered heterocycloalkyl is, e.g.,

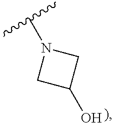 [SMILES: *N1CC(O)C1]

wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e} (e.g., substituted with 1 R^{e});

each R^{d} is independently hydroxy, halogen, C₁₋₄ alkyl, —NR^{d1}R^{d2} (e.g., —N(CH₃)₂) or C₁₋₄ alkoxy (e.g., methoxy);

each R^{e} is independently hydroxy, halogen, C₁₋₄ alkyl, —NR^{e1}R^{e2} or C₁₋₄ alkoxy (e.g., methoxy);

each R^{a1}, each R^{a2}, each R^{c1}, each R^{c2}, each R^{d1}, each R^{d2}, each R^{e1} and each R^{e2} are each independently hydrogen or C₁₋₄ alkyl (e.g., methyl);

each R^{g} is independently hydrogen or C₁₋₄ alkyl (e.g., methyl);

when a carbon atom marked by * has chirality, the carbon atom is in S configuration, R configuration, or a mixture thereof;

the number of heteroatoms in the 4-6 membered heterocycloalkyl and the number of heteroatoms in the 5-6 membered heteroaryl are independently 1, 2 or 3, and each heteroatom is independently selected from N, O and S.

In some embodiments, each R⁷ is independently hydrogen, a substituted or unsubstituted C₁₋₆ alkyl, a substituted or unsubstituted phenyl, a substituted or unsubstituted 5-6 membered heteroaryl, —OR^{7a} or —NR^{7b}R^{7c}, wherein the substituted C₁₋₆ alkyl, the substituted phenyl and the substituted 5-6 membered heteroaryl mean that the C₁₋₆ alkyl, the phenyl and the 5-6 membered heteroaryl are each independently substituted with 1, 2, 3 or 4 R^{7d}.

In some embodiments, the compound of formula I is defined as follows:

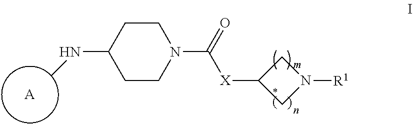

wherein, ring A is

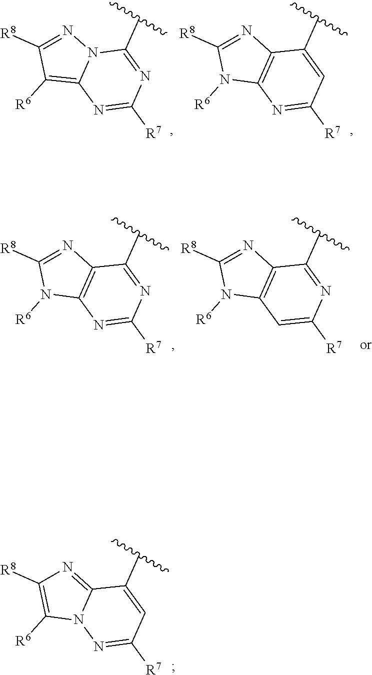

X is O or NR^{a};

R^{a} is hydrogen, methyl or ethyl;

m is 1 or 2;

n is 1, 2 or 3;

R¹ is

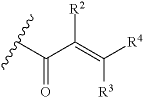

(e.g.,

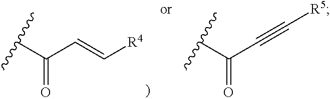

R² is hydrogen or C₁₋₆ alkyl;

R³ is hydrogen or C₁₋₆ alkyl;

R⁴ is hydrogen, halogen, C₁₋₆ alkyl or —CH₂—NR^{4a}R^{4b} (e.g., —CH₂—N(CH₃)₂);

R^{4a} and R^{4b} are each independently hydrogen or C₁₋₆ alkyl (e.g., methyl); or, R^{4a} and R^{4b}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{b};

each R^{b} is independently halogen, hydroxy or C₁₋₄ alkyl;

R⁵ is hydrogen or C₁₋₆ alkyl (e.g., methyl);

each R⁶ is independently hydrogen, cyclopropyl or C₁₋₆ alkyl (e.g., methyl, ethyl, n-propyl or isopropyl, preferably isopropyl);

each R⁷ is independently hydrogen, C₁₋₆ alkyl, phenyl, 5-6 membered heteroaryl (e.g., pyrazolyl, such as

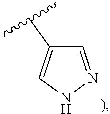 [SMILES: *c1cn[nH]c1]

—OR^{7a} or —NR^{7b}R^{7c} (e.g.,

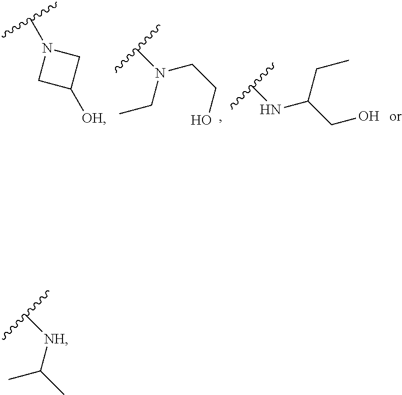

wherein

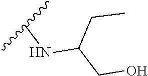 [SMILES: *NC(CC)CO]

is, e.g.,

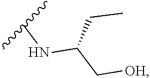 [SMILES: *N[C@H](CC)CO]

wherein the number of heteroatoms in the 5-6 membered heteroaryl is 1, 2 or 3, and each heteroatom is independently selected from N, O and S;

R^{7a} is hydrogen or a substituted or unsubstituted C₁₋₆ alkyl, wherein the substituted C₁₋₆ alkyl means that the C₁₋₆ alkyl is substituted with 1, 2, 3 or 4 R^{c};

each R^{c} is independently hydroxy, halogen, C₁₋₄ alkyl or C₁₋₄ alkoxy;

R^{7b} and R^{7c} are each independently hydrogen or a substituted or unsubstituted C₁₋₄ alkyl (the substituted or unsubstituted C₁₋₄ alkyl is, e.g., ethyl, isopropyl,

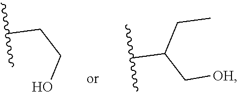

wherein

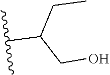 [SMILES: *C(CC)CO]

is, e.g.,

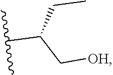 [SMILES: *[C@H](CC)CO]

wherein the substituted C₁₋₄ alkyl means that the C₁₋₄ alkyl is substituted with 1, 2, 3 or 4 R^{d};

or, R^{7b} and R^{7c}, together with a nitrogen atom attached thereto, form a substituted or unsubstituted 4-6 membered heterocycloalkyl (the 4-6 membered heterocycloalkyl is, e.g., azetidinyl), wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e};

each R^{d} is independently hydroxy, halogen, C₁₋₄ alkyl or C₁₋₄ alkoxy;

each R^{e} is independently hydroxy, halogen, C₁₋₄ alkyl or C₁₋₄ alkoxy;

each R^{g} is independently hydrogen or C₁₋₄ alkyl (e.g., methyl);

when a carbon atom marked by * has chirality, the carbon atom is in S configuration, R configuration, or a mixture thereof.

In some embodiments, in the compound of formula I, ring A is

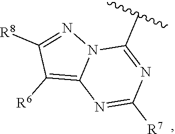

and the other variables are defined as in any of the embodiments of the present invention.

In some embodiments, in the compound of formula I, ring A is

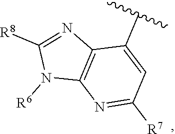

and the other variables are defined as in any of the embodiments of the present invention.

In some embodiments, in the compound of formula I, ring A is

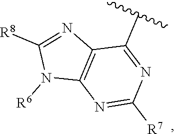

and the other variables are defined as in any of the embodiments of the present invention.

In some embodiments, in the compound of formula I, ring A is

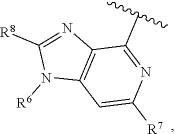

and the other variables are defined as in any of the embodiments of the present invention.

In some embodiments, in the compound of formula I, ring A is

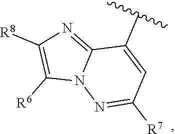

and the other variables are defined as in any of the embodiments of the present invention.

In some embodiments, in the compound of formula I, ring A is

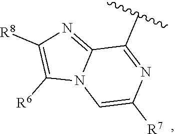

and the other variables are defined as in any of the embodiments of the present invention.

In some embodiments, in the compound of formula I, ring A is

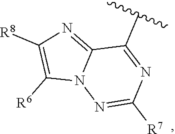

and the other variables are defined as in any of the embodiments of the present invention.

In some embodiments, in the compound of formula I, ring A is preferably

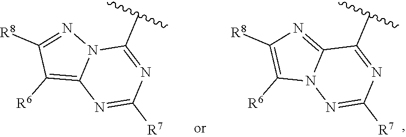

and the other variables are defined as in any of the embodiments of the present invention.

In some embodiments, in the compound of formula I, R^{a} is hydrogen, and the other variables are defined as in any of the embodiments of the present invention.

In some embodiments, in the compound of formula I described in any of the embodiments above, n is 1, and the other variables are defined as in any of the embodiments of the present invention.

In some embodiments, in the compound of formula I, R² is hydrogen, and the other variables are defined as in any of the embodiments of the present invention.

In some embodiments, in the compound of formula I, R³ is hydrogen, and the other variables are defined as in any of the embodiments of the present invention.

In some embodiments, in the compound of formula I, R⁴ is —CH₂—NR^{4a}R^{4b}, and the other variables are defined as in any of the embodiments of the present invention.

In some embodiments, in the compound of formula I, R^{4a} is C₁₋₆ alkyl, and the other variables are defined as in any of the embodiments of the present invention.

In some embodiments, in the compound of formula I, R^{4b} is C₁₋₆ alkyl, and the other variables are defined as in any of the embodiments of the present invention.

In some embodiments, in the compound of formula I, R⁴ is —CH₂—N(CH₃)₂, and the other variables are defined as in any of the embodiments of the present invention.

In some embodiments, in the compound of formula I, R⁵ is C₁₋₆ alkyl, and the other variables are defined as in any of the embodiments of the present invention.

In some embodiments, in the compound of formula I, each R^{7d} is independently hydroxy, —NR^{a1}R^{a2} or C₁₋₄ alkoxy, and the other variables are defined as in any of the embodiments of the present invention.

In some embodiments, in the compound of formula I, R^{7d} is hydroxy.

In some embodiments, in the compound of formula I, R^{a1} and R^{a2} are C₁₋₄ alkyl, and the other variables are defined as in any of the embodiments of the present invention.

In some embodiments, in the compound of formula I, each R^{c} is independently hydroxy, —NR^{c1}R^{c2} or C₁₋₄ alkoxy, and the other variables are defined as in any of the embodiments of the present invention.

In some embodiments, in the compound of formula I, R^{c1} and R^{c2} are C₁₋₄ alkyl, and the other variables are defined as in any of the embodiments of the present invention.

In some embodiments, in the compound of formula I, R^{C} is hydroxy, and the other variables are defined as in any of the embodiments of the present invention.

In some embodiments, in the compound of formula I, each R^{d} is independently hydroxy, —NR^{d1}R^{d2} or C₁₋₄ alkoxy, and the other variables are defined as in any of the embodiments of the present invention.

In some embodiments, in the compound of formula I, R^{d1} and R^{d2} are C₁₋₄ alkyl, and the other variables are defined as in any of the embodiments of the present invention.

In some embodiments, in the compound of formula I, R^{d} is hydroxy, and the other variables are defined as in any of the embodiments of the present invention.

In some embodiments, in the compound of formula I, each R^{e} is independently hydroxy, —NR^{e1}R^{e2} or C₁₋₄ alkoxy, and the other variables are defined as in any of the embodiments of the present invention.

In some embodiments, in the compound of formula I, R^{e1} and R^{e2} are C₁₋₄ alkyl, and the other variables are defined as in any of the embodiments of the present invention.

In some embodiments, in the compound of formula I, R^{e} is hydroxy, and the other variables are defined as in any of the embodiments of the present invention.

In some embodiments, in the compound of formula I, each R⁶ is independently hydrogen or C₁₋₆ alkyl, and the other variables are defined as in any of the embodiments of the present invention.

In some embodiments, in the compound of formula I, each R⁷ is independently hydrogen, a substituted or unsubstituted C₁₋₆ alkyl, a substituted or unsubstituted 5-6 membered heteroaryl, —OR^{7a} or —NR^{7b}R^{7c}, wherein the substituted C₁₋₆ alkyl and the substituted 5-6 membered heteroaryl mean that the C₁₋₆ alkyl and the 5-6 membered heteroaryl are each independently substituted with 1, 2, 3 or 4 R^{7d} (e.g., substituted with 1 R^{7d}), and the other variables are defined as in any of the embodiments of the present invention.

In some embodiments, in the compound of formula I, R^{7a} is hydrogen, a substituted or unsubstituted C₁₋₆ alkyl, or a substituted or unsubstituted C₃₋₆ cycloalkyl, wherein the substituted C₁₋₆ alkyl and the substituted C₃₋₆ cycloalkyl mean that the C₁₋₆ alkyl and the C₃₋₆ cycloalkyl are each independently substituted with 1, 2, 3 or 4 R^{c} (e.g., substituted with 1 R^{c}), and the other variables are defined as in any of the embodiments of the present invention.

In some embodiments, in the compound of formula I, R^{7b} and R^{7c} are each independently hydrogen, a substituted or unsubstituted C₁₋₄ alkyl, a substituted or unsubstituted C₃₋₆ cycloalkyl, or a substituted or unsubstituted 5-6 membered heteroaryl, wherein the substituted C₁₋₄ alkyl, the substituted C₃₋₆ cycloalkyl and the substituted 5-6 membered heteroaryl mean that the C₁₋₄ alkyl, the C₃₋₆ cycloalkyl and the 5-6 membered heteroaryl are each independently substituted with 1, 2, 3 or 4 R^{d} (e.g., substituted with 1 R^{d}), and the other variables are as defined in any embodiment of the present invention.

In some embodiments, in the compound of formula I, one of R^{7b} and R^{7c} is hydrogen, and the other variables are as defined in any embodiment of the present invention.

In some embodiments, in the compound of formula I, each R⁷ is independently hydrogen, C₁₋₆ alkyl, C₃₋₆ cycloalkyl, 5-6 membered heteroaryl or —NR^{7b}R^{7c}, and the other variables are as defined in any embodiment of the present invention.

In some embodiments, in the compound of formula I in which ring A is

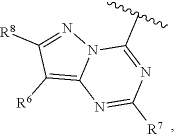

R⁶, R⁷ and R⁸ are defined as follows:

R⁶ is C₁₋₆ alkyl (e.g., isopropyl);

R⁷ is a substituted or unsubstituted C₁₋₆ alkyl (the C₁₋₆ alkyl is, e.g., methyl; further, the substituted or unsubstituted C₁₋₆ alkyl is, e.g., methyl), —OR^{7a} (e.g.,

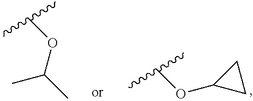

or —NR^{7b}R^{7c} (e.g.,

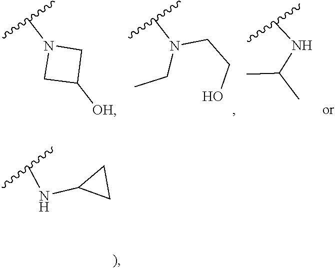

wherein the substituted C₁₋₆ alkyl means that the C₁₋₆ alkyl is substituted with 1, 2, 3 or 4 R^{7d} (e.g., substituted with 1 R^{7d});

R^{7a} is hydrogen, a substituted or unsubstituted C₁₋₆ alkyl (the C₁₋₆ alkyl is, e.g., isopropyl; further, the substituted or unsubstituted C₁₋₆ alkyl is, e.g., isopropyl), or a substituted or unsubstituted C₃₋₆ cycloalkyl (the C₃₋₆ cycloalkyl is, e.g., cyclopropyl; further, the substituted or unsubstituted C₃₋₆ cycloalkyl is, e.g., cyclopropyl), wherein the substituted C₁₋₆ alkyl and the substituted C₃₋₆ cycloalkyl mean that the C₁₋₆ alkyl and the C₃₋₆ cycloalkyl are each independently substituted with 1, 2. 3 or 4 R^{c} (e.g., substituted with 1 R^{c});

R^{7b} and R^{7c} are each independently hydrogen, a substituted or unsubstituted C₁₋₄ alkyl (the C₁₋₄ alkyl is, e.g., ethyl or isopropyl; further, the substituted or unsubstituted C₁₋₄ alkyl is, e.g., ethyl, isopropyl or

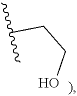 [SMILES: *CCO]

or a substituted or unsubstituted C₃₋₆ cycloalkyl (the C₃₋₆ cycloalkyl is, e.g., cyclopropyl; further, the substituted or unsubstituted C₃₋₆ cycloalkyl is, e.g., cyclopropyl), wherein the substituted C₁₋₄ alkyl and the substituted C₃₋₆ cycloalkyl mean that the C₁₋₄ alkyl and the C₃₋₆ cycloalkyl are substituted with 1, 2, 3 or 4 R^{d} (e.g., substituted with 1 R^{d});

or, R^{7b} and R^{7c}, together with a nitrogen atom attached thereto, form a substituted or unsubstituted 4-6 membered heterocycloalkyl (the 4-6 membered heterocycloalkyl is, e.g., azetidinyl; further, the substituted or unsubstituted 4-6 membered heterocycloalkyl is, e.g.,

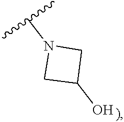 [SMILES: *N1CC(O)C1]

wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e} (e.g., substituted with 1 R^{e});

R^{g} is hydrogen;

the other variables are defined as in any of the embodiments of the present invention.

In some embodiments, in the compound of formula I in which ring A is

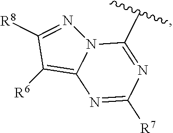

R⁶, R⁷ and R⁸ are defined as follows:

R⁶ is C₁₋₆ alkyl (e.g., isopropyl);

R⁷ is C₁₋₆ alkyl (e.g., methyl) or —NR^{7b}R^{7c} (e.g.,

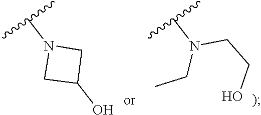

R^{7b} and R^{7c} are each independently hydrogen or a substituted or unsubstituted C₁₋₄ alkyl, wherein the substituted C₁₋₄ alkyl means that the C₁₋₄ alkyl is substituted with 1, 2, 3 or 4 R^{d};

or, R^{7b} and R^{7c}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e};

R^{g} is hydrogen;

the other variables are defined as in any of the embodiments of the present invention.

In some embodiments, in the compound of formula I in which ring A is

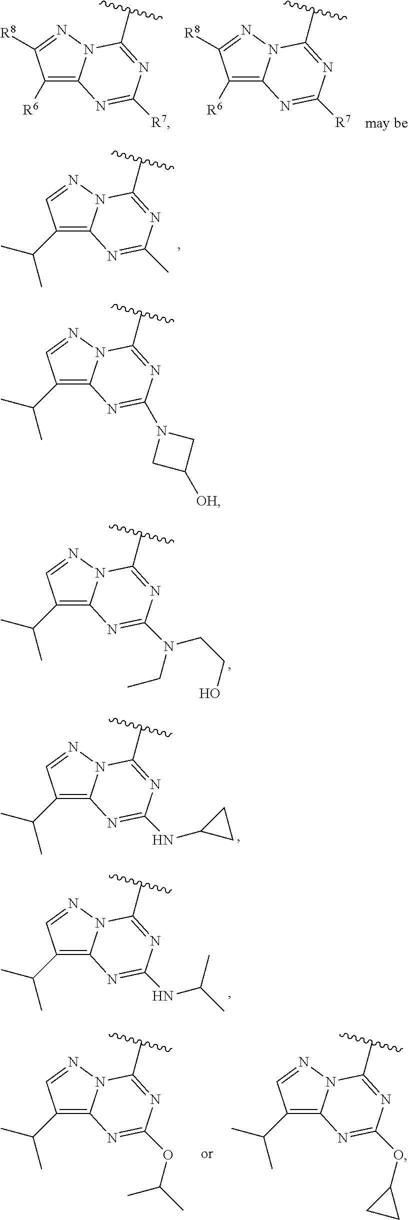

and the other variables are defined as in any of the embodiments of the present invention.

In some embodiments, in the compound of formula I in which ring A is

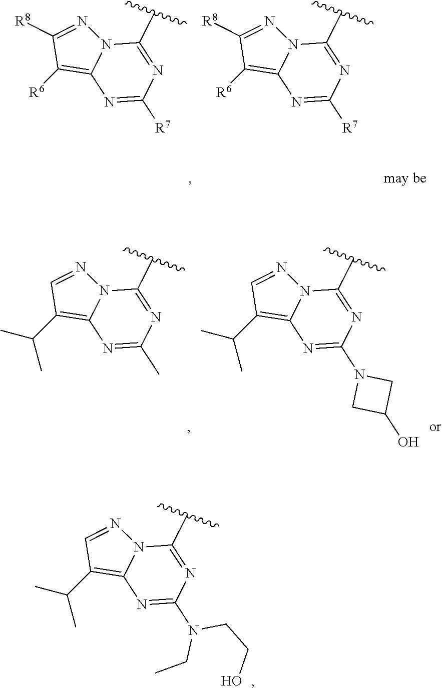

and the other variables are defined as in any of the embodiments of the present invention.

In some embodiments, in the compound of formula I in which ring A is

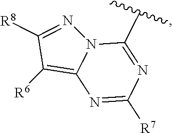

R⁶, R⁷ and R⁸ are defined preferably as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

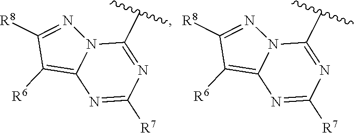

is preferably.

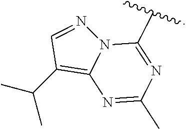

In some embodiments, in the compound of formula I in which ring A is

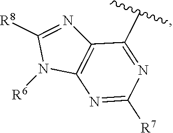

R⁶, R⁷ and R⁸ are defined as follows:

R⁶ is C₁₋₆ alkyl (e.g., ethyl or isopropyl);

R⁷ is hydrogen, a substituted or unsubstituted C₁₋₆ alkyl (the C₁₋₆ alkyl is, e.g., methyl or ethyl; further, the substituted or unsubstituted C₁₋₆ alkyl is, e.g., methyl, ethyl or —CH₂OH), a substituted or unsubstituted 5-6 membered heteroaryl (the 5-6 membered heteroaryl is, e.g., pyrazolyl, such as

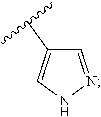 [SMILES: *c1cn[nH]c1]

further, the substituted or unsubstituted 5-6 membered heteroaryl is, e.g.,

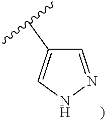 [SMILES: *c1cn[nH]c1]

or —NR^{7b}R^{7c} (e.g.,

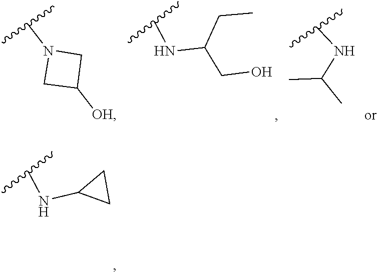

wherein

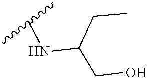 [SMILES: *NC(CC)CO]

e.g.,

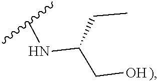 [SMILES: *N[C@H](CC)CO]

wherein the substituted C₁₋₆ alkyl and the substituted 5-6 membered heteroaryl mean that the C₁₋₆ alkyl and the 5-6 membered heteroaryl are each independently substituted with 1, 2, 3 or 4 R^{7d} (e.g., substituted with 1 R^{7d});

R^{7b} and R^{7c} are each independently hydrogen, a substituted or unsubstituted C₁₋₄ alkyl (the C₁₋₄ alkyl is, e.g., isopropyl or sec-butyl; further, the substituted or unsubstituted C₁₋₄ alkyl is, e.g., isopropyl or

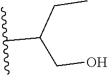

wherein

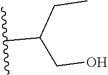 [SMILES: *C(CC)CO]

is, e.g.,

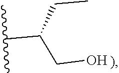 [SMILES: *[C@H](CC)CO]

or a substituted or unsubstituted C₃₋₆ cycloalkyl (the C₃₋₆ cycloalkyl is, e.g., cyclopropyl; further, the substituted or unsubstituted C₃₋₆ cycloalkyl is, e.g., cyclopropyl), wherein the substituted C₁₋₄ alkyl and the substituted C₃₋₆ cycloalkyl mean that the C₁₋₄ alkyl and the C₃₋₆ cycloalkyl are each independently substituted with 1, 2, 3 or 4 R^{d} (e.g., substituted with 1 R^{d}); preferably, one of R^{7b} and R^{7c} is hydrogen;

or, R^{7b} and R^{7c}, together with a nitrogen atom attached thereto, form a substituted or unsubstituted 4-6 membered heterocycloalkyl (the 4-6 membered heterocycloalkyl is, e.g., azetidinyl; further, the substituted or unsubstituted 4-6 membered heterocycloalkyl is, e.g.,

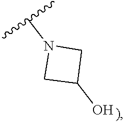 [SMILES: *N1CC(O)C1]

wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e} (e.g., substituted with 1 R^{e});

R^{g} is hydrogen or C₁₋₄ alkyl (e.g., methyl), preferably hydrogen;

the other variables are defined as in any of the embodiments of the present invention.

In some embodiments, in the compound of formula I in which ring A is

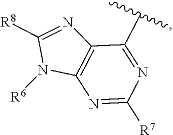

R⁶, R⁷ and R⁸ are defined as follows:

R⁶ is C₁₋₆ alkyl (e.g., ethyl or isopropyl);

R⁷ is hydrogen, C₁₋₆ alkyl (e.g., methyl), 5-6 membered heteroaryl (e.g., pyrazolyl, such as

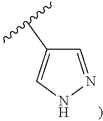 [SMILES: *c1cn[nH]c1]

or —NR^{7b}R^{7c} (e.g.,

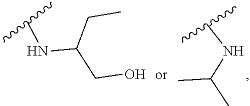

wherein

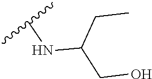 [SMILES: *NC(CC)CO]

is, e.g.,

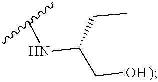 [SMILES: *N[C@H](CC)CO]

R^{7a} is hydrogen or a substituted or unsubstituted C₁₋₆ alkyl, wherein the substituted C₁₋₆ alkyl means that the C₁₋₆ alkyl is substituted with 1, 2, 3 or 4 R^{c};

R^{7b} and R^{7c} are each independently hydrogen or a substituted or unsubstituted C₁₋₄ alkyl, wherein the substituted C₁₋₄ alkyl means that the C₁₋₄ alkyl is substituted with 1, 2, 3 or 4 R^{d};

or, R^{7b} and R^{7c}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e};

R^{g} is hydrogen or C₁₋₄ alkyl;

the other variables are defined as in any of the embodiments of the present invention.

In some embodiments, in the compound of formula I in which ring A is

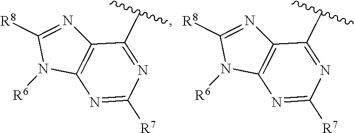

may be

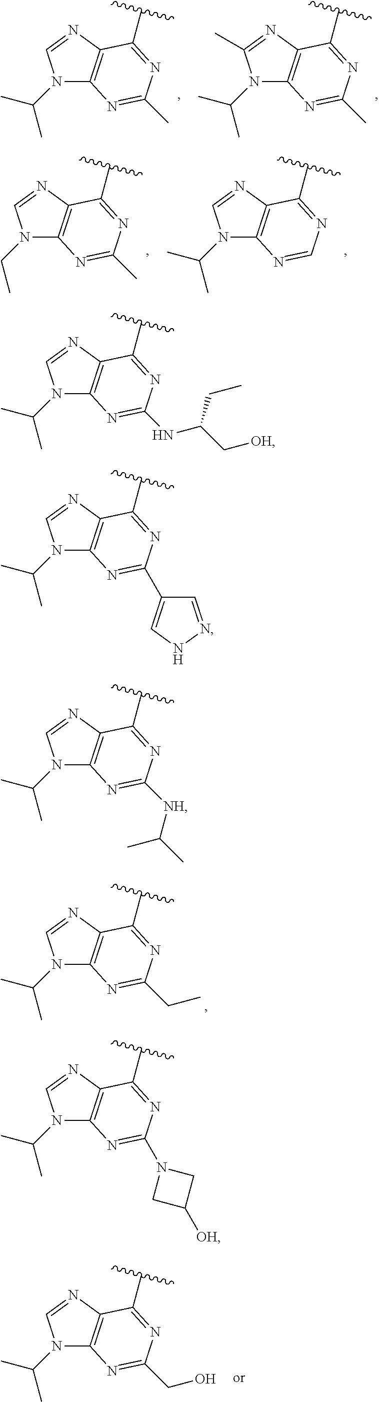

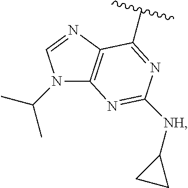 [SMILES: *c1nc(NC2CC2)nc2c1ncn2C(C)C]

and the other variables are defined as in any of the embodiments of the present invention.

In some embodiments, in the compound of formula I in which ring A is

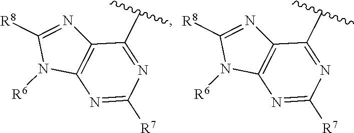

may be

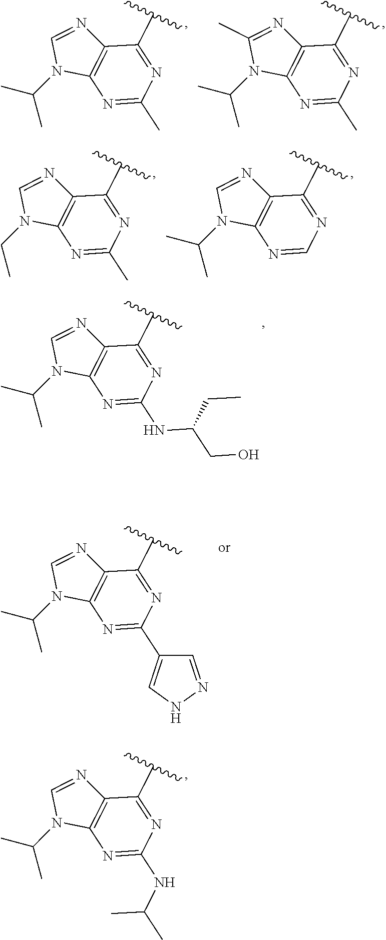

and the other variables are defined as in any of the embodiments of the present invention.

In some embodiments, in the compound of formula I in which ring A is

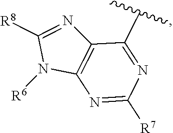

R⁶, R⁷ and R⁸ are defined preferably as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

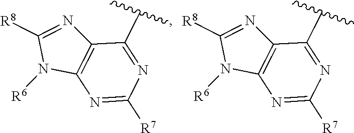

is preferably

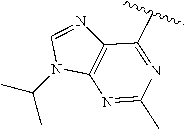

In some embodiments, in the compound of formula I in which ring A is

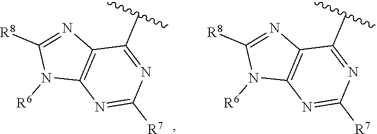

is preferably

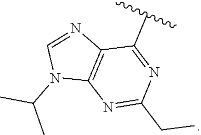 [SMILES: *c1nc(CC)nc2c1ncn2C(C)C]

In some embodiments, in the compound of formula I in which ring A is

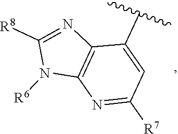

R⁶, R⁷ and R⁸ are defined as follows:

R⁶ is C₁₋₆ alkyl (e.g., ethyl or isopropyl), preferably isopropyl;

R⁷ is hydrogen, a substituted or unsubstituted C₁₋₆ alkyl (e.g., the C₁₋₆ alkyl is, e.g., methyl or isopropyl; further, the substituted or unsubstituted C₁₋₆ alkyl is, e.g., methyl or isopropyl) or —NR^{7b}R^{7c} (e.g.,

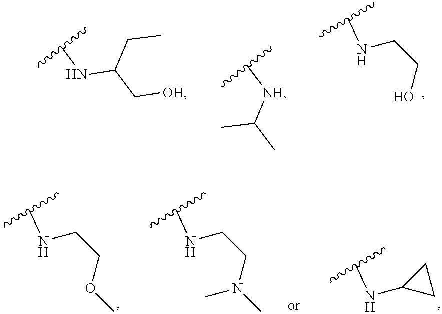

wherein

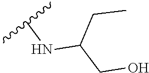 [SMILES: *NC(CC)CO]

is, e.g.,

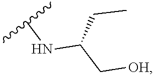 [SMILES: *N[C@H](CC)CO]

wherein the substituted C₁₋₆ alkyl means that the C₁₋₆ alkyl is substituted with 1, 2, 3 or 4 R^{7d} (e.g., substituted with 1 R^{7d});

R^{7b} and R^{7c} are each independently hydrogen, a substituted or unsubstituted C₁₋₄ alkyl (the C₁₋₄ alkyl is, e.g., ethyl, isopropyl or sec-butyl; further, the substituted or unsubstituted C₁₋₄ alkyl is, e.g., ethyl, isopropyl,

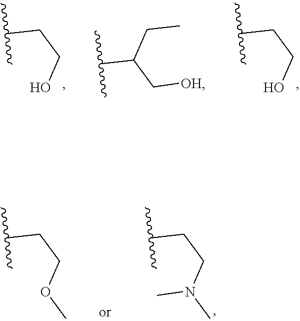

wherein

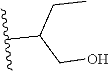 [SMILES: *C(CC)CO]

is, e.g.,

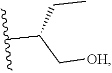 [SMILES: *[C@H](CC)CO]

or a substituted or unsubstituted C₃₋₆ cycloalkyl (the C₃₋₆ cycloalkyl is, e.g., cyclopropyl; further, the substituted or unsubstituted C₃₋₆ cycloalkyl is, e.g., cyclopropyl), wherein the substituted C₁₋₄ alkyl and the substituted C₃₋₆ cycloalkyl mean that the C₁₋₄ alkyl and the C₃₋₆ cycloalkyl are each independently substituted with 1, 2, 3 or 4 R^{d}(e.g., substituted with 1 R^{d}); preferably, one of R^{7b} and R^{7c} is hydrogen;

R^{g} is hydrogen or C₁₋₄ alkyl (e.g., methyl), preferably hydrogen;

the other variables are defined as in any of the embodiments of the present invention.

In some embodiments, in the compound of formula I in which ring A is

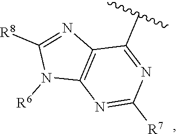

R⁶, R⁷ and R⁸ are defined as follows:

R⁶ is C₁₋₆ alkyl (e.g., ethyl or isopropyl);

R⁷ is hydrogen or C₁₋₆ alkyl (e.g., methyl);

R⁸ is hydrogen or C₁₋₄ alkyl (e.g., methyl);

the other variables are defined as in any of the embodiments of the present invention.

In some embodiments, in the compound of formula I in which ring A is

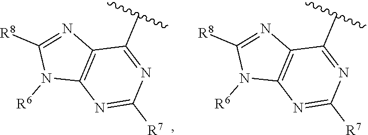

maybe

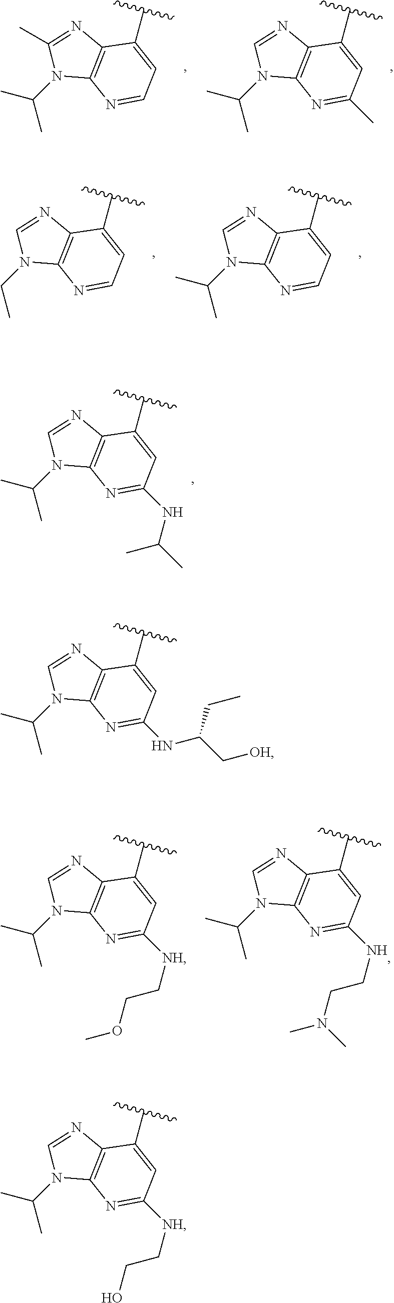

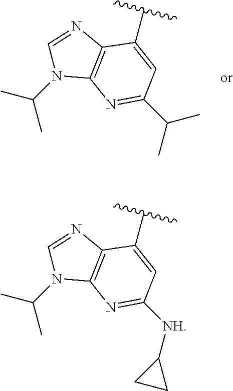

In some embodiments, in the compound of formula I in which ring A is

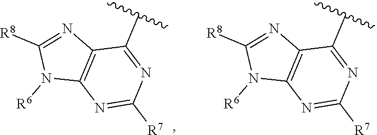

may be

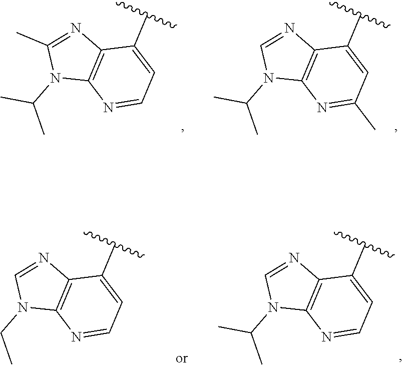 [SMILES: *c1ccnc2c1nc(C)n2C(C)C ; *c1cc(C)nc2c1ncn2C(C)C ; *c1ccnc2c1ncn2CC ; *c1ccnc2c1ncn2C(C)C]

and the other variables are defined as in any of the embodiments of the present invention.

In some embodiments, in the compound of formula I in which ring A is

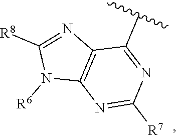

R⁶, R⁷ and R⁸ are defined preferably as follows:

R⁶ is C₁₋₆ alkyl;

R⁷ is C₁₋₆ alkyl;

R⁸ is hydrogen;

the other variables are defined as in any of the embodiments of the present invention.

In some embodiments, in the compound of formula I in which ring A is

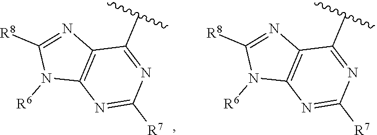

is preferably

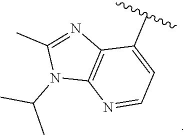 [SMILES: *c1ccnc2c1nc(C)n2C(C)C]

In some embodiments, in the compound of formula I in which ring A is

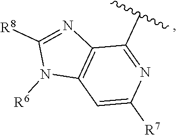

R⁶, R⁷ and R⁸ are defined as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

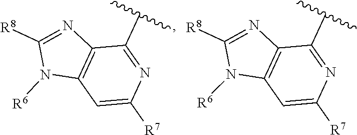

may be

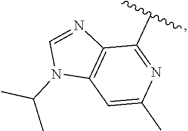 [SMILES: *c1nc(C)cc2c1ncn2C(C)C]

and the other variables are defined as in any of the embodiments of the present invention.

In some embodiments, in the compound of formula I in which ring A is

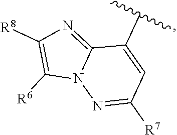

R⁶, R⁷ and R⁸ are defined as follows:

R⁶ is C₁₋₆ alkyl (e.g., isopropyl);

R⁷ is a substituted or unsubstituted C₁₋₆ alkyl (the C₁₋₆ alkyl is, e.g., methyl; further, the substituted or unsubstituted C₁₋₆ alkyl is, e.g., methyl) or —NR^{7b}R^{7c} (e.g.,

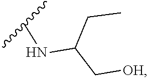 [SMILES: *NC(CC)CO]

such as

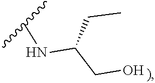 [SMILES: *N[C@H](CC)CO]

wherein the substituted C₁₋₆ alkyl means that the C₁₋₆ alkyl is substituted with 1, 2, 3 or 4 R^{7d} (e.g., substituted with 1 R^{7d});

R^{7b} and R^{7c} are each independently hydrogen or a substituted or unsubstituted C₁₋₄ alkyl (the C₁₋₄ alkyl is, e.g., isobutyl; further, the substituted or unsubstituted C₁₋₄ alkyl is, e.g.,

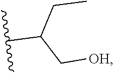 [SMILES: *C(CC)CO]

such as

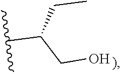 [SMILES: *[C@H](CC)CO]

wherein the substituted C₁₋₄ alkyl means that the C₁₋₄ alkyl is substituted with 1, 2, 3 or 4 R^{d} (e.g., substituted with 1 R^{d}); preferably, one of R^{7b} and R^{7c} is hydrogen;

R⁸ is hydrogen;

the other variables are defined as in any of the embodiments of the present invention.

In some embodiments, in the compound of formula I in which ring A is

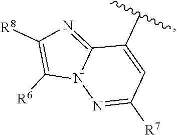

R⁶, R⁷ and R⁸ are defined as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

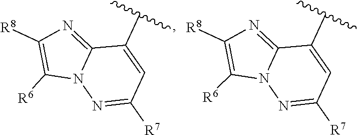

may be

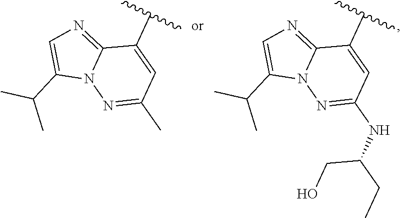 [SMILES: *c1cc(C)nn2c(C(C)C)cnc12 ; *c1cc(N[C@H](CC)CO)nn2c(C(C)C)cnc12]

and the other variables are defined as in any of the embodiments of the present invention.

In some embodiments, in the compound of formula I in which ring A is

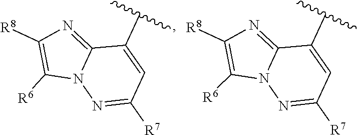

may be

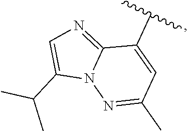 [SMILES: *c1cc(C)nn2c(C(C)C)cnc12]

and the other variables are defined as in any of the embodiments of the present invention.

In some embodiments, in the compound of formula I in which ring A is

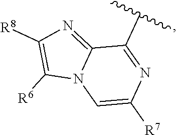

R⁶, R⁷ and R⁸ are defined as follows:

R⁶ is C₁₋₆ alkyl (e.g., isopropyl);

R⁷ is C₁₋₆ alkyl (e.g., methyl) or —NR^{7b}R^{7c} (e.g., or

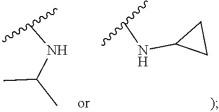

R^{7b} and R^{7c} are each independently hydrogen, C₁₋₄ alkyl or C₃₋₆ cycloalkyl (e.g., the C₃₋₆ cycloalkyl is, e.g., cyclopropyl); preferably, one of R^{7b} and R^{7c} is hydrogen;

R⁸ is hydrogen;

the other variables are defined as in any of the embodiments of the present invention.

In some embodiments, in the compound of formula I in which ring A is

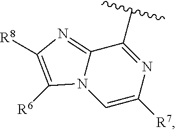

R⁶, R⁷ and R⁸ are defined as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

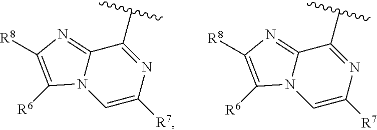

may be

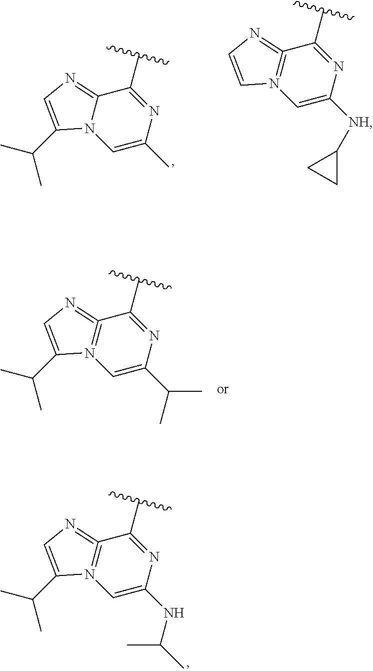

and the other variables are defined as in any of the embodiments of the present invention.

In some embodiments, in the compound of formula I in which ring A is

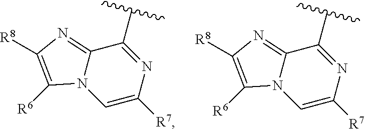

may be

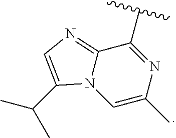 [SMILES: *c1nc(C)cn2c(C(C)C)cnc12]

In some embodiments, in the compound of formula I in which ring A is

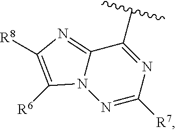

R⁶, R⁷ and R⁸ are defined as follows:

R⁶ is C₁₋₆ alkyl (e.g., isopropyl);

R⁷ is a substituted or unsubstituted C₁₋₆ alkyl (the C₁₋₆ alkyl is, e.g., methyl; further, the substituted or unsubstituted C₁₋₆ alkyl is, e.g., methyl), —OR^{7a} (e.g.,

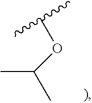 [SMILES: *OC(C)C]

or —NR^{7b}R^{7c} (e.g.,

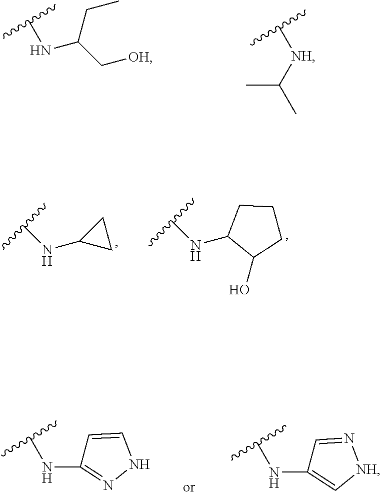

wherein

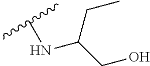 [SMILES: *NC(CC)CO]

is, e.g.,

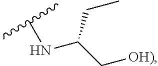

wherein the substituted C₁₋₆ alkyl means that the C₁₋₆ alkyl is substituted with 1, 2, 3 or 4 R^{7d} (e.g., substituted with 1 R^{7d});

R^{7a} is hydrogen or a substituted or unsubstituted C₁₋₆ alkyl (the C₁₋₆ alkyl is, e.g., isopropyl; further, the substituted or unsubstituted C₁₋₆ alkyl is, e.g., isopropyl), wherein the substituted C₁₋₆ alkyl means that the C₁₋₆ alkyl is substituted with 1, 2, 3 or 4 R^{c} (e.g., substituted with 1 R^{c});

R^{7b} and R^{7c} are each independently hydrogen, a substituted or unsubstituted C₁₋₄ alkyl (the C₁₋₄ alkyl is, e.g., isopropyl or sec-butyl; further, the substituted or unsubstituted C₁₋₄ alkyl is, e.g., isopropyl or

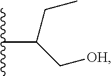

wherein

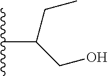 [SMILES: *C(CC)CO]

is, e.g.,

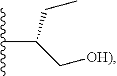

a substituted or unsubstituted C₃₋₆ cycloalkyl (the C₃₋₆ cycloalkyl is, e.g., cyclopropyl or cyclopentyl; further, the substituted or unsubstituted C₃₋₆ cycloalkyl is, e.g., cyclopropyl or

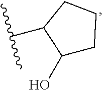

wherein

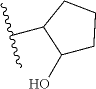 [SMILES: *C1CCCC1O]

is, e.g.,

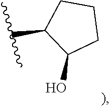 [SMILES: *[C@H]1CCC[C@H]1O]

or a substituted or unsubstituted 5-6 membered heteroaryl (the 5-6 membered heteroaryl is, e.g., pyrazolyl, such as

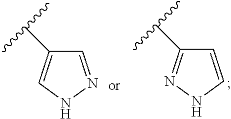 [SMILES: *c1cn[nH]c1 ; *c1cc[nH]n1]

further, the substituted or unsubstituted 5-6 membered heteroaryl is, e.g.,

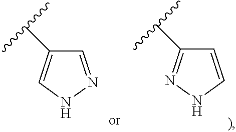 [SMILES: *c1cn[nH]c1 ; *c1cc[nH]n1]

wherein the substituted C₁₋₄ alkyl, the substituted C₃₋₆ cycloalkyl and the substituted 5-6 membered heteroaryl mean that the C₁₋₄ alkyl, the C₃₋₆ cycloalkyl and the 5-6 membered heteroaryl are each independently substituted with 1, 2, 3, or 4 R^{d} (e.g., substituted with 1 R^{d}); preferably, one of R^{7b} and R^{7c} is hydrogen;

R⁸ is hydrogen;

the other variables are defined as in any of the embodiments of the present invention.

In some embodiments, in the compound of formula I in which ring A is

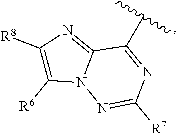

R⁶, R⁷ and R⁸ are defined as follows:

R⁶ is C₁₋₆ alkyl (e.g., isopropyl);

R⁷ is C₁₋₆ alkyl (e.g., methyl);

R⁸ is hydrogen;

the other variables are defined as in any of the embodiments of the present invention.

In some embodiments, in the compound of formula I in which ring A is

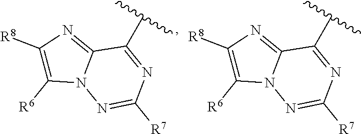

may be

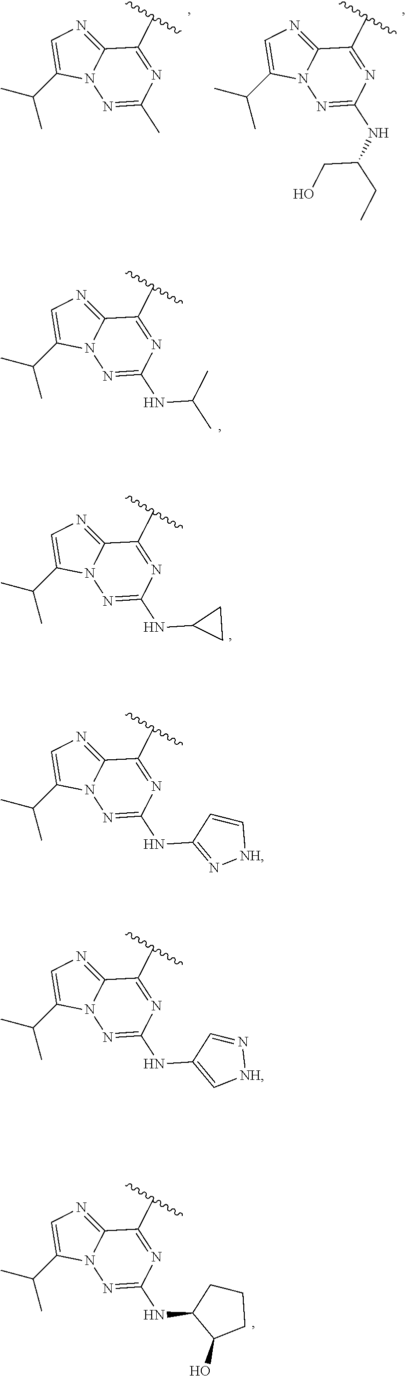

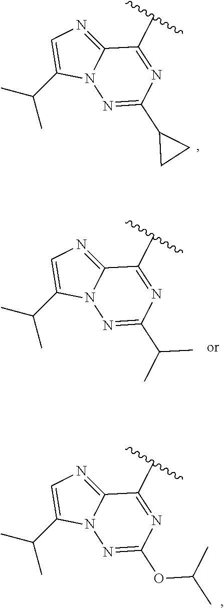

and the other variables are defined as in any of the embodiments of the present invention.

In some embodiments, in the compound of formula I in which ring A is

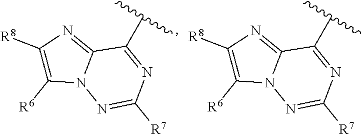

is preferably

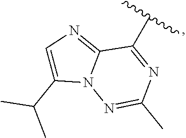 [SMILES: *c1nc(C)nn2c(C(C)C)cnc12]

and the other variables are defined as in any of the embodiments of the present invention.

In some embodiments, in the compound of formula I, X is O, and the other variables are defined as in any of the embodiments of the present invention.

In some embodiments, in the compound of formula I, m is 2, n is 1, and the other variables are defined as in any of the embodiments of the present invention.

In some embodiments, in the compound of formula I, R¹ is

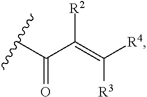

and the other variables are defined as in any of the embodiments of the present invention.

In some embodiments, in the compound of formula I, each R⁶ is independently C₁₋₆ alkyl, preferably isopropyl, and the other variables are defined as in any of the embodiments of the present invention.

In some embodiments, in the compound of formula I, each R⁷ is independently C₁₋₆ alkyl, and the other variables are defined as in any of the embodiments of the present invention.

In some embodiments, in the compound of formula I, R⁸ is hydrogen, and the other variables are defined as in any of the embodiments of the present invention.

In some embodiments, in the compound of formula I, the groups are defined as follows: wherein, ring A is

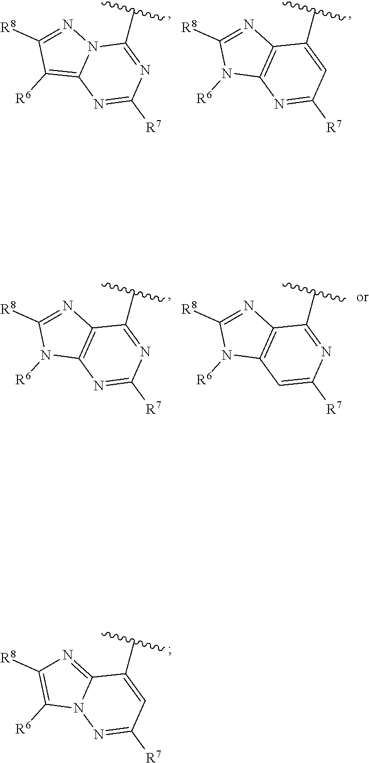

X is O;

m is 2;

n is 1;

R¹ is

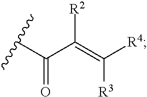

R² is hydrogen;

R³ is hydrogen;

R⁴ is —CH₂—NR^{4a}R^{4b} (e.g., —CH₂—N(CH₃)₂);

R^{4a} and R^{4b} are each independently hydrogen or C₁₋₆ alkyl (e.g., methyl);

each R⁶ is independently C₁₋₆ alkyl (e.g., methyl, ethyl, n-propyl or isopropyl, preferably isopropyl);

each R⁷ is independently C₁₋₆ alkyl;

R⁸ is hydrogen;

a carbon atom marked by * is in S configuration, R configuration, or a mixture thereof.

In some embodiments, the compound of formula I has any of the following structures:

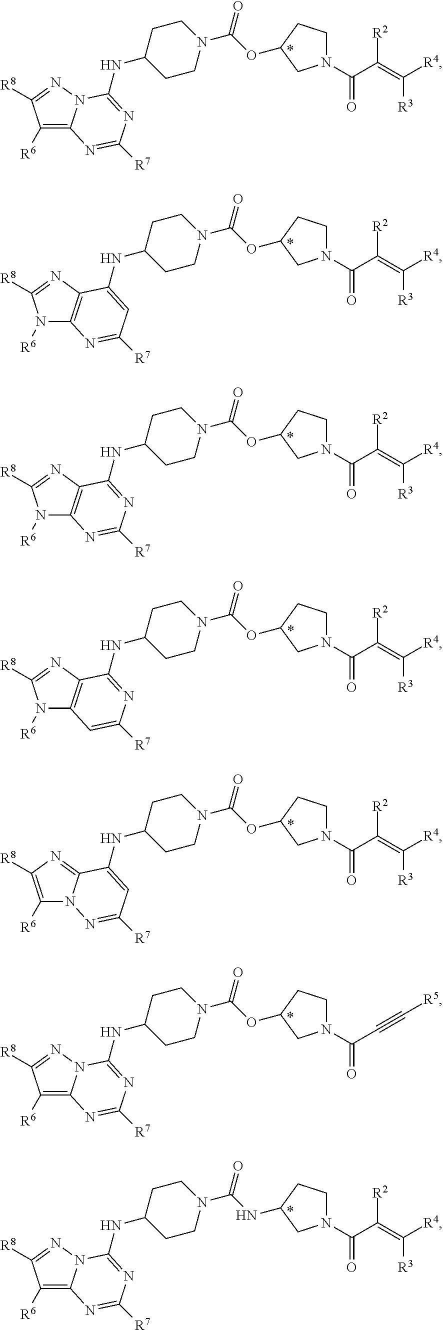

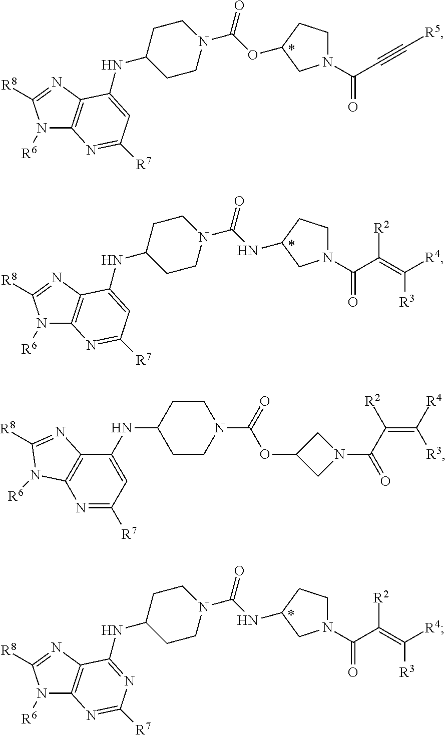

wherein, R², R³, R⁴, R⁵, R⁶, R⁷, R⁸ and * are defined as in any of the aforementioned embodiments.

In some embodiments, the compound of formula I has any of the following structures:

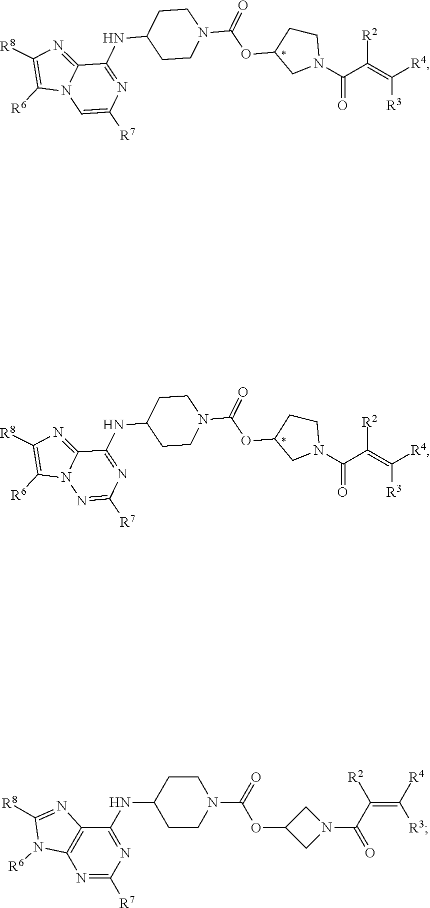

wherein, R², R³, R⁴, R⁵, R⁶, R⁷, R⁸ and * are defined as in any of the aforementioned embodiments.

In some embodiments, the compound of formula I has any of the following structures:

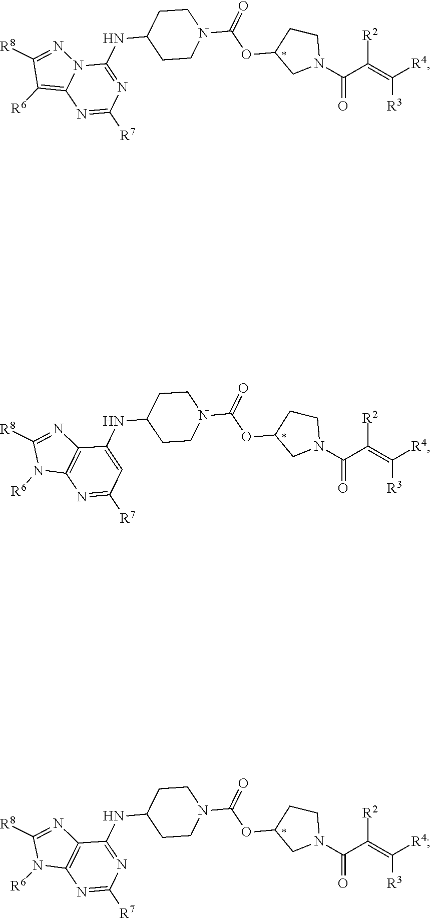

wherein, R², R³, R⁴, R⁶, R⁷, R⁸ and * are defined as in any of the aforementioned embodiments.

In some embodiments, in the compound of formula I, in consideration of the inhibitory activity against CDK7, ring A is preferably

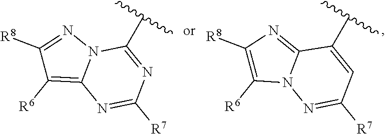

and further preferably

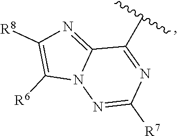

and the other variables are defined as in any of the embodiments of the present invention.

In some embodiments, in the compound of formula I, in consideration of the inhibitory activity against HCC70 cells, ring A is preferably

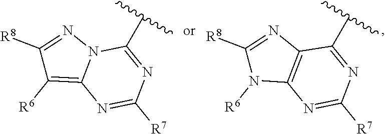

and further preferably

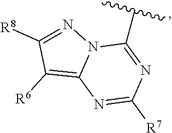

and the other variables are defined as in any of the embodiments of the present invention.

In some embodiments, in the compound of formula I, in consideration of the inhibitory activity against OVCAR3 cells, ring A is preferably

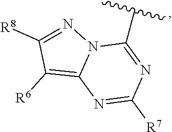

and the other variables are defined as in any of the embodiments of the present invention.

In some embodiments, in the compound of formula I as described in any of the aforementioned embodiments, a carbon atom marked by * is in S configuration.

In some embodiments, in the compound of formula I as described in any of the aforementioned embodiments, a carbon atom marked by * is in R configuration.

In some embodiments, the compound of formula I has any of the following structures:

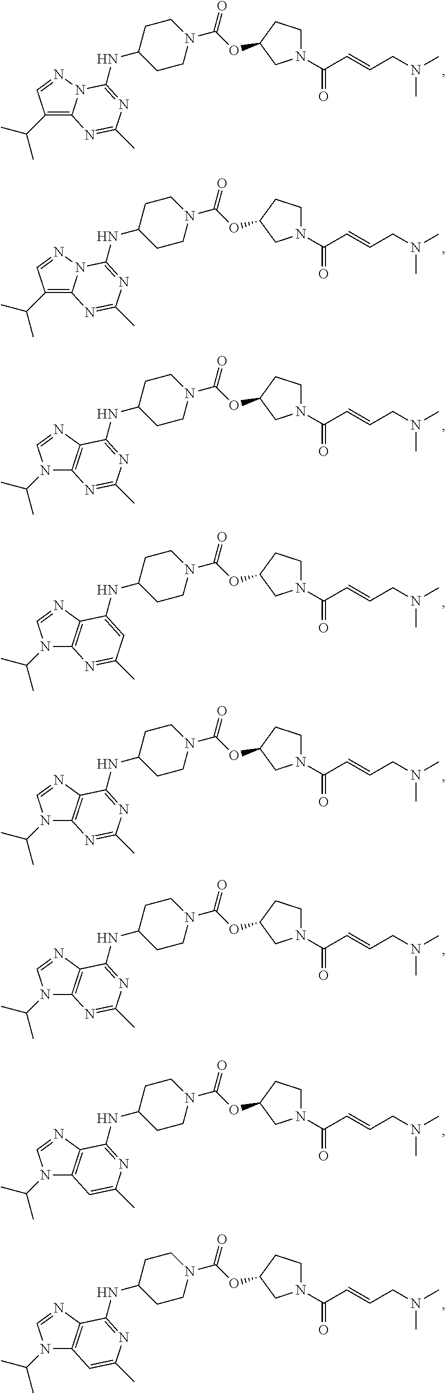

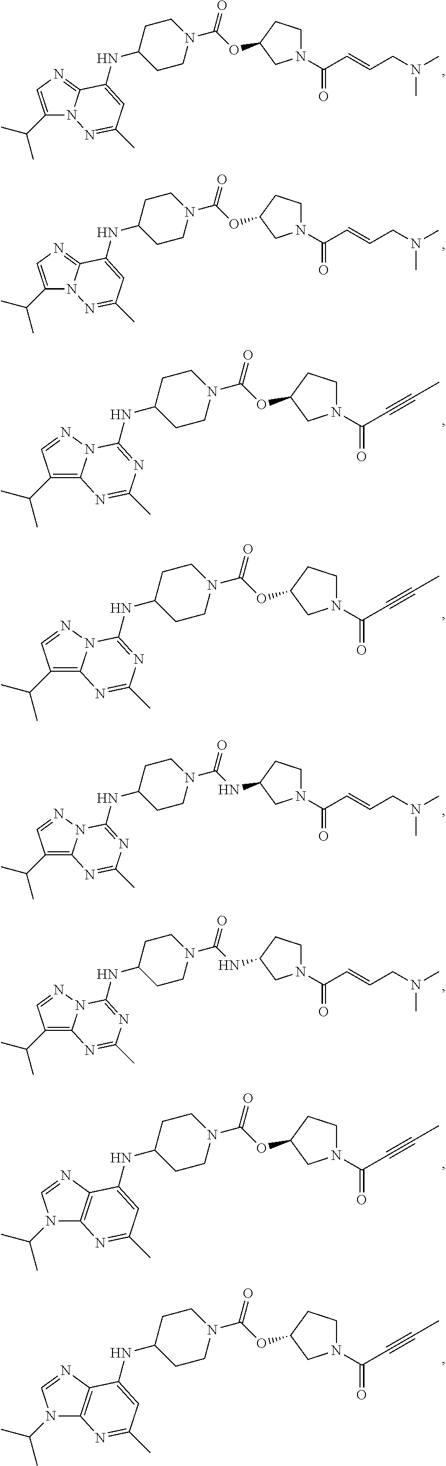

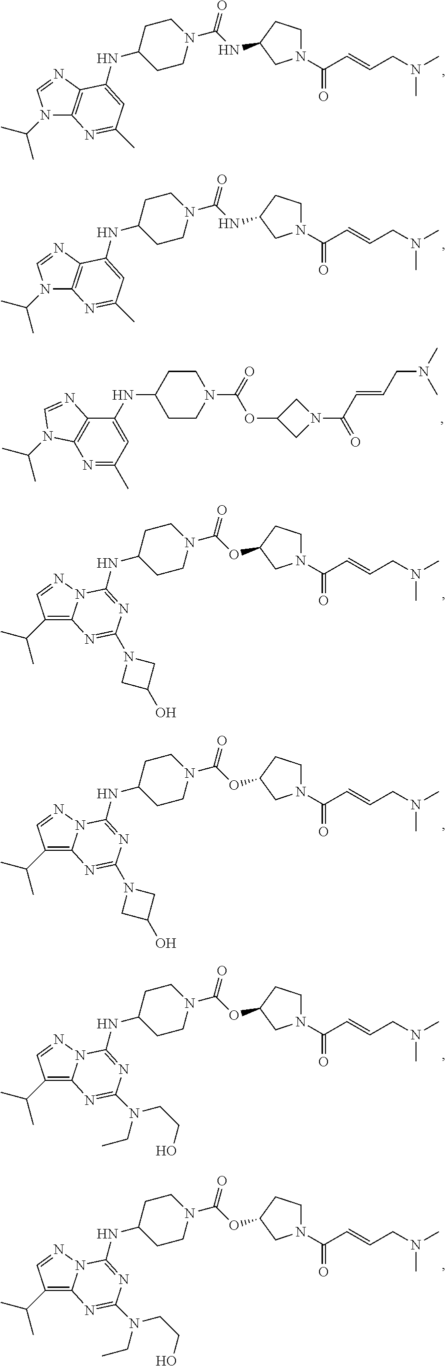

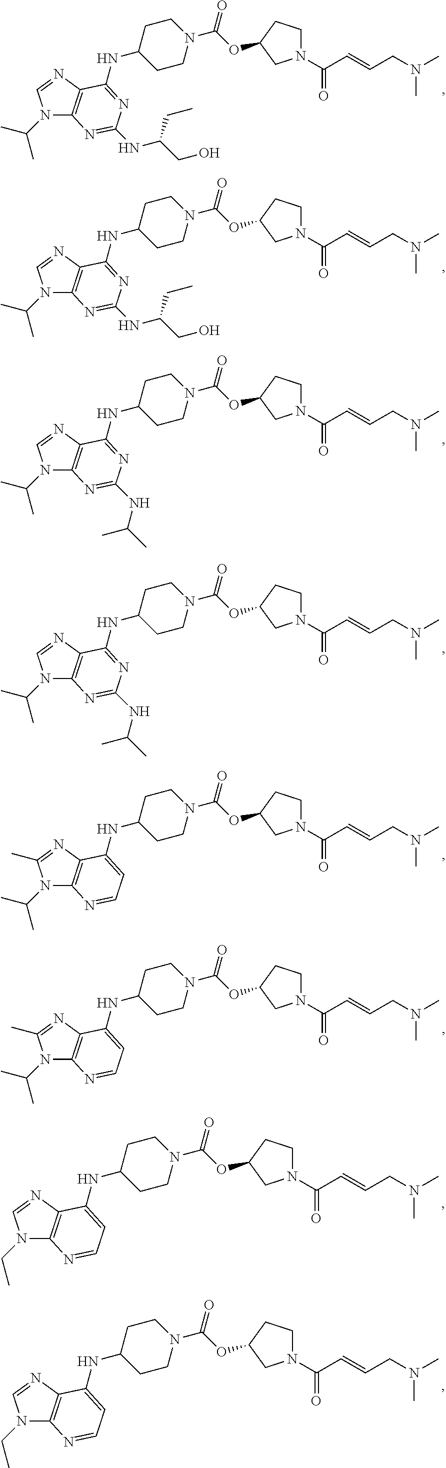

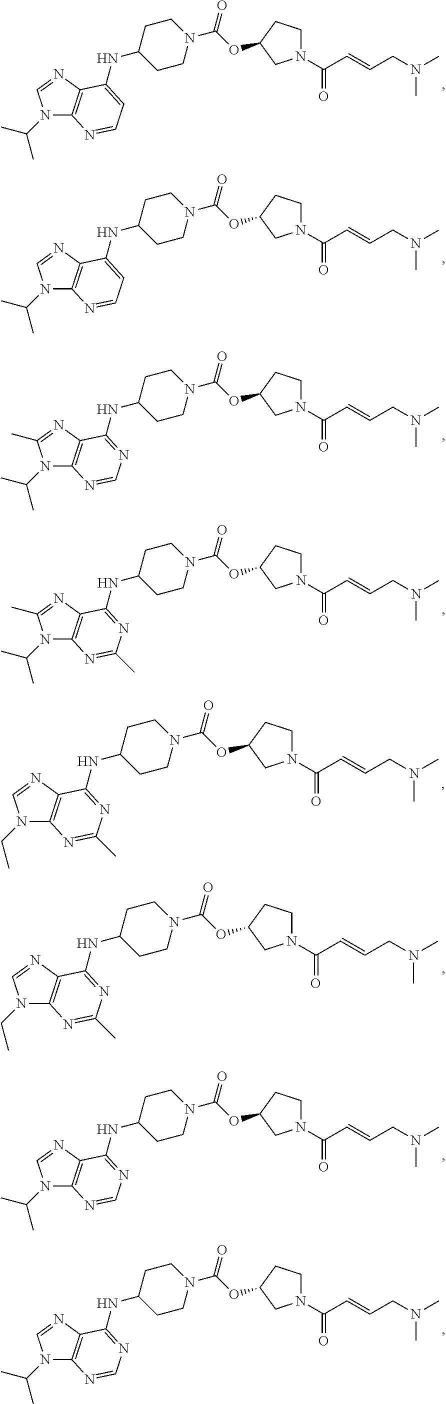

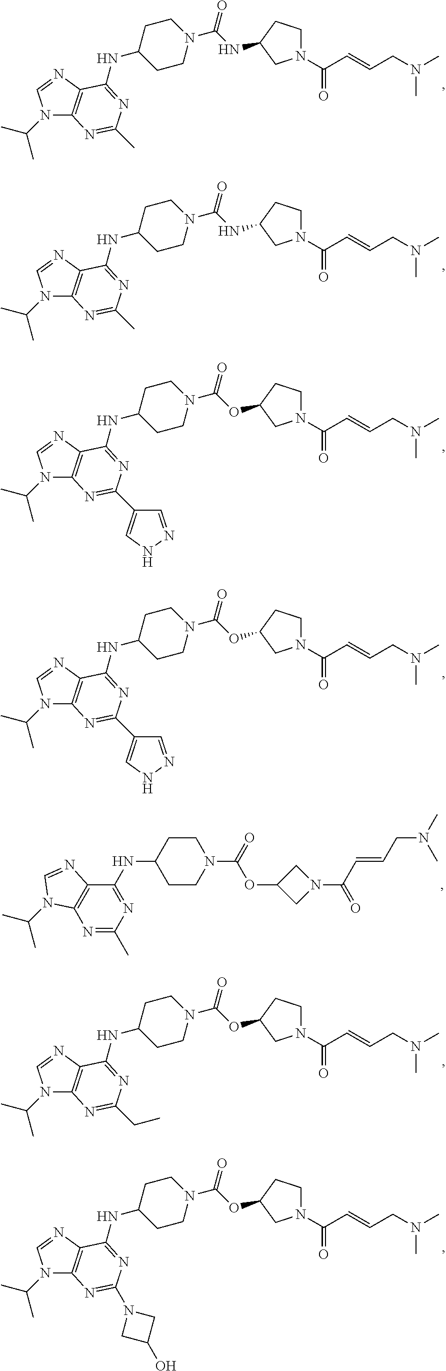

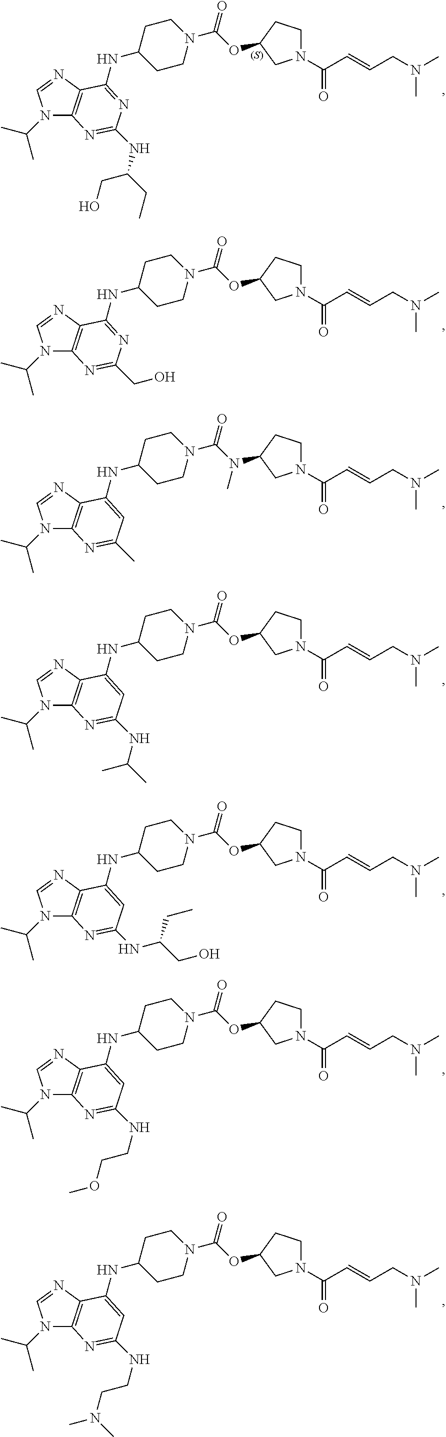

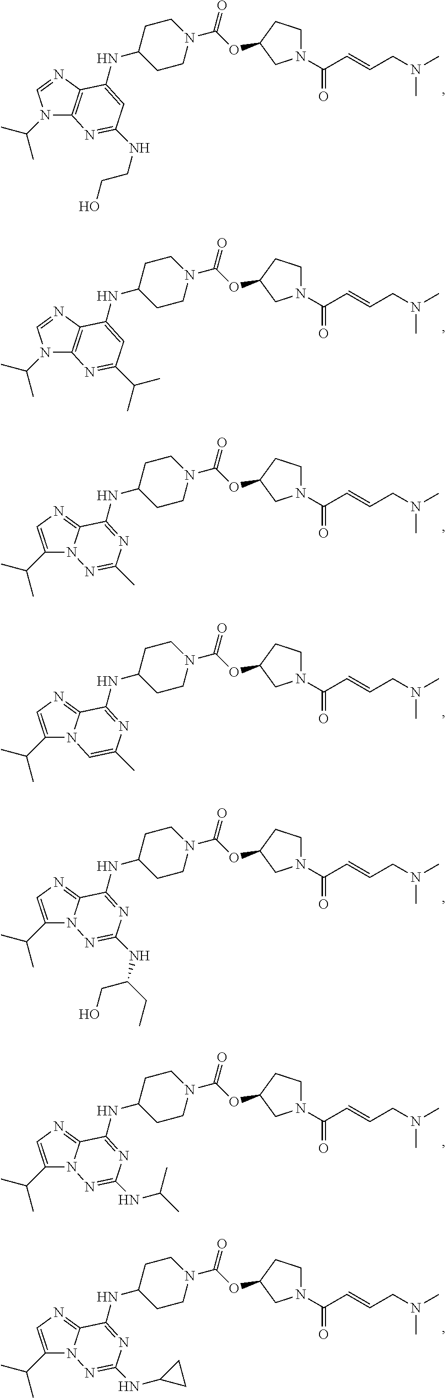

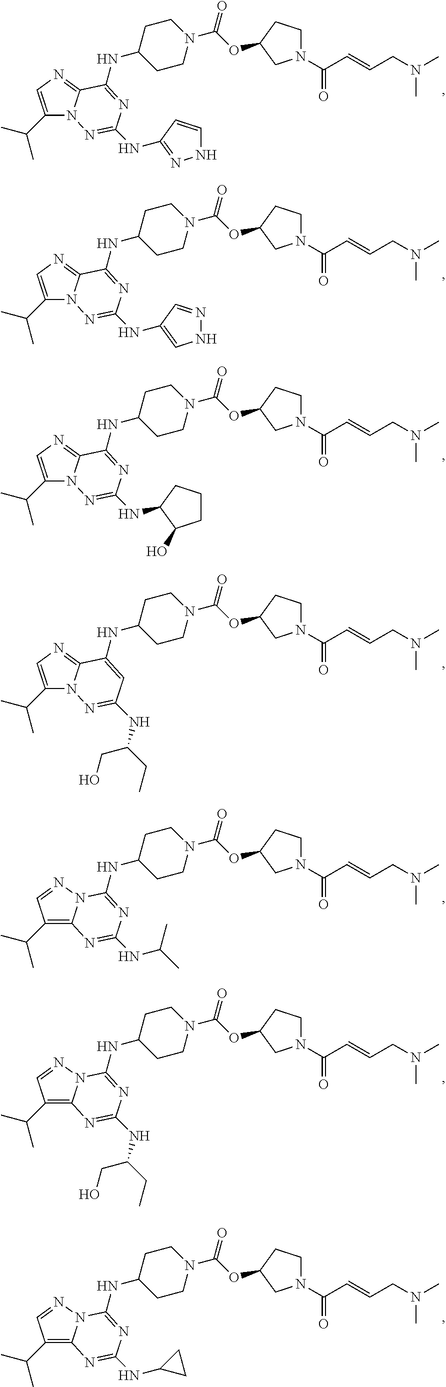

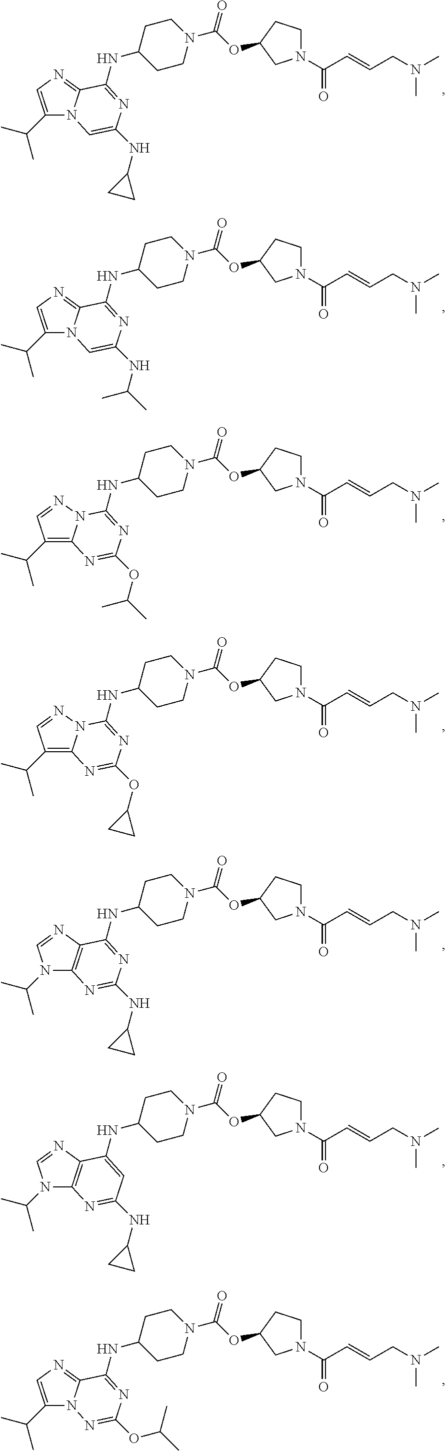

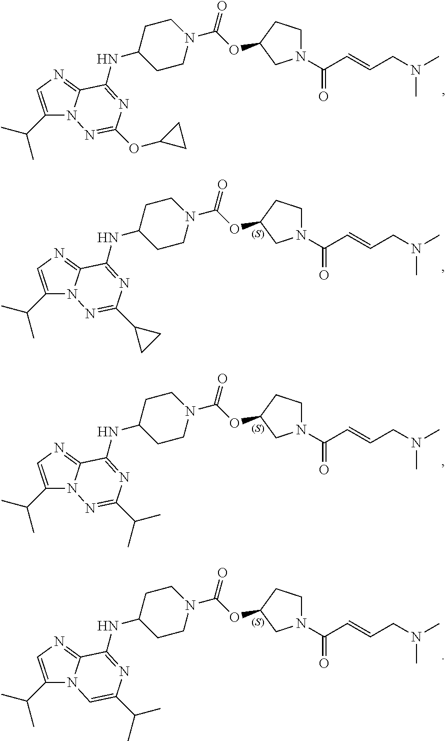

The present invention also provides a method for preparing the compound of formula I described above, which comprises: subjecting a compound of formula II and

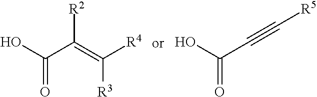

to a condensation reaction shown below in an organic solvent (e.g., DMF and/or THF) in the presence of a condensing agent (e.g., one or more of HATU, PyBOP and T3P) and a base (e.g., TEA and/or DIPEA) to obtain the compound of formula I, wherein ring A, X, m, n, R¹, R², R³, R⁴, R⁵ and * are defined as above;

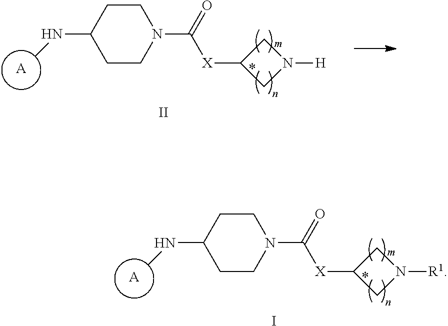

In some embodiments, a method for preparing the compound of formula II may comprise: subjecting a compound of formula III to a Boc deprotection reaction shown below in an organic solvent (e.g., DCM and/or dioxane) in the presence of an acid (e.g., HCl and/or TFA) to obtain the compound of formula II, wherein ring A, X, m, n and * are defined as above;

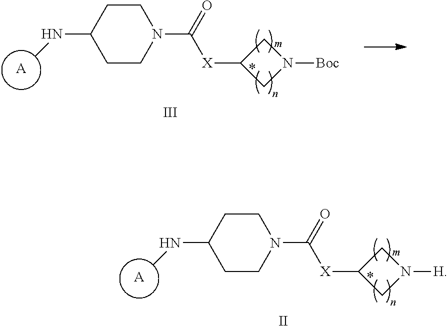

In some embodiments, a method for preparing the compound of formula III may comprise: subjecting a compound of formula IV-1 and a compound of formula IV-2 to a condensation reaction shown below in an organic solvent (e.g., one or more of THF, DCM and CH₃CN) in the presence of a base (e.g., TEA and/or DIPEA) to obtain the compound of formula III, wherein LG is a leaving group (e.g., chloro or p-nitrophenyl), and ring A, X, m, n and * are defined as above;

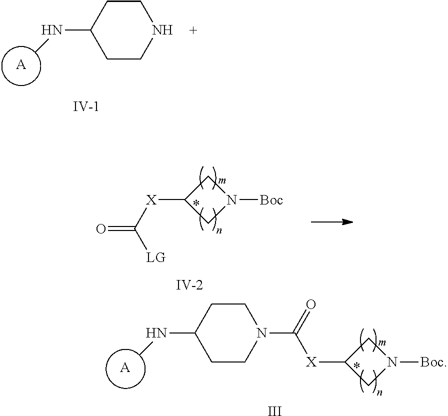

In some embodiments, a method for preparing the compound of formula IV-1 may comprise: subjecting a compound of formula V to a Boc deprotection reaction shown below in an organic solvent (e.g., DCM and/or dioxane) in the presence of an acid (e.g., HCl and/or TFA) to obtain the compound of formula IV-1, wherein ring A is defined as above;

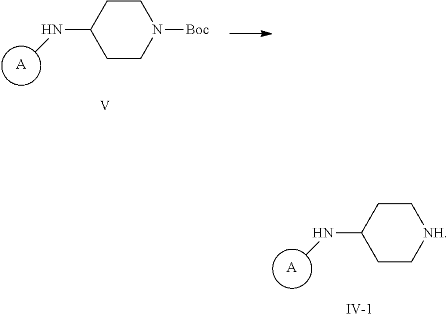

The present invention also provides a compound or a tautomer, a stereoisomer or an isotopic derivative thereof, or a pharmaceutically acceptable salt of any of the foregoing, or a crystalline form or a solvate of any of the foregoing, wherein the compound is selected from any of the following structures:

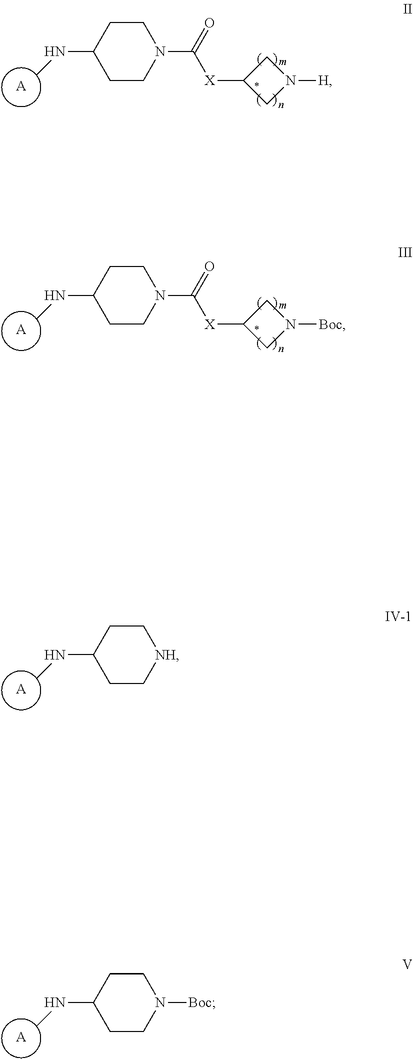

wherein ring A, X, m, n and * are defined as above.

The present invention also provides a pharmaceutical composition, which comprises:

(i) the compound of formula I or the tautomer, the stereoisomer or the isotopic derivative thereof, or the pharmaceutically acceptable salt of any of the foregoing, or the crystalline form or the solvate of any of the foregoing; and

(ii) at least one pharmaceutical adjuvant.

The present invention also provides use of the compound of formula I or the tautomer, the stereoisomer or the isotopic derivative thereof, or the pharmaceutically acceptable salt of any of the foregoing, or the crystalline form or the solvate of any of the foregoing in preparing a CDK7 inhibitor.

The present invention also provides use of the compound of formula I or the tautomer, the stereoisomer or the isotopic derivative thereof, or the pharmaceutically acceptable salt of any of the foregoing, or the crystalline form or the solvate of any of the foregoing in preparing a medicament.

In some embodiments, the medicament is a medicament for preventing and/or treating a CDK7-mediated disease, e.g., a tumor, such as breast cancer, ovarian cancer, small cell lung cancer, acute myeloid leukemia, acute lymphocytic leukemia, bladder cancer, colon cancer, prostate cancer, epithelial sarcoma and soft tissue sarcoma.

In some embodiments, the medicament is a medicament for preventing and/or treating a tumor, such as breast cancer, ovarian cancer, small cell lung cancer, acute myeloid leukemia or acute lymphocytic leukemia.

The present invention also provides a method for preventing and/or treating a CDK7-mediated disease, which comprises administering to a subject in need thereof a therapeutically effective amount of the compound of formula I or the tautomer, the stereoisomer or the isotopic derivative thereof, or the pharmaceutically acceptable salt of any of the foregoing, or the crystalline form or the solvate of any of the foregoing.

In some embodiments, the CDK7-mediated disease may be a tumor, such as breast cancer, ovarian cancer, small cell lung cancer, acute myeloid leukemia, acute lymphocytic leukemia, bladder cancer, colon cancer, prostate cancer, epithelial sarcoma and soft tissue sarcoma.

The present invention also provides a method for preventing and/or treating a tumor, which comprises administering to a subject in need thereof a therapeutically effective amount of the compound of formula I or the tautomer, the stereoisomer or the isotopic derivative thereof, or the pharmaceutically acceptable salt of any of the foregoing, or the crystalline form or the solvate of any of the foregoing.

In some embodiments, the tumor can be breast cancer, ovarian cancer, small cell lung cancer, acute myeloid leukemia, acute lymphocytic leukemia, bladder cancer, colon cancer, prostate cancer, epithelial sarcoma and soft tissue sarcoma.

Unless otherwise stated, terms used herein have the following definitions, and definitions of terms not referred to hereinafter are the same as those generally understood by those skilled in the art to which the present invention pertains.

The term “tautomer” refers to functional isomers resulting from the rapid movement of an atom in a molecule between two positions. For example, acetone and 1-propen-2-ol can be mutually converted when hydrogen atom rapidly migrates between oxygen and a-carbon atoms.

The term “stereoisomer” refers to isomers of a molecule having the same order of atoms or radicals but different spatial arrangement, such as cis-trans isomer, optical isomer or atropisomer. Such stereoisomers can be separated, purified and enriched by asymmetric synthesis or chiral resolution (including but not limited to thin layer chromatography, centrifugal partition chromatography, column chromatography, gas chromatography, and high pressure liquid chromatography), and can also be obtained by chiral resolution via bonding (such as chemical bonding) or salification (such as physical bonding) with other chiral compounds. Optical isomers include enantiomers and diastereoisomers.

The term “isotopic derivative” refers to a compound in which one or more atoms are replaced by one or more atoms having a specific atomic mass or mass number. Examples of isotopes that can be incorporated into the compound include, but are not limited to, isotopes of hydrogen, carbon, nitrogen, oxygen, fluorine, sulfur, and chlorine (such as ²H, ³H, ¹³C,¹C, ¹⁵N, ¹⁸O, ¹⁷O, ¹⁸F, ³⁵S and ³⁶Cl). An isotopic compound can generally be prepared by replacing a non-isotopically labeled reagent with an isotopically labeled reagent according to the methods described herein. Typical examples of isotopic derivatives include deuterated compounds.

The term “pharmaceutically acceptable salt” refers to a salt prepared with the compound and a relatively non-toxic and pharmaceutically acceptable acid or base. When the compound contains a relatively acidic functional group, a base addition salt can be obtained by contacting a neutral form of the compound with a sufficient amount of a pharmaceutically acceptable base in a pure solution or a suitable inert solvent. Pharmaceutically acceptable base addition salts include, but are not limited to: lithium salt, sodium salt, potassium salt, calcium salt, aluminum salt, magnesium salt, zinc salt, bismuth salt, ammonium salt, and diethanolamine salt. When the compound disclosed herein contains a relatively basic functional group, an acid addition salt can be obtained by contacting a neutral form of the compound with a sufficient amount of a pharmaceutically acceptable acid in a pure solution or a suitable inert solvent. The pharmaceutically acceptable acid includes inorganic acids, including, but not limited to: hydrochloric acid, hydrobromic acid, hydroiodic acid, nitric acid, carbonic acid, phosphoric acid, phosphorous acid, and sulfuric acid. The pharmaceutically acceptable acids include organic acids, including, but not limited to: acetic acid, propionic acid, oxalic acid, isobutyric acid, maleic acid, malonic acid, benzoic acid, succinic acid, suberic acid, boletic acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, bitartrate, ascorbic acid, gentisic acid, fumaric acid, gluconic acid, saccharic acid, formic acid, ethanesulfonic acid, pamoic acid (i.e., 4,4′-methylene-bis(3-hydroxy-2-naphthoic acid)), amino acids (such as glutamic acid and arginine), and the like. When a compound contains both relatively acidic functional group and relatively basic functional group, it can be converted to either a base addition salt or an acid addition salt. For details, see Berge et al., “Pharmaceutical Salts”, Journal of Pharmaceutical Science 66:1-19 (1977), or Handbook of Pharmaceutical Salts: Properties, Selection, and Use (P. Heinrich Stahl and Camille G. Wermuth, ed., Wiley-VCH, 2002).

The term “crystalline form” means that ions or molecules in it are arranged strictly periodically in a three-dimensional space in a certain manner and have a periodic recurring pattern at certain intervals; due to the difference in above periodic arrangements, there may be multiple crystalline forms, i.e., polymorphism.

The term “solvate” refers to a substance formed by combining a molecule with a stoichiometric or non-stoichiometric amount of a solvent. Solvent molecules in the solvate may be present in ordered or unordered arrangements. The solvents include, but are not limited to, water, methanol, ethanol, and the like.

The term “halogen” refers to fluorine, chlorine, bromine or iodine.

The term “alkyl” refers to a linear or branched saturated monovalent hydrocarbyl having a specified number of carbon atoms. For example, C₁₋₄ alkyl refers to alkyl having 1-4 carbon atoms. Examples of alkyl include, but are not limited to, methyl, ethyl, n-propyl, isopropyl, n-butyl, isobutyl, sec-butyl, tert-butyl, and pentyl. In some embodiments, C₁₋₄ alkyl can be methyl, ethyl, n-propyl, isopropyl, n-butyl, isobutyl, sec-butyl, or tert-butyl. In some embodiments, C₁₋₆ alkyl can be C₁₋₄ alkyl, such as methyl, ethyl, n-propyl, isopropyl, n-butyl, isobutyl, sec-butyl, or tert-butyl.

The term “alkoxy” refers to —O—R^{X}, wherein R^{X} is the alkyl defined above. In some embodiments, C₁₋₄ alkoxy can be methoxy, ethoxy, n-propoxy, isopropoxy, n-butoxy, isobutoxy, sec-butoxy, or tert-butoxy.

The term “cycloalkyl” refers to saturated monocyclic or polycyclic (e.g., fused, spiro or bridged) hydrocarbyl formed by carbon atoms. In some embodiments, cycloalkyl is a monocyclic group. In some embodiments, C₃₋₆ cycloalkyl can be cyclopropyl, cyclobutyl, cyclopentyl, or cyclohexyl.

The term “heterocycloalkyl” refers to a non-aromatic, saturated or partially unsaturated, and monocyclic or polycyclic (e.g., fused, spiro or bridged) cyclic group formed by carbon atoms and at least one heteroatom selected from N, O and S. In some embodiments, heterocycloalkyl is a saturated cyclic group. In some embodiments, heterocycloalkyl is a monocyclic group. In some embodiments, heterocycloalkyl is a saturated monocyclic group. Heterocycloalkyl can be connected to the rest of the molecule via a heteroatom or a carbon atom in the ring. Examples of heterocycloalkyl include, but are not limited to, 1-piperidinyl, 2-piperidinyl, 3-piperidinyl, 4-morpholinyl, 3-morpholinyl, tetrahydrofuran-2-yl, tetrahydrothiophen-2-yl, tetrahydrothiophen-3-yl, 1-piperazinyl, and 2-piperazinyl. 4-6 membered heterocycloalkyl can be 4, 5 or 6 membered heterocycloalkyl. The 4 membered heterocycloalkyl is, e.g., azetidinyl. The 5 membered heterocycloalkyl is, e.g., tetrahydrofuranyl, tetrahydropyrrolyl or tetrahydrothienyl. The 6 membered heterocycloalkyl is, e.g., piperidinyl, morpholinyl or piperazinyl. In some cases, in —NR^{g}R^{h} or similar groups, if it is defined that R^{g} and R^{h}, together with a nitrogen atom attached thereto, form a substituted or unsubstituted 4-6 membered heterocycloalkyl and the 4-6 membered heterocycloalkyl has 0, 1 or 2 additional heteroatoms independently selected from N, O and S, the meaning of “additional” is clear, and namely it refers to other heteroatoms besides the N atom in the —NR^{g}R^{h} (where the nitrogen atom is underlined). For example, if

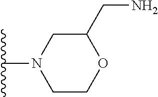 [SMILES: *N1CCOC(CN)C1]

is formed by —NR^{g}R^{h}, the additional heteroatom is 1 oxygen atom; if

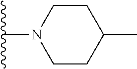 [SMILES: *N1CCC(C)CC1]

is formed by —NR^{g}R^{h}, there's 0 additional heteroatom.

The term “heteroaryl” refers to an aromatic cyclic group formed by carbon atoms and at least one heteroatom selected from N, O and S. 5 membered heteroaryl is, e.g., furanyl, thienyl, pyrrolyl, pyrazolyl, oxazolyl, thiazolyl, imidazolyl or triazolyl; 6-membered heteroaryl is, e.g., pyrazinyl, pyridazinyl, pyridinyl or pyrimidinyl.

As used herein, “x-y membered” for describing a cyclic group means that the number of atoms in the ring is x-y. For example, cyclopropyl is 3 membered, tetrahydropyrrolyl is 5 membered, and piperidinyl is 6 membered.

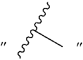

used herein in the structural formulas describing groups means that the corresponding group is attached via that site to other fragments and groups of the compound. For example, in

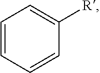

when R′ is

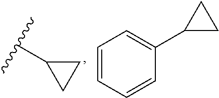

is formed.

When any variable (e.g., R) occurs more than once in the constitution or structure of a compound, the variable is independently defined in each case. Thus, for example, if a group is substituted with 0-2 R, the group can be optionally substituted with up to two R, and the definition of R in each case is independent. Furthermore, a combination of a substituent and/or a variable is permissible only if the combination can result in a stable compound. For example, if in

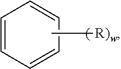

w is 0, 1 or 2, and each R is independently methyl or fluoro,

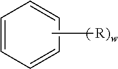

includes

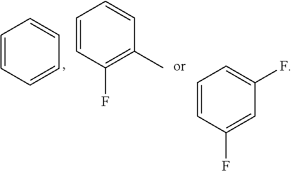

The term “pharmaceutical adjuvant” refers to excipients and additives used in the manufacture of pharmaceutical products and in the formulation of pharmaceutical formulas, and it refers to all substances contained in the pharmaceutical preparation except for the active ingredient. See Volume 4 of Pharmacopoeia of The People's Republic of China (2015 Edition), or Handbook of Pharmaceutical Excipients (Raymond C Rowe, 2009 Sixth Edition).

The term “treat” “treating” or “treatment” refers to therapeutic therapy. When specific conditions are involved, “treat” “treating” or “treatment” refers to: (1) relieving one or more biological manifestations of a disease or disorder, (2) interfering with (a) one or more points in a biological cascade that causes or results in a disorder or (b) one or more biological manifestations of a disorder, (3) ameliorating one or more symptoms, effects or side effects associated with a disorder, or ameliorating one or more symptoms, effects or side effects associated with a disorder or treatment thereof, or (4) slowing the progression of one or more biological manifestations of a disease or disorder.

The term “therapeutically effective amount” refers to an amount of a compound that, when administered to a patient, is sufficient to effectively treat or prevent a disease or disorder described herein. The “therapeutically effective amount” will vary depending on the compound, the disorder and the severity thereof, and the age of the patient to be treated, but can be adjusted as desired by those skilled in the art.

The term “subject” refers to any animal, preferably a mammal, most preferably a human, that is about to receive administration of or has received administration of a compound or composition. The term “mammal” includes any mammal. Examples of mammals include, but are not limited to, cows, horses, sheep, pigs, cats, dogs, mice, rats, rabbits, guinea pigs, monkeys, humans, and the like, with humans being most preferred.

All patents and publications referred to herein are incorporated by reference in their entirety.

The biological activity of the compounds disclosed herein can be assessed by using any conventionally known method. Appropriate detection methods are well known in the art. For example, affinity activity, agonistic activity and/or antagonistic activity of the compound disclosed herein for a dopamine receptor, pharmacokinetic activity and/or liver microsome stability of the compound disclosed herein, and the like can be detected by an appropriate conventional method. The detection methods provided by the present invention are presented as examples only and do not limit the present invention. The compounds disclosed herein are active in at least one of the detection methods provided by the present invention.

The above preferred conditions may be combined arbitrarily to obtain preferred embodiments of the present invention without departing from the general knowledge in the art.

The reagents and starting materials used in the present invention are commercially available.

The abbreviations referred to herein have the following meanings: PE represents petroleum ether; EA represents ethyl acetate; ACN represents acetonitrile; Sphos represents 2-dicyclohexylphosphino-2′,6′-dimethoxybiphenyl; Pd₂(dba)₃ represents tris(dibenzylideneacetone) dipalladium; HATU represents 2-(7-azabenzotriazol)-N,N,N′,N′-tetramethyluronium hexafluorophosphate; LDA represents lithium diisopropylamide; THE represents tetrahydrofuran; PyBOP represents benzotriazol-1-yl-oxytripyrrolidinophosphonium hexafluorophosphate; DBU represents 1,8-diazabicycloundec-7-ene; TEA represents triethylamine; DIPEA represents N,N-diisopropylethylamine; DCM represents dichloromethane; DMF represents N,N-dimethylformamide; DMSO represents dimethyl sulfoxide; TFA represents trifluoroacetic acid; DMAP represents 4-dimethylaminopyridine; BINAP represents 2,2′-bis(diphenylphosphino)-1,1′-binaphthyl; T3P represents 1-propylphosphoric anhydride; pd(dppf)Cl₂ represents [1,1′-bis(diphenylphosphino) ferrocene]dichloropalladium(II); NBS represents N-bromosuccinimide; NCS represents N-chlorosuccinimide; Xantphos represents 4,5-bis(diphenylphosphino)-9,9-dimethylxanthene; DIAD represents diisopropyl azodicarboxylate; Sphos Pd G3 represents (2-dicyclohexylphosphino-2′,6′-dimethoxybiphenyl)[2-(2′-amino-1,1′-biphenyl)]palladium (II) mesylate.

The compound disclosed herein has CDK7 inhibitory activity, and it can be used as a reference substance for in vitro test of inhibitory activity of a compound against CDK7, and can also be used for treating diseases such as a tumor.

### BRIEF DESCRIPTION OF THE DRAWINGS

FIG. 1 shows change in the body weight of experimental animals versus time of administration in Efficacy Example 6.

FIG. 2 shows the growth curve of tumor in Efficacy Example 6.

### DETAILED DESCRIPTION

The present invention is further illustrated by the following examples, which are not intended to limit the present invention. Experimental procedures without specified conditions in the following examples were performed in accordance with conventional procedures and conditions, or in accordance with instructions.

### Example 1 SZ-015095: (S,E)-1-(4-(dimethylamino)but-2-enoyl)pyrrolidin-3-yl 4-((3-isopropyl-5-methyl-3H-imidazo[4,5-b]pyridin-7-yl)amino)piperidine-1-carboxylate

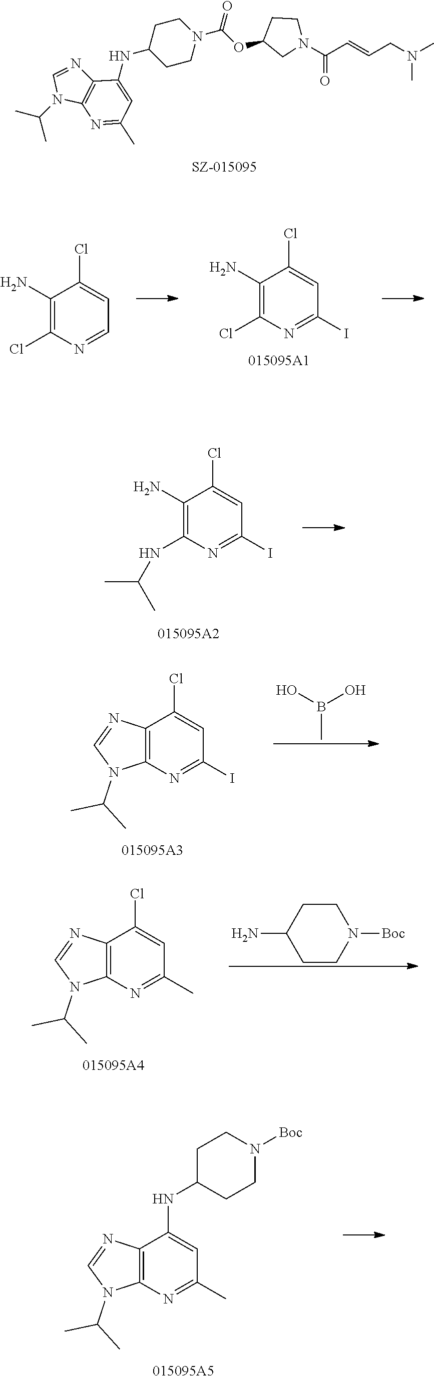

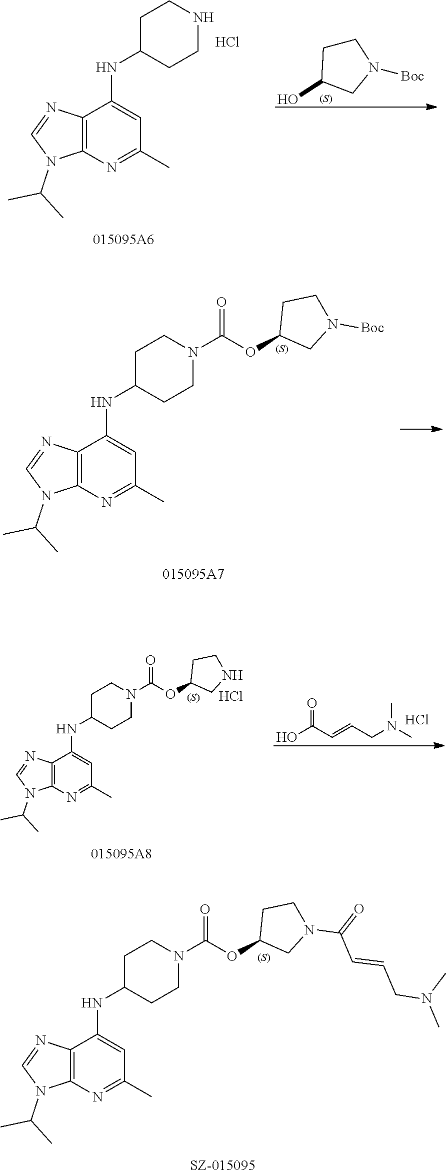

Step 1: 015095A1

Trifluoroacetic acid (1.9 g, 16.6 mmol) and N-iodosuccinimide (13.6 g, 60.7 mmol) were added to a solution of 3-amino-2,4-dichloropyridine (9.0 g, 55.2 mmol) in acetonitrile (100 mL) at room temperature, and the reaction solution was stirred at 40° C. overnight. After the reaction was completed, sodium thiosulfate solution (50 mL) was added to the reaction solution, and the resulting reaction solution was extracted with ethyl acetate (30 mL×3). The organic phases were combined, dried over anhydrous sodium sulfate and filtered, and then the residue obtained after concentration was purified by column chromatography to give 015095A1 (13.7 g, 87% yield, a yellow solid).

By LCMS (M+H)⁺ m/z, the calculation value was 288.9, and the measurement value was 288.9 [M+H]⁺.

Step 2: 015095A2

Compound 015095A1(5.0 g, 17.3 mmol) was dissolved in n-butanol (30 mL), and isopropylamine (5.1 g, 86.5 mmol) was added. The reaction solution was stirred at 180° C. for 18 h. After the reaction was completed, the reaction solution was concentrated. Water (10 mL) was added to the residue, and ethyl acetate (30 mL×3) was added for extraction. The organic phases were combined, dried over anhydrous sodium sulfate and filtered, and then the residue obtained after concentration was purified and concentrated by column chromatography to give 015095A2 (3.8 g, 70% yield, a red solid).

¹H NMR (CDCl₃, 400 MHz): δ 6.98 (s, 1H), 4.22-4.15 (m, 1H), 1.24-1.22 (d, J=8 Hz, 6H).

By LCMS (M+H)⁺ m/z, the calculation value was 312.0, and the measurement value was 312.1 [M+H]⁺.

Step 3: 015095A3

Triethyl orthoformate (1.92 g, 12.9 mmol) was added to a solution of 015095A2 (1.35 g, 4.33 mmol) in formic acid (60 mL) at room temperature, and the reaction solution was stirred at 100° C. for 4 h. After the reaction was completed, the reaction solution was cooled to room temperature and concentrated to remove the solvent. The residue was diluted with saturated aqueous sodium bicarbonate solution (150 mL) and stirred for 30 min, and ethyl acetate (150 mL) was added. The mixed solution was filtered, and the filter cake was washed with ethyl acetate to give filtrate. The aqueous phase was then separated out and extracted with ethyl acetate (50 mL×2), and the organic phases were combined, washed with saturated brine, dried over anhydrous sodium sulfate, filtered, and concentrated. The residue was purified and concentrated by column chromatography to give 015095A3 (710 mg, 51% yield, a red solid).

By LCMS (M+H)⁺ m/z, the calculation value was 322.0, and the measurement value was 321.9 [M+H]⁺.

Step 4: 015095A4

Compound 015095A3 (2.3 g, 7.16 mmol), methylboronic acid (1.29 g, 21.5 mmol), potassium carbonate (4.94 g, 35.8 mmol) and bis(triphenylphosphine)palladium(II) dichloride (503 mg, 0.176 mmol) were dissolved in DMF at room temperature. The reaction solution was stirred at 100° C. overnight. After the reaction was completed, the reaction solution was then concentrated. The residue was purified by silica gel column chromatography to give 015095A4 (1.0 g, 67% yield, a yellow solid).

¹H NMR (CDCl₃, 400 MHz): δ 8.10 (s, 1H), 7.16 (s, 1H), 5.04-4.94 (m, 1H), 1.65-1.63 d, J=8 Hz, 6H). By LCMS (M+H)⁺ m/z, the calculation value was 210.1, and the measurement value was 210.2 [M+H]⁺.

Step 5: 015095A5

Compound 015095A4 (400 mg, 1.89 mmol), N-Boc-4-aminopiperidine (758 mg, 3.79 mmol), potassium phosphate (803 mg, 3.79 mmol), Sphos (77.5 mg, 0.189 mmol) and Pd₂(dba)₃ (173 mg, 0.189 mmol) were dissolved in toluene at room temperature. The reaction solution was stirred at 110° C. overnight. After the reaction was completed, the reaction solution was then concentrated. The residue was purified by silica gel column chromatography to give 015095A5 (604 mg, 85% yield, a yellow solid).

¹H NMR (CDCl₃, 400 MHz): δ 7.78 (s, 1H), 6.21 (s, 1H), 5.09-5.07 (m, 1H), 4.96-4.91 (m, 1H), 4.13-4.06 (m, 2H), 3.70-3.68 (m, 2H), 3.01-2.95 (m, 2H), 2.53 (s, 3H), 2.09-2.04 (m, 2H), 1.61 (d, J=8 Hz, 6H), 1.48 (s, 9H). By LCMS (M+H)⁺ m/z, the calculation value was 374.2, and the measurement value was 374.3 [M+H]⁺.

Step 6: 015097A6

The crude product of 015095A5 (400 mg, 1.07 mmol) obtained in the previous step was dissolved in 5 N dioxane hydrochloride solution (5 mL) at room temperature, and the reaction solution was stirred at room temperature for 1 h. After the reaction was completed, the reaction solution was concentrated to give a crude product of 015095A6 (315 mg, >100%, a yellow solid), which was directly used in the next step without purification.

By LCMS (M+H)⁺ m/z, the calculation value was 274.2, and the measurement value was 274.2 [M+H]⁺.

Step 7: 015095A7

Triethylamine (288 mg, 2.85 mmol) and triphosgene (423 mg, 1.45 mmol) were added to a solution of (S)-1-N-tert-butoxycarbonyl-3-hydroxypyrrolidine (213 mg, 1.14 mmol) in tetrahydrofuran (10 mL) at 0° C., and the reaction solution was stirred at room temperature for 45 min. The reaction solution was concentrated to remove the solvent, and the residue was dissolved in dichloromethane (10 mL). The aforementioned dichloromethane solution was added to a solution of a crude product of 015095A6 (315 mg, 0.95 mmol) obtained in the previous step in dichloromethane (12 mL) and triethylamine (0.4 mL) at 0° C., and the reaction solution was stirred at room temperature overnight. After the reaction was completed, water (20 mL) was added to the reaction solution, and dichloromethane (15 mL×3) was added for extraction. The organic phases were combined, washed with saturated brine and concentrated. The residue was purified by silica gel column chromatography to give 015095A7 (101 mg, 19.3% yield, a yellow solid).

By LCMS (M+H)⁺ m/z, the calculation value was 487.3, and the measurement value was 487.4 [M+H]⁺.

Step 8: 015095A8

015095A7 (101 mg, 0.207 mmol) was dissolved in 5 N dioxane hydrochloride solution (10 mL) at room temperature, and the reaction solution was stirred at room temperature for 1 h. After the reaction was completed, the reaction solution was concentrated to give a crude product of 015095A8 (65 mg, 81%, a yellow oil), which was directly used in the next step without purification.

By LCMS (M+H)⁺ m/z, the calculation value was 387.2, and the measurement value was 387.3 [M+H]⁺.

Step 9: SZ-015095

Trans-4-dimethylaminocrotonic acid hydrochloride (29.9 mg, 0.180 mmol), 015095A8 (35 mg, 0.09 mmol), triethylamine (0.2 mL) and HATU (41.2 mg, 0.108 mmol) were added to N,N-dimethylformamide (5 mL), and the reaction solution was stirred at room temperature for 1 h. After the reaction was completed, water (40 mL) was added to the reaction solution, and ethyl acetate (15 mL×3) was added for extraction. The organic phases were combined, washed with saturated brine and concentrated. The residue was purified by preparative high performance liquid chromatography to give SZ-015095 (5.4 mg, 12% yield, a white solid).

By LCMS (M+H)⁺ m/z, the calculation value was 498.3, and the measurement value was 498.3 [M+H]⁺.

¹H NMR (CDCl₃, 400 MHz): δ 7.78 (s, 1H), 6.98-6.91 (m, 1H), 6.33-6.21 (m, 2H), 5.31 (s, 1H), 5.15-5.08 (m, 1H), 5.13-5.11 (m, 1H), 4.96-4.89 (m, 1H), 4.15-4.00 (m, 2H), 3.80-3.56 (m, 5H), 3.11-3.02 (m, 4H), 2.54 (s, 3H), 2.27-2.26 (m, 6H), 2.21-2.17 (m, 1H), 2.11-2.04 (m, 3H), 1.58-1.56 (m, 6H), 1.49-1.48 (m, 2H).

### Example 2 SZ-015256: (S,E)-1-(4-(dimethylamino)but-2-enoyl)pyrrolidin-3-yl 4-((8-isopropyl-2-methylpyrazolo[1,5-a][1,3,5]triazin-4-yl)amino)piperidine-1-carboxylate

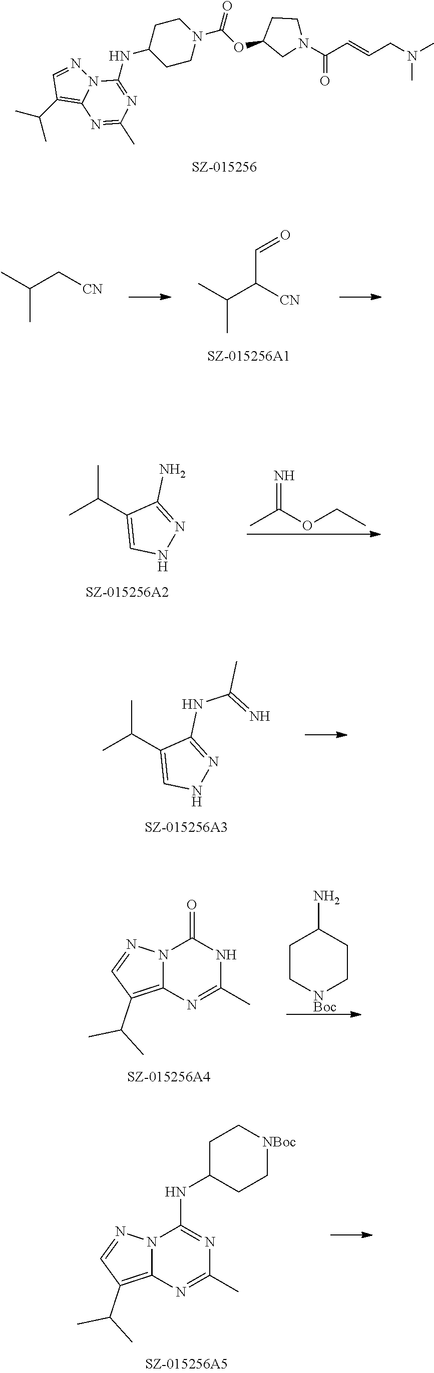

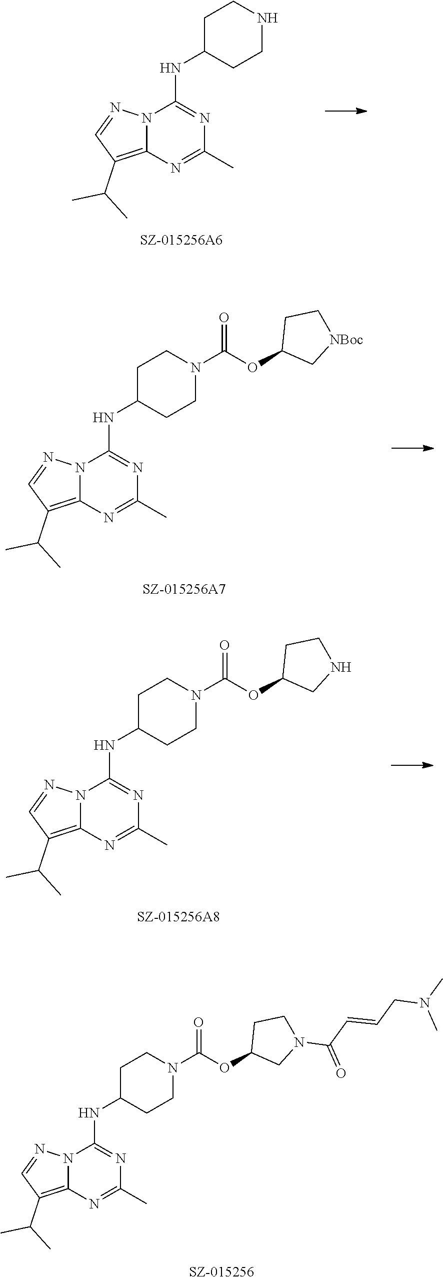 [SMILES: Cc1nc(NC2CCNCC2)n2ncc(C(C)C)c2n1 ; Cc1nc(NC2CCN(C(=O)O[C@H]3CCN(C(=O)OC(C)(C)C)C3)CC2)n2ncc(C(C)C)c2n1 ; Cc1nc(NC2CCN(C(=O)O[C@H]3CCNC3)CC2)n2ncc(C(C)C)c2n1 ; Cc1nc(NC2CCN(C(=O)O[C@H]3CCN(C(=O)/C=C/CN(C)C)C3)CC2)n2ncc(C(C)C)c2n1]

### Step 1: 2-formyl-3-methylbutyronitrile

Isovaleronitrile (20 g, 0.24 mol) was added dropwise to LDA (130 mL, 2 M in THF) at −70° C. under argon atmosphere, and the reaction solution was stirred for 40 min after dropwise addition was completed. A solution of ethyl formate (22 mL) in THE (100 mL) was then added dropwise to the reaction solution at this temperature, and the resulting reaction solution was stirred at room temperature overnight after dropwise addition was completed. 4 N hydrochloric acid was added to the reaction solution until the pH was about 3, and ethyl acetate (200 mL) was added for extraction. The organic phase was dried over anhydrous sodium sulfate, filtered and concentrated by rotary evaporation to give SZ-015256A1.

### Step 2: 4-isopropyl-1H-pyrazol-3-amine

SZ-015256A1 obtained in the previous step, acetic acid (22 mL) and hydrazine hydrate (17 mL) were dissolved in ethanol (400 mL), and the reaction solution was refluxed overnight. Ethanol was removed by rotary evaporation, and the pH was adjusted to about 9 with saturated sodium bicarbonate. Dichloromethane (300 mL) was added for extraction, and the organic phase was dried over anhydrous sodium sulfate and concentrated by rotary evaporation to give SZ-015256A2 (20 g).

### Step 3: N-(4-isopropyl-1H-pyrazol-3-yl)acetimidamide

SZ-015256A2 (20 g) and ethyl acetimidate hydrochloride (20 g) were dissolved in acetonitrile (200 mL) and acetic acid (10 g), and the reaction solution was stirred at room temperature overnight. Undissolved solids were filtered out, and the filtrate was concentrated by rotary evaporation. The residue was washed by flash silica gel column chromatography (MeOH:EA=10% to 100%) to give SZ-015256A3 (6 g, an oil). LCMS: [M+H]⁺ 167.31.

### Step 4: 8-isopropyl-2-methylpyrazolo[1,5-a][1,3,5]triazin-4(3H)-one

SZ-015256A3 (6 g), 21% sodium ethoxide/ethanol (120 mL), and diethyl carbonate (30 mL) were mixed and stirred at 110° C. for 16 h. Ethanol was removed by rotary evaporation, and the residue was purified by flash silica gel column chromatography (PE:EA=1:4; Rf=0.2) to give SZ-015256A4 (2.5 g, a foamy solid). LCMS: [M+H]⁺ 193.11.

### Step 5: tert-butyl 4-((8-isopropyl-2-methylpyrazolo[1,5-a][1,3,5]triazin-4-yl)amino) piperidine-1-carboxylate

SZ-015256A4 (1.7 g, 8.9 mmol), PyBOP (3.2 g, 10.6 mmol), DBU (1.0 g, 10.6 mmol) and 1-Boc-4-aminopiperidine (1.2 g, 10.6 mmol) were mixed in acetonitrile (20 mL), and the reaction solution was stirred at room temperature for 4 h. After the reaction was completed, the reaction solution was concentrated by rotary evaporation, and the residue was purified by flash silica gel column chromatography (PE:EA=1:4; Rf=0.6) to give SZ-015256A5 (600 mg, a foamy solid). LCMS: [M+H]⁺ 375.25.

### Step 6: 8-isopropyl-2-methyl-N-(piperidin-4-yl)pyrazolo[1,5-a][1,3,5]triazin-4-amine

The product obtained in the previous step was dissolved in dichloromethane (6 mL), and trifluoroacetic acid (5 mL) was slowly added dropwise. The reaction solution was stirred at room temperature for about 2 h, and then directly concentrated by rotary evaporation to give a crude product of SZ-015256A6 (500 mg). LCMS: [M+H]⁺ 275.26.

### Step 7: (S)-1-(tert-butoxycarbonyl)pyrrolidin-3-yl 4-((8-isopropyl-2-methylpyrazolo [1,5-a][1,3,5]triazin-4-yl)amino)piperidine-1-carboxylate

Tert-butyl (S)-3-hydroxypyrrolidine-1-carboxylate (177 mg, 0.93 mmol),p-nitrophenyl chloroformate (205 mg, 0.51 mmol) and triethylamine (0.5 mL, 3.24 mmol) were mixed in acetonitrile (5 mL), and the reaction solution was stirred at 40° C. for 2 h. Then SZ-015256A6 (200 mg, 0.72 mmol) was added, and the resulting reaction solution was stirred at room temperature for 2 h. The reaction solution was directly concentrated by rotary evaporation, and the residue was purified by flash silica gel column chromatography (PE:EA=1:1; Rf=0.3) to give SZ-015256A7 (158 mg, a foamy solid). LCMS: [M+H]⁺ 488.34.

### Step 8

SZ-015256A7 (158 mg) was dissolved in dichloromethane (2 mL), and trifluoroacetic acid (2 mL) was slowly added dropwise. The reaction solution was stirred at room temperature for about 2 h, and then directly concentrated by rotary evaporation to give a crude product of SZ-015256A8 (200 mg).LCMS: [M+H]⁺ 390.31.

### Step 9

SZ-015256A8 obtained in the previous step, trans-4-dimethylaminocrotonic acid hydrochloride (65 mg, 0.40 mmol), DIPEA (0.2 mL, 1.36 mmol) and HATU (188 mg, 0.49 mmol) were mixed in tetrahydrofuran (3 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015256 (66 mg, a light yellow solid). LCMS: [M+H]⁺ 499.09.

SZ-015256: ¹H NMR (400 MHz, DMSO-d₆) 8.52 (d, J=8.4 Hz, 1H), 8.03 (s, 1H), 6.74-6.63 (m, 2H), 5.23-5.18 (m, 1H), 4.30-4.28 (m, 1H), 4.02-3.79 (m, 5H), 3.69-3.40 (m, 3H), 3.13-3.16 (m, 1H), 2.98-2.95 (m, 2H), 2.80 (s, 6H), 2.41 (s, 3H), 2.12-2.09 (m, 2H), 1.86-1.84 (m, 2H), 1.70-1.64 (m, 2H), 1.29 (d, J=7.2 Hz, 6H).

### Example 3 SZ-015264: (S,E)-1-(4-(dimethylamino)but-2-enoyl)pyrrolidin-3-yl 4-((9-isopropyl-2-methyl-9H-purin-6-yl)amino)piperidine-1-carboxylate

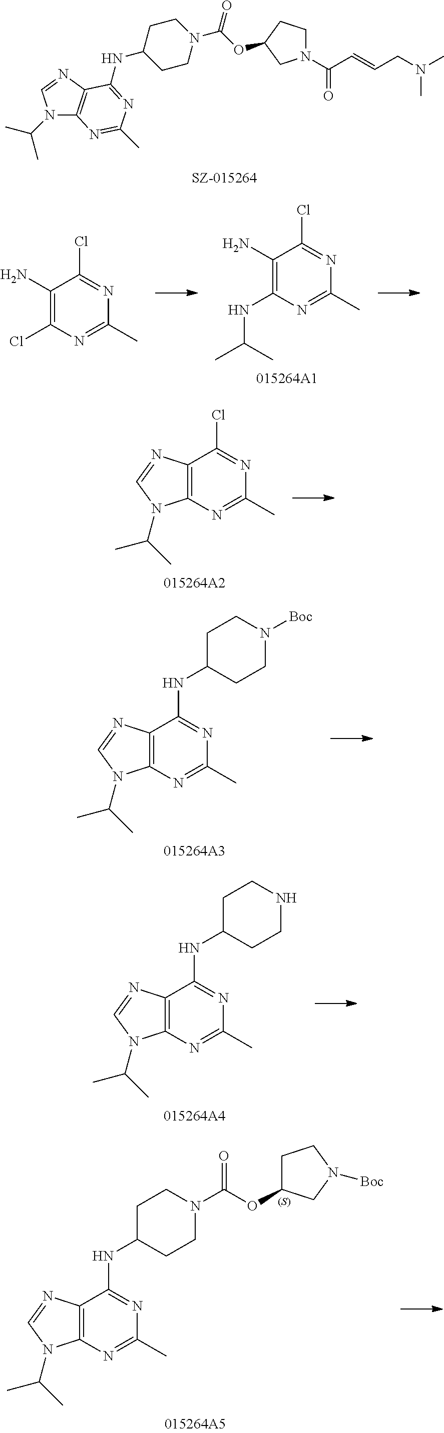

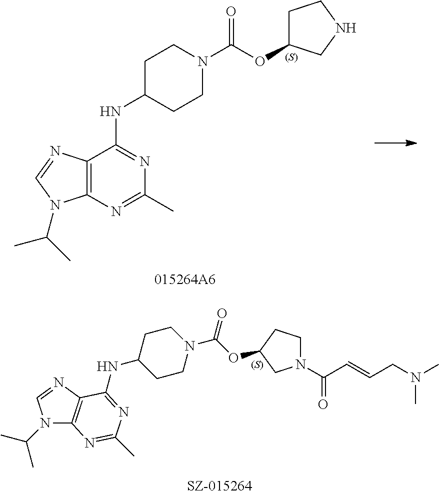

### Step 1: 6-chloro-N⁴-isopropyl-2-methylpyrimidin-4,5-diamine

4,6-dichloro-2-methylpyrimidin-5-amine (1.0 g, 5.7 mmol), isopropylamine (3.4 g, 57 mmol) and isopropanol (10 mL) were added to a microwave flask, and the reaction solution was heated to 120° C. and reacted for 8 h under a microwave condition. The reaction solution was cooled to room temperature and distilled under reduced pressure to give the product 015264A1 (1 g, 87%, a light yellow solid). LCMS: [M+H]⁺ 201.38.

### Step 2: 6-chloro-9-isopropyl-2-methyl-9H-purine

015264A1 (1.0 g, 5 mmol), trimethyl orthoformate (9 mL) and acetic acid (4 mL) were added to a microwave flask, and the reaction solution was heated to 100° C. and reacted for 3 h under a microwave condition. After the reaction was completed, the reaction solution was cooled to room temperature, and the solvent was removed by rotary evaporation. The residue was concentrated under reduced pressure to give a crude product, which was purified by flash silica gel column chromatography to give 015264A2 (1.0 g, 95%, a white solid). LCMS: [M+H]⁺ 211.34.

### Step 3: tert-butyl 4-((9-isopropyl-2-methyl-9H-purin-6-yl)amino)piperidine-1-carboxylate

Compound 015264A2 (1.0 g, 4.76 mmol) was dissolved in isopropanol (10 mL), and tert-butyl 4-aminopiperidine-1-carboxylate (1.05 g, 5.23 mmol) was added. The reaction solution was heated to 100° C. and stirred for 6 h, and then the reaction solution was cooled to room temperature and concentrated by rotary evaporation under reduced pressure to give a crude product of 015264A3, which was purified by flash silica gel column chromatography to give 015264A3 (540 mg, 31%, a white solid). LCMS: [M+H]⁺ 375.15.

### Step 4: 9-isopropyl-2-methyl-N-(piperidin-4-yl)-9H-purin-6-amine

Compound 015264A3 (540 mg, 1.44 mmol) was dissolved in dichloromethane (9 mL), and trifluoroacetic acid (3 mL) was added. The reaction solution was stirred at room temperature for 1 h and concentrated by rotary evaporation under reduced pressure to give 015264A4 trifluoroacetate (540 mg, 100%, a colorless oily liquid). LCMS: [M+H]⁺ 275.12.

### Step 5: (S)-1-(tert-butoxycarbonyl)pyrrolidin-3-yl 4-((9-isopropyl-2-methyl-9H-purin-6-yl)amino)piperidine-1-carboxylate

Compound 015264A4 trifluoroacetate (540 mg, 1.44 mmol) was dissolved in acetonitrile (9 mL), and triethylamine (303 mg, 3 mmol) and tert-butyl (S)-3-(((4-nitrophenoxy)carbonyl)oxo)pyrrolidine-1-carboxylate (507 mg, 1.44 mmol) were added successively. The reaction solution was stirred at room temperature for 2 h and concentrated by rotary evaporation under reduced pressure to give a crude product of 015264A5, which was purified by flash silica gel column chromatography to give 015264A5 (240 mg, 34%, a white solid). LCMS: [M+H]⁺ 488.34.

### Step 6: (S)-pyrrolidin-3-yl-4-((9-isopropyl-2-methyl-9H-purin-6-yl)amino)piperidine-1-carboxylate

Compound 015264A5 (240 mg, 0.49 mmol) was dissolved in dichloromethane (6 mL), and trifluoroacetic acid (2 mL) was added. The reaction solution was stirred at room temperature for 0.5 h and concentrated by rotary evaporation under reduced pressure to give 015264A6 trifluoroacetate (240 mg, 100%, a colorless oily liquid). LCMS: [M+H]⁺ 388.16.

### Step 7: (S,E)-1-(4-(dimethylamino)but-2-enoyl)pyrrolidin-3-yl 4-((9-isopropyl-2-methyl-9H-purin-6-yl)amino)piperidine-1-carboxylate

Compound 015264A5 trifluoroacetate (240 mg, 0.49 mmol) was dissolved in DMF (5 mL), and DIPEA (260 mg, 2 mmol), (E)-4-(dimethylamino)but-2-enoic acid hydrochloride (83 mg, 0.49 mmol) and HATU (228 mg, 0.6 mmol) were added successively. The reaction solution was stirred at room temperature for 0.5 h and concentrated by rotary evaporation under reduced pressure to give a crude product of SZ-015264, which was subjected to reverse phase column chromatography (acetonitrile/water with 0.1% formic acid) and lyophilized to give SZ-015264 (5 mg, 2%, a white solid). LCMS: [M+H]⁺ 499.14.

¹H NMR (400 MHz, DMSO-d₆) δ 8.15 (s, 1H), 7.43 (d, J=8.5 Hz, 1H), 6.62 (ddd, J=16.3, 10.4, 6.1 Hz, 1H), 6.36 (dd, J=21.7, 15.4 Hz, 1H), 5.15 (d, J=23.7 Hz, 1H), 4.70 (dt, J=13.3, 6.6 Hz, 1H), 3.96 (s, 2H), 3.85-3.67 (m, 1H), 3.64-3.40 (m, 3H), 3.39-3.30 (m, 1H), 3.05-2.91 (m, 4H), 2.41 (s, 3H), 2.12-1.99 (m, 8H), 1.92-1.85 (m, 2H), 1.48 (d, J=6.8 Hz, 6H), 1.52-1.50 (m, 2H).

### Example 4 SZ-015272: (S,E)-1-(4-(dimethylamino)but-2-enoyl)pyrrolidin-3-yl 4-((1-isopropyl-6-methyl-1H-imidazo[4,5-c]pyridin-4-yl)amino)piperidine-1-carboxylate

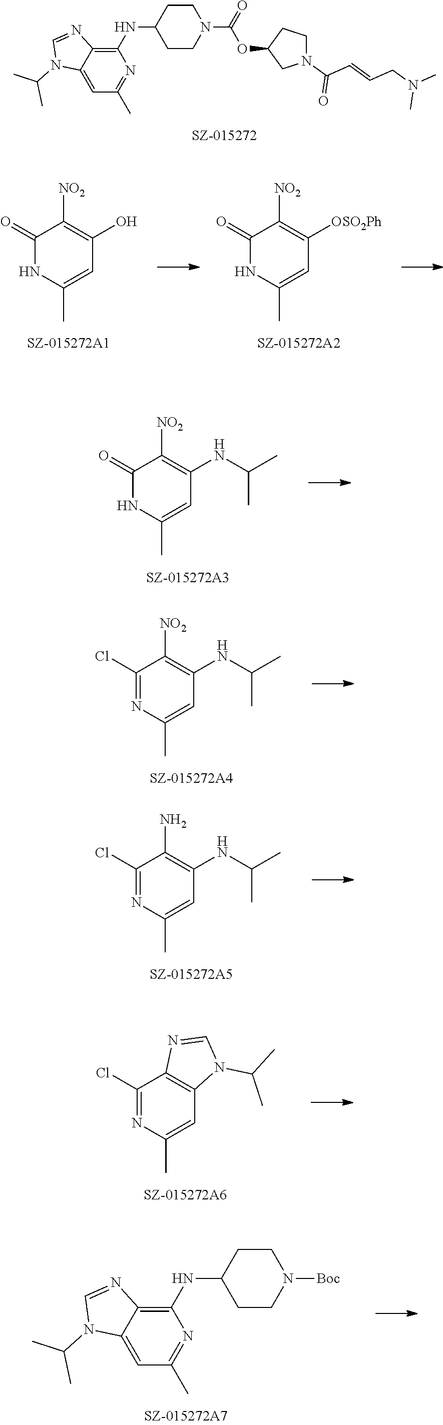

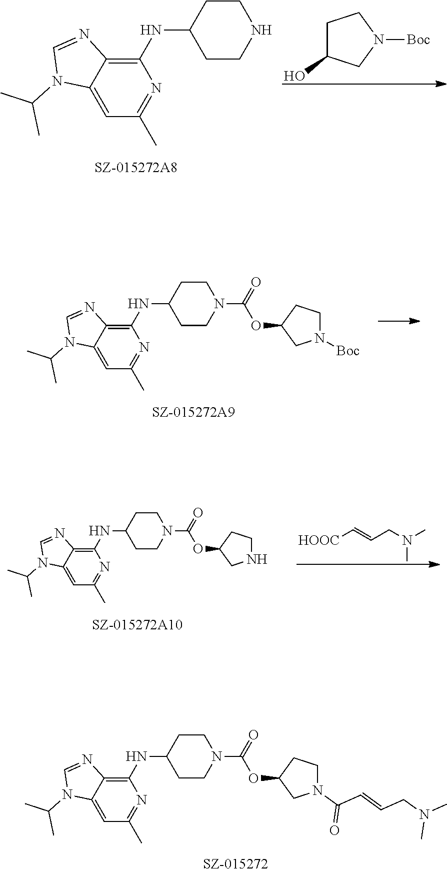

### Step 1: 6-methyl-3-nitro-2-oxo-1,2-dihydropyridin-4-p-methylbenzenesulfonate

THE (100 mL), SZ-015272A1 (10.21 g), triethylamine (9.216 mL) and DMAP (368 mg) were added to a 500 mL reaction flask and stirred under reflux for 2 h, and then a solution of benzenesulfonyl chloride (10.60 g) in THE (50 mL) was added dropwise to the reaction solution about 30 min later. The resulting reaction system was further stirred under reflux for 4 h. After the reaction was completed, as detected by LCMS, the reaction solution was used in the next step without any treatment. LCMS: [M+1]⁺ 310.97.

### Step 2: 4-(isopropylamino)-6-methyl-3-nitropyridin-2(1H)-one

Triethylamine (9.216 mL) was added to the reaction solution in the step 1, and isopropylamine (5.11 mL) was added while stirring under reflux. The resulting reaction solution was further stirred under reflux for 2 h, and then the reaction solution was stirred overnight after the temperature was adjusted to 40° C. After the reaction was completed, the reaction solution was filtered to give a residue, which was repeatedly washed with water and dichloromethane to give an off-white solid, and the off-white solid was dried with an oil pump to give SZ-015272A3 (5.66 g). LCMS: [M+1]⁺ 212.12.

### Step 3: 2-chloro-N-isopropyl-6-methyl-3-nitropyridin-4-amine

SZ-015272A3 (5.66 g), acetonitrile (10 mL), POCl₃ (2.5 mL) and DMF (0.5 mL) were added to a 100 mL reaction flask and stirred at 50° C. for 2 h. After the reaction was completed, a saturated NaHCO₃ solution was added to quench the reaction, and the reaction solution was adjusted to be alkaline and extracted with ethyl acetate. The organic phases were combined, and the solvent was removed by rotary evaporation. The residue was purified by silica gel column chromatography (ethyl acetate:petroleum ether=1:2) to give SZ-015272A4 (4.325 g). LCMS: [M+1]⁺ 229.94.

### Step 4: 2-chloro-N-isopropyl-6-methylpyridin-3,4-diamine

SZ-015272A4 (3.3372 g), reduced iron powder (4.2 g), ammonium chloride (4.01 g), ethanol (55 mL) and water (11 mL) were added to a 250 mL reaction flask and stirred under reflux at 80° C. overnight. After the reaction was completed, the solvent was removed by rotary evaporation. The residue was diluted with ethanol, and the solid was removed by filtration. The filtrate was concentrated by rotary evaporation to give a crude product of SZ-015272A5 (2.787 g), which was directly used in the next step without separation. LCMS: [M+1]⁺ 200.08.

### Step 5: 4-chloro-1-isopropyl-6-methyl-1H-imidazo[4,5-c]pyridine

SZ-015272A5 (2.787 g) and triethyl orthoformate (100 mL) were added to a 250 mL reaction flask, DMF was added dropwise until the solution became clear, and 12 N concentrated hydrochloric acid (1.7 mL) was then added. The reaction solution was stirred at room temperature overnight under nitrogen atmosphere. After the reaction was completed, the reaction solution was concentrated by rotary evaporation, and the residue was separated by a silica gel column (ethyl acetate:petroleum ether=1:2) to give SZ-015272A6 (2.596 g). LCMS: [M+1]⁺ 210.11.

### Step 6: tert-butyl 4-((1-isopropyl-6-methyl-1H-imidazo[4,5-c]pyridin-4-yl)amino) piperidine-1-carboxylate

SZ-015272A6 (836 mg), 1-Boc-4-aminopiperidine (2.4 g), BINAP (373.6 mg), Pd₂(dba)₃ (183.2 mg), sodium tert-butoxide (576 mg) and toluene (50 mL) were added to a 250 mL reaction flask, and refluxed at 110° C. overnight under nitrogen atmosphere. After the reaction was completed, the reaction solution was filtered through celite, washed with ethyl acetate and concentrated, and the residue was separated by a silica gel column (ethyl acetate:petroleum ether=50%-100%) to give a crude product of SZ-015272A7 (1.408 g), which was directly used in the next step. LCMS:[M+1]⁺ 374.27.

### Step 7: 1-isopropyl-6-methyl-N-(piperidin-4-yl)-1H-imidazo[4,5-c]pyridin-4-amine

SZ-015272A7 (560 mg), dichloromethane (5 mL) and a solution of hydrogen chloride in methanol (4 M, 10 mL) were added to a 100 mL reaction flask and stirred at room temperature for 3 h. After the reaction was completed, the reaction solution was concentrated by rotary evaporation to give a crude product of SZ-015272A8, which was directly used in the next step without separation. LCMS: [M+1]⁺ 274.08.

### Step 8: (S)-1-(tert-butoxycarbonyl)pyrrolidin-3-yl 4-((1-isopropyl-6-methyl-1H-imidazo [4,5-c]pyridin-4-yl)amino)piperidine-1-carboxylate

(S)-1-N-tert-butoxycarbonyl-3-hydroxypyrrolidine (365.2 mg), acetonitrile (10 mL), triethylamine (1.5 mL) and p-nitrophenyl chloroformate (362.8 mg) were added to a 100 mL reaction flask and stirred at 40° C. for 2 h. The crude product in the previous step was dissolved in acetonitrile (10 mL), triethylamine was added to adjust the solution to be alkaline, and then the reaction solution of (S)-1-N-tert-butoxycarbonyl-3-hydroxypyrrolidine was added dropwise to the solution of SZ-015272A8 in acetonitrile. The resulting reaction solution was stirred at room temperature for 4 h. After the reaction was completed, the solvent was removed by rotary evaporation, and the residue was separated by a short silica gel column (ethyl acetate:petroleum ether=20%-100%) to give SZ-015272A9 (597.6 mg). LCMS: [M+1]+487.33.

### Step 9: (S)-pyrrolidin-3-yl 4-((1-isopropyl-6-methyl-1H-imidazo[4,5-c]pyridin-4-yl)amino)piperidine-1-formic acid

SZ-015272A9 (130 mg), dichloromethane (5 mL) and a solution of hydrogen chloride in methanol (4 M, 10 mL) were added to a 100 mL reaction flask and stirred at room temperature for 3 h. After the reaction was completed, the solvent was removed by rotary evaporation to give a crude product of SZ-015272A10, which was directly used in the next step without separation. LCMS: [M+1]⁺ 387.02

### Step 10: (S,E)-1-(4-(dimethylamino)but-2-enoyl)pyrrolidin-3-yl 4-((1-isopropyl-6-methyl-1H-imidazo[4,5-c]pyridin-4-yl)amino)piperidine-1-carboxylate

The crude product of SZ-015272A10 in the previous step, (2E)-4-(dimethylamino)-2-butenoic acid hydrochloride (66.24 mg), DIPEA (0.3 mL), T₃P (318 mg) and tetrahydrofuran (10 mL) were added to a 100 mL reaction flask and stirred at room temperature for 2 h. After the reaction was completed, the solvent was removed by rotary evaporation, and the residue was separated by a reverse phase column (water:acetonitrile) to give SZ-015272 (23 mg). LCMS: [M+1]⁺ 498.30.

¹H NMR (DMSO-d₆, 400 MHz): 8.10 (s, 1H), 6.69-6.61 (m, 1H), 6.67 (s, 1H), 6.44-6.32 (m, 2H), 5.18 (d, J=23.9 Hz, 1H), 4.60(hept, J=7.0 Hz, 1H), 4.35-4.22 (m, 1H), 4.05-3.82 (m, 2H), 3.81-3.70 (m, 1H), 3.57-3.47 (m, 3H), 3.05 (d, J=5.9 Hz, 2H), 2.95 (br, 2H), 2.36 (s, 3H), 2.17 (s, 6H), 2.20-2.00 (m, 2H), 1.90-1.88 (m, 2H), 1.56-1.47 (m, 2H), 1.50 (d, J=6.7 Hz, 6H).

### Example 5 SZ-015273: (S,E)-1-(4-(dimethylamino)but-2-enoyl)pyrrolidin-3-yl 4-((3-isopropyl-6-methylimidazo[1,2-b]pyridazin-8-yl)amino)piperidin-1-carboxylate

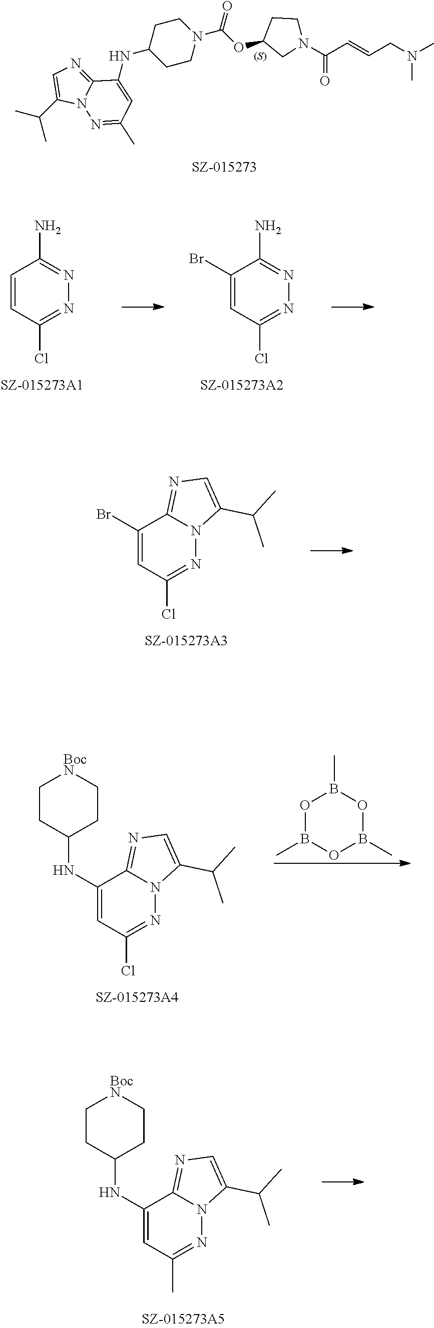

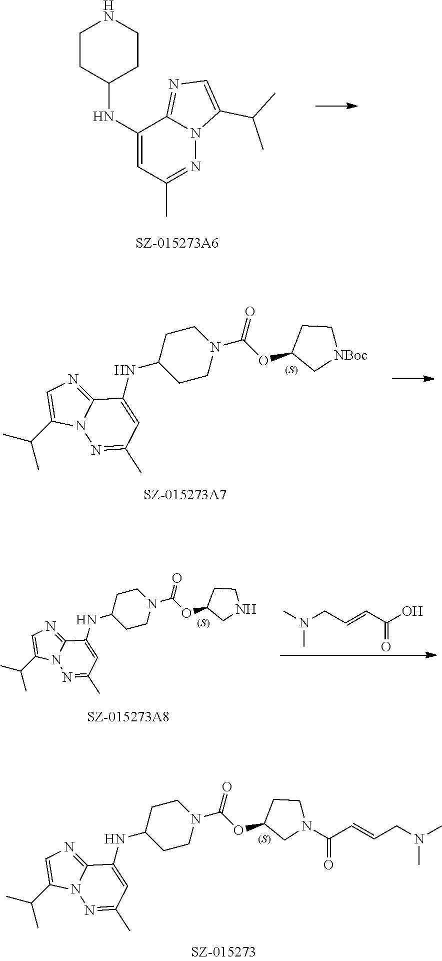

### Step 1: 4-bromo-6-chloropyridazin-3-amine

SZ-015273A1 (25 g, 193 mmol) and sodium bicarbonate (32 g, 387 mmol) were dissolved in methanol (350 mL), and liquid bromine (31 g, 193 mmol) was slowly added dropwise under an ice-water bath. After the addition was completed, the reaction solution was stirred at room temperature overnight. The reaction solution was washed with saturated aqueous sodium bicarbonate solution (200 mL) and saturated sodium thiosulfate solution (100 mL), and extracted with ethyl acetate (300 mL). The organic phase was dried over sodium sulfate and concentrated by rotary evaporation to give SZ-015273A2 (20 g, a brown solid). LCMS: [M+H]⁺ 207.94.

### Step 2: 8-bromo-6-chloro-3-isopropylimidazo[1,2-b]pyridazine

SZ-015273A2 (12.7 g, 60 mmol) and 2-bromo-3-methylbutanal (10 g, 60 mmol) were dissolved in ethanol (100 mL), and the reaction solution was refluxed at 100° C. overnight. After the reaction was completed, the reaction solution was concentrated by rotary evaporation, and the residue was purified by flash silica gel column chromatography (PE:EA=1:1; Rf=0.6) to give SZ-015273A3 (1.6 g, a foamy solid). LCMS: [M+H]⁺ 273.98.

### Step 3: tert-butyl 4-((6-chloro-3-isopropylimidazo[1,2-b]pyridazin-8-yl)amino) piperidine-1-carboxylate

SZ-015273A3 (1.5 g, 5.4 mmol), triethylamine (2.5 mL, 13.7 mmol) and tert-butyl 4-aminopiperidin-1-carboxylate (1.5 g, 7.1 mmol) were dissolved in dioxane (20 mL), and the reaction solution was stirred at 80° C. for 24 h. After the reaction was completed, the reaction solution was concentrated by rotary evaporation, and the residue was purified by flash silica gel column chromatography (PE:EA=2:1; Rf=0.3) to give SZ-015273A4 (1.3 g, a foamy solid). LCMS: [M+H]⁺ 394.25.

### Step 4: tert-butyl 4-((3-isopropyl-6-methylimidazo[1,2-b]pyridazin-8-yl)amino) piperidine-1-carboxylate

SZ-015273A4 (1.1 g, 2.8 mmol), 2,4,6-trimethyl-1,3,5,2,4,6-trioxatriborinane (2.3 mL, 3.5 mol/L), pd(dppf)Cl₂.CH₂Cl₂ (200 mg) and potassium carbonate (1.1 g, 8.4 mmol) were mixed in dioxane (3 mL) and water (1 mL) under argon atmosphere, and then the reaction solution was heated to 100° C. and reacted for 3 h under a microwave condition. After the reaction was completed, the reaction solution was concentrated by rotary evaporation, and the residue was purified by flash silica gel column chromatography (PE:EA=2:1; Rf=0.3) to give SZ-015273A5 (200 mg, a foamy solid). LCMS: [M+H]⁺ 374.41.

### Step 5: 3-isopropyl-6-methyl-N-(piperidin-4-yl)imidazo[1,2-b]pyridazin-8-amine

SZ-015273A5 (200 mg) was dissolved in dichloromethane (2 mL), and then TFA (1 mL) was added. The reaction solution was stirred at room temperature for 2 h. After the reaction was completed, the reaction solution was concentrated by rotary evaporation and directly used in the next step. [M+H]⁺274.32.

### Step 6: (S)-1-(tert-butoxycarbonyl)pyrrolidin-3-yl 4-((3-isopropyl-6-methylimidazo [1,2-b]pyridazin-8-yl)amino)piperidine-1-carboxylate

Tert-butyl (S)-3-hydroxypyrrolidine-1-carboxylate (100 mg, 0.54 mmol), p-nitrophenyl chloroformate (110 mg, 0.59 mmol) and triethylamine (0.2 mL) were mixed in acetonitrile (3 mL), and the reaction solution was stirred at 40° C. for 2 h. Then SZ-015273A6 (150 mg, 0.41 mmol) was added, and the resulting reaction solution was stirred at room temperature for 2 h. The reaction solution was directly concentrated by rotary evaporation, and the residue was purified by flash silica gel column chromatography (PE:EA=1:1; Rf=0.3) to give SZ-015273A7 (80 mg, a foamy solid). LCMS: [M+H]⁺487.55.

### Step 7: (S)-pyrrolidin-3-yl 4-((3-isopropyl-6-methylimidazo [1,2-b]pyridazin-8-yl)amino)piperidine-1-carboxylate

SZ-015273A7 (80 mg) was dissolved in dichloromethane (2 mL), and trifluoroacetic acid (1 mL) was slowly added dropwise. The reaction solution was stirred at room temperature for about 2 h, and then directly concentrated by rotary evaporation to give a crude product of SZ-015273A8.LCMS: [M+H]⁺ 387.51.

### Step 8

SZ-015273A8 obtained in the previous step, trans-4-dimethylaminocrotonic acid hydrochloride (42 mg, 0.25 mmol), DIPEA (0.2 mL, 1.36 mmol) and HATU (120 mg, 0.31 mmol) were mixed in DMF (3 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015273 (14 mg, a light yellow solid). LCMS: [M+H]⁺ 498.35.

SZ-015273: ¹H NMR (400 MHz, DMSO-d₆) 7.22 (s, 1H), 6.97-6.95 (m, 1H), 6.68-6.51 (m, 2H), 6.09 (s, 1H), 5.22-5.16 (m, 1H), 4.08-3.96 (m, 2H), 3.85-3.48 (m, 8H), 2.96-2.91 (m, 2H), 2.53 (s, 6H), 2.38 (s, 3H), 2.21-2.03 (m, 2H), 1.86-1.84 (m, 2H), 1.52-1.50 (m, 2H), 1.29 (d, J=7.2 Hz, 6H).

### Example 6 SZ-015268: (S,E)-1-(4-(dimethylamino)but-2-enoyl)pyrrolidin-3-yl 4-((7-isopropyl-2-methylimidazo[2,1-f][1,2,4]triazin-4-yl)amino)piperidin-1-carboxylate

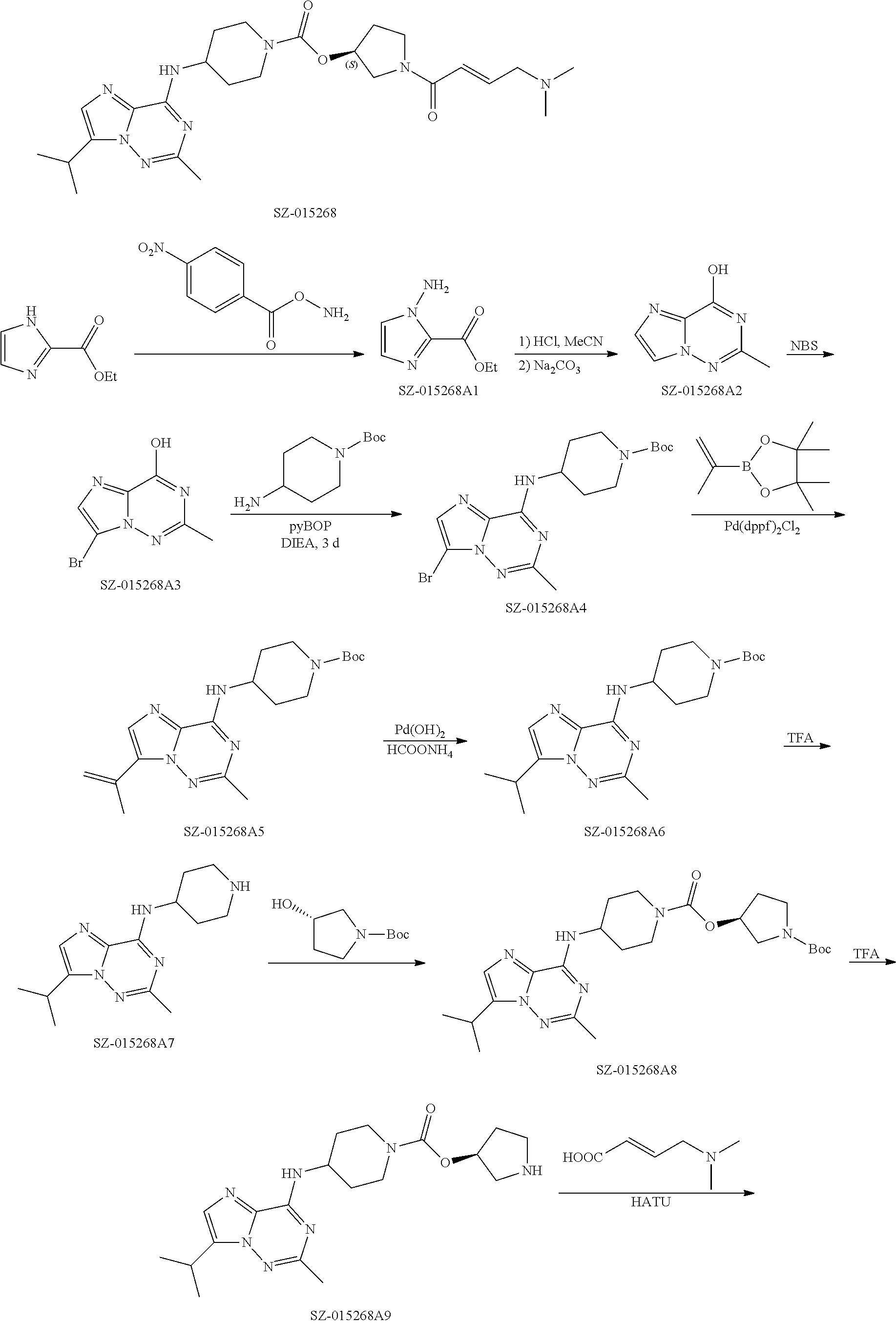

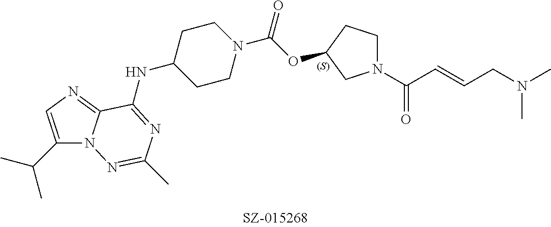

Step 1:

Ethyl imidazole-2-carboxylate (6 g) was dissolved in DMF (400 mL) in a reaction flask, and a solution of potassium tert-butoxide (5.282 g) in DMF (60 mL) was added dropwise to the reaction solution with stirring. The resulting reaction solution was stirred at room temperature for 1 h. O-p-nitrobenzoyl hydroxylamine (7.795 g) was dissolved in DMIF (100 mL), and the solution was added dropwise to the reaction solution. The reaction solution changed from dark blue to brown and finally to orange as the dropwise addition proceeded. The reaction solution was stirred at room temperature overnight. After the reaction was completed, the reaction solution was concentrated, saturated sodium bicarbonate solution (400 mL) was added to quench the reaction, and dichloromethane (500 mL×4) was added for extraction. The organic phases were combined, dried over anhydrous sodium sulfate and filtered, and the solvent was removed by rotary evaporation to give a crude product of SZ-015268A1 (4.33 g). LCMS: [M+1]⁺156.05.

Step 2:

Dry SZ-015268A1 (4.33 g) and anhydrous acetonitrile (50 mL) were added to a three-necked flask and bubbled for 10 min with nitrogen introduced through a conduit. The reaction solution was cooled to 0° C. and stirred, and then dry hydrogen chloride gas was introduced for 20 min. The reaction solution was then stirred at 80° C. for 1 h. After the reaction was completed, the reaction solution was concentrated, and the remaining solid was slurried with diethyl ether and filtered to give a crude product as an intermediate, which was directly used in the next step without purification. The intermediate, 1,4-dioxane (40 mL), water (30 mL) and sodium carbonate (2.352 g) were added to a reaction flask and stirred at 100° C. for 1 h. After the reaction was completed, the solvent was removed under reduced pressure, and the remaining solid was diluted with acetonitrile and filtered to give a crude product of SZ-015268A2 (5 g, a white solid) containing sodium chloride. LCMS: [M+1]⁺ 150.98.

Step 3:

SZ-015268A2 (2.1 g), NBS (3 g) and DMF (50 mL) were added to a reaction flask and stirred at 80° C. for 1 h. The reaction solution was concentrated by rotary evaporation under reduced pressure to remove the solvent, and the residue was diluted with dichloromethane and filtered to give a crude product of SZ-015268A3 (2.182 g, a white solid) containing sodium chloride. LCMS: [M+1]⁺ 230.89.

Step 4:

SZ-015268A3 (2.182 g), 1-Boc-4-aminopiperidine (4 g), PyBOP (7.36 g), 1,2-dichloroethane (50 mL) and DIPEA (1.71 mL) were added to a reaction flask and stirred at room temperature for 3 d. After the reaction was completed, the reaction solution was filtered and washed with dichloromethane. The organic phase was concentrated by rotary evaporation to remove the solvent, and the resulting crude product was purified by flash silica gel column chromatography (EA/PE=0%-40%) to give product SZ-015268A4 (1.645 g). LCMS: [M+1]+413.09.

Step 5:

SZ-015268A4 (1.645 g), isopropenylboronic acid pinacol ester (1.3 g), Pd(dppf)Cl₂ (530 mg), potassium carbonate (1.6 g), 1,4-dioxane (40 mL) and water (7 mL) were added to a reaction flask and stirred at 80° C. for 3 h under nitrogen atmosphere. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015268A5 (1.659 g). LCMS: [M+1]⁺ 373.27.

Step 6:

SZ-015268A5 (800 mg), Pd(OH)₂ (100 mg), ammonium formate (1 g) and methanol (10 mL) were added to a reaction flask and stirred at 80° C. for 1 h. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015268A6 (597 mg). LCMS: [M+1]⁺ 375.28.

Step 7:

SZ-015268A6 (597 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015268A7, which was directly used in the next step without separation. LCMS: [M+1]+275.15.

Step 8:

(S)-1-N-tert-butoxycarbonyl-3-hydroxypyrrolidine (318 mg), acetonitrile (5 mL), triethylamine (1 mL) and p-nitrophenyl chloroformate (342 mg) were added successively to a reaction flask and stirred at 40° C. for 2 h. The crude product of SZ-015268A7 in the previous step was dissolved in acetonitrile (5 mL), triethylamine was added to adjust the solution to be alkaline, and then the reaction solution of (S)-1-N-tert-butoxycarbonyl-3-hydroxypyrrolidine was added dropwise to the solution of SZ-015268A7 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-5%) to give SZ-015268A8 (422 mg). LCMS: [M+1]⁺488.20

Step 9:

SZ-015268A8 (150 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015268A9, which was directly used in the next step without separation. LCMS: [M+1]+388.25.

Step 10:

The crude product of SZ-015268A9 in the previous step, (2E)-4-(dimethylamino)-2-butenoic acid hydrochloride (66 mg), DIPEA (0.5 mL), HATU (190 mg) and DMF (10 mL) were added to a reaction flask and stirred at room temperature for 2 h. After the reaction was completed, the solvent was removed by rotary evaporation, and the residue was separated by a reverse phase column (acetonitrile-water with 0.01% formic acid) to give SZ-015268 (81 mg). LCMS: [M+1]⁺499.22.

¹H NMR (400 MHz, DMSO-d₆) δ 8.49 (d, J=8.2 Hz, 1H), 7.33 (s, 1H), 6.73-6.49 (m, 2H), 5.20 (d, J=20.3 Hz, 1H), 4.38-4.32 (m, 1H), 4.03=3.99 (m, 2H), 3.90-3.77 (m, 3H), 3.69-3.49 (m, 3H), 3.32-3.25 (m, 1H), 2.95-2.94 (m, 2H), 2.78 (s, 6H), 2.38 (s, 3H), 2.21-2.09 (m, 2H), 1.84-1.83 (m, 2H), 1.61-1.57 (m, 2H), 1.33 (d, J=6.9 Hz, 6H).

### Example 7 SZ-015274: (S,E)-1-(4-(dimethylamino)but-2-enoyl)tetrahydropyrrol-3-yl 4-((3-isopropyl-6-methyl-3H-imidazo[1,2-a]pyridin-8-yl)amino)piperidine-1-carboxylate

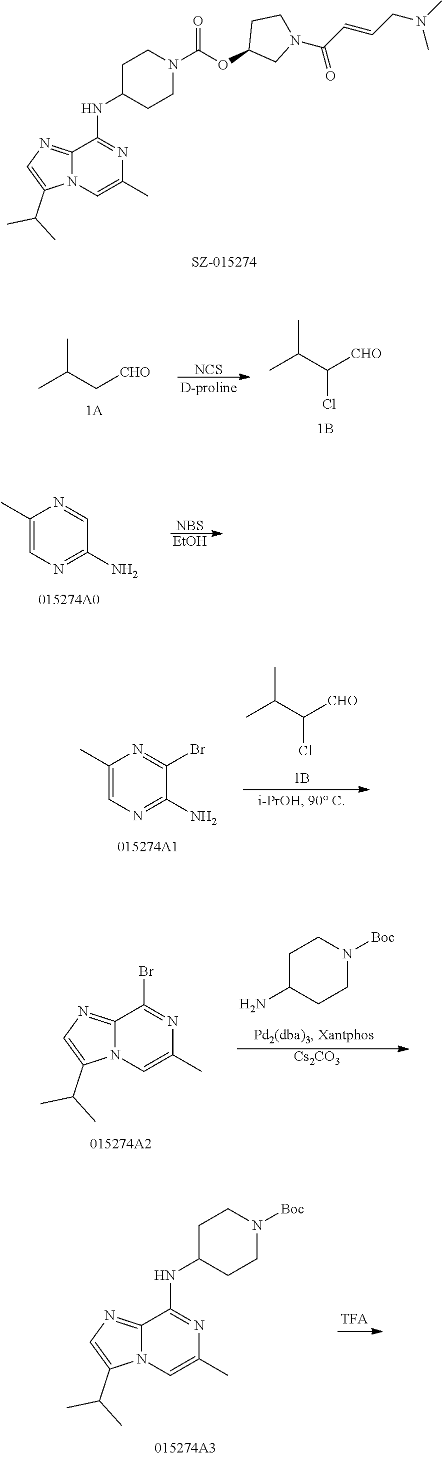

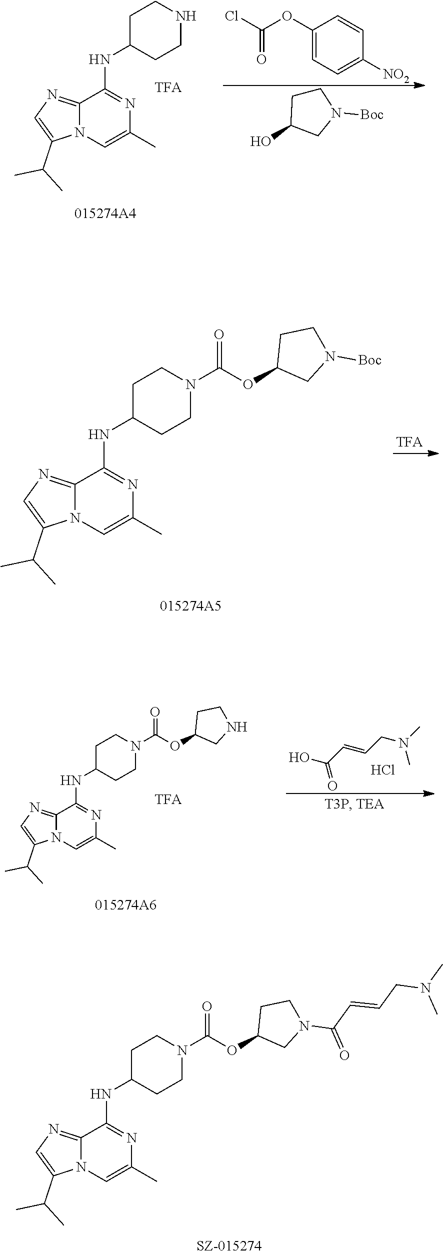

Step 1:

NBS (18.6 g) was added in portions to a solution of compound 015274A0 (9.9 g, 90.8 mmol) in ethanol (110 mL) at 1-3° C., and the resulting yellow suspension was stirred at 1-16° C. for 16 h. The resulting reaction solution was concentrated by rotary evaporation to remove the solvent, and water and ethyl acetate were added for liquid separation. After the organic phase was separated out, the aqueous phase was extracted with ethyl acetate (0.3 L×3). The organic phases were combined, dried over anhydrous MgSO₄ and filtered, and the organic solvent was removed by rotary evaporation. The resulting oil was purified by column chromatography to give compound 015274A1 (4.1 g, a yellow solid). LCMS: [M+H]⁺ 188.09/190.16

Step 2:

NCS (9.65 g, 71.8 mmol) was added in portions to a solution of compound 1A (5.6 g, 65 mmol) in dichloromethane (110 mL) at 18° C., and then D-proline (1.36 g, 11.8 mmol) was added in portions. The resulting reaction solution was stirred at 18° C. for 3 h. The reaction solution was diluted with petroleum ether and filtered through celite. The filtrates were combined, and the solvent was removed by rotary evaporation to give compound 1B (7.86 g, a crude product, a yellow liquid), which was directly used in the next step.

Step 3:

A solution of compound 015274A1 (4.1 g, 21.6 mmol) and compound 1B (7.86 g) in isopropanol (90 mL) was heated to 89° C. and stirred for 36 h. After the resulting reaction solution was concentrated by rotary evaporation to remove the organic solvent, the residue was dissolved in ethyl acetate, and the resulting solution was purified by column chromatography using a mixture of basic alumina and silica gel to give impure 015274A2 (1 g: LCMS for assisting in detection), which was directly used in the next step. LCMS: [M+H]⁺ 254.11/256.16.

Step 4:

Pd₂(dba)₃ (110 mg, 0.39 mmol) and Xantphos (69 mg, 0.39 mmol) were added successively to a suspension of compound 015274A2 (1 g, 3.9 mmol), tert-butyl 4-aminopiperidin-1-carboxylate (1.1 g, 5.21 mmol) and cesium carbonate (3.9 g, 11.9 mmol) in 1,4-dioxane (16 mL). The resulting reaction solution was purged with nitrogen three times with stirring at room temperature, and then heated to 110° C. and further stirred for 16 h. The resulting dark brown reaction solution was cooled to room temperature, water was added to quench the reaction, and ethyl acetate (60 mL×3) was added for extraction. The organic phases were combined, dried over anhydrous MgSO₄ and filtered, and the organic solvent was removed by rotary evaporation. The residue was purified by column chromatography to give compound 015274A3 (360 mg). LCMS: [M+H]⁺ 374.26.

Step 5:

Trifluoroacetic acid (3 mL) was added in portions to compound 015274A3 (360 mg) at 21° C. The resulting reaction solution was heated to 90° C. and stirred for 1 h. The reaction solution was concentrated by rotary evaporation to remove the solvent, and then the residue was dried with an oil pump for 3 min to give compound 015274A4 (190 mg), which was directly used in the next step. LCMS: [M+H]⁺ 274.11.

Step 6:

Triethylamine (9 mL) was added in portions to a solution of p-nitrophenyl chloroformate (260 mg, 1.3 mmol) and tert-butyl (S)-3-hydroxypyrrolidine-1-carboxylate (230 mg, 1.3 mmol) in 1,4-dioxane (6 mL) at 21° C. The resulting reaction solution was heated to 31° C. and stirred for 1 h. The reaction solution was added in portions to compound 015274A4 (190 mg) in the previous step. The resulting reaction solution was heated to 69° C. and further stirred for 16 h. The resulting yellow solution was cooled to room temperature, and water was added to quench the reaction. Solid sodium carbonate was added to the mixed solution until it was saturated, and ethyl acetate (30 mL×3) was added for extraction. The organic phases were combined, dried over anhydrous MgSO₄ and filtered, and the organic solvent was removed by rotary evaporation. The residue was purified by column chromatography to give compound 015274A5 (190 mg). LCMS: [M+H]⁺ 487.06.

Step 7:

Trifluoroacetic acid (3 mL) was added in portions to compound 015274A5 (190 mg) at 21° C. The resulting reaction solution was stirred at 21° C. for 1 h. The reaction solution was concentrated by rotary evaporation to remove the organic solvent, and then the residue was dried with an oil pump for 3 min to give compound 015274A6 (160 mg), which was directly used in the next step. LCMS: [M+H]⁺ 387.19.

Step 8:

A solution of T₃P (6.39 mL, 6 mmol, 50 wt % in EtOAc) was added in portions to a suspension of compound 015274A6 (160 mg, 0.39 mmol), trans-4-dimethylaminocrotonic acid hydrochloride (160 mg, 1 mmol) and triethylamine (3.6 mL, 25.8 mmol) in acetonitrile (6 mL) at 21° C. The resulting reaction solution was stirred at 18-21° C. for 16 h. Water was added to the reaction solution to quench the reaction, solid sodium carbonate was then added to the reaction solution until it was saturated, and ethyl acetate (60 mL×3) was added for extraction. The organic phases were combined, dried over anhydrous MgSO₄ and filtered, and the organic solvent was removed by rotary evaporation. The residue was purified by column chromatography to give impure SZ-015274 (160 mg), which was then purified by preparative high performance liquid chromatography to give compound SZ-015274 (31 mg, an off-white solid). LCMS: [M+H]⁺ 498.31.

¹H NMR (400 MHz, DMSO-d₆) δ 7.44 (s, 1H), 7.19 (s, 1H), 7.13 (d, J=8.0 Hz, 1H), 6.65-6.61 (m, 1H), 6.49-6.39 (m, 1H), 5.16 (d, J=24.0 Hz, 1H), 3.91-3.79 (m, 1H), 3.79-3.69 (m, 2H), 3.68-3.49 (m, 3H), 3.39-3.33 (m, 1H), 3.29-3.13 (m, 3H), 3.01-2.63 (m, 2H), 2.26 (d, J=4.0 Hz, 6H), 2.21 (s, 3H), 2.16-1.89 (m, 2H), 1.86-1.76 (m, 2H), 1.58-1.39 (m, 2H), 1.27 (d, J=8.0 Hz, 6H).

### Example 8 SZ-015282: (R,E)-1-(4-(dimethylamino)but-2-enoyl)pyrrolidin-3-yl 4-((8-isopropyl-2-methylpyrazolo[1,5-a][1,3,5]triazin-4-yl)amino)piperidine-1-carboxylate

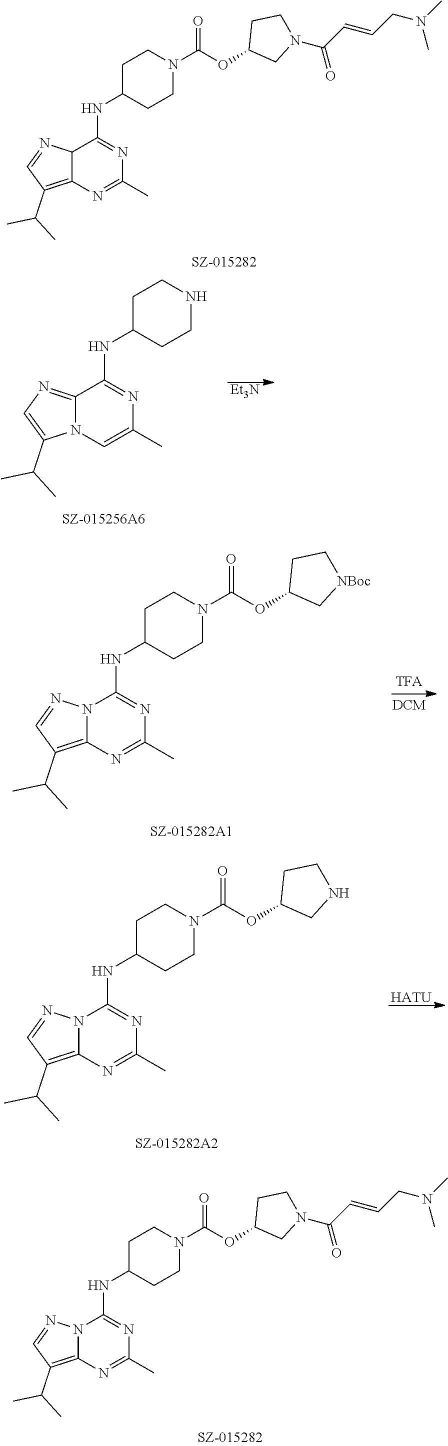

Step 1:

Tert-butyl (R)-3-hydroxypyrrolidine-1-carboxylate (219 mg, 0.8 mmol), p-nitrophenyl chloroformate (225 mg, 0.8 mmol) and triethylamine (0.6 mL) were mixed in acetonitrile (5 mL), and the reaction solution was stirred at 40° C. for 2 h. Then SZ-015256A6 (200 mg, 0.8 mmol) was added, and the resulting reaction solution was stirred at room temperature for 2 h. The reaction solution was directly concentrated by rotary evaporation, and the residue was purified by flash silica gel column chromatography (PE:EA=1:1; Rf=0.3) to give SZ-015282A1 (200 mg, a foamy solid). LCMS: [M+H]⁺ 488.37.

Step 2:

SZ-015282A1 (110 mg) was dissolved in dichloromethane (5 mL), and trifluoroacetic acid (3 mL) was slowly added dropwise. The reaction solution was stirred at room temperature for about 2 h, and then directly concentrated by rotary evaporation to give a crude product of SZ-015282A2 (200 mg).LCMS: [M+H]⁺ 388.49.

Step 3:

SZ-015282A2 obtained in the previous step, trans-4-dimethylaminocrotonic acid hydrochloride (37 mg, 0.22 mmol), DIPEA (0.3 mL) and HATU (111 mg, 0.29 mmol) were mixed in DMF (3 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015282 (36 mg, a light yellow solid). LCMS: [M+H]⁺ 499.53.

¹H NMR (400 MHz, DMSO-d₆) δ 8.54-8.52 (m, 1H), 8.01 (s, 1H), 6.68-6.63 (m, 1H), 6.47-6.39 (m, 1H), 5.22-5.16 (m, 1H), 4.29-4.01 (m, 4H), 3.90-3.39 (m, 3H), 3.15-3.06 (m, 3H), 2.96-2.92 (m, 2H), 2.41 (s, 3H), 2.23 (s, 6H), 2.10-2.02 (m, 2H), 1.85-1.83 (m, 2H), 1.67-1.66 (m, 2H), 1.29 (d, J=7.2 Hz, 6H).

### Example 9 SZ-015294: (S)-1-(but-2-ynoyl)pyrrolidin-3-yl 4-((8-isopropyl-2-methylpyrazolo[1,5-a][1,3,5]triazin-4-yl)amino)piperidine-1-carboxylate

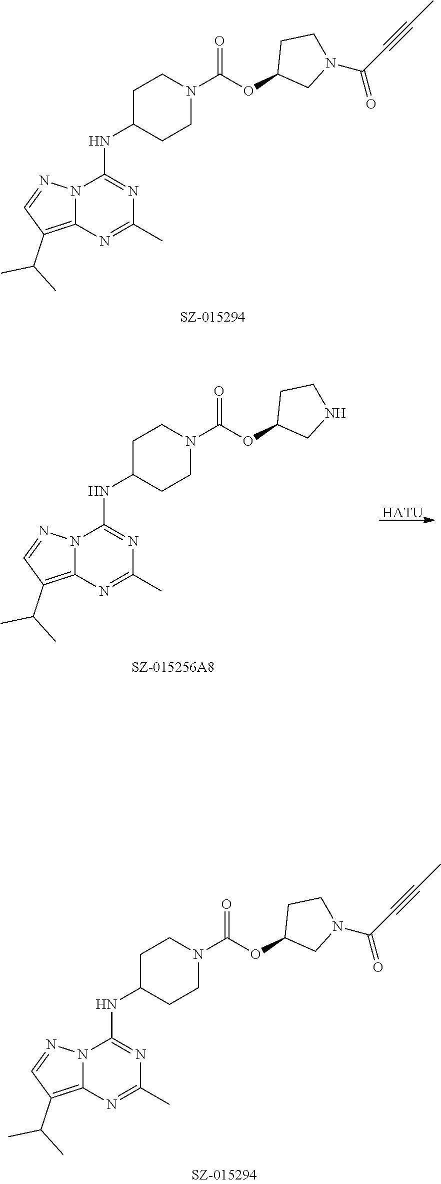

SZ-015256A8 (156 mg, 0.41 mmol), 2-butynoic acid (34 mg, 0.41 mmol), HATU (236 mg, 0.49 mmol) and DIPEA (0.4 mL) were mixed in anhydrous DMF (3 mL) under argon atmosphere, and the reaction solution was stirred at room temperature for 2 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015294 (105 mg, a white solid). LCMS: [M+H]⁺ 454.33.

¹H NMR (400 MHz, DMSO-d₆) δ 8.62-8.60 (m, 1H), 8.03 (s, 1H), 5.18-5.17 (m, 1H), 4.31-4.29 (m, 1H), 4.06-4.01 (m, 2H), 3.85-3.77 (m, 1H), 3.69-3.532 (m, 3H), 3.14-3.07 (m, 1H), 2.97-2.96 (m, 2H), 2.43 (s, 3H), 2.20-2.05 (m, 2H), 2.05 (s, 3H), 1.87-1.84 (m, 2H), 1.71-1.62 (m, 2H), 1.29 (d, J=7.2 Hz, 6H).

### Example 10 SZ-015284: (R)-1-(but-2- ynoyl)tetrahydropyrrol-3-yl 4-((3-isopropyl-5-methyl-3H-imidazo[4,5-b]pyridin-7-yl)amino)piperidine-1-carboxylate

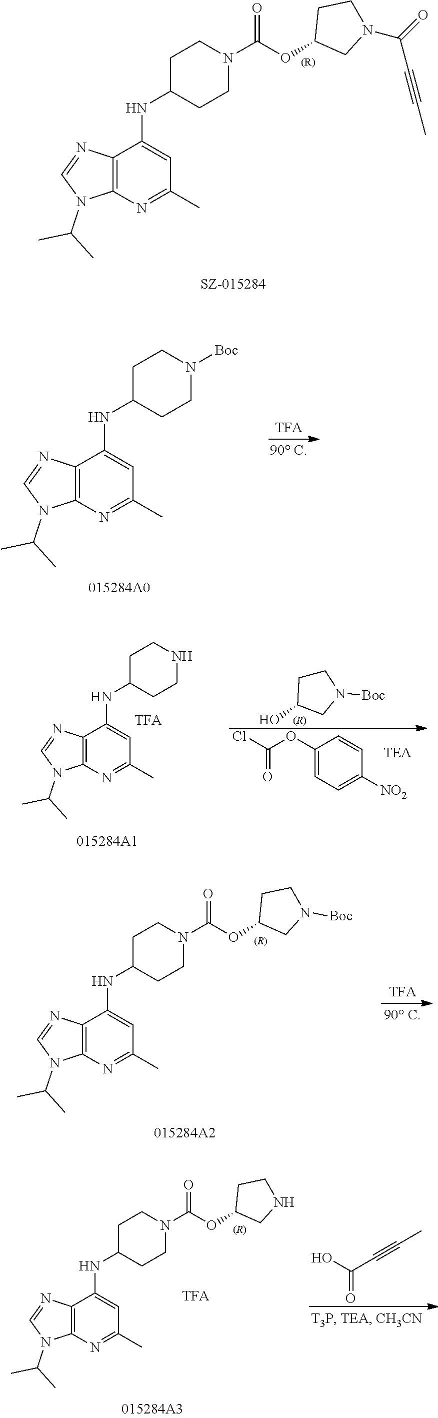

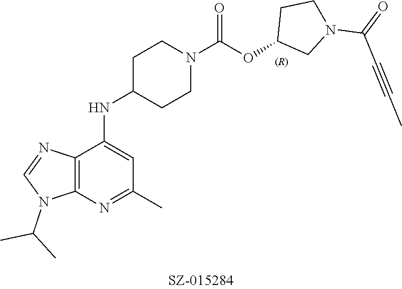

Step 1:

Trifluoroacetic acid (6 mL) was added to compound 015284A0 (015095A5) (630 mg, 1.69 mmol), and the resulting reaction solution was heated to 90° C. and stirred for 1 h. The resulting reaction solution was concentrated by rotary evaporation to remove the solvent, and then the residue was dried for 3 min with an oil pump to give product 015284A1 (580 mg, a crude product, a tawny oil), which was directly used in the next step. LCMS: [M+H]⁺ 274.03.

Step 2:

P-nitrophenyl chloroformate (1.26 g, 6 mmol) and tert-butyl (R)-3-hydroxypyrrolidine-1-carboxylate (1.26 g, 6.66 mmol) were dissolved in 1,4-dioxane (6 mL), and triethylamine (3.6 mL, 25.8 mmol) was added in portions to the reaction solution. The resulting reaction solution was heated to 31° C. and stirred for 3 h to give a light yellow suspension. The resulting reaction suspension was added in portions to a round-bottom flask containing compound 015284A1 (580 mg), and the resulting mixture was heated to 69° C. and stirred for 3 h. The resulting reaction solution was cooled to room temperature, and water was added to quench the reaction. Then solid sodium carbonate was added until the reaction solution was saturated, and ethyl acetate (60 mL×3) was added for extraction. The organic phases were combined, dried over anhydrous MgSO₄ and filtered, and the organic solvent was removed by rotary evaporation. The resulting residue was purified by column chromatography to give compound 015284A2 (390 mg, 80 wt %, a light yellow oil). LCMS: [M+H]⁺ 487.16.

Step 3:

Trifluoroacetic acid (6 mL) was added to compound 015284A2 (390 mg), and the resulting reaction solution was heated to 90° C. and stirred for 1 h. The resulting reaction solution was concentrated by rotary evaporation to remove the solvent, and then the residue was dried for 3 min with an oil pump to give product 015284A3 (360 mg, a crude product, a tawny oil), which was directly used in the next step. LCMS: [M+H]⁺ 387.11.

Step 4:

Triethylamine (9 mL) was added in portions to a suspension of compound 015284A3 (360 mg) and 2-butynoic acid (110 mg, 1.28 mmol) in acetonitrile (6 mL), and the resulting reaction solution was stirred at 16° C. A solution of T₃P (1.8 mL, 3 mmol, 50 wt % in EtOAc) was then added in portions to the above reaction solution. The resulting reaction solution was heated to 31° C. and stirred for 16 h. Water was added to the reaction solution to quench the reaction, solid sodium carbonate was then added to the reaction solution until it was saturated, and ethyl acetate (60 mL×3) was added for extraction. The organic phases were combined, dried over anhydrous MgSO₄ and filtered, and the organic solvent was removed by rotary evaporation. The resulting residue was purified by column chromatography to give impure SZ-015284, which was then further purified by preparative high performance liquid chromatography to give compound SZ-015284 (62.3 mg, a white solid). LCMS: [M+H]⁺ 453.09.

¹H NMR (400 MHz, CD₃OD) δ 8.09 (s, 1H), 6.38 (d, J=8.0 Hz, 1H), 6.27 (s, 1H), 5.13 (s, 1H), 4.86-4.69 (m, 1H), 3.96 (br, 3H), 3.79-3.69 (s, 1H), 3.63-3.46 (m, 2H), 3.39-3.33 (m, 1H), 2.94 (br, 2H), 2.37 (s, 3H), 2.18-2.06 (m, 2H), 2.01 (s, 3H), 1.9 (br, 2H), 1.48 (d, J=4.0 Hz, 6H), 1.39-1.31 (m, 2H).

### Example 11 SZ-015289: (S)-1-(but-2-ynoyl)tetrahydropyrrol-3-yl 4-((3-isopropyl-5-methyl-3H-imidazo[4,5-b]pyridin-7-yl)amino)piperidine-1-carboxylate

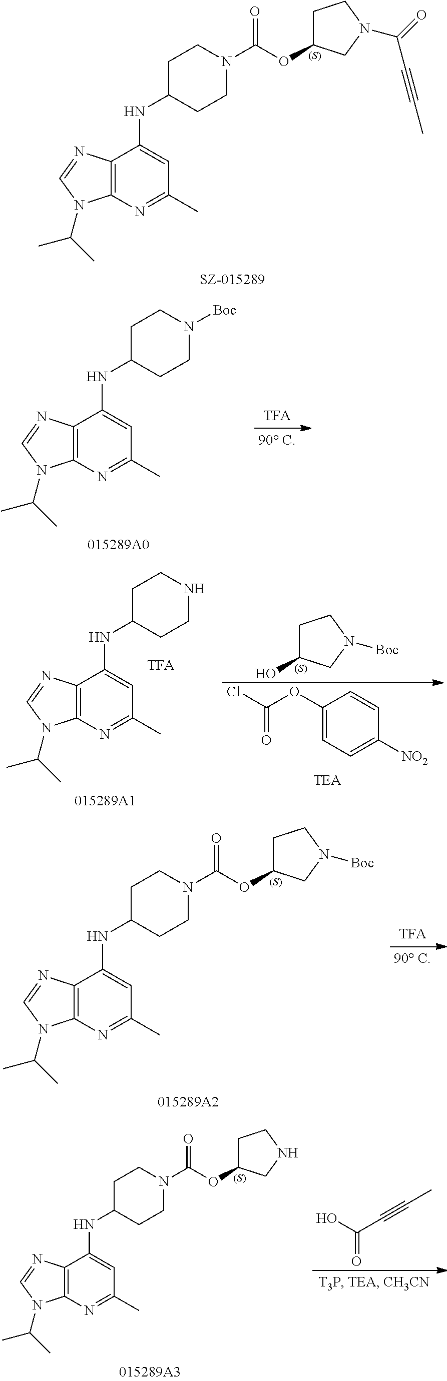

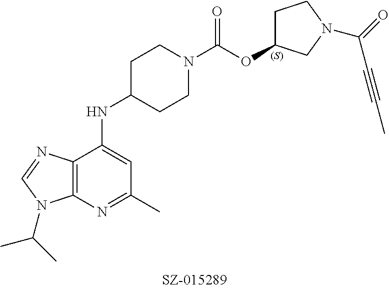

Step 1:

Trifluoroacetic acid (9 mL) was added to compound 015289A0 (015095A5) (1.6 g, 4.29 mmol), and the resulting reaction solution was heated to 90° C. and stirred for 1 h. The resulting reaction solution was concentrated by rotary evaporation to remove the solvent, and then the residue was dried for 3 min with an oil pump to give product 015289A1 (1.6 g, a crude product, a tawny oil), which was directly used in the next step. LCMS: [M+H]⁺ 274.09.

Step 2:

P-nitrophenyl chloroformate (1.1 g, 5 mmol) and tert-butyl (S)-3-hydroxypyrrolidine-1-carboxylate (930 mg, 5 mmol) were dissolved in 1,4-dioxane (26 mL), and triethylamine (3.6 mL, 25.8 mmol) was added in portions to the reaction solution. The resulting reaction solution was heated to 31° C. and stirred for 3 h to give a light yellow suspension. The resulting reaction suspension was added in portions to a round-bottom flask containing compound 015289A1 (1.6 g), and the resulting mixture was heated to 69° C. and stirred for 3 h. The resulting reaction solution was cooled to room temperature, and water was added to quench the reaction. Then solid sodium carbonate was added until the reaction solution was saturated, and ethyl acetate (60 mL×3) was added for extraction. The organic phases were combined, dried over anhydrous MgSO₄ and filtered, and the organic solvent was removed by rotary evaporation. The resulting residue was purified by column chromatography to give compound 015289A2 (1.9 g, 80 wt %, a light yellow oil). LCMS: [M+H]⁺ 487.13.

Step 3:

Trifluoroacetic acid (9 mL) was added to compound 015289A2 (1.9 g), and the resulting reaction solution was heated to 90° C. and stirred for 1 h. The resulting reaction solution was concentrated by rotary evaporation to remove the solvent, and then the residue was dried for 3 min with an oil pump to give product 015289A3 (1.3 g, a crude product, a tawny oil), which was directly used in the next step. LCMS: [M+H]⁺ 387.21.

Step 4:

Triethylamine (16 mL) was added to a suspension of compound 015289A3 (310 mg, 0.63 mmol) and 2-butynoic acid (110 mg, 1.28 mmol) in acetonitrile (9 mL), and the resulting reaction solution was stirred at 16° C. A solution of T₃P (1.8 mL, 3 mmol, 50 wt % in EtOAc) was then added in portions to the above reaction solution. The resulting reaction solution was heated to 31° C. and stirred for 16 h. Water was added to the reaction solution to quench the reaction, solid sodium carbonate was then added to the reaction solution until it was saturated, and ethyl acetate (60 mL×3) was added for extraction. The organic phases were combined, dried over anhydrous MgSO₄ and filtered, and the organic solvent was removed by rotary evaporation. The resulting residue was purified by column chromatography to give impure SZ-015289 (330 mg), which was then further purified by preparative high performance liquid chromatography to give compound SZ-015289 (21.1 mg, a white solid). LCMS: [M+H]⁺ 453.13.

¹H NMR (400 MHz, CD₃OD) δ 8.11 (s, 1H), 6.39 (s, 1H), 6.39 (br, 1H), 5.23 (s, 1H), 4.91-4.86 (m, 1H), 4.04 (m, 2H), 3.86-3.71 (s, 3H), 3.69-3.52 (m, 2H), 3.23-3.06 (m, 2H), 2.49 (s, 3H), 2.23-2.11 (m, 2H), 2.08-2.05 (m, 2H), 2.03 (s, 3H), 1.56 (d, J=4.0 Hz, 6H), 1.53-1.39 (m, 2H).

### Example 12 SZ-015285: (S,E)-1-(4-(dimethylamino)but-2-enoyl)pyrrolidin-3-yl 4-((3-ethyl-3H-imidazo[4,5-b]pyridin-7-yl)amino)piperidine-1-carboxylate

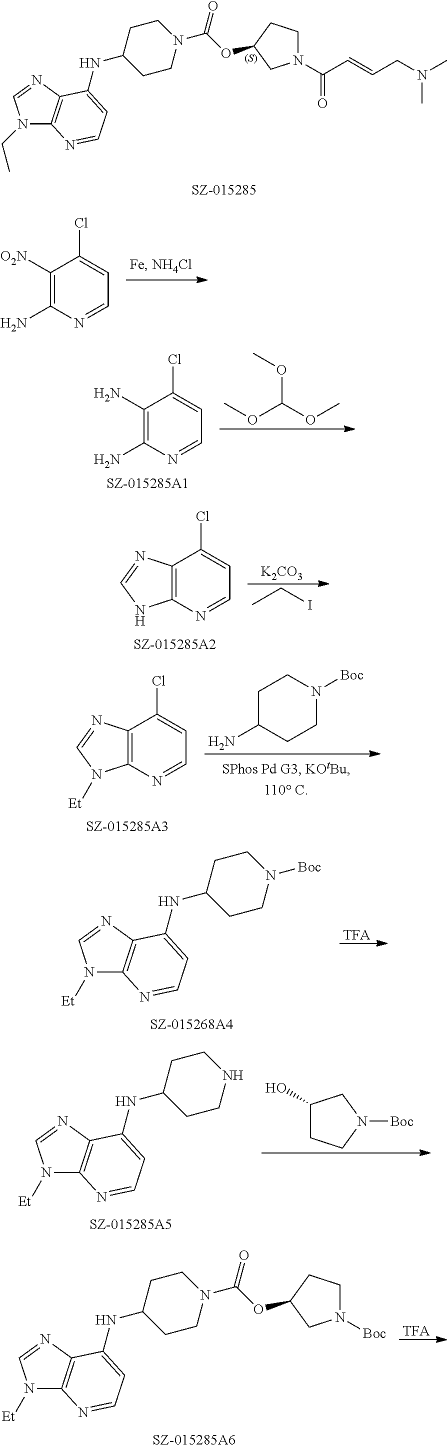

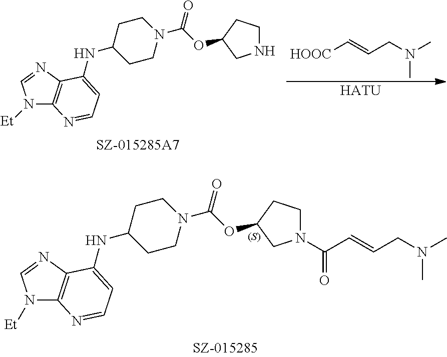

Step 1:

2-amino-3-nitro-4-chloropyridine (3.471 g), iron powder (5.6 g), ammonium chloride (5.349 g), isopropanol (50 mL) and water (10 mL) were added to a reaction flask and stirred at 80° C. for 4 h. After the reaction was completed, the solvent was removed by rotary evaporation, and the residue was dissolved in ethyl acetate (100 mL). The resulting solution was stirred and filtered. The filtrate was collected, and the solvent was removed by rotary evaporation to give a crude product of SZ-015285A1 (a colorless oil), which was directly used in the next step without purification. LCMS: [M+1]⁺143.96.

Step 2:

The crude product of SZ-015285A1 and triethyl orthoformate (100 mL) were added to a reaction flask, DMF was added dropwise until the suspended solids were completely dissolved, and then 12 N hydrochloric acid (2 mL) was added. The reaction solution was stirred at room temperature overnight. After the reaction was completed, the solvent was removed by rotary evaporation, and the residue was purified by flash silica gel column chromatography (EA/PE=0%-40%) to give product SZ-015285A2 (2.1 g). LCMS: [M+1]+153.90.

Step 3:

SZ-015285A2 (1 g), iodoethane (6.08 g), potassium carbonate (5.39 g) and DMSO (6 mL) were added to a reaction flask and stirred at room temperature for 12 h. After the reaction was completed, the solvent was removed by rotary evaporation, water (50 mL) was added, and ethyl acetate (50 mL×3) was added for extraction. The organic phase was collected and dried over anhydrous sodium sulfate, and the solvent was removed by rotary evaporation. The resulting crude product was purified by flash silica gel column chromatography (EA/DCM=0%-40%) to give product SZ-015285A3 (423 mg, 36%). LCMS: [M+1]⁺181.97.

Step 4:

SZ-015285A3 (274.9 mg), 1-Boc-4-aminopiperidine (900 mg), potassium tert-butoxide (504 mg), SPhos Pd G3 (131.25 mg) and tert-amyl alcohol (20 mL) were added to a reaction flask and stirred at 110° C. for 6 h under nitrogen atmosphere. After the reaction was completed, the solvent was removed by rotary evaporation and the resulting crude product was purified by flash silica gel column chromatography (methanol/dichloromethane=0%-5%) to give product SZ-015285A4 (341 mg, 65%). LCMS: [M+1]⁺346.20.

Step 5:

SZ-015285A4 (542 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015285A5, which was directly used in the next step without separation. LCMS: [M+1]+245.96.

Step 6:

(S)-1-N-tert-butoxycarbonyl-3-hydroxypyrrolidine (294.9 mg), acetonitrile (10 mL), triethylamine (1 mL) and p-nitrophenyl chloroformate (302.3 mg) were added successively to a reaction flask and stirred at 40° C. for 2 h. The crude product of SZ-015285A5 in the previous step was dissolved in acetonitrile (10 mL), triethylamine was added to adjust the solution to be alkaline, and then the reaction solution of (S)-1-N-tert-butoxycarbonyl-3-hydroxypyrrolidine was added dropwise to the solution of SZ-015285A5 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-10%) to give SZ-015285A6 (512.3 mg). LCMS: [M+1]⁺459.15.

Step 7:

SZ-015285A6 (160 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015285A7, which was directly used in the next step without separation. LCMS: [M+1]+359.25.

Step 8:

The crude product of SZ-015285A7 in the previous step, (2E)-4-(dimethylamino)-2-butenoic acid hydrochloride (66.24 mg), DIPEA (0.3 mL), T₃P (318 mg) and tetrahydrofuran (10 mL) were added to a reaction flask and stirred at room temperature for 2 h. After the reaction was completed, the solvent was removed by rotary evaporation, and the residue was separated by a reverse phase column (acetonitrile-10 mmol/L aqueous ammonium bicarbonate solution) to give SZ-015285 (21 mg). LCMS: [M+1]⁺470.19.

¹H NMR (400 MHz, DMSO-d₆) δ 8.13 (s, 1H), 7.90 (d, J=5.5 Hz, 1H), 6.65-6.61 (m, 1H), 6.55 (d, J=8.5 Hz, 1H), 6.44-6.35 (m, 2H), 5.18 (d, J=23.9 Hz, 1H), 4.20 (q, J=7.2 Hz, 2H), 3.99-3.97 (m, 0.3H), 3.83-3.73 (m, 0.3H), 3.65-3.50 (m, 2H), 3.05-3.04 (m, 2H), 2.99-2.89 (m, 2H), 2.16 (s, 6H), 2.16-1.92 (m, 4H), 1.46-1.40 (m, 5H).

### Example 13 SZ-015286: (S,E)-1-(4-(dimethylamino)but-2-enoyl)pyrrolidin-3-yl 4-((3-isopropyl-3H-imidazo[4,5-b]pyridin-7-yl)amino)piperidine-1-carboxylate

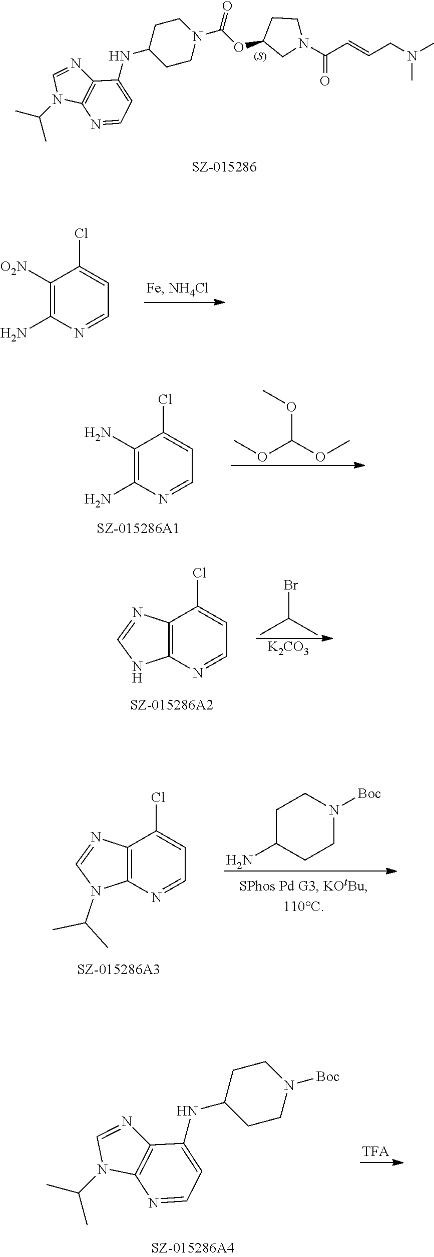

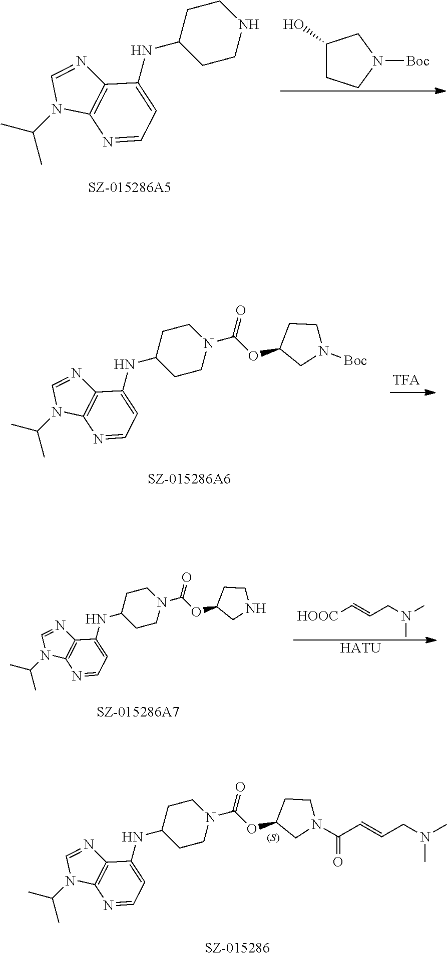

Step 1:

2-amino-3-nitro-4-chloropyridine (3.471 g), iron powder (5.6 g), ammonium chloride (5.349 g), isopropanol (50 mL) and water (10 mL) were added to a reaction flask and stirred at 80° C. for 4 h. After the reaction was completed, the solvent was removed by rotary evaporation, and the residue was dissolved in ethyl acetate (100 mL). The resulting solution was stirred and filtered. The filtrate was collected, and the solvent was removed by rotary evaporation to give a crude product of SZ-015286A1 (a colorless oil), which was directly used in the next step without purification. LCMS: [M+1]⁺142.96.

Step 2:

The crude product of SZ-015286A1 and triethyl orthoformate (100 mL) were added to a reaction flask, DMF was added dropwise until the suspended solids were completely dissolved, and then 12 N hydrochloric acid (2 mL) was added. The reaction solution was stirred at room temperature overnight. After the reaction was completed, the solvent was removed by rotary evaporation, and the residue was purified by flash silica gel column chromatography (EA/PE=0%-40%) to give product SZ-015286A2 (2.1 g). LCMS: [M+1]+153.90.

Step 3:

SZ-015286A2 (1 g), 2-bromopropane (2.5 mL), potassium carbonate (2.8 g) and DMSO (10 mL) were added to a reaction flask and stirred at room temperature for 12 h. After the reaction was completed, the solvent was removed by rotary evaporation, water (50 mL) was added, and ethyl acetate (50 mL×3) was added for extraction. The organic phase was collected and dried over anhydrous sodium sulfate, and the solvent was removed by rotary evaporation. The resulting crude product was purified by flash silica gel column chromatography (EA/DCM=0%-40%) to give product SZ-015286A3 (410 mg). LCMS: [M+1]⁺196.04.

Step 4:

SZ-015286A3 (1.44 g), 1-Boc-4-aminopiperidine (2.8 g), potassium tert-butoxide (1.57 g), SPhos Pd G3 (463 mg) and tert-amyl alcohol (40 mL) were added to a reaction flask and stirred at 110° C. for 6 h under nitrogen atmosphere. After the reaction was completed, the solvent was removed by rotary evaporation, and the resulting crude product was purified by flash silica gel column chromatography (methanol:dichloromethane=0%-5%) to give SZ-015286A4 (1.81 g, 69%). LCMS: [M+1]⁺360.23.

Step 5:

SZ-015286A4 (1.81 g), dichloromethane (10 mL) and TFA (3 mL) were added to a reaction flask and stirred at room temperature for 1 h. After the reaction was completed, the reaction solution was concentrated by rotary evaporation to give a crude product of SZ-015286A5, which was directly used in the next step without separation. LCMS: [M+1]+260.11.

Step 6:

(S)-1-N-tert-butoxycarbonyl-3-hydroxypyrrolidine (973.7 mg), acetonitrile (10 mL), triethylamine (2 mL) and p-nitrophenyl chloroformate (1 g) were added successively to a reaction flask and stirred at 40° C. for 2 h. The crude product of SZ-015286A5 in the previous step was dissolved in acetonitrile (10 mL), triethylamine was added to adjust the solution to be alkaline, and then the reaction solution of (S)-1-N-tert-butoxycarbonyl-3-hydroxypyrrolidine was added dropwise to the solution of SZ-015286A5 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-10%) to give SZ-015286A6 (2.18 g). LCMS: [M+1]⁺473.22.

Step 7:

SZ-015286A6 (300 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015286A7, which was directly used in the next step without separation. LCMS: [M+1]+372.97.

Step 8:

The crude product of SZ-015286A7 in the previous step, (2E)-4-(dimethylamino)-2-butenoic acid hydrochloride (165 mg), DIPEA (0.3 mL), HATU (385 mg) and DMF (5 mL) were added to a reaction flask and stirred at room temperature for 2 h. After the reaction was completed, the solvent was removed by rotary evaporation, and the residue was separated by a reverse phase column (acetonitrile/0.1% ammonium hydroxide solution) to give SZ-015286 (9 mg). LCMS: [M+1]⁺484.28.

¹H NMR (400 MHz, DMSO-d₆) δ 8.20 (s, 1H), 7.89 (d, J=5.4 Hz, 1H), 6.69-6.61 (m, 1H), 6.54 (d, J=8.3 Hz, 1H), 6.49-6.33 (m, 2H), 5.18 (d, J=23.5 Hz, 1H), 4.84-4.74 (m, 1H), 3.99 (br, 3H), 3.87-3.68 (m, 1H), 3.70-3.49 (m, 3H), 3.12 (d, J=5.9 Hz, 2H), 2.96 (br, 2H), 2.21 (s, 6H), 2.16-1.83 (m, 4H), 1.53 (d, J=6.7 Hz, 6H), 145-1.42 (m, 2H).

### Example 14 SZ-015306: (S)-1-((E)-4-(dimethylamino)but-2-enoyl)pyrrolidin-3-yl 4-((5-(((R)-1-hydroxybutan-2-yl)amino)-3-isopropyl-3H-imidazo[4,5-b]pyridin-7-yl)amino)piperidine-1-carboxylate

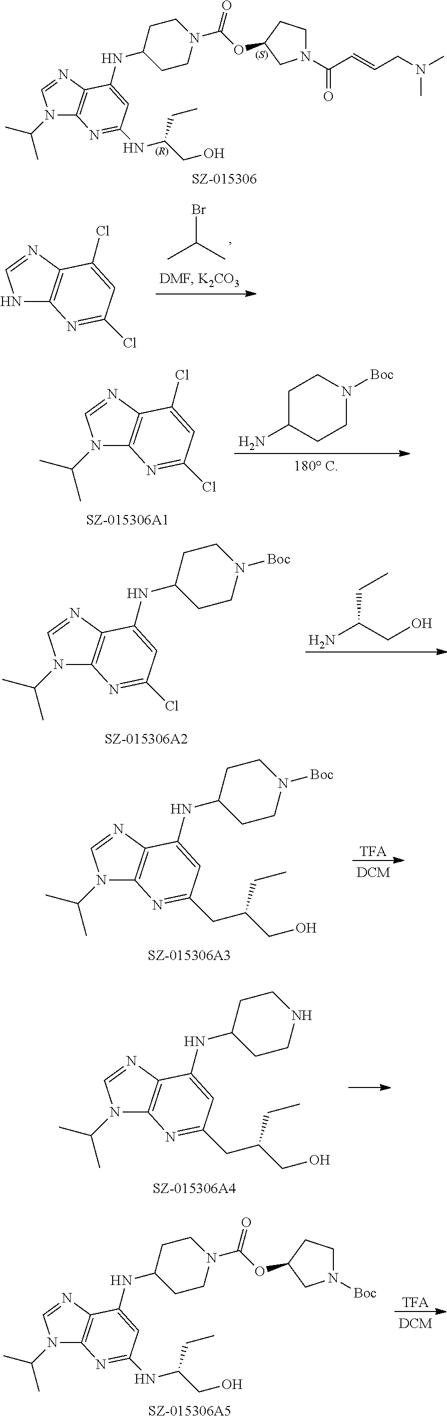

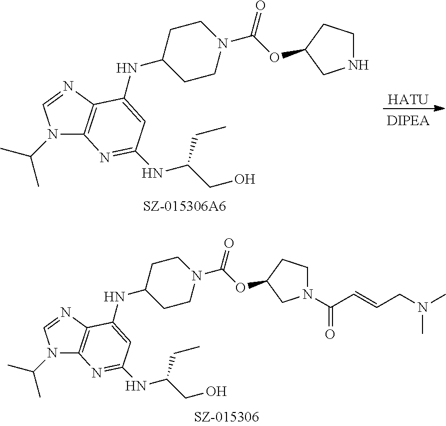

Step 1:

5,7-dichloro-1H-imidazo[4,5-b]pyridine (2.256 g), 2-bromopropane (3.37 mL), potassium carbonate (4.968 g) and DMF (30 mL) were added to a reaction flask and stirred at 60° C. for 12 h. The reaction solution was concentrated by rotary evaporation to remove the solvent, and the residue was diluted with dichloromethane and filtered. The organic phase was concentrated by rotary evaporation to remove the solvent, and the resulting crude product was purified by flash silica gel column chromatography (ethyl acetate/petroleum ether=00%-30%) to give product SZ-015306A1 (2.11 g). LCMS: [M+1]⁺229.02.

Step 2:

SZ-015306A1 (2.11 g) and 1-Boc-4-aminopiperidine (5 g) were added to a reaction flask and stirred at 180° C. for 2 h. After the reaction was completed, the remaining solid was purified by flash silica gel column chromatography (EA/PE=0%-40%) to give product SZ-015306A2 (1.025 g). LCMS: [M+1]⁺394.15.

Step 3:

SZ-015306A2 (492 mg), (R)-2-aminobutanol (223 mg), palladium acetate (14.6 mg), BINAP (40 mg), potassium tert-butoxide (280 mg) and toluene (20 mL) were added to a reaction flask, and the reaction solution was purged with nitrogen three times and stirred at 110° C. for 6 h. After the reaction was completed, the reaction solution was filtered and washed with dichloromethane The organic phase was concentrated by rotary evaporation to remove the solvent, and the resulting crude product was purified by flash silica gel column chromatography (methanol/dichloromethane=0%-5%) to give product SZ-015306A3 (374 mg). LCMS: [M+1]⁺447.32.

Step 4:

SZ-015306A3 (310 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015306A4, which was directly used in the next step without separation. LCMS: [M+1]+347.16.

Step 5:

(S)-1-N-tert-butoxycarbonyl-3-hydroxypyrrolidine (132 mg), acetonitrile (10 mL), triethylamine (1 mL) and p-nitrophenyl chloroformate (142 mg) were added successively to a reaction flask and stirred at 40° C. for 2 h. The crude product of SZ-015306A4 in the previous step was dissolved in acetonitrile (5 mL), triethylamine was added to adjust the solution to be alkaline, and then the reaction solution of (S)-1-N-tert-butoxycarbonyl-3-hydroxypyrrolidine was added dropwise to the solution of SZ-015306A4 in acetonitrile. The resulting reaction solution was stirred at room temperature for 4 h. After the reaction was completed, the solvent was removed by rotary evaporation, and the residue was purified by flash silica gel column chromatography (methanol/dichloromethane=0%-5%) to give SZ-015306A5 (199 mg). LCMS: [M+1]⁺560.22.

Step 6:

SZ-015306A5 (180 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015306A6, which was directly used in the next step without separation. LCMS: [M+1]+460.30.

Step 7:

The crude product of SZ-015306A6 in the previous step, (2E)-4-(dimethylamino)-2-butenoic acid hydrochloride (66 mg), DIPEA (0.5 mL), HATU (152 mg) and DMF (5 mL) were added to a reaction flask and stirred at room temperature for 2 h. After the reaction was completed, the solvent was removed by rotary evaporation, and the residue was separated by a reverse phase column (acetonitrile-water with 0.1% formic acid) to give SZ-015306 (12 mg). LCMS: [M+1]⁺571.27.

1H NMR (400 MHz, DMSO-d₆) δ 9.95 (s, 1H), 6.73-6.57 (m, 2H), 5.84 (s, 1H), 5.18 (d, J=20.4 Hz, 1H), 4.77-4.66 (m, 1H), 4.01-3.71 (m, 7H), 3.70-3.54 (m, 5H), 3.54-3.30 (m, 3H), 7,7 3.11-2.87 (m, 2H), 2.77 (s, 6H), 2.24-1.88 (m, 4H), 1.70-1.60 (m, 1H), 1.59-1.49 (d, J=6.4 Hz, 6H), 1.49-1.33 (m, 3H), 0.9 (t, J=7.4 Hz, 3H).

### Example 15 SZ-015303: (S,E)-1-(4-(dimethylamino)but-2-enoyl)pyrrolidin-3-yl 4-((3-isopropyl-5-(isopropylamino)-3H-imidazo[4,5-b]pyridin-7-yl)amino)piperidine-1-carboxylate

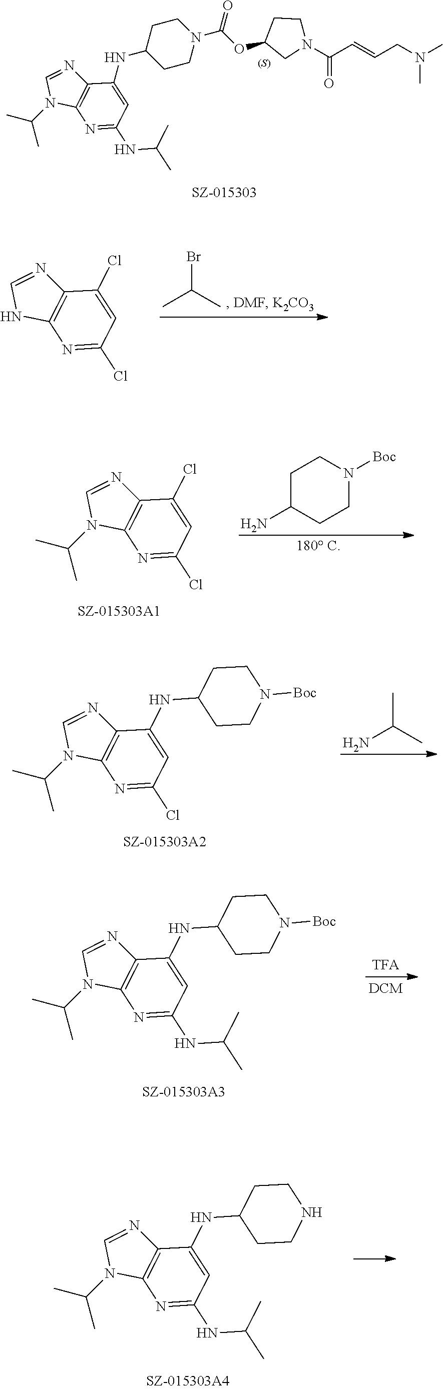

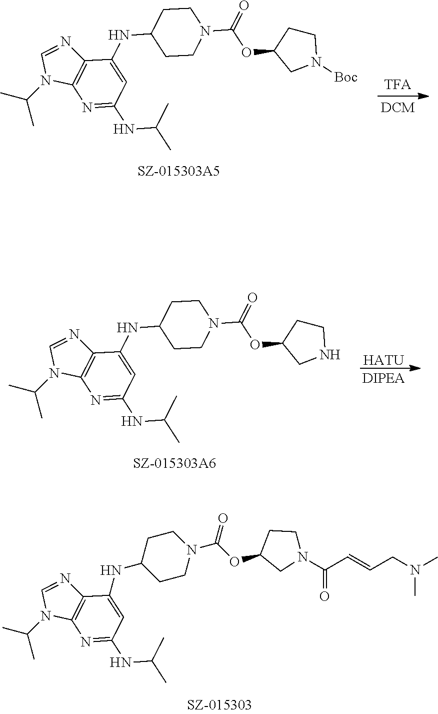

Step 1:

5,7-dichloro-1H-imidazo[4,5-b]pyridine (2.256 g), 2-bromopropane (3.37 mL), potassium carbonate (4.968 g) and DMF (30 mL) were added to a reaction flask and stirred at 60° C. for 12 h. The reaction solution was concentrated by rotary evaporation to remove the solvent, and the residue was diluted with dichloromethane and filtered. The organic phase was concentrated by rotary evaporation to remove the solvent, and the resulting crude product was purified by flash silica gel column chromatography (ethyl acetate/petroleum ether=0%-30%) to give product SZ-015303A1 (2.11 g). LCMS: [M+1]⁺229.02.

Step 2:

SZ-015303A1 (2.11 g) and 1-Boc-4-aminopiperidine (5 g) were added to a reaction flask and stirred at 180° C. for 2 h. After the reaction was completed, the remaining solid was purified by flash silica gel column chromatography (EA/PE=0%-40%) to give product SZ-015303A2 (1.025 g). LCMS: [M+1]⁺394.15.

Step 3:

SZ-015303A2 (170 mg), isopropylamine (150 mg), palladium acetate (5 mg), BINAP (13.7 mg), potassium tert-butoxide (75 mg) and toluene (20 mL) were added to a reaction flask, and the reaction solution was purged with nitrogen three times and stirred at 110° C. for 6 h. After the reaction was completed, the reaction solution was filtered and washed with dichloromethane The organic phase was concentrated by rotary evaporation to remove the solvent, and the resulting crude product was purified by flash silica gel column chromatography (methanol/dichloromethane=0%-5%) to give product SZ-015303A3 (220 mg). LCMS: [M+1]⁺417.34.

Step 4:

SZ-015303A3 (220 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015303A4, which was directly used in the next step without separation. LCMS: [M+1]+317.05.

Step 5:

(S)-1-N-tert-butoxycarbonyl-3-hydroxypyrrolidine (112.3 mg), acetonitrile (10 mL), triethylamine (1 mL) and p-nitrophenyl chloroformate (120.93 mg) were added successively to a reaction flask and stirred at 40° C. for 2 h. The crude product of SZ-015303A4 in the previous step was dissolved in acetonitrile (5 mL), triethylamine was added to adjust the solution to be alkaline, and then the reaction solution of (S)-1-N-tert-butoxycarbonyl-3-hydroxypyrrolidine was added dropwise to the solution of SZ-015303A4 in acetonitrile. The resulting reaction solution was stirred at room temperature for 4 h. After the reaction was completed, the solvent was removed by rotary evaporation, and the residue was purified by flash silica gel column chromatography (methanol/dichloromethane=0%-5%) to give SZ-015303A5 (217 mg). LCMS: [M+1]⁺530.22.

Step 6:

SZ-015303A5 (217 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015303A6, which was directly used in the next step without separation. LCMS: [M+1]+430.15.

Step 7:

The crude product of SZ-015303A6 in the previous step, (2E)-4-(dimethylamino)-2-butenoic acid hydrochloride (67 mg), DIPEA (0.5 mL), HATU (155 mg) and DMF (5 mL) were added to a reaction flask and stirred at room temperature for 2 h. After the reaction was completed, the solvent was removed by rotary evaporation, and the residue was separated by a reverse phase column (acetonitrile-water with 0.1% formic acid) to give SZ-015303 (41 mg). LCMS: [M+1]⁺541.02.

¹H NMR (400 MHz, DMSO-d₆) δ 9.66 (br, 1H), 6.84-6.56 (m, 3H), 5.78 (s, 1H), 5.21 (d, J=19.6 Hz, 1H), 4.73 (br, 1H), 4.10-3.79 (m, 7H), 3.72-3.51 (m, 4H), 3.04 (br, 1H), 2.80 (s, 6H), 2.32-1.82 (m, 4H), 1.57 (d, J=6.4 Hz, 6H), 1.51 (d, J=6.8 Hz, 1H), 1.45=1.42 (m, 2H), 1.20 (d, J=6.1 Hz, 6H).

### Example 16 SZ-015307: (E)-1-(4-(dimethylamino)but-2-enoyl)azetidin-3-yl 4-((3-isopropyl-5-methyl-3H-imidazo[4,5-b]pyridin-7-yl)amino)piperidine-1-carboxylate

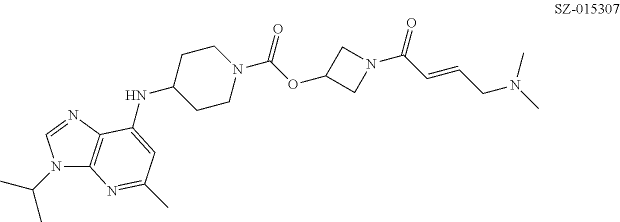 [SMILES: Cc1cc(NC2CCN(C(=O)OC3CN(C(=O)/C=C/CN(C)C)C3)CC2)c2ncn(C(C)C)c2n1]

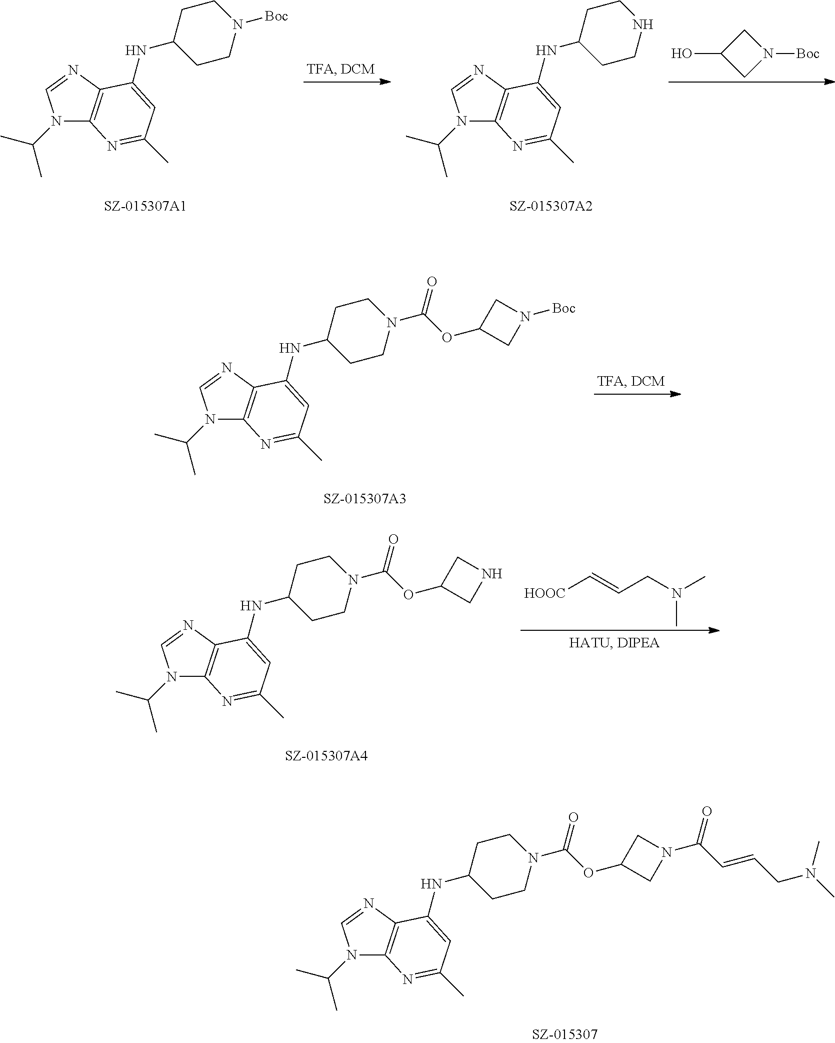

### Step 1: 3-isopropyl-5-methyl-N-(piperidin-4-yl)-3H-imidazo[4,5-b]pyridin-7-amine

SZ-015307A1 (015095A5) (597 mg), dichloromethane (5 mL) and TFA (1 mL) were added to a reaction flask and stirred at room temperature for 1 h. After the reaction was completed, the solvent was removed by rotary evaporation, and the resulting crude product of SZ-015307A2 was directly used in the next step without separation. LCMS: [M+H]⁺ 274.10.

### Step 2: 1-(tert-butoxycarbonyl)azetidin-3-yl 4-((3-isopropyl-5-methyl-3H-imidazo [4,5-b]pyridin-7-yl)amino)piperidine-1-carboxylate

1-N-tert-butoxycarbonyl-3-hydroxyazetidine (260 mg), acetonitrile (5 mL), triethylamine (1 mL) and p-nitrophenyl chloroformate (303 mg) were added successively to a reaction flask and stirred at 40° C. for 2 h. The crude product of SZ-015307A2 in the previous step was dissolved in acetonitrile (5 mL), triethylamine was added to adjust the solution to be alkaline, and then the reaction solution of (S)-1-N-tert-butoxycarbonyl-3-hydroxyazetidine was added dropwise to the solution of SZ-015307A2 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-5%) to give SZ-015307A3 (532 mg). LCMS: [M+H]⁺ 472.28.

### Step 3: azetidin-3-yl 4-((3-isopropyl-5-methyl-3H-imidazo[4,5-b]pyridin-7-yl)amino) piperidin-1-carboxylate

SZ-015307A3 (389 mg), dichloromethane (5 mL) and TFA (1 mL) were added to a reaction flask and stirred at room temperature for 1 h. After the reaction was completed, the solvent was removed by rotary evaporation, and the resulting crude product of SZ-015307A4 was directly used in the next step without separation. LCMS: [M+H]⁺ 373.20.

### Step 4

The crude product of SZ-015307A4, (2E)-4-(dimethylamino)-2-butenoic acid hydrochloride (165 mg), DIPEA (0.5 mL), HATU (456 mg) and DMF (20 mL) were added to a reaction flask and stirred at room temperature for 2 h. After the reaction was completed, the solvent was removed by rotary evaporation, and the residue was separated by a reverse phase column (methanol/0.2% ammonium hydroxide solution) and lyophilized to give SZ-015307 (17 mg, 23%, a white solid). LCMS: [M+H]⁺ 484.25.

¹H NMR (400 MHz, DMSO-d₆) δ 8.13 (s, 1H), 6.61 (dt, J=15.3, 6.2 Hz, 1H), 6.42 (d, J=8.6 Hz, 1H), 6.30 (s, 1H), 6.13 (d, J=15.4 Hz, 1H), 5.17-5.03 (m, 1H), 4.84-4.70 (m, 1H), 4.63-4.45 (m, 1H), 4.23 (dd, J=10.9, 6.9 Hz, 1H), 4.15 (dd, J=9.9, 3.6 Hz, 1H), 4.03 (br, 3H), 3.85 (dd, J=11.1, 3.5 Hz, 1H), 3.05-2.95 (m, 4H), 2.40 (s, 3H), 2.17 (s, 6H), 1.99-1.86 (m, 2H), 1.54-1.48 (m, 8H).

### Example 17 SZ-015311: (S,E)-1-(4-(dimethylamino)but-2-enoyl)pyrrolidin-3-yl 4-((3-isopropyl-5-((2-methoxyethyl)amino)-3H-imidazo[4,5-b]pyridin-7-yl)amino)piperidine-1-carboxylate

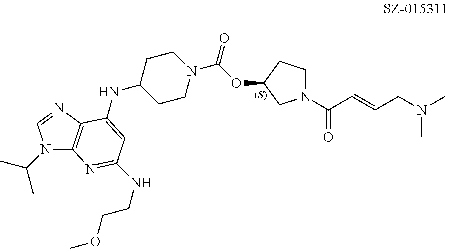

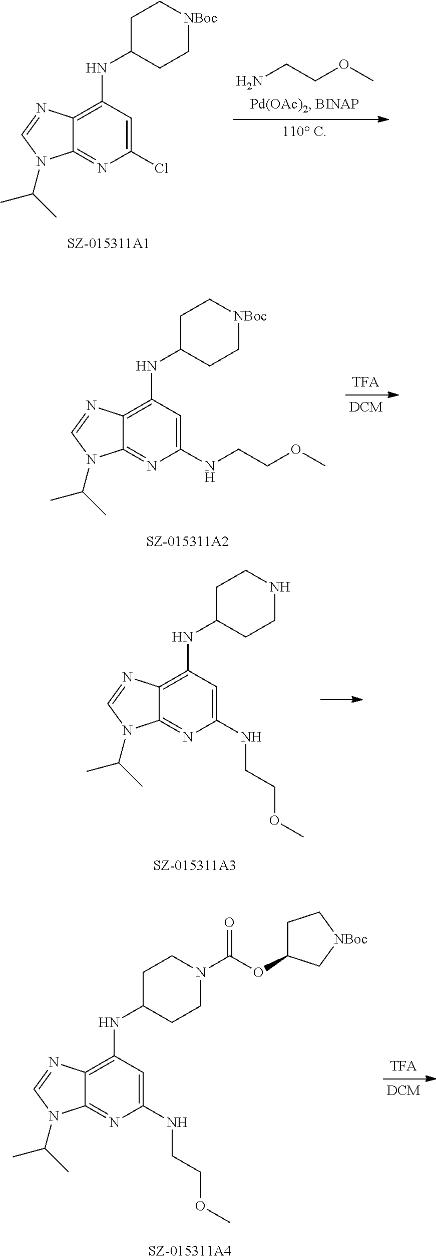

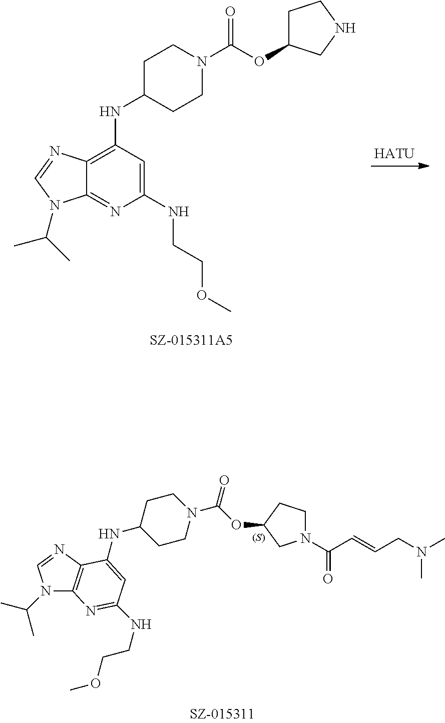

Step 1:

SZ-015311A1 (015306A2) (300 mg, 0.76 mmol), 2-methoxyethylamine (190 mg, 2.5 mmol), potassium tert-butoxide (180 mg, 1.6 mmol), palladium acetate (50 mg) and BINAP (50 mg) were mixed in anhydrous toluene (10 mL) under argon atmosphere, and the reaction solution was stirred at 110° C. for 4 h. The reaction solution was directly concentrated by rotary evaporation, and the residue was purified by flash silica gel column chromatography (PE:EA=1:2; Rf=0.3) to give SZ-015311A2 (340 mg, a foamy solid). LCMS: [M+H]⁺ 433.32.

Step 2:

SZ-015311A2 (340 mg) was dissolved in dichloromethane (5 mL), and trifluoroacetic acid (32 mL) was slowly added dropwise. The reaction solution was stirred at room temperature for about 2 h, and then directly concentrated by rotary evaporation to give a crude product of SZ-015311A3 (200 mg).LCMS: [M+H]⁺ 333.31.

Step 3:

Tert-butyl (S)-3-hydroxypyrrolidine-1-carboxylate (147 mg, 0.78 mmol), p-nitrophenyl chloroformate (158 mg, 0.78 mmol) and triethylamine (0.5 mL) were mixed in acetonitrile (4 mL), and the reaction solution was stirred at 40° C. for 2 h. Then the crude product of SZ-015311A3 was added, and the resulting reaction solution was stirred at room temperature overnight. The reaction solution was directly concentrated by rotary evaporation, and the residue was purified by flash silica gel column chromatography (PE:EA=1:4; Rf=0.2) to give SZ-015311A4 (220 mg, a foamy solid). LCMS: [M+H]⁺ 546.21.

Step 4:

SZ-015311A4 (220 mg) was dissolved in dichloromethane (4 mL), and trifluoroacetic acid (2 mL) was slowly added dropwise. The reaction solution was stirred at room temperature for about 2 h, and then directly concentrated by rotary evaporation to give a crude product of SZ-015311A5 (200 mg).LCMS: [M+H]⁺ 446.26.

Step 5:

SZ-015311A5 obtained in the previous step, trans-4-dimethylaminocrotonic acid hydrochloride (55 mg, 0.33 mmol), Et₃N (0.2 mL) and HATU (150 mg, 0.39 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015311 (26 mg, a light yellow solid). LCMS: [M+H]⁺ 557.37.

¹H NMR (400 MHz, DMSO-d₆) 7.76 (s, 1H), 6.69-6.61 (m, 1H), 6.41-6.34 (m, 1H), 5.98-5.95 (m, 2H), 5.59 (s, 1H), 5.20-5.14 (m, 1H), 4.65-4.58 (m, 1H), 3.98-3.91 (m, 2H), 3.84-3.72 (m, 2H), 3.64-3.46 (m, 5H), 3.43-3.40 (m, 2H), 3.29 (s, 3H), 3.06-3.05 (m, 2H), 2.93-2.92 (m, 2H), 2.17 (s, 6H), 2.09-1.90 (m, 4H), 1.50-1.48 (d, J=6.8 Hz, 6H), 1.43-1.37 (m, 2H).

### Example 18 SZ-015301: (S,E)-N-(1-(4-(dimethylamino)but-2-enoyl)pyrrolidin-3-yl)-4-((3-isopropyl-5-methyl-3H-imidazo[4,5-b]pyridin-7-yl)amino)piperidine-1-carboxamide

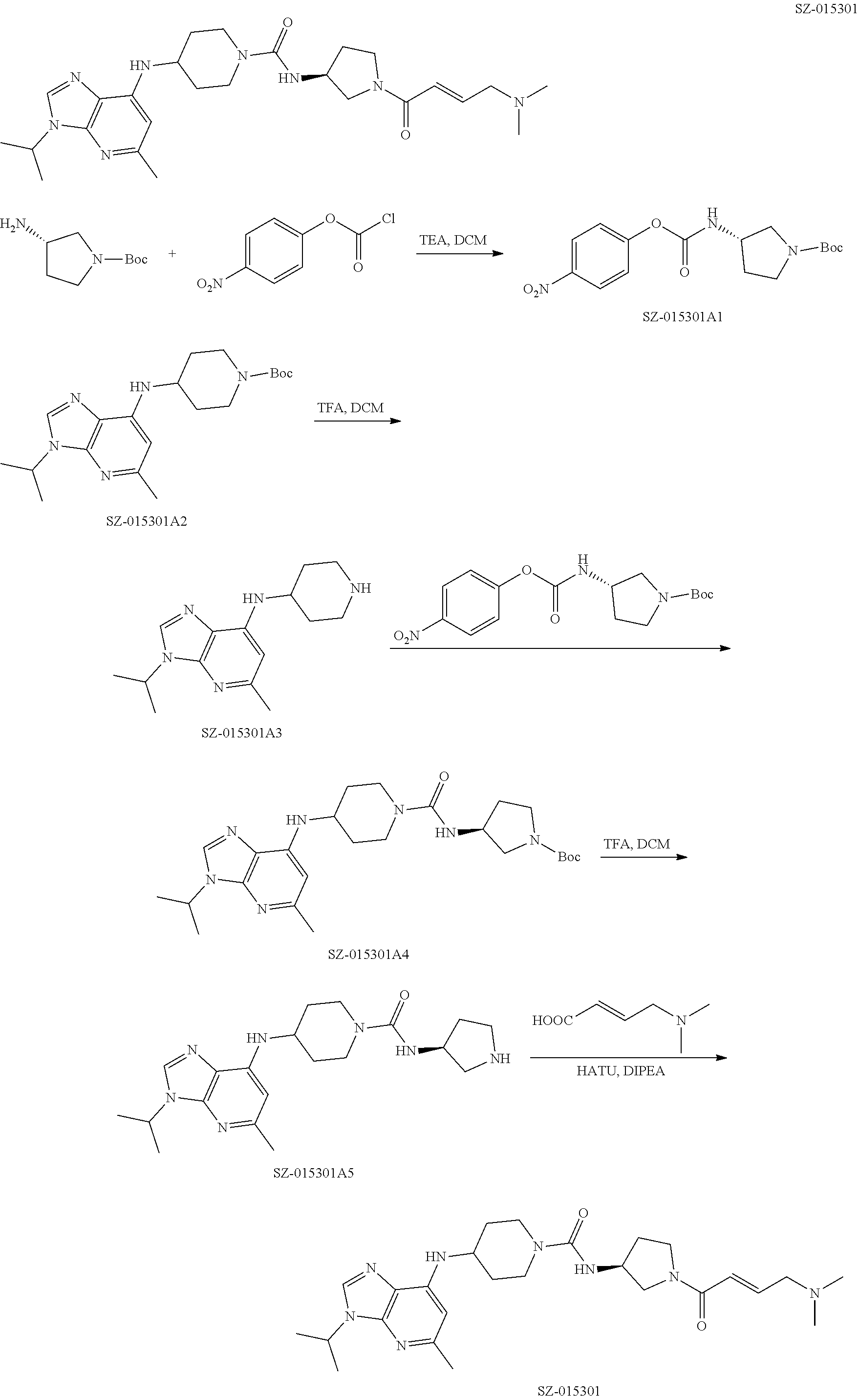

Step 1:

(S)-1-tert-butoxycarbonyl-3-aminopyrrolidine (838 mg), dichloromethane (15 mL) and triethylamine (1.5 mL) were added to a reaction flask. P-nitrophenyl chloroformate (1.088 g) was added with stirring, and the resulting reaction solution was stirred at 60° C. for 12 h. After the reaction was completed, the solvent was removed by rotary evaporation, and the residue was purified by flash silica gel column chromatography (EA/PE=50%-100%) to give product SZ-015301A1 (319 mg, 22%, a light yellow oil). LCMS: [M+1]⁺251.98, 295.96.

Step 2:

SZ-015301A2 (015095A5) (373 mg), dichloromethane (5 mL) and TFA (1 mL) were added to a reaction flask and stirred at room temperature for 1 h. After the reaction was completed, the solvent was removed by rotary evaporation, and the resulting crude product of SZ-015301A3 was directly used in the next step without separation. LCMS: [M+1]⁺274.10.

Step 3:

SZ-015301A3 (crude product), SZ-015301A1 (319 mg) and pyridine (10 mL) were added to a reaction flask and stirred at 100° C. for 12 h. After the reaction was completed, the solvent was removed by rotary evaporation, and the residue was purified by flash silica gel column chromatography (MeOH/DCM=0%-10%) to give product SZ-015301A4 (151 mg, 34%, a light yellow oil). LCMS: [M+1]⁺ 486.29.

Step 4:

SZ-015301A4 (150 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015301A5, which was directly used in the next step without separation. LCMS: [M+1]+386.19.

Step 5:

The crude product of SZ-015301A5, (2E)-4-(dimethylamino)-2-butenoic acid hydrochloride (66.24 mg), DIPEA (0.3 mL), HATU (190 mg) and DMF (5 mL) were added to a reaction flask and stirred at room temperature for 2 h. After the reaction was completed, the solvent was removed by rotary evaporation, and the residue was separated by a reverse phase column (acetonitrile-0.1% aqueous ammonium hydroxide solution) and lyophilized to give SZ-015301 (34 mg, 23%, a white solid). LCMS: [M+1]⁺ 497.26.

¹H NMR (400 MHz, DMSO-d₆) δ 8.12 (s, 1H), 6.77-6.46 (m, 2H), 6.44-6.21 (m, 3H), 4.80-4.74 (m, 1H), 4.22-4.12 (m, 1H), 4.01 (d, J=12.9 Hz, 3H), 3.78 (dd, J=10.2, 6.7 Hz, 1H), 3.31-3.22 (m, 1H), 3.05 (d, J=6.0 Hz, 2H), 2.82 (t, J=12.6 Hz, 2H), 2.39 (s, 3H), 2.17 (s, 6H), 2.01 (m, 1H), 1.85 (m, 3H), 1.50 (d, J=6.7 Hz, 6H), 1.41-1.28 (m, 2H).

### Example 19 SZ-015302: (S,E)-N-(1-(4-(dimethylamino)but-2-enoyl)pyrrolidin-3-yl)-4-((3-isopropyl-5-methyl-3H-imidazo[4,5-b]pyridin-7-yl)amino)-N-methylpiperidine-1-carboxamide

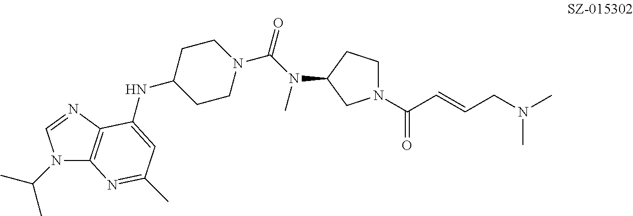 [SMILES: Cc1cc(NC2CCN(C(=O)N(C)[C@H]3CCN(C(=O)/C=C/CN(C)C)C3)CC2)c2ncn(C(C)C)c2n1]

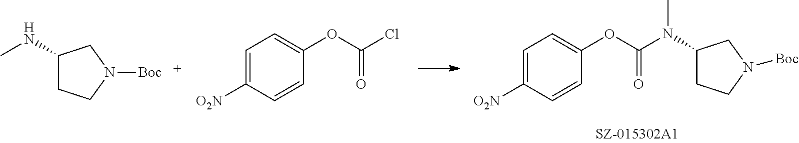 [SMILES: CN[C@H]1CCN(C(=O)OC(C)(C)C)C1 ; O=C(Cl)Oc1ccc([N+](=O)[O-])cc1 ; CN(C(=O)Oc1ccc([N+](=O)[O-])cc1)[C@H]1CCN(C(=O)OC(C)(C)C)C1]

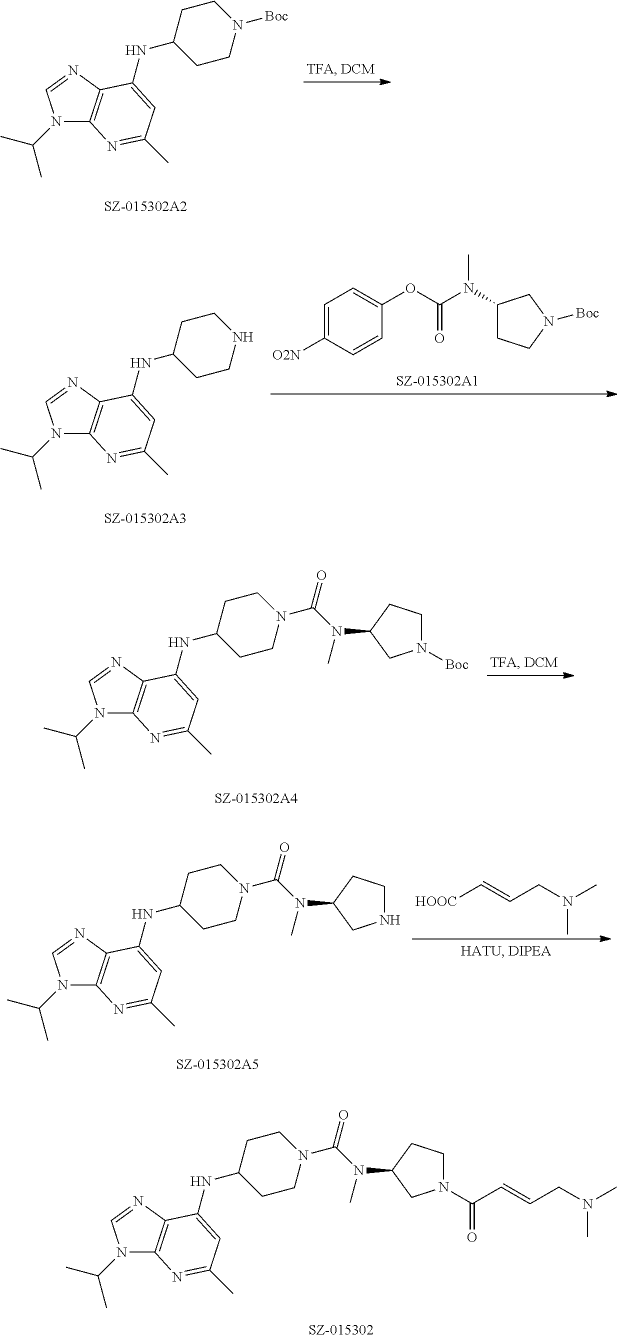

Step 1:

(S)-1-Boc-3-(methylamino)pyrrolidine (300 mg), dichloromethane (15 mL) and triethylamine (1.5 mL) were added to a reaction flask. P-nitrophenyl chloroformate (362 mg) was added with stirring, and the resulting reaction solution was stirred at 60° C. for 12 h. After the reaction was completed, the solvent was removed by rotary evaporation, and the residue was purified by flash silica gel column chromatography (EA/PE=0%-50%) to give product SZ-015302A1 (555 mg). LCMS: [M+1]⁺266.03, 310.01.

Step 2:

SZ-015302A2 (015095A5) (522 mg), dichloromethane (5 mL) and TFA (1 mL) were added to a reaction flask and stirred at room temperature for 1 h. After the reaction was completed, the solvent was removed by rotary evaporation, and the resulting crude product of SZ-015302A3 was directly used in the next step without separation. LCMS: [M+1]⁺274.10.

Step 3:

SZ-015302A3 (crude product), SZ-015302A1 (555 mg) and pyridine (20 mL) were added to a reaction flask and stirred at 100° C. for 12 h. After the reaction was completed, the solvent was removed by rotary evaporation, and the residue was purified by flash silica gel column chromatography (MeOH/DCM=0%-5%) to give product SZ-015302A4 (219 mg). LCMS: [M+1]⁺500.29.

Step 4:

SZ-015302A4 (100 mg), dichloromethane (5 mL) and TFA (1 mL) were added to a reaction flask and stirred at room temperature for 1 h. After the reaction was completed, the reaction solution was concentrated by rotary evaporation to give a crude product of SZ-015302A5, which was directly used in the next step without separation. LCMS: [M+1]+400.26.

Step 5:

The crude product of SZ-015302A5 in the previous step, (2E)-4-(dimethylamino)-2-butenoic acid hydrochloride (41.25 mg), DIPEA (0.3 mL), HATU (114 mg) and DMF (5 mL) were added to a reaction flask and stirred at room temperature for 2 h. After the reaction was completed, the solvent was removed by rotary evaporation, and the residue was separated by a reverse phase column (acetonitrile/10 mmol/L aqueous ammonium acetate solution) and lyophilized to give SZ-015302 (31 mg). LCMS: [M+1]⁺511.28.

¹H NMR (400 MHz, DMSO-d₆) δ 8.22 (s, 1H), 6.66-6.60 (m, 1H), 6.43-6.36 (m, 2H), 6.29 (s, 1H), 4.79-4.75 (m, 1H), 4.31-4.13 (m, 1H), 4.04 (s, 1H), 3.81-3.60 (m, 4H), 3.54-3.43 (m, 1H), 3.25-3.23 (m, 1H), 3.09 (d, J=6.1 Hz, 2H), 2.90-2.85 (m, 2H), 2.75 (d, J=4.7 Hz, 3H), 2.40 (s, 3H), 2.20 (s, 6H), 2.15-1.85 (m, 4H), 1.51 (m, 8H).

### Example 20 SZ-015292: (S,E)-N-(1-(4-(dimethylamino)but-2-enoyl)pyrrolidin-3-yl)-4-((9-isopropyl-2-methyl-9H-purin-6-yl)amino)piperidine-1-carboxamide

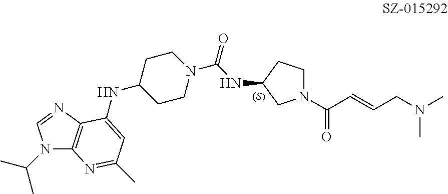

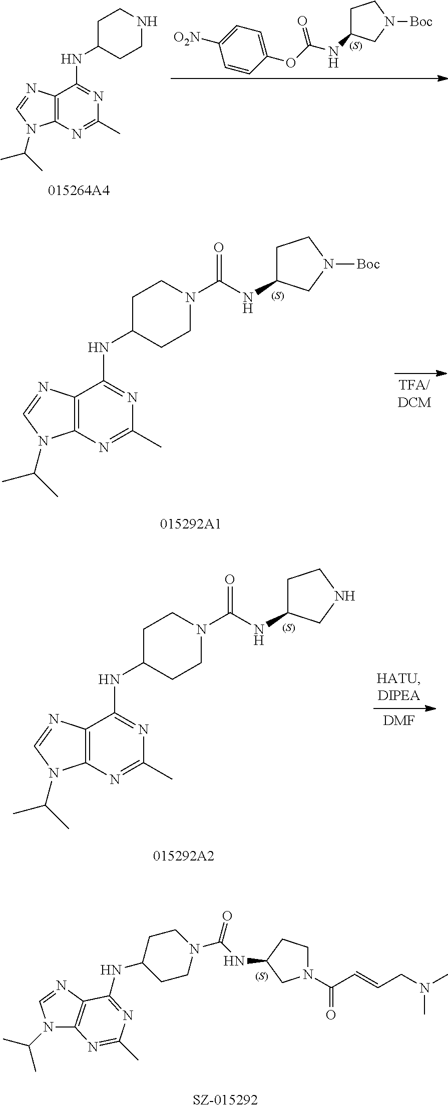

Step 1:

Compound 015264A4 trifluoroacetate (750 mg, 2 mmol) was dissolved in pyridine (15 mL), and tert-butyl (S)-3-(((4-nitrophenoxy)carbonyl)amino)pyrrolidine-1-carboxylate (720 mg, 2 mmol) was added. The reaction solution was heated to 100° C., stirred for 6 h, and concentrated by rotary evaporation under reduced pressure to give a crude product of 015292A1, which was purified by flash silica gel column chromatography to give 015292A1 (500 mg, a white solid). LCMS: [M+H]⁺ 487.5.

Step 2:

Compound 015292A1 (500 mg, 1 mmol) was dissolved in dichloromethane (6 mL), and trifluoroacetic acid (2 mL) was added. The reaction solution was stirred at room temperature for 0.5 h and concentrated by rotary evaporation under reduced pressure to give 015292A2 trifluoroacetate (500 mg, 100%, a colorless oily liquid). LCMS: [M+H]⁺ 387.4.

Step 3:

Compound 015292A2 trifluoroacetate (500 mg, 1 mmol) was dissolved in DMF (5 mL), and DIPEA (390 mg, 3 mmol), (E)-4-(dimethylamino)but-2-enoic acid hydrochloride (166 mg, 1 mmol) and HATU (456 mg, 1.2 mmol) were added successively. The reaction solution was stirred at room temperature for 0.5 h and concentrated by rotary evaporation under reduced pressure to give a crude product of SZ-015292, which was subjected to reverse phase column chromatography (acetonitrile/water with 0.1% formic acid) and lyophilized to give SZ-015292 (40 mg, a white solid). LCMS: [M+H]⁺ 498.2.

¹H NMR (400 MHz, DMSO-d₆) δ 9.60 (s, 1H), 8.41 (s, 1H), 6.75-6.45 (m, 3H), 4.75 (s, 1H), 4.35-3.85 (m, 7H), 3.72-3.49 (m, 6H), 2.77 (d, J=4.4 Hz, 8H), 2.16-1.95 (m, 1H), 1.98-1.78 (m, 3H), 1.51 (d, J=6.7 Hz, 8H).

### Example 21 SZ-015293: (S,E)-1-(4-(dimethylamino)but-2-enoyl)pyrrolidin-3-yl 4-((9-isopropyl-2,8-dimethyl-9H-purin-6-yl)amino)piperidine-1-carboxylate

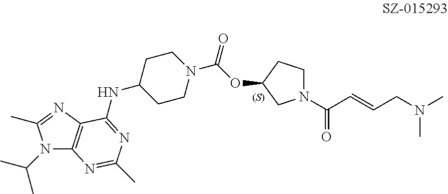

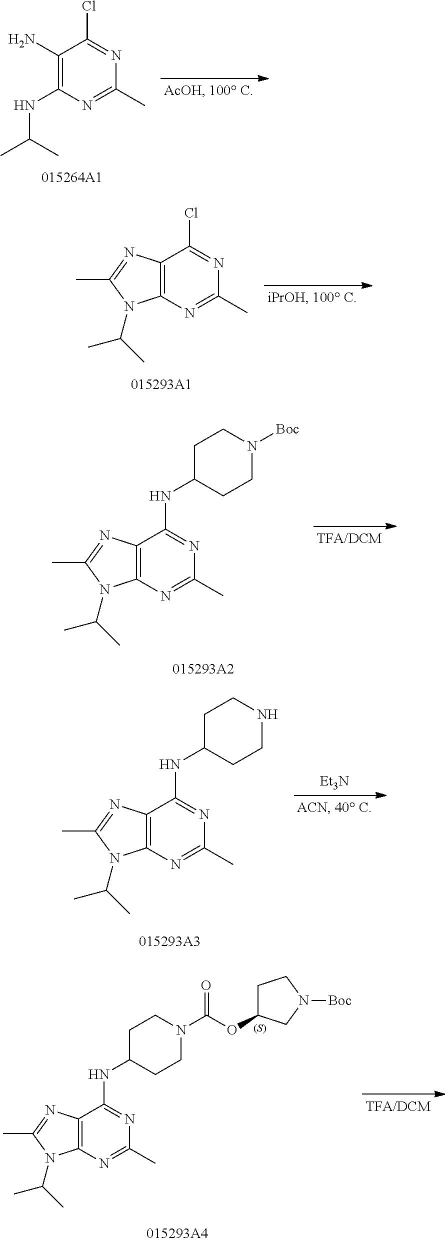

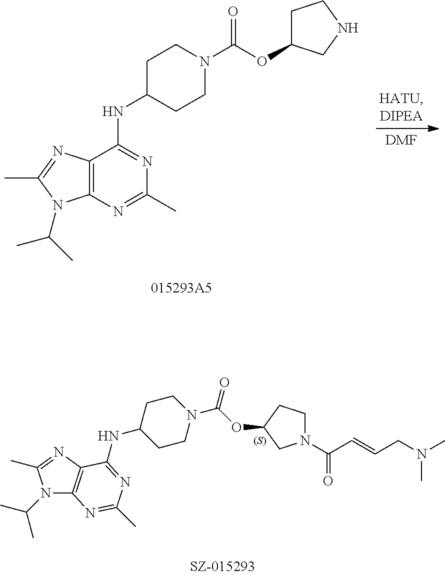

Step 1:

015264A1 (2.0 g, 10 mmol), trimethyl orthoacetate (9 mL) and acetic acid (4 mL) were added to a microwave flask, and the reaction solution was heated to 100° C. and reacted for 3 h under a microwave condition. After the reaction was completed, the reaction solution was cooled to room temperature, and the solvent was removed by rotary evaporation. The residue was concentrated under reduced pressure to give a crude product, which was purified by flash silica gel column chromatography to give 015293A1 (500 mg, a white solid). LCMS: [M+H]⁺225.6.

Step 2:

Compound 015293A1 (500 mg, 2.23 mmol) was dissolved in isopropanol (10 mL), and tert-butyl-4-aminopiperidine-1-carboxylate (445 mg, 2.23 mmol) was added. The reaction solution was heated to 100° C. and stirred for 6 h, and then the reaction solution was cooled to room temperature and concentrated by rotary evaporation under reduced pressure to give a crude product of 015293A2, which was purified by flash silica gel column chromatography to give 015293A2 (500 mg, a white solid). LCMS: [M+H]⁺ 389.4.

Step 3:

Compound 015293A2 (500 mg, 1.29 mmol) was dissolved in dichloromethane (9 mL), and trifluoroacetic acid (3 mL) was added. The reaction solution was stirred at room temperature for 1 h and concentrated by rotary evaporation under reduced pressure to give 015293A3 trifluoroacetate (500 mg, 100%, a colorless oily liquid). LCMS: [M+H]⁺ 289.3.

Step 4:

Compound 015293A3 trifluoroacetate (500 mg, 1.29 mmol) was dissolved in acetonitrile (9 mL), and triethylamine (393 mg, 3.9 mmol) and tert-butyl (S)-3-(((4-nitrophenoxy)carbonyl)oxo)pyrrolidine-1-carboxylate (507 mg, 1.44 mmol) were added successively. The reaction solution was stirred at room temperature for 2 h and concentrated by rotary evaporation under reduced pressure to give a crude product of 015293A4, which was purified by flash silica gel column chromatography to give 015293A4 (150 mg, a white solid). LCMS: [M+H]⁺ 502.5.

Step 5:

Compound 015293A4 (150 mg, 0.3 mmol) was dissolved in dichloromethane (6 mL), and trifluoroacetic acid (2 mL) was added. The reaction solution was stirred at room temperature for 0.5 h and concentrated by rotary evaporation under reduced pressure to give 015293A5 trifluoroacetate (150 mg, 100%, a colorless oily liquid). LCMS: [M+H]⁺ 402.4.

Step 6:

Compound 015293A5 trifluoroacetate (150 mg, 0.3 mmol) was dissolved in DMF (5 mL), and DIPEA (130 mg, 1 mmol), (E)-4-(dimethylamino)but-2-enoic acid hydrochloride (50 mg, 0.3 mmol) and HATU (137 mg, 0.36 mmol) were added successively. The reaction solution was stirred at room temperature for 0.5 h and concentrated by rotary evaporation under reduced pressure to give a crude product of SZ-015293, which was subjected to reverse phase column chromatography (acetonitrile/water with 0.1% formic acid) and lyophilized to give SZ-015293 (21.2 mg, a white solid). LCMS: [M+H]⁺ 513.17.

¹H NMR (400 MHz, DMSO-d₆) δ 7.26 (d, J=8.1 Hz, 1H), 6.62 (ddd, J=16.0, 10.5, 5.8 Hz, 1H), 6.37 (dd, J=20.3, 15.5 Hz, 1H), 5.15 (d, J=23.0 Hz, 1H), 4.65 (dt, J=13.3, 6.6 Hz, 1H), 4.45-4.29 (m, 1H), 3.93-3.30 (m, 6H), 3.05 (d, J=5.8 Hz, 2H), 2.95-2.90 (m, 2H), 2.51 (s, 3H), 2.49 (s, 3H), 2.15 (s, 6H), 2.02 (dd, J=21.6, 8.3 Hz, 2H), 1.79 (s, 2H), 1.52-1.48 (t, J=17.6 Hz, 8H).

### Example 22 SZ-015296: (S,E)-1-(4-(dimethylamino)but-2-enoyl)pyrrolidin-3-yl 4-((9-ethyl-2-methyl-9H-purin-6-yl)amino)piperidine-1-carboxylate

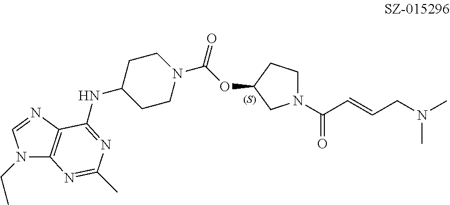

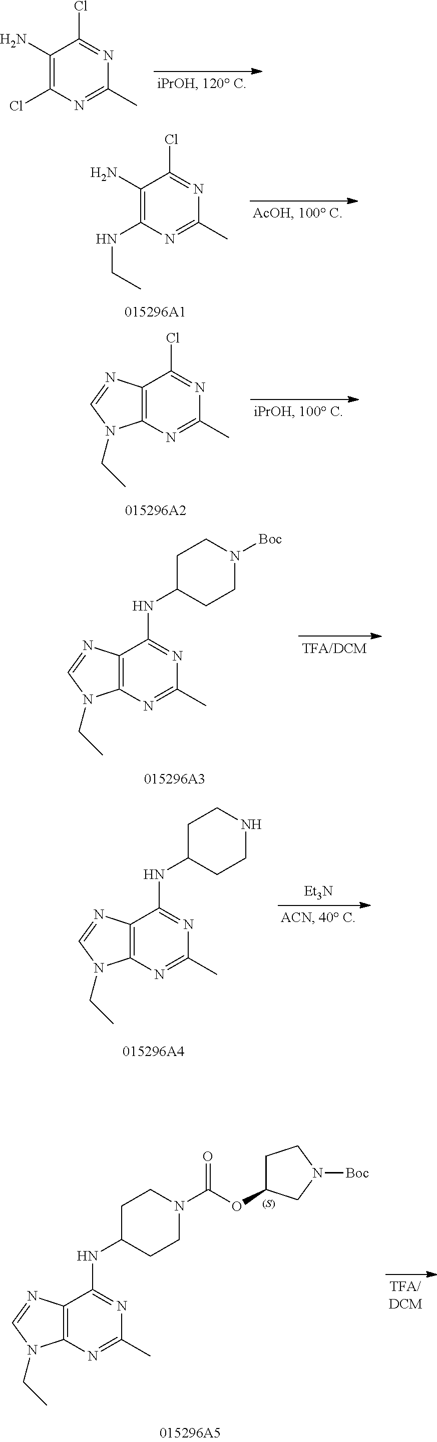

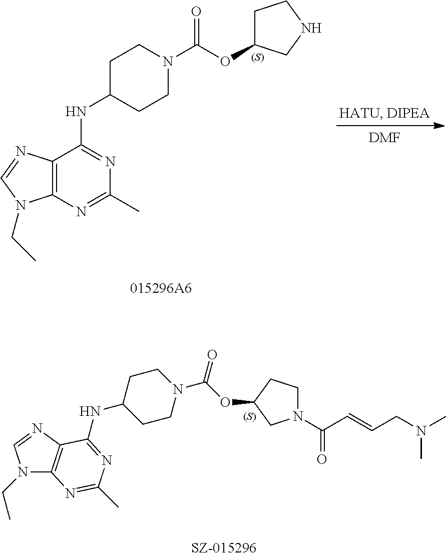

Step 1:

4,6-dichloro-2-methylpyrimidin-5-amine (1.0 g, 5.7 mmol), ethylamine (2.6 g, 57 mmol) and isopropanol (10 mL) were added to a microwave flask, and the reaction solution was heated to 120° C. and reacted for 8 h under a microwave condition. The reaction solution was cooled to room temperature and distilled under reduced pressure to give the product 015296A1 (0.8 g, 75%, a light yellow solid). LCMS: [M+H]⁺ 187.5.

Step 2:

015296A1 (0.8 g, 4.3 mmol), trimethyl orthoformate (9 mL) and acetic acid (4 mL) were added to a microwave flask, and the reaction solution was heated to 100° C. and reacted for 3 h under a microwave condition. After the reaction was completed, the reaction solution was cooled to room temperature, and the solvent was removed by rotary evaporation. The residue was concentrated under reduced pressure to give a crude product, which was purified by flash silica gel column chromatography to give 015296A2 (0.8 g, 95%, a white solid). LCMS: [M+H]⁺ 197.5.

Step 3:

Compound 015296A2 (0.8 g, 4.08 mmol) was dissolved in isopropanol (10 mL), and tert-butyl-4-aminopiperidine-1-carboxylate (0.82 g, 4.08 mmol) was added. The reaction solution was heated to 100° C. and stirred for 6 h, and then the reaction solution was cooled to room temperature and concentrated by rotary evaporation under reduced pressure to give a crude product of 015296A3, which was purified by flash silica gel column chromatography to give 015296A3 (800 mg, 56%, a white solid). LCMS: [M+H]⁺ 361.3.

Step 4:

Compound 015296A3 (800 mg, 2.22 mmol) was dissolved in dichloromethane (9 mL), and trifluoroacetic acid (3 mL) was added. The reaction solution was stirred at room temperature for 1 h and concentrated by rotary evaporation under reduced pressure to give 015296A4 trifluoroacetate (800 mg, 100%, a colorless oily liquid). LCMS: [M+H]⁺ 261.2.

Step 5:

Compound 015296A4 trifluoroacetate (800 mg, 2.22 mmol) was dissolved in acetonitrile (9 mL), and triethylamine (672 mg, 6.66 mmol) and tert-butyl (S)-3-(((4-nitrophenoxy) carbonyl)oxo)pyrrolidine-1-carboxylate (782 mg, 2.22 mmol) were added successively. The reaction solution was stirred at room temperature for 2 h and concentrated by rotary evaporation under reduced pressure to give a crude product of 015296A5, which was purified by flash silica gel column chromatography to give 015296A5 (600 mg, 57%, a white solid). LCMS: [M+H]⁺ 473.5.

Step 6:

Compound 015296A5 (400 mg, 0.85 mmol) was dissolved in dichloromethane (6 mL), and trifluoroacetic acid (2 mL) was added. The reaction solution was stirred at room temperature for 0.5 h and concentrated by rotary evaporation under reduced pressure to give 015296A6 trifluoroacetate (400 mg, 100%, a colorless oily liquid). LCMS: [M+H]⁺ 374.4.

Step 7:

Compound 015296A6 trifluoroacetate (400 mg, 0.85 mmol) was dissolved in DMF (5 mL), and DIPEA (260 mg, 2 mmol), (E)-4-(dimethylamino)but-2-enoic acid hydrochloride (141 mg, 0.85 mmol) and HATU (388 mg, 1.02 mmol) were added successively. The reaction solution was stirred at room temperature for 0.5 h and concentrated by rotary evaporation under reduced pressure to give a crude product of SZ-015296, which was subjected to reverse phase column chromatography (acetonitrile/water with 0.1% formic acid) and lyophilized to give SZ-015296 (240 mg, 58%, a white solid). LCMS: [M+H]⁺ 485.23.

¹H NMR (400 MHz, DMSO-d₆) δ 9.60 (s, 1H), 8.37 (s, 1H), 6.80-6.50 (m, 2H), 5.18 (d, J=19.6 Hz, 1H), 4.34-3.57 (m, 10H), 2.88 (s, 3H), 2.77 (d, J=3.9 Hz, 6H), 2.57 (s, 3H), 2.24-190 (m, 4H), 1.62-1.49 (m, 2H), 1.40 (t, J=7.3 Hz, 3H).

### Example 23 SZ-015291: (S,E)-1-(4-(dimethylamino)but-2-enoyl)pyrrolidin-3-yl 4-((2-(3-hydroxyazetidin-1-yl)-9-isopropyl-9H-purin-6-yl)amino)piperidine-1-carboxylate

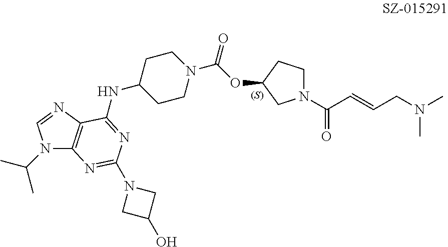

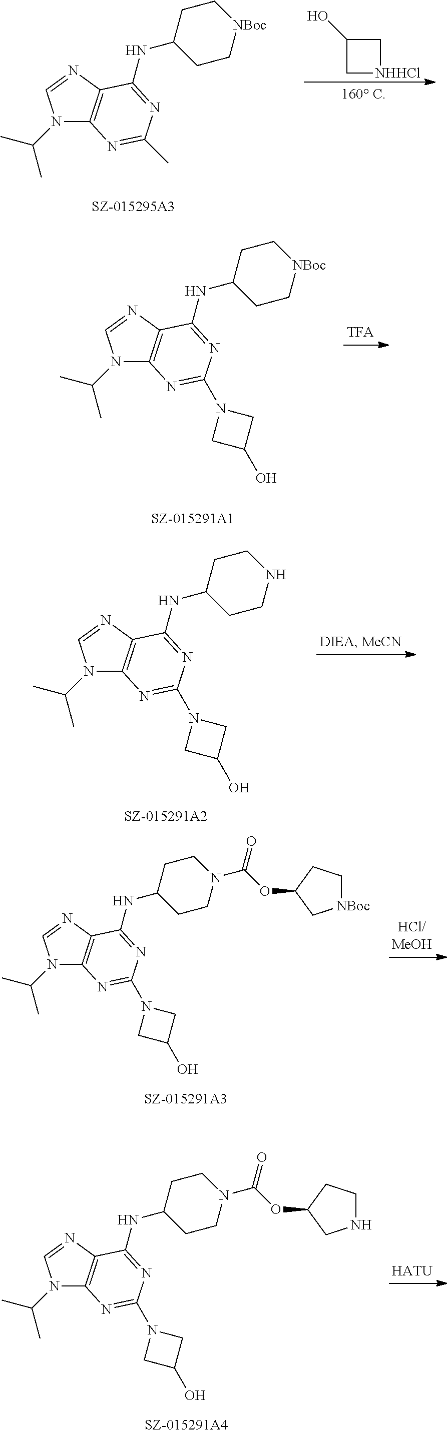

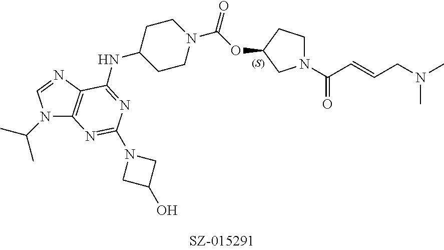

Step 1:

SZ-015295A3 (2 g, 5.07 mmol), azetidin-3-ol hydrochloride (4 g, 10.14 mmol) and potassium carbonate (3 g, 22.05 mmol) were mixed in N-methylpyrrolidone (10 mL), and the reaction solution was heated to 160° C. and stirred for 8 h. The reaction solution was directly concentrated by rotary evaporation, and the residue was purified by flash silica gel column chromatography (PE:EA=1:4; Rf=0.2) to give SZ-015291A1 (300 mg, a foamy solid). LCMS: [M+H]⁺ 432.44.

Step 2:

SZ-015291A1 (250 mg, 0.58 mmol) was dissolved in dichloromethane (4 mL), and trifluoroacetic acid (2 mL) was then added. The reaction solution was stirred at room temperature overnight. After the reaction was completed, the reaction solution was concentrated by rotary evaporation to give a crude product of SZ-015291A2.

Step 3:

Tert-butyl (S)-3-hydroxypyrrolidine-1-carboxylate (108 mg, 0.58 mmol),p-nitrophenyl chloroformate (116 mg, 0.58 mmol) and triethylamine (0.2 mL) were mixed in acetonitrile (5 mL), and the reaction solution was stirred at 40° C. for 2 h. Then the crude product of SZ-015291A2 was added, and the resulting reaction solution was stirred at room temperature overnight. The reaction solution was directly concentrated by rotary evaporation, and the residue was purified by flash silica gel column chromatography (PE:EA=1:4; Rf=0.3) to give SZ-015291A3 (200 mg, a foamy solid). LCMS: [M+H]⁺ 545.35.

Step 4:

SZ-015291A3 (100 mg) was dissolved in saturated methanolic hydrochloride solution, and the reaction solution was stirred at room temperature for about 2 h, and then directly concentrated by rotary evaporation to give a crude product of SZ-015291A4 (100 mg). LCMS: [M+H]⁺ 444.39.

Step 5:

SZ-015291A4 obtained in the previous step, trans-4-dimethylaminocrotonic acid hydrochloride (30 mg, 0.18 mmol), DIPEA (0.13 mL, 0.40 mmol) and HATU (83 mg, 0.21 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015291 (20 mg, a light yellow solid). LCMS: [M+H]⁺ 556.31.

¹H NMR (400 MHz, DMSO-d₆) 7.89 (s, 1H), 7.27 (s, 1H), 6.68-6.65 (m, 2H), 5.57-5.56 (m, 1H), 5.22-5.17 (m, 1H), 4.60-4.47 (m, 2H), 4.16-4.13 (m, 2H), 3.98-3.86 (m, 2H), 3.80-3.50 (m, 8H), 2.91-2.90 (m, 2H), 2.71 (s, 6H), 2.19-2.03 (m, 2H), 1.87-1.80 (m, 2H), 1.48-1.46 (m, 8H).

### Example 24 SZ-015295: (S)-1-((E)-4-(dimethylamino)but-2-enoyl)pyrrolidin-3-yl 4-((2-(((R)-1-hydroxybutan-2-yl)amino)-9-isopropyl-9H-purin-6-yl)amino)piperidine-1-carboxylate

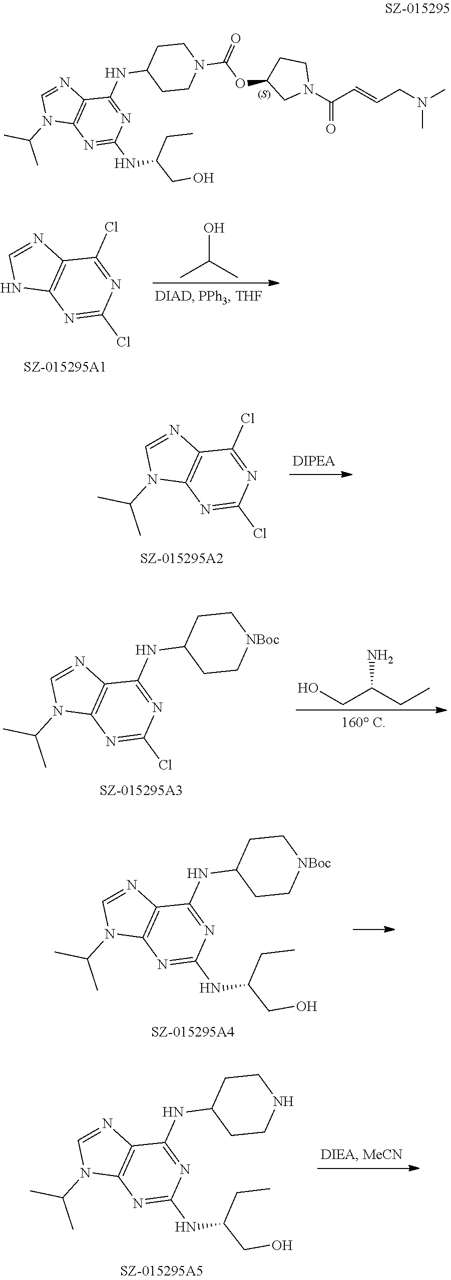

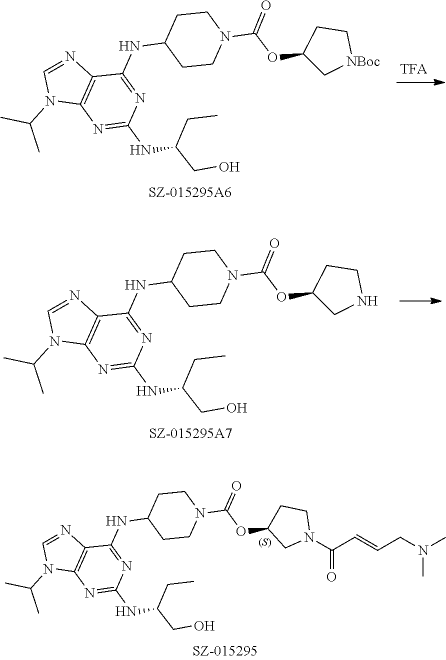

Step 1:

SZ-015295A1(10 g, 52 mmol), isopropanol (3.1 g, 52 mmol) and triphenylphosphine (13.8 g, 58 mmol) were mixed in tetrahydrofuran (150 mL), and then DIAD (11.7 g, 43 mmol) was added dropwise under an ice-water bath. The resulting reaction solution was stirred at room temperature overnight. The reaction solution was directly concentrated by rotary evaporation, and the residue was purified by flash silica gel column chromatography (PE:EA=2:1; Rf=0.3) to give SZ-015295A2 (6 g, a foamy solid). LCMS: [M+H]⁺ 231.11.

Step 2:

SZ-015295A2 (1 g, 4.2 mmol), tert-butyl 4-aminopiperidine-1-carboxylate (1.1 g, 5.5 mmol) and DIPEA (1.5 mL) were mixed in isopropanol (10 mL), and the reaction solution was stirred at 30° C. overnight. After the reaction was completed, the reaction solution was directly concentrated by rotary evaporation, and the residue was purified by flash silica gel column chromatography (PE:EA=1:2; Rf=0.4) to give SZ-015295A3 (1.1 g, a foamy solid). LCMS: [M+H]⁺ 395.25.

Step 3:

SZ-015295A3 (300 mg, 0.76 mmol) and (R)-2-aminobutan-1-ol (500 mg, 6.01 mmol) were mixed and then stirred at 160° C. for 8 h. After the reaction was completed, the reaction solution was dissolved in dichloromethane and purified by flash silica gel column chromatography (PE:EA=1:4; Rf=0.2) to give SZ-015295A4 (150 mg, a foamy solid). LCMS: [M+H]⁺ 448.42.

Step 4:

SZ-015295A4 (150 mg, 0.33 mmol) was dissolved in dichloromethane (4 mL), and trifluoroacetic acid (2 mL) was then added. The reaction solution was stirred at room temperature overnight. After the reaction was completed, the reaction solution was concentrated by rotary evaporation to give a crude product of SZ-015295A5.

Step 5:

Tert-butyl (S)-3-hydroxypyrrolidine-1-carboxylate (80 mg, 0.42 mmol),p-nitrophenyl chloroformate (92 mg, 0.45 mmol) and triethylamine (0.5 mL) were mixed in acetonitrile (5 mL), and the reaction solution was stirred at 40° C. for 2 h. Then the crude product of SZ-015295A5 was added, and the resulting reaction solution was stirred at room temperature overnight. The reaction solution was directly concentrated by rotary evaporation, and the residue was purified by flash silica gel column chromatography (PE:EA=1:4; Rf=0.2) to give SZ-015295A6 (80 mg, a foamy solid). LCMS: [M+H]⁺ 561.35.

Step 6:

SZ-015295A6 (80 mg) was dissolved in dichloromethane (4 mL), and trifluoroacetic acid (2 mL) was slowly added dropwise. The reaction solution was stirred at room temperature for about 2 h, and then directly concentrated by rotary evaporation to give a crude product of SZ-015295A7 (200 mg).LCMS: [M+H]⁺ 461.38.

Step 7:

SZ-015295A7 obtained in the previous step, trans-4-dimethylaminocrotonic acid hydrochloride (18.3 mg, 0.11 mmol), DIPEA (0.15 mL, 0.44 mmol) and HATU (50 mg, 0.13 mmol) were mixed in DMF (3 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015295 (14 mg, a light yellow solid). LCMS: [M+H]⁺ 572.35.

¹H NMR (400 MHz, DMSO-d₆) 7.81 (s, 1H), 6.68-6.65 (m, 2H), 6.43-6.34 (m, 1H), 5.26-5.17 (m, 1H), 4.56-4.53 (m, 1H), 3.86-3.48 (m, 7H), 2.73-2.71 (m, 7H), 1.87-1.85 (m, 2H), 1.67-1.63 (m, 2H), 1.53-1.44 (m, 10H), 0.89 (t, J=7.2 Hz, 3H).

### Example 25 SZ-015298: (S,E)-1-(4-(dimethylamino)but-2-enoyl)pyrrolidin-3-yl 4-((9-isopropyl-2-(1H-pyrazol-4-yl)-9H-purin-6-yl)amino)piperidine-1-carboxylate

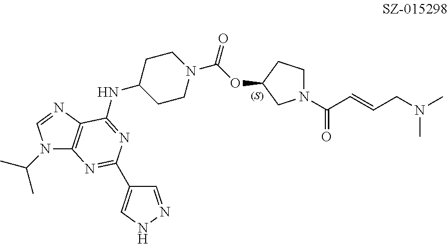

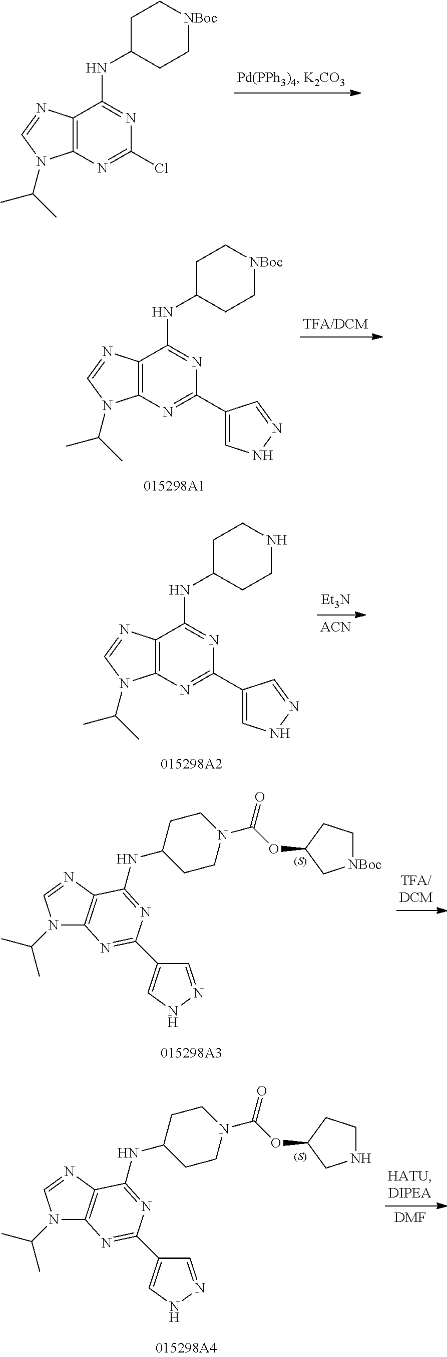

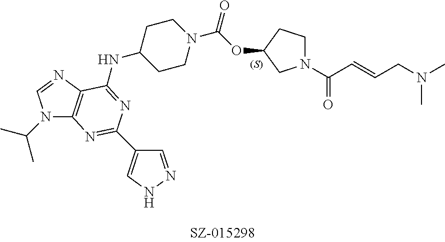

### Step 1

Tert-butyl 4-((2-chloro-9-isopropyl-9H-purin-6-yl)amino)piperidine-1-carboxylate (0.7 g, 1.78 mmol), 4-pyrazoleboronic acid pinacol ester (689 mg, 3.56 mmol), Pd(PPh₃)₄(102 mg, 5%) and potassium carbonate (737 mg, 5.34 mmol) were mixed in dioxane (6 mL) and water (2 mL) under argon atmosphere, and the reaction solution was stirred at 125° C. for 5 h under a microwave condition and then concentrated by rotary evaporation. The residue was purified by flash silica gel column chromatography to give 015298A1 (500 mg, 66%, a white solid). LCMS: [M+H]⁺ 427.4.

### Step 2

Compound 015298A1 (500 mg, 1.17 mmol) was dissolved in dichloromethane (9 mL), and trifluoroacetic acid (3 mL) was added. The reaction solution was stirred at room temperature for 1 h and concentrated by rotary evaporation under reduced pressure to give 015298A2 trifluoroacetate (500 mg, 100%, a colorless oily liquid). LCMS: [M+H]⁺ 326.3.

### Step 3

Compound 015298A2 trifluoroacetate (500 mg, 1.17 mmol) was dissolved in acetonitrile (9 mL), and triethylamine (355 mg, 3.51 mmol) and tert-butyl (S)-3-(((4-nitrophenoxy) carbonyl)oxo)pyrrolidine-1-carboxylate (412 mg, 1.17 mmol) were added successively. The reaction solution was stirred at room temperature for 2 h and concentrated by rotary evaporation under reduced pressure to give a crude product of 015298A3, which was purified by flash silica gel column chromatography to give 015298A3 (330 mg, 53%, a white solid). LCMS: [M+H]⁺ 540.5.

### Step 4

Compound 015298A3 (330 mg, 0.61 mmol) was dissolved in dichloromethane (6 mL), and trifluoroacetic acid (2 mL) was added. The reaction solution was stirred at room temperature for 0.5 h and concentrated by rotary evaporation under reduced pressure to give 015298A4 trifluoroacetate (330 mg, 100%, a colorless oily liquid). LCMS: [M+H]⁺ 440.4.

### Step 5: (S,E)-1-(4-(dimethylamino)but-2-enoyl)pyrrolidin-3-yl 4-((9-isopropyl-2-(1H-pyrazol-4-yl)-9H-purin-6-yl)amino)piperidine-1-carboxylate

Compound 015298A4 trifluoroacetate (330 mg, 0.61 mmol) was dissolved in DMF (5 mL), and DIPEA (260 mg, 2 mmol), (E)-4-(dimethylamino)but-2-enoic acid hydrochloride (101 mg, 0.61 mmol) and HATU (278 mg, 0.73 mmol) were added successively. The reaction solution was stirred at room temperature for 0.5 h and concentrated by rotary evaporation under reduced pressure to give a crude product of SZ-015298, which was subjected to reverse phase column chromatography (acetonitrile/water with 0.1% formic acid) and lyophilized to give SZ-015298 (65 mg, 19%, a white solid). LCMS: [M+H]⁺ 551.25.

¹H NMR (400 MHz, DMSO-d₆) δ 12.99 (s, 1H), 8.10 (d, J=63.8 Hz, 3H), 7.52 (s, 1H), 6.62 (dtd, J=10.4, 6.2, 4.3 Hz, 1H), 6.36 (dd, J=21.2, 15.2 Hz, 1H), 5.16 (d, J=23.2 Hz, 1H), 4.77 (dt, J=13.4, 6.7 Hz, 1H), 4.38 (s, 1H), 4.00 (s, 2H), 3.85-3.67 (m, 1H), 3.67-3.45 (m, 3H), 3.00-2.8 (m, 4H), 2.22-1.83 (m, 10H), 1.53 (d, J=6.8 Hz, 8H).

### Example 26 SZ-015310: (S,E)-1-(4-(dimethylamino)but-2-enoyl)pyrrolidin-3-yl 4-((2-ethyl-9-isopropyl-9H-purin-6-yl)amino)piperidine-1-carboxylate

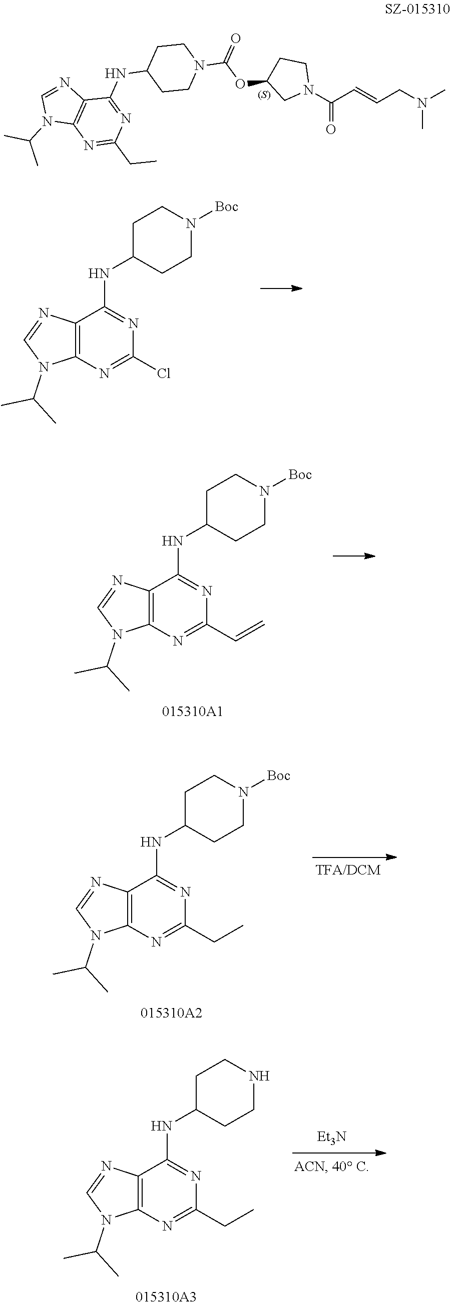

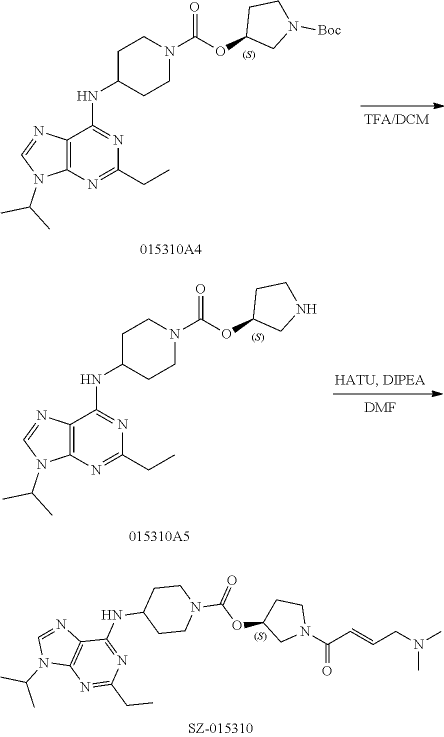

Step 1:

Tert-butyl 4-((2-chloro-9-isopropyl-9H-purin-6-yl)amino)piperidine-1-carboxylate (1.2 g, 3 mmol), vinylboronic acid pinacol ester (924 mg, 6 mmol), Pd(PPh₃)₄(173 mg, 5%) and potassium carbonate (1.24 g, 9 mmol) were mixed in dioxane (9 mL) and water (3 mL) under argon atmosphere, and the reaction solution was stirred at 125° C. for 5 h under a microwave condition and then concentrated by rotary evaporation. The residue was purified by flash silica gel column chromatography to give 015310A1 (800 mg, a white solid). LCMS: [M+H]⁺ 387.4.

Step 2:

015310A1 (800 mg, 2.07 mmol) was dissolved in methanol (10 mL), and ammonium formate (1304 mg, 20.7 mmol) and palladium hydroxide/carbon (80 mg) were added. The reaction solution was stirred at 60° C. for 3 h and filtered, and the organic phase was concentrated by rotary evaporation under reduced pressure to give a crude product, which was purified by flash silica gel column chromatography to give 015310A2 (600 mg, a white solid). LCMS: [M+H]⁺ 389.4.

Step 3:

Compound 015310A2 (600 mg, 1.54 mmol) was dissolved in dichloromethane (9 mL), and trifluoroacetic acid (3 mL) was added. The reaction solution was stirred at room temperature for 1 h and concentrated by rotary evaporation under reduced pressure to give 015310A3 trifluoroacetate (600 mg, 100%, a colorless oily liquid). LCMS: [M+H]⁺ 289.3.

Step 4:

Compound 015310A3 trifluoroacetate (600 mg, 1.54 mmol) was dissolved in acetonitrile (9 mL), and triethylamine (466 mg, 4.62 mmol) and tert-butyl (S)-3-(((4-nitrophenoxy) carbonyl)oxo)pyrrolidine-1-carboxylate (542 mg, 1.54 mmol) were added successively. The reaction solution was stirred at room temperature for 2 h and concentrated by rotary evaporation under reduced pressure to give a crude product of 015310A4, which was purified by flash silica gel column chromatography to give 015310A4 (80 mg, a white solid). LCMS: [M+H]⁺ 502.5.

Step 5:

Compound 015310A4 (80 mg, 0.16 mmol) was dissolved in dichloromethane (6 mL), and trifluoroacetic acid (2 mL) was added. The reaction solution was stirred at room temperature for 0.5 h and concentrated by rotary evaporation under reduced pressure to give 015310A5 trifluoroacetate (80 mg, 100%, a colorless oily liquid). LCMS: [M+H]⁺ 402.4.

Step 6:

Compound 015310A4 trifluoroacetate (80 mg, 0.16 mmol) was dissolved in DMF (5 mL), and DIPEA (62 mg, 0.48 mmol), (E)-4-(dimethylamino)but-2-enoic acid hydrochloride (26.5 mg, 0.16 mmol) and HATU (73 mg, 0.192 mmol) were added successively. The reaction solution was stirred at room temperature for 0.5 h and concentrated by rotary evaporation under reduced pressure to give a crude product of SZ-015310, which was subjected to reverse phase column chromatography (acetonitrile/water with 0.1% formic acid) and lyophilized to give SZ-015310 (40 mg, a white solid). LCMS: [M+H]⁺ 513.34.

¹H NMR (400 MHz, DMSO-d₆) δ 8.16 (s, 1H), 7.45 (d, J=6.8 Hz, 1H), 6.62 (d, J=8.9 Hz, 2H), 5.17 (d, J=20.6 Hz, 1H), 4.71 (dt, J=13.4, 6.6 Hz, 1H), 4.05-3.42 (m, 9H), 2.91 (s, 2H), 2.65 (t, J=8.7 Hz, 8H), 2.23-1.78 (m, 4H), 1.49 (d, J=6.7 Hz, 8H), 1.23 (t, J=7.5 Hz, 3H).

### Example 27 SZ-015308: (E)-1-(4-(dimethylamino)but-2-enoyl)azetidin-3-yl 4-((9-isopropyl-2-methyl-9H-purin-6-yl)amino)piperidine-1-carboxylate

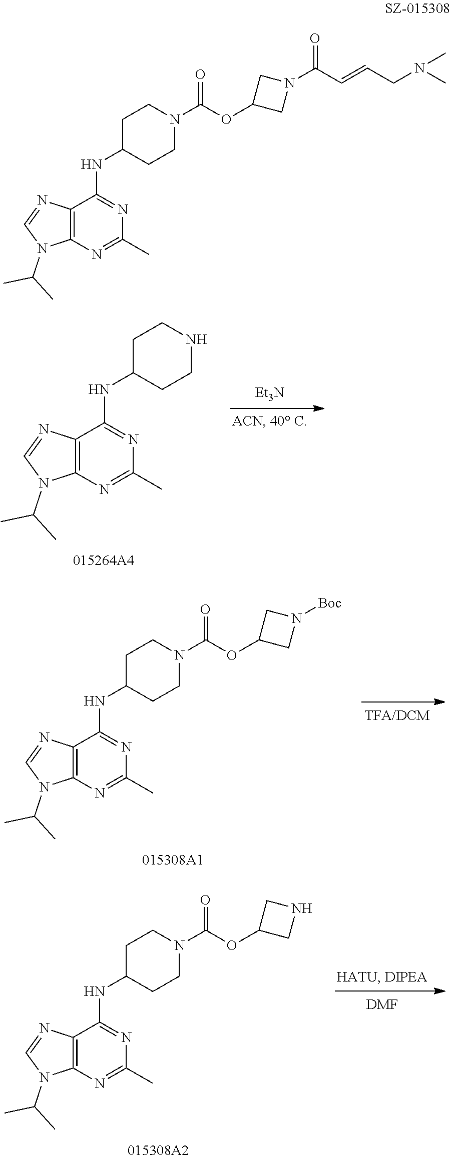

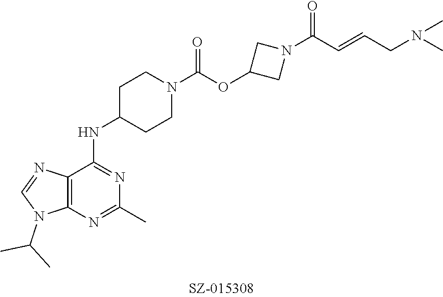 [SMILES: Cc1nc(NC2CCN(C(=O)OC3CN(C(=O)/C=C/CN(C)C)C3)CC2)c2ncn(C(C)C)c2n1]

Step 1:

Compound 015264A4 trifluoroacetate (374 mg, 1 mmol) was dissolved in acetonitrile (9 mL), and triethylamine (303 mg, 3 mmol) and tert-butyl 3-(((4-nitrophenoxy)carbonyl) oxo)azetidine-1-carboxylate (338 mg, 1 mmol) were added successively. The reaction solution was stirred at room temperature for 2 h and concentrated by rotary evaporation under reduced pressure to give a crude product of 015308A1, which was purified by flash silica gel column chromatography to give 015308A1 (474 mg, a white solid). LCMS: [M+H]⁺ 474.4.

Step 2:

Compound 015308A1 (474 mg, 1 mmol) was dissolved in dichloromethane (6 mL), and trifluoroacetic acid (2 mL) was added. The reaction solution was stirred at room temperature for 0.5 h and concentrated by rotary evaporation under reduced pressure to give 015308A2 trifluoroacetate (474 mg, 100%, a colorless oily liquid). LCMS: [M+H]⁺ 374.3.

Step 3:

Compound 015308A2 trifluoroacetate (474 mg, 1 mmol) was dissolved in DMF (5 mL), and DIPEA (520 mg, 4 mmol), (E)-4-(dimethylamino)but-2-enoic acid hydrochloride (166 mg, 1 mmol) and HATU (456 mg, 1.2 mmol) were added successively. The reaction solution was stirred at room temperature for 0.5 h and concentrated by rotary evaporation under reduced pressure to give a crude product of SZ-015308, which was subjected to reverse phase column chromatography (acetonitrile/water with 0.1% formic acid) and lyophilized to give SZ-015308 (180 mg, a white solid). LCMS: [M+H]⁺ 485.11.

¹H NMR (400 MHz, DMSO-d₆) δ 8.17 (s, 1H), 7.49 (d, J=7.8 Hz, 1H), 6.58 (m, 1H), 6.38 (d, J=15.4 Hz, 1H), 5.15-5.10 (m, 1H), 4.69 (dd, J=13.3, 6.9 Hz, 1H), 4.59-4.48 (m, 1H), 4.25 (dd, J=11.4, 6.8 Hz, 1H), 4.17 (d, J=6.4 Hz, 1H), 4.01 (s, 2H), 3.86 (d, J=6.9 Hz, 3H), 3.02 (s, 1H), 2.96-2.83 (m, 1H), 2.76 (s, 7H), 2.42 (s, 3H), 1.86 (s, 2H), 1.48 (d, J=6.7 Hz, 8H).

### Example 28 SZ-015317: (S)-1-((E)-4-(dimethylamino)but-2-enoyl)pyrrolidin-3-yl 4-((6-(((R)-1-hydroxybutan-2-yl)amino)-3-isopropylimidazo[1,2-b]pyridazin-8-yl)amino) piperidine-1-carboxylate

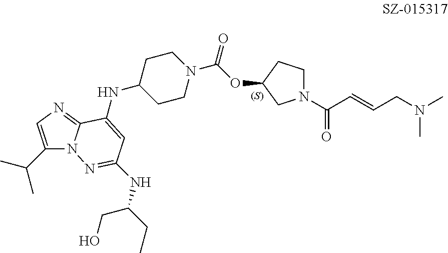

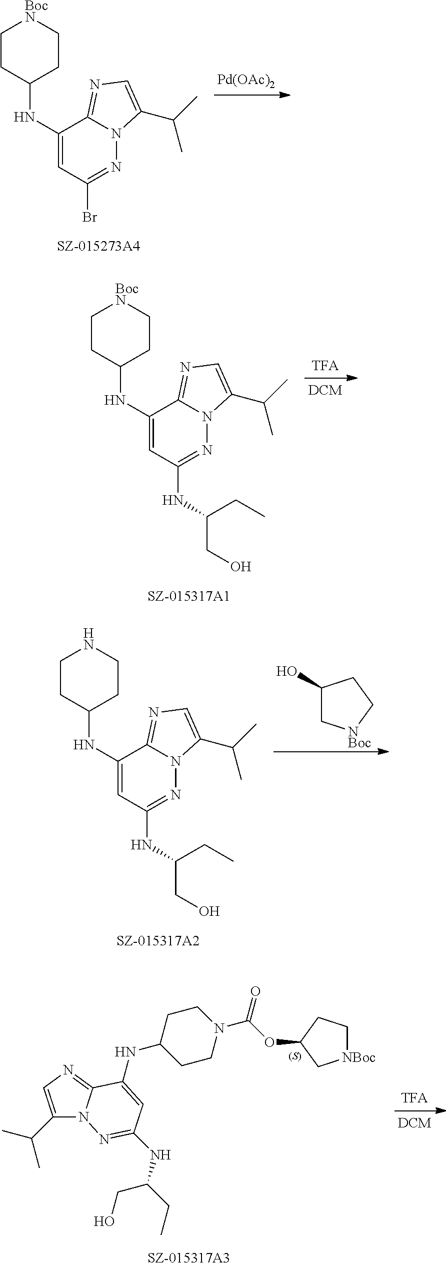

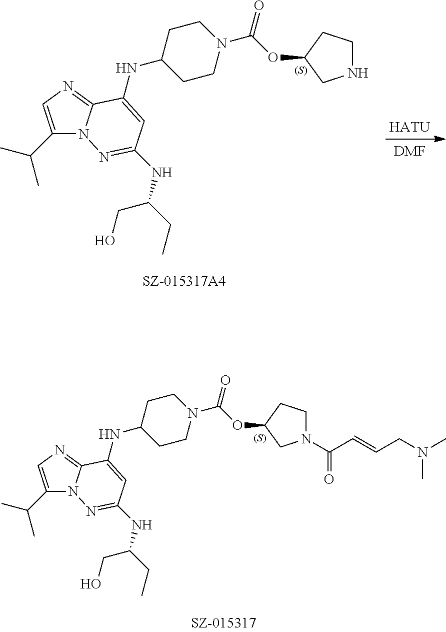

Step 1:

SZ-015273A4 (700 mg, 1.59 mmol), palladium acetate (200 mg), R-BINAP (300 mg, 0.48 mmol), (R)-2-aminobutanol (500 mg), and sodium tert-butoxide (500 mg, 5.2 mmol) were mixed in anhydrous toluene (30 mL) under argon atmosphere, and the reaction solution was stirred at 130° C. for 2 h. The reaction solution was directly concentrated by rotary evaporation, and the residue was purified by flash silica gel column chromatography (PE:EA=1:2; Rf=0.2) to give SZ-015317A1 (600 mg, a foamy solid). LCMS: [M+H]⁺ 447.35.

Step 2:

The product obtained in the previous step was dissolved in dichloromethane (10 mL), and trifluoroacetic acid (5 mL) was slowly added dropwise. The reaction solution was stirred at room temperature for about 1 h, and then directly concentrated by rotary evaporation to give a crude product of SZ-015317A2 (500 mg). LCMS: [M+H]⁺ 346.23.

Step 3:

Tert-butyl (S)-3-hydroxypyrrolidine-1-carboxylate (251 mg, 1.34 mmol),p-nitrophenyl chloroformate (269 mg, 1.34 mmol) and triethylamine (1 mL, 6.03 mmol) were mixed in acetonitrile (10 mL), and the reaction solution was stirred at 40° C. for 2 h. Then SZ-015317A2 (500 mg) was added, and the resulting reaction solution was stirred at room temperature overnight. The reaction solution was directly concentrated by rotary evaporation, and the residue was purified by flash silica gel column chromatography (EA: 100%; Rf=0.3) to give SZ-015317A3 (250 mg, a foamy solid). LCMS: [M+H]⁺ 559.31.

Step 4:

SZ-015317A3 (250 mg) was dissolved in dichloromethane (4 mL), and trifluoroacetic acid (2 mL) was slowly added dropwise. The reaction solution was stirred at room temperature for about 1 h, and then directly concentrated by rotary evaporation to give a crude product of SZ-015317A4 (300 mg).LCMS: [M+H]⁺ 459.29.

Step 5:

SZ-015317A4 obtained in the previous step, trans-4-dimethylaminocrotonic acid hydrochloride (74 mg, 0.44 mmol), triethylamine (0.3 mL) and HATU (203 mg, 0.52 mmol) were mixed in DMF (3 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015317 (70 mg, a light yellow solid). LCMS: [M+H]⁺ 570.40.

SZ-015317: ¹H NMR (400 MHz, DMSO-d₆) 6.97 (s, 1H), 6.67-6.62 (m, 1H), 6.43-6.34 (m, 2H), 5.89-5.87 (d, J=7.6 Hz, 1H), 5.64 (s, 1H), 5.21-5.15 (m, 1H), 4.62-4.59 (m, 1H), 4.01-3.99 (m, 2H), 3.81-3.41 (m, 8H), 3.19-3.16 (m, 1H), 3.05-3.04 (m, 2H), 2.93 (bs, 2H), 2.16 (s, 6H), 2.16-1.99 (m, 2H), 1.93-1.90 (m, 2H), 1.70-1.65 (m, 1H), 1.54-1.45 (m, 3H), 1.32 (d, J=6.8 Hz, 6H), 0.92 (t, J=7.6 Hz, 3H).

### Example 29 SZ-015319: (S,E)-1-(4-(dimethylamino)but-2-enoyl)pyrrolidin-3-yl 4-((3,5-diisopropyl-3H-imidazo[4,5-b]pyridin-7-yl)amino)piperidine-1-carboxylate

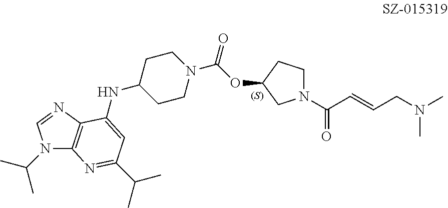

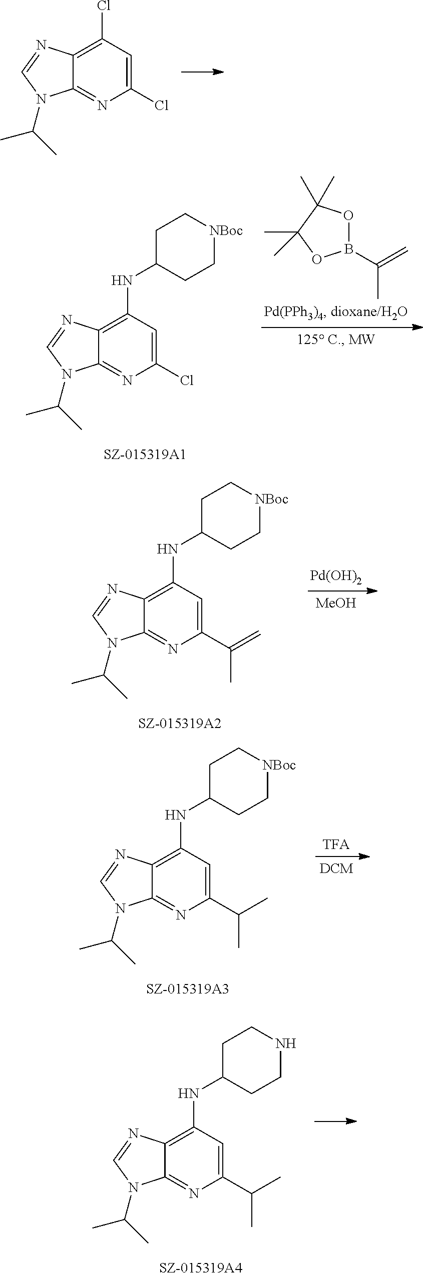

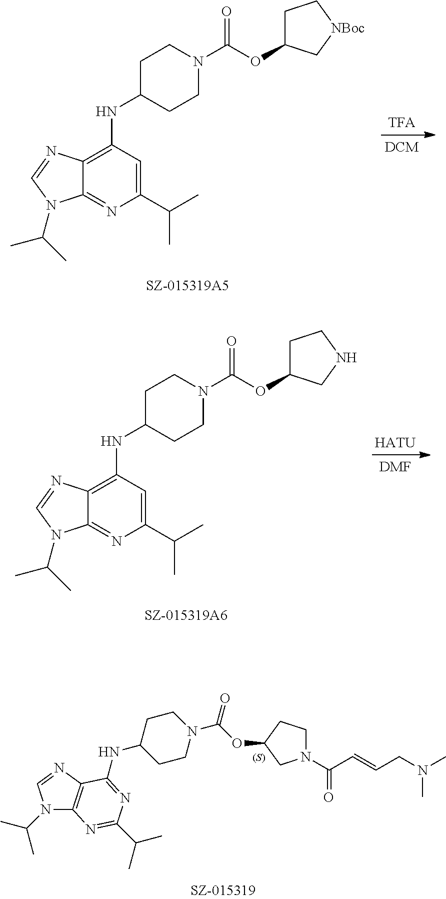

Step 1:

5,7-dichloro-3-isopropyl-3H-imidazo[4,5-b]pyridine (2 g, 7.3 mmol, see US 20100280065 for synthesis) and 1-Boc-4-aminopiperidine (4.0 g) were mixed, and the reaction solution was stirred under melting at 160° C. for 7 h. After the reaction was completed, water (20 mL) and dichloromethane (20 mL) were added, and the pH was adjusted to about 8 with saturated sodium bicarbonate. The organic phase was directly concentrated by rotary evaporation, and the residue was purified by flash silica gel column chromatography (EA:PE=2:1; Rf=0.3) to give SZ-015319A1 (2.1 g, a foamy solid). LCMS: [M+H]⁺ 394.35.

Step 2:

SZ-015319A1 (500 mg, 1.27 mmol), isopropenylboronic acid pinacol ester (500 mg, 2.97 mmol), potassium carbonate (500 mg, 3.67 mmol) and tetrakis(triphenylphosphine) palladium (100 mg) were mixed in dioxane (6 mL) and water (2 mL) under argon atmosphere, and the reaction solution was stirred at 125° C. for 3 h under a microwave condition. After the reaction was completed, the reaction solution was directly concentrated by rotary evaporation, and the residue was purified by flash silica gel column chromatography (EA:PE=2:1; Rf=0.2) to give SZ-015319A2 (500 mg, a foamy solid). LCMS: [M+H]⁺ 400.31.

Step 3:

SZ-015319A2 (500 mg, 1.25 mmol) and palladium hydroxide (500 mg) were mixed in methanol (10 mL), and ammonium formate (500 mg) was then added. The reaction solution was stirred at 70° C. for 1 h. After the reaction was completed, the reaction solution was cooled and filtered. The filtrate was concentrated by rotary evaporation, and the residue was purified by flash silica gel column chromatography (EA:PE=2:1; Rf=0.3) to give SZ-015319A3 (500 mg, a foamy solid). LCMS: [M+H]⁺ 402.31.

Step 4:

SZ-015319A3 (500 mg, 1.25 mmol) and trifluoroacetic acid (5 mL) were mixed in dichloromethane (10 mL), and the reaction solution was stirred at room temperature for 1 h. After the reaction was completed, the reaction solution was directly concentrated by rotary evaporation without further treatment. LCMS: [M+H]⁺ 302.29.

Step 5:

Tert-butyl (S)-3-hydroxypyrrolidine-1-carboxylate (233 mg, 1.25 mmol), p-nitrophenyl chloroformate (251 mg, 1.25 mmol) and triethylamine (0.7 mL) were mixed in acetonitrile (10 mL), and the reaction solution was stirred at 40° C. for 2 h. Then SZ-015319A4 was added, and the resulting reaction solution was stirred at room temperature overnight. The reaction solution was directly concentrated by rotary evaporation, and the residue was purified by flash silica gel column chromatography (EA: 100%; Rf=0.2) to give SZ-015319A5 (270 mg, a foamy solid). LCMS: [M+H]⁺ 515.22.

Step 6:

SZ-015319A5 (270 mg) was dissolved in dichloromethane (6 mL), and trifluoroacetic acid (3 mL) was slowly added dropwise. The reaction solution was stirred at room temperature for about 1 h, and then directly concentrated by rotary evaporation to give a crude product of SZ-015319A6 (300 mg).LCMS: [M+H]⁺ 415.29.

Step 7:

SZ-015319A6 obtained in the previous step, trans-4-dimethylaminocrotonic acid hydrochloride (87 mg, 0.52 mmol), triethylamine (0.3 mL) and HATU (240 mg, 0.78 mmol) were mixed in DMF (4 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015319 (72 mg, a light yellow solid). LCMS: [M+H]⁺ 526.35.

SZ-015319: ¹H NMR (400 MHz, DMSO-d₆) 8.11 (s, 1H), 6.68-6.61 (m, 1H), 6.40-6.35 (m, 2H), 6.29 (s, 1H), 5.21-5.15 (m, 1H), 4.81-4.74 (m, 1H), 4.13-3.97 (m, 3H), 3.84-3.73 (m, 1H), 3.64-3.43 (m, 3H), 3.05 (d, J=6.0 Hz, 2H), 2.99-2.89 (m, 3H), 2.17 (s, 6H), 2.11-1.94 (m, 4H), 1.53 (d, J=6.4 Hz, 1H), 1.47-1.41 (m, 2H), 1.25 (d, J=7.2 Hz, 6H).

### Example 30 SZ-015326: (S)-1-((E)-4-(dimethylamino)but-2-enoyl)pyrrolidin-3-yl 4-((2-(((R)-1-hydroxybutan-2-yl)amino)-8-isopropylpyrazolo[1,5-a][1,3,5]triazin-4-yl) amino)piperidine-1-carboxylate

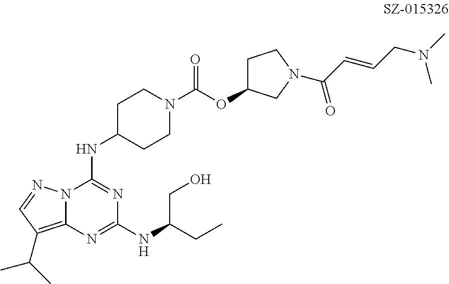 [SMILES: CC[C@H](CO)Nc1nc(NC2CCN(C(=O)O[C@H]3CCN(C(=O)/C=C/CN(C)C)C3)CC2)n2ncc(C(C)C)c2n1]

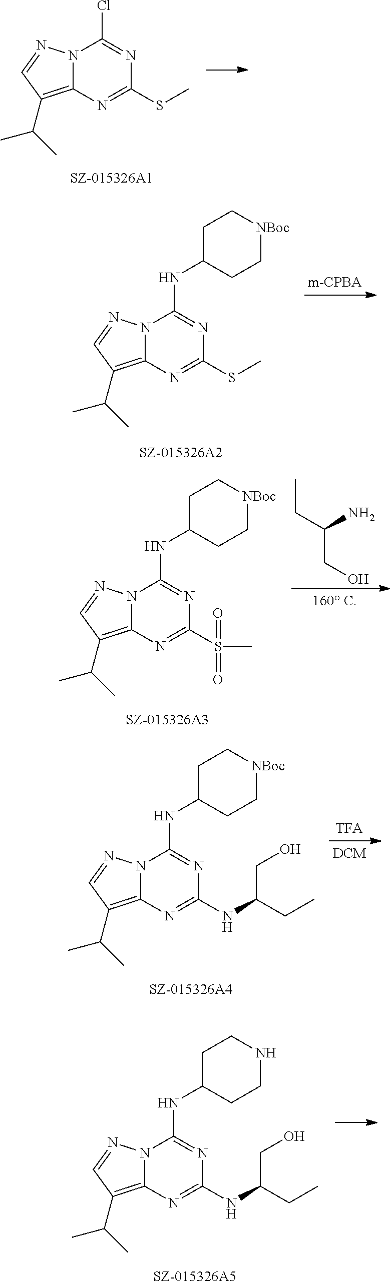

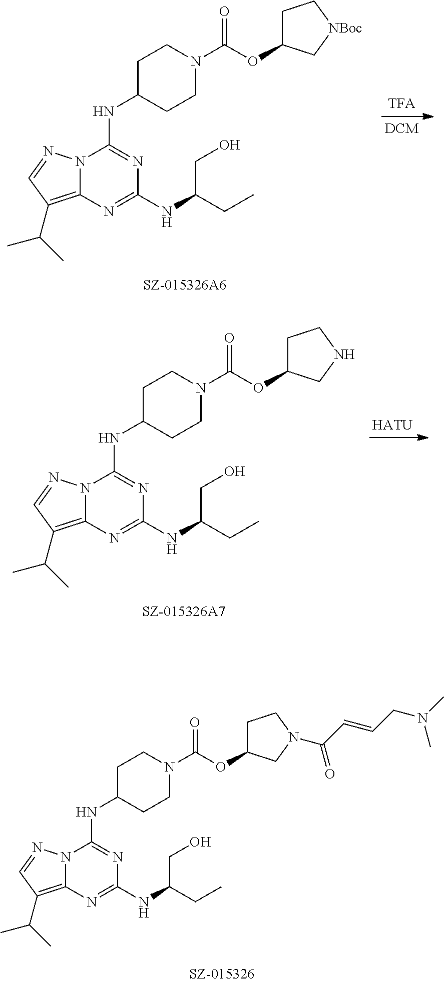

Step 1:

SZ-015326A1 (3.6 g, 15.1 mmol, see WO2019197549 for synthesis) was dissolved in acetonitrile (50 mL), and 1-Boc-4-aminopiperidine (4.5 g, 22.5 mmol) and sodium bicarbonate (6.2 g, 45.3 mmol) were added successively. The reaction solution was stirred at 75° C. overnight. After the reaction was completed, water (50 mL) and ethyl acetate (100 mL) were added, and the organic phase was separated out and directly concentrated by rotary evaporation. The residue was purified by flash silica gel column chromatography (EA:PE=1:7; Rf=0.4) to give SZ-015326A2 (4.4 g, a white solid). LCMS: [M+H]⁺ 407.32.

Step 2:

SZ-015326A2 (4.4 g, 10.8 mmol) was dissolved in dichloromethane, and then m-CPBA (5.5 g, 32.4 mmol) was added. The reaction solution was stirred at room temperature for 2 h. After the reaction was completed, saturated sodium thiosulfate (70 mL) and dichloromethane (50 mL) were added, and the organic phase was washed with saturated sodium chloride three times and then concentrated by rotary evaporation to give SZ-015326A3 (5.5 g). LCMS: [M+H]⁺ 439.31.

Step 3:

SZ-015326A3 (1.0 g) was suspended in (R)-2-aminobutanol (2.0 g), and the suspension was stirred at 160° C. for 2 h. After the reaction was completed, the reaction solution was directly purified by flash silica gel column chromatography (EA:PE=1:1; Rf=0.3) to give SZ-015326A4 (600 mg, a white solid). LCMS: [M+H]⁺ 448.31.

Step 4:

The product obtained in the previous step was dissolved in dichloromethane (8 mL), and trifluoroacetic acid (4 mL) was slowly added dropwise. The reaction solution was stirred at room temperature for about 1 h, and then directly concentrated by rotary evaporation to give a crude product of SZ-015326A5. LCMS: [M+H]⁺ 348.29.

Step 5:

Tert-butyl (S)-3-hydroxypyrrolidine-1-carboxylate (251 mg, 1.34 mmol), p-nitrophenyl chloroformate (270 mg, 1.34 mmol) and triethylamine (1 mL, 6.03 mmol) were mixed in acetonitrile (5 mL), and the reaction solution was stirred at 40° C. for 2 h. Then SZ-015326A5 was added, and the resulting reaction solution was stirred at room temperature overnight. The reaction solution was directly concentrated by rotary evaporation, and the residue was purified by flash silica gel column chromatography (EA:PE=2:1; Rf=0.3) to give SZ-015326A6 (390 mg, a foamy solid). LCMS: [M+H]⁺ 561.24.

Step 6:

SZ-015326A6 (390 mg) was dissolved in dichloromethane (10 mL), and trifluoroacetic acid (6 mL) was slowly added dropwise. The reaction solution was stirred at room temperature for about 1 h, and then directly concentrated by rotary evaporation to give a crude product of SZ-015326A7 (300 mg).LCMS: [M+H]⁺ 461.35.

Step 5:

SZ-015326A7 obtained in the previous step, trans-4-dimethylaminocrotonic acid hydrochloride (118 mg, 0.71 mmol), triethylamine (0.4 mL) and HATU (325 mg, 0.85 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015326 (184 mg, alight yellow solid). LCMS: [M+H]⁺ 572.41.

SZ-015326: ¹H NMR (400 MHz, DMSO-d₆) 7.98 (s, 1H), 7.69 (s, 1H), 6.69-6.61 (m, 1H), 6.53-6.49 (m, 1H), 6.44-6.34 (m, 1H), 5.21-5.15 (m, 1H), 4.63 (bs, 1H), 4.18-3.99 (m, 3H), 3.84-3.73 (m, 2H), 3.64-3.37 (m, 5H), 3.07-3.05 (m, 2H), 2.95-2.82 (m, 3H), 2.17 (s, 6H), 2.14-1.99 (m, 2H), 1.86-1.83 (m, 2H), 1.69-1.43 (m, 4H), 1.25 (d, J=6.4 Hz, 6H), 0.89 (t, J=7.6 Hz, 3H).

### Example 31 SZ-015330: (S,E)-1-(4-(dimethylamino)but-2-enoyl)pyrrolidin-3-yl 4-((8-isopropyl-2-(isopropylamino)pyrazolo[1,5-a][1,3,5]triazin-4-yl)amino)piperidine-1-carboxylate

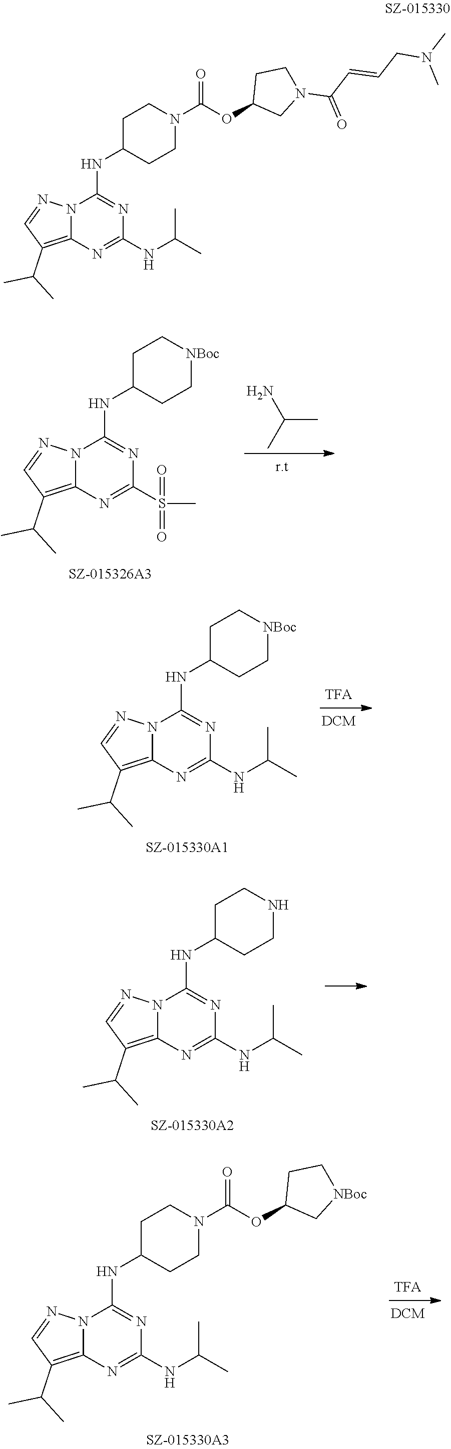

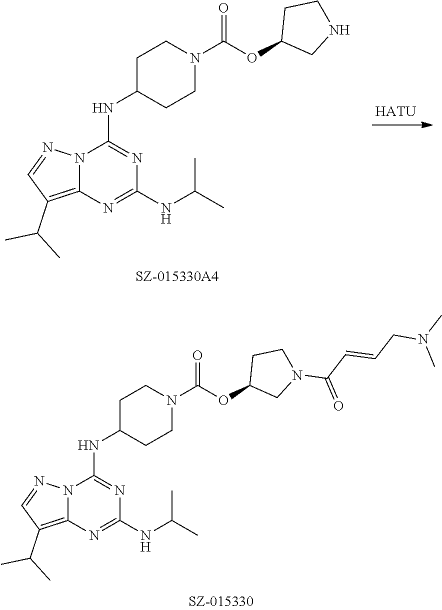 [SMILES: CC(C)Nc1nc(NC2CCN(C(=O)O[C@H]3CCNC3)CC2)n2ncc(C(C)C)c2n1 ; "HATU" ; CC(C)Nc1nc(NC2CCN(C(=O)O[C@H]3CCN(C(=O)/C=C/CN(C)C)C3)CC2)n2ncc(C(C)C)c2n1]

Step 1:

SZ-015326A3 (1.0 g) was suspended in isopropylamine (2.5 g) and stirred at room temperature overnight. After the reaction was completed, isopropylamine was directly removed by rotary evaporation, and the residue was purified by flash silica gel column chromatography (EA:PE=1:3; Rf=0.3) to give SZ-015330A1 (520 mg, a foamy solid). LCMS: [M+H]⁺ 418.26.

Step 2:

The product obtained in the previous step was dissolved in dichloromethane (8 mL), and trifluoroacetic acid (6 mL) was slowly added dropwise. The reaction solution was stirred at room temperature for about 1 h, and then directly concentrated by rotary evaporation to give a crude product of SZ-015330A2. LCMS: [M+H]⁺ 318.29.

Step 3:

Tert-butyl (S)-3-hydroxypyrrolidine-1-carboxylate (224 mg, 1.2 mmol), p-nitrophenyl chloroformate (241 mg, 1.2 mmol) and triethylamine (0.75 mL) were mixed in acetonitrile (10 mL), and the reaction solution was stirred at 40° C. for 2 h. Then SZ-015330A2 was added, and the resulting reaction solution was stirred at room temperature overnight. The reaction solution was directly concentrated by rotary evaporation, and the residue was purified by flash silica gel column chromatography (EA:PE=2:1; Rf=0.3) to give SZ-015330A3 (100 mg, a foamy solid). LCMS: [M+H]⁺ 531.40.

Step 4:

SZ-015330A3 (100 mg) was dissolved in dichloromethane (4 mL), and trifluoroacetic acid (2 mL) was slowly added dropwise. The reaction solution was stirred at room temperature for about 1 h, and then directly concentrated by rotary evaporation to give a crude product of SZ-015330A4 (300 mg).LCMS: [M+H]⁺ 431.56.

Step 5:

SZ-015330A4 obtained in the previous step, trans-4-dimethylaminocrotonic acid hydrochloride (33 mg, 0.2 mmol), triethylamine (0.2 mL) and HATU (90 mg, 0.23 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015330 (54 mg, a light yellow solid). LCMS: [M+H]⁺ 542.41.

SZ-015330: ¹H NMR (400 MHz, DMSO-d₆) 7.95 (s, 1H), 7.69 (s, 1H), 6.70-6.61 (m, 1H), 6.53-6.49 (m, 1H), 6.44-6.34 (m, 1H), 5.21-5.15 (m, 1H), 4.15-3.99 (m, 4H), 3.84-3.73 (m, 1H), 3.64-3.37 (m, 3H), 3.07-3.05 (m, 2H), 2.95-2.82 (m, 3H), 2.17 (s, 6H), 2.14-1.99 (m, 2H), 1.86-1.83 (m, 2H), 1.69-1.43 (m, 2H), 1.25 (d, J=6.4 Hz, 6H), 1.25 (d, J=6.4 Hz, 6H).

### Example 32 SZ-015331: (S,E)-1-(4-(dimethylamino)but-2-enoyl)pyrrolidin-3-yl 4-((2-(cyclopropylamino)-8-isopropylpyrazolo[1,5-a][1,3,5]triazin-4-yl)amino)piperidine-1-carboxylate

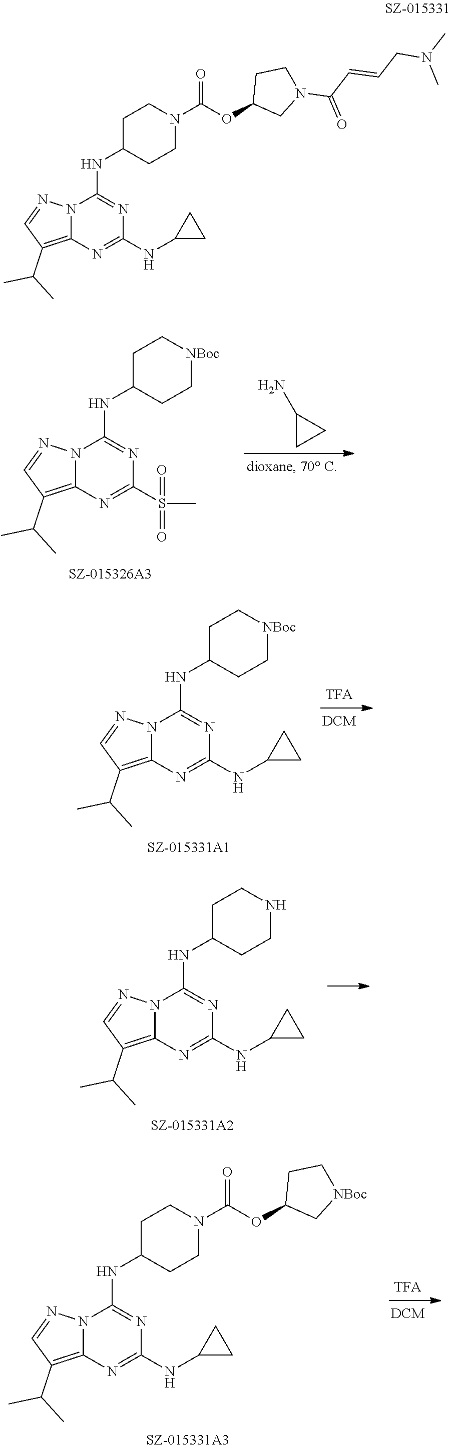

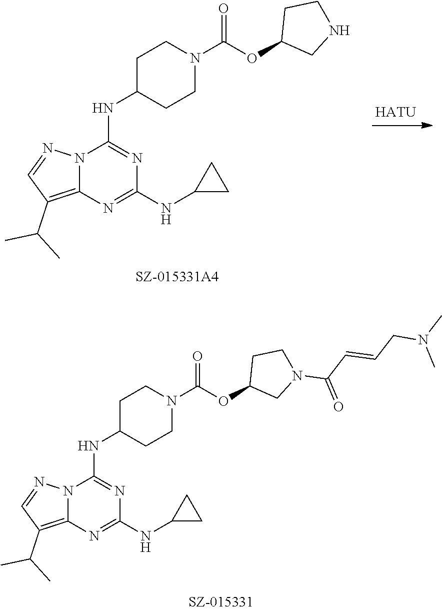 [SMILES: CC(C)c1cnn2c(NC3CCN(C(=O)O[C@H]4CCNC4)CC3)nc(NC3CC3)nc12 ; "HATU" ; CC(C)c1cnn2c(NC3CCN(C(=O)O[C@H]4CCN(C(=O)/C=C/CN(C)C)C4)CC3)nc(NC3CC3)nc12]

Step 1:

SZ-015326A3 (1.0 g) was suspended in a solution of cyclopropylamine (1.0 g) in dioxane (20 mL), and the suspension was stirred at 70° C. overnight. After the reaction was completed, the solvent was directly removed by rotary evaporation, and the residue was purified by flash silica gel column chromatography (EA:PE=1:3; Rf=0.6) to give SZ-015331A1 (540 mg, a foamy solid). LCMS: [M+H]⁺ 416.33.

Step 2:

The product obtained in the previous step was dissolved in dichloromethane (10 mL), and trifluoroacetic acid (6 mL) was slowly added dropwise. The reaction solution was stirred at room temperature for about 1 h, and then directly concentrated by rotary evaporation to give a crude product of SZ-015331A2. LCMS: [M+H]⁺ 316.25.

Step 3:

Tert-butyl (S)-3-hydroxypyrrolidine-1-carboxylate (243 mg, 1.3 mmol), p-nitrophenyl chloroformate (261 mg, 1.3 mmol) and triethylamine (0.8 mL) were mixed in acetonitrile (10 mL), and the reaction solution was stirred at 40° C. for 2 h. Then SZ-015331A2 was added, and the resulting reaction solution was stirred at room temperature overnight. The reaction solution was directly concentrated by rotary evaporation, and the residue was purified by flash silica gel column chromatography (EA:PE=2:1; Rf=0.3) to give SZ-015331A3 (300 mg, a foamy solid). LCMS: [M+H]⁺ 529.32.

Step 4:

SZ-015331A3 (300 mg) was dissolved in dichloromethane (4 mL), and trifluoroacetic acid (2 mL) was slowly added dropwise. The reaction solution was stirred at room temperature for about 1 h, and then directly concentrated by rotary evaporation to give a crude product of SZ-015331A4 (300 mg).LCMS: [M+H]⁺ 429.29.

Step 5:

SZ-015331A4 obtained in the previous step, trans-4-dimethylaminocrotonic acid hydrochloride (93 mg, 0.6 mmol), triethylamine (0.4 mL) and HATU (214 mg, 0.6 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015331 (177 mg, a light yellow solid). LCMS: [M+H]⁺ 540.41.

SZ-015331: ¹H NMR (400 MHz, DMSO-d₆) 8.03 (s, 1H), 7.71 (s, 1H), 7.09 (s, 1H), 6.70-6.61 (m, 1H), 6.43-6.34 (m, 1H), 5.21-5.15 (m, 1H), 4.14-4.02 (m, 3H), 3.84-3.73 (m, 1H), 3.64-3.34 (m, 3H), 3.07-3.05 (m, 2H), 2.98-2.75 (m, 4H), 2.17 (s, 6H), 2.14-1.99 (m, 2H), 1.86-1.83 (m, 2H), 1.66-1.63 (m, 2H), 1.25 (d, J=6.4 Hz, 6H), 0.67-0.62 (m, 2H), 0.51-0.47 (m, 2H).

### Example 33 SZ-015332: (S,E)-1-(4-(dimethylamino)but-2-enoyl)pyrrolidin-3-yl 4-((2-isopropoxy-8-isopropylpyrazolo[1,5-a][1,3,5]triazin-4-yl)amino)piperidine-1-carboxylate

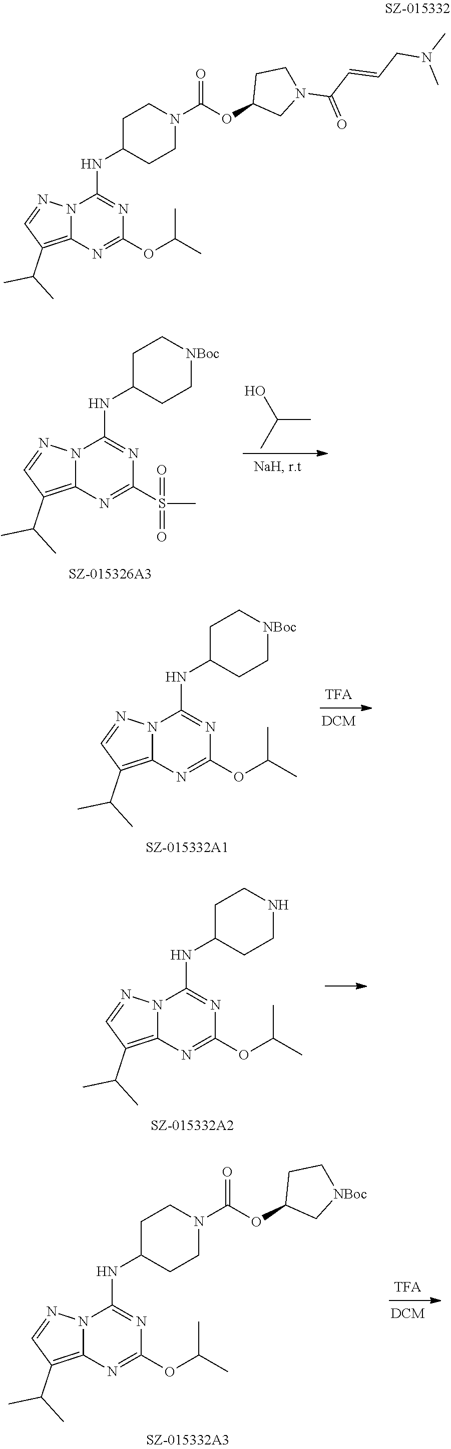

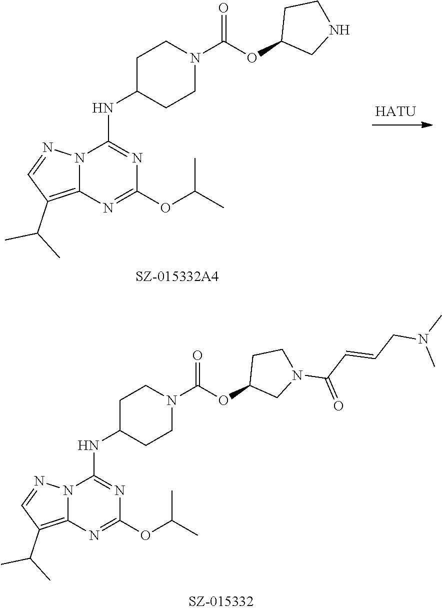 [SMILES: CC(C)Oc1nc(NC2CCN(C(=O)O[C@H]3CCNC3)CC2)n2ncc(C(C)C)c2n1 ; "HATU" ; CC(C)Oc1nc(NC2CCN(C(=O)O[C@H]3CCN(C(=O)/C=C/CN(C)C)C3)CC2)n2ncc(C(C)C)c2n1]

Step 1:

Sodium hydride (0.9 g, 22.8 mmol) was added to a solution of isopropanol (1.4 g, 22.8 mmol) in tetrahydrofuran (20 mL), and the reaction solution was stirred for 1 h. Then SZ-015326A3 (1.0 g, 2.3 mmol) was added, and the resulting reaction solution was stirred at room temperature for 2 h. After the reaction was completed, the solvent was directly removed by rotary evaporation, and the residue was purified by flash silica gel column chromatography (EA:PE=1:2; Rf=0.5) to give SZ-015332A1 (270 mg, a foamy solid). LCMS: [M+H]⁺ 419.31.

Step 2:

The product obtained in the previous step was dissolved in dichloromethane (5 mL), and trifluoroacetic acid (3 mL) was slowly added dropwise. The reaction solution was stirred at room temperature for about 1 h, and then directly concentrated by rotary evaporation to give a crude product of SZ-015332A2. LCMS: [M+H]⁺ 319.32.

Step 3:

Tert-butyl (S)-3-hydroxypyrrolidine-1-carboxylate (120 mg, 0.64 mmol), p-nitrophenyl chloroformate (129 mg, 0.64 mmol) and triethylamine (0.4 mL) were mixed in acetonitrile (5 mL), and the reaction solution was stirred at 40° C. for 2 h. Then SZ-015332A2 was added, and the resulting reaction solution was stirred at room temperature overnight. The reaction solution was directly concentrated by rotary evaporation, and the residue was purified by flash silica gel column chromatography (EA:PE=1:1; Rf=0.3) to give SZ-015332A3 (190 mg, a foamy solid). LCMS: [M+H]⁺ 532.36.

Step 4:

SZ-015332A3 (300 mg) was dissolved in dichloromethane (4 mL), and trifluoroacetic acid (2 mL) was slowly added dropwise. The reaction solution was stirred at room temperature for about 1 h, and then directly concentrated by rotary evaporation to give a crude product of SZ-015332A4 (300 mg).LCMS: [M+H]⁺ 432.32.

Step 5:

SZ-015332A4 obtained in the previous step, trans-4-dimethylaminocrotonic acid hydrochloride (59 mg, 0.4 mmol), triethylamine (1 mL) and HATU (135 mg, 0.4 mmol) were mixed in DMF (2 mL), and the reaction solution was stirred at room temperature for 1 h. After the reaction was completed, the solvent was removed by rotary evaporation under reduced pressure to give a crude product. The crude product was subjected to reverse phase column chromatography (acetonitrile/water with 0.1% formic acid) and lyophilized to give SZ-015332 (92 mg, a light yellow solid). LCMS: [M+H]⁺ 543.41.

SZ-015332: ¹H NMR (400 MHz, DMSO-d₆) 8.57-8.54 (m, 1H), 7.91 (s, 1H), 6.69-6.61 (m, 1H), 6.43-6.34 (m, 1H), 5.21-5.15 (m, 1H), 4.20-3.95 (m, 3H), 3.84-3.73 (m, 1H), 3.64-3.34 (m, 3H), 3.31 (s, 1H), 3.07-2.92 (m, 5H), 2.17 (s, 6H), 2.14-1.99 (m, 2H), 1.86-1.83 (m, 2H), 1.69-1.59 (m, 2H), 1.33 (d, J=6.4 Hz, 6H), 1.29 (d, J=6.8 Hz, 6H).

### Example 34 SZ-015342: (S,E)-1-(4-(dimethylamino)but-2-enoyl)pyrrolidin-3-yl 4-((3,6-diisopropylimidazo[1,2-a]pyrazin-8-yl)amino)piperidine-1-carboxylate

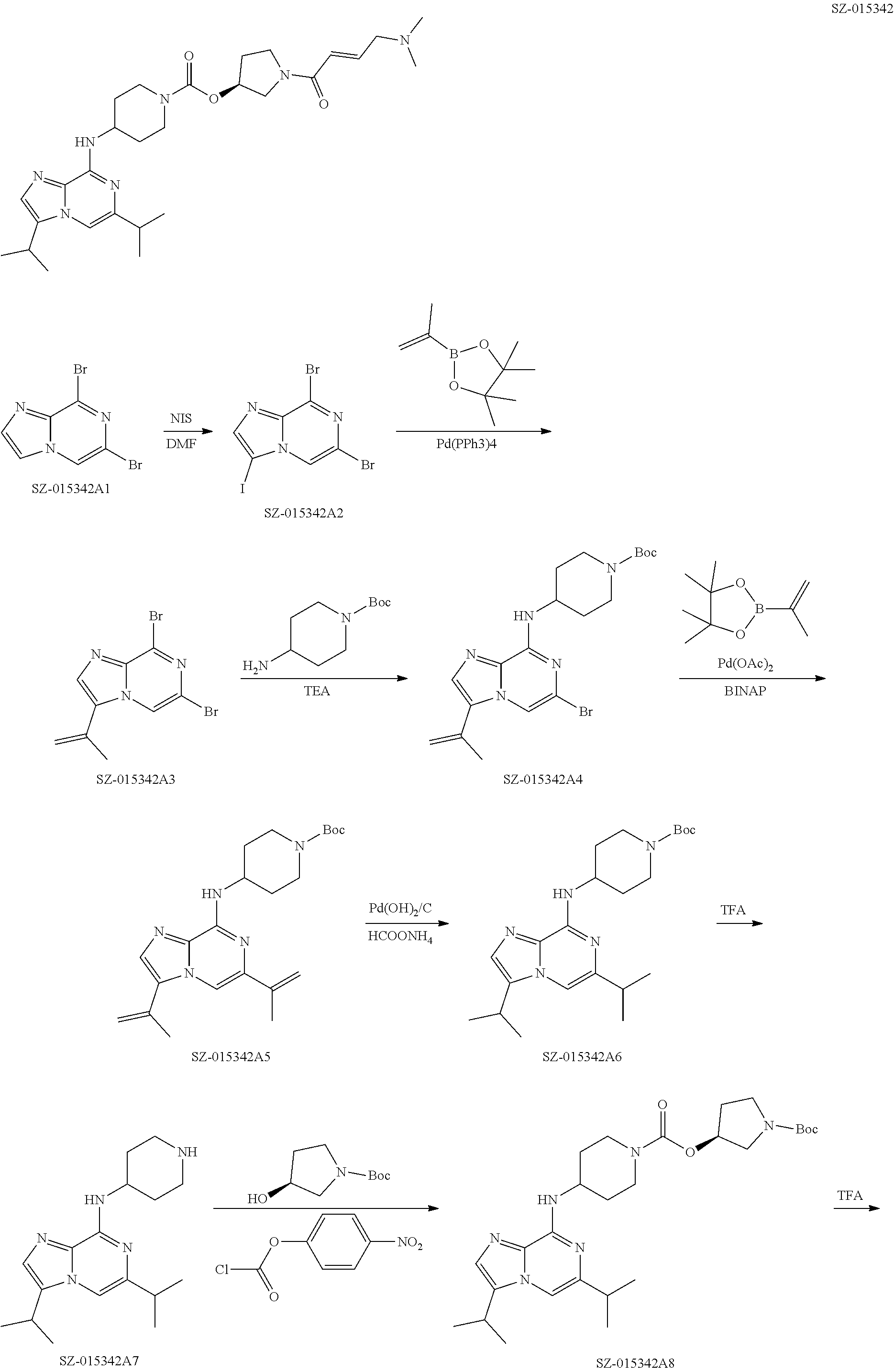

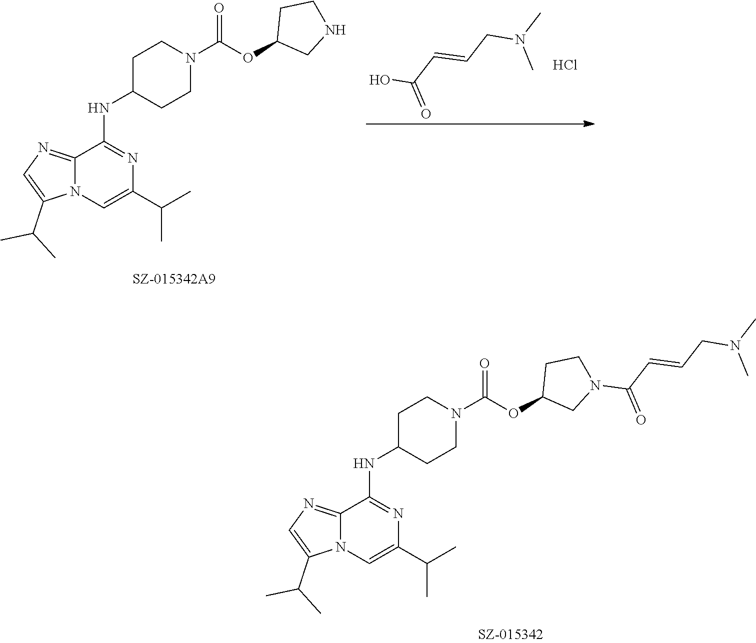

Step 1:

Compound NIS (13.1 g, 58 mmol) was added in portions to a solution of compound 015342A1 (15 g, 53.9 mmol) in red-yellow DMF (260 mL) at 26° C. The resulting reaction solution was heated to 63° C. and stirred for 16 h. The resulting reactant was then concentrated by rotary evaporation to remove the solvent. Ethyl acetate (180 mL) and water (0.3 L) was added to the residue for liquid separation, and ethyl acetate (0.3 L×3) was then added for extraction. The extracts were combined, dried over anhydrous MgSO₄ and filtered, and the solvent was removed by rotary evaporation. The residue was purified by column chromatography to give compound 015342A2 (5.6 g, a viscous liquid), which was solidified after being placed for a period of time. LCMS: [M+H]⁺ 402.1/404.1.

Step 2:

Powder of compound Pd(PPh₃)₄(1.26 g, 1.1 mmol) was added in portions to a suspension of compound 015342A2 (5.6 g, 13.9 mmol), isopropenylboronic acid pinacol ester (11.9 g, 69 mmol), potassium carbonate (1.96 g, 13.9 mmol), toluene (130 mL), ethanol (69 mL) and water (30 mL) at 26° C. The resulting reaction solution was stirred and purged with nitrogen three times at room temperature, and then heated to 50° C. and further stirred for 16 h. The resulting reaction solution was concentrated by rotary evaporation to remove the solvent, and ethyl acetate and water were added to the residue for liquid separation, and ethyl acetate (0.3 L×3) was then added for extraction. The extracts were combined, dried over anhydrous MgSO₄ and filtered, and the organic solvent was removed by rotary evaporation. The resulting residue was purified by column chromatography to give compound 015342A3 (3.6 g). LCMS: [M+H]⁺ 316.1/318.1.

Step 3:

A mixture of compound 015342A3 (2.8 g, 8.9 mmol), tert-butyl 4-aminopiperidine-1-carboxylate (3 g, 15 mmol), triethylamine (3.6 mL, 63 mmol) and 1,4-dioxane (36 mL) was heated to 110° C. and stirred for 16 h. The resulting reaction solution was cooled to room temperature, water was added to quench the reaction, and then ethyl acetate (60 mL×3) was added for extraction. The extracts were combined, dried over anhydrous MgSO₄ and filtered, and the solvent was removed by rotary evaporation. The residue was purified by column chromatography to give product 015342A4 (2.6 g, a yellow oil). LCMS: [M+H]⁺ 435.9/437.9.

Step 4:

Compound Pd(PPh₃)₄(0.69 g, 0.6 mmol) was added in portions to a suspension of compound 015342A4 (1.9 g, 4.3 mmol), isopropenylboronic acid pinacol ester (2.3 g, 13.3 mmol), sodium carbonate (1.39 g, 13 mmol), 1,4-dioxane (36 mL) and water (13 mL) at 29° C. The resulting reaction solution was stirred and purged with nitrogen three times at 29° C., and then heated to 110° C. and further stirred for 16 h. The resulting reaction solution was cooled to room temperature, water was added to quench the reaction, and then ethyl acetate (60 mL×3) was added for extraction. The extracts were combined, dried over anhydrous MgSO₄ and filtered, and the organic solvent was removed by rotary evaporation. The resulting residue was purified by column chromatography to give product 015342A5 (1.6 g, a brown oil). LCMS: [M+H]⁺ 398.1.

Step 5:

Compound 015342A5 (1.6 g) was added to a black suspension of Pd(OH)₂/C (1 g) in methanol (130 mL), and then solid ammonium formate was added (9 g). The resulting reaction solution was heated to 90° C. and stirred for 3 h. The resulting reaction solution was cooled to room temperature and filtered through celite, and the filter cake was washed with methanol. The filtrates were combined and then concentrated by rotary evaporation, to remove the solvent, and ethyl acetate and water were added for liquid separation. The suspension was extracted with ethyl acetate (60 mL×3). The filtrates were combined, dried over anhydrous MgSO₄ and filtered, and the solvent was removed by rotary evaporation. The residue was dried for 3 min with an oil pump to give product 015342A6 (1.36 g, a yellow oil). LCMS: [M+H]⁺ 402.3.

Step 6:

A mixture of compound 015342A6 (1.36 g, 3.39 mmol) and trifluoroacetic acid (6 mL) was heated to 90° C. and stirred for 1 h. The resulting dark brown solution was concentrated by rotary evaporation to remove the solvent, and the residue was dried for 3 min with an oil pump to give product 015342A7 (1.36 g, a brown-yellow viscous oil). LCMS: [M+H]⁺ 302.1.

Step 7:

The compound triethylamine (13 mL, 39 mmol) was added in portions to a solution of compounds p-nitrophenyl chloroformate (0.78 g, 3.9 mmol) and (S)-1-N-tert-butoxycarbonyl-3-hydroxypyrrolidine (0.78 g, 3.9 mmol) in 1,4-dioxane (16 mL) at 31° C. (the reaction system rapidly formed a light yellow suspension). The resulting reaction mixture was heated to 69° C. and stirred for 3 h (this suspension was directly used in the next reaction). Compound triethylamine (13 mL, 39 mmol) was added in portions to a solution of compound 015342A7 (1.3 g, 3.3 mmol) in 1,4-dioxane (6 mL) at 31° C. to basify the reaction system. Then, the suspension prepared above was added dropwise to the basic reaction system at 31° C. The resulting reaction solution was heated to 69° C. and stirred for 16 h. The resulting reaction solution was cooled to room temperature, water was added to quench the reaction, and ethyl acetate (60 mL×3) was added for extraction. The extracts were combined, dried over anhydrous MgSO₄ and filtered, and the solvent was removed by rotary evaporation. The residue was purified by column chromatography to give product 015342A8 (0.9 g, a yellow oil). LCMS: [M+H]⁺ 515.3.

Step 8:

A mixture of compound 015342A8 (0.9 g) and trifluoroacetic acid (6 mL) was stirred at 31° C. for 1 h. The resulting dark brown solution was concentrated by rotary evaporation to remove the solvent, and the residue was dried for 3 min with an oil pump to give product 015342A9 (0.9 g, a brown-yellow viscous oil). LCMS: [M+H]⁺ 415.3.

Step 9:

A solution of compound T₃P in ethyl acetate (6.9 mL, 11 mmol, 50 wt %) was added dropwise to a solution of compound 015342A9 (0.9 g, 1.8 mmol), (2E)-4-(dimethylamino)-2-butenoic acid hydrochloride (1 g, 6 mmol) and triethylamine (16 mL, 115 mmol) in acetonitrile (16 mL) at 31° C. The resulting reaction solution was stirred at 23-31° C. for 16 h. Water was added to the reaction solution to quench the reaction, and then solid sodium carbonate was added to basify and saturate the reaction solution. Then ethyl acetate (60 mL×3) was added for extraction. The extracts were combined, dried over anhydrous MgSO₄ and filtered, and the solvent was removed by rotary evaporation. The residue was purified by column chromatography to give a crude product of 015342 (800 mg). This crude product was further purified by preparative chromatography to give compound 015342 (163 mg, a light yellow solid).

LCMS: [M+H]⁺ 526.3.

¹H NMR (CD₃OD, 400 MHz): δ 7.37 (s, 1H), 7.28 (s, 1H), 6.81-6.71 (m, 2H), 5.33 (d, 1H, J=16.0 Hz), 4.36-4.28 (m, 1H), 4.19-3.98 (m, 2H), 3.93-3.49 (m, 6H), 3.29-3.16 (m, 3H), 2.86-2.83 (m, 1H), 2.85 (s, 6H), 2.33-2.26 (m, 1H), 2.26-2.13 (m, 3H), 1.63-1.53 (m, 2H), 1.40 (d, 6H, J=4.0 Hz), 1.32 (d, 6H, J=4.0 Hz).

### Example 35 SZ-015328: (S,E)-1-(4-(dimethylamino)but-2-enoyl)pyrrolidin-3-yl 4-((7-isopropyl-2-(isopropylamino)imidazo[2,1-f][1,2,4]triazin-4-yl)amino)piperidine-1-carboxylate

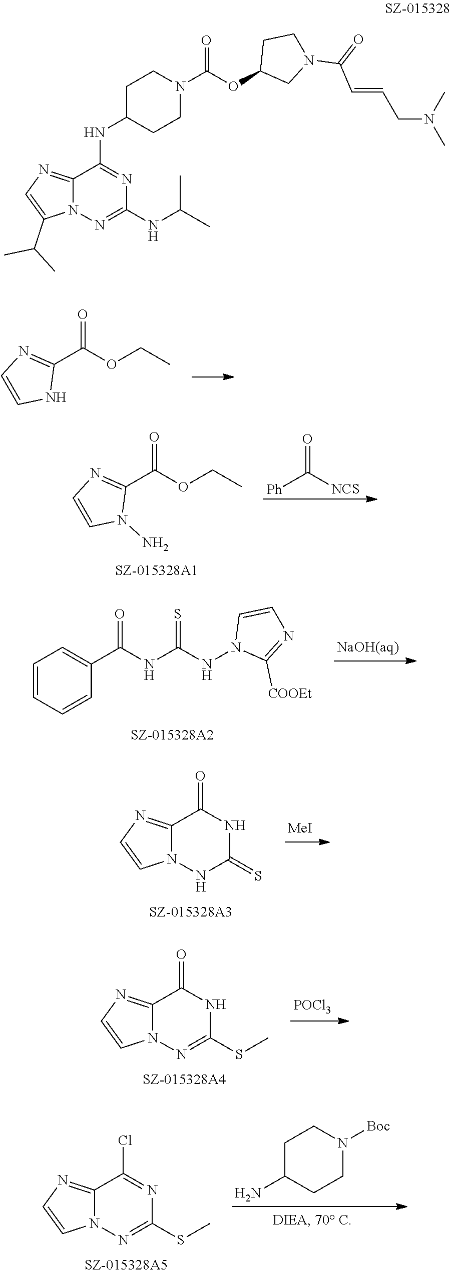

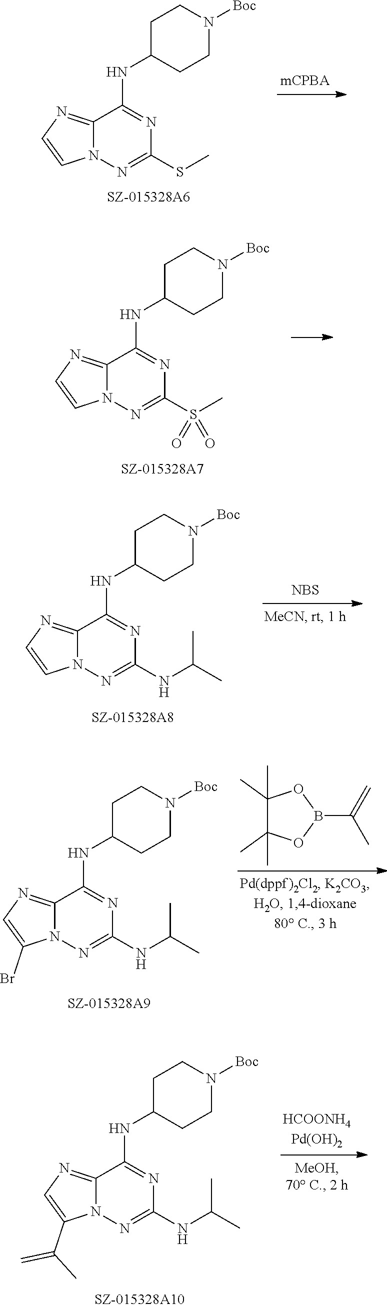

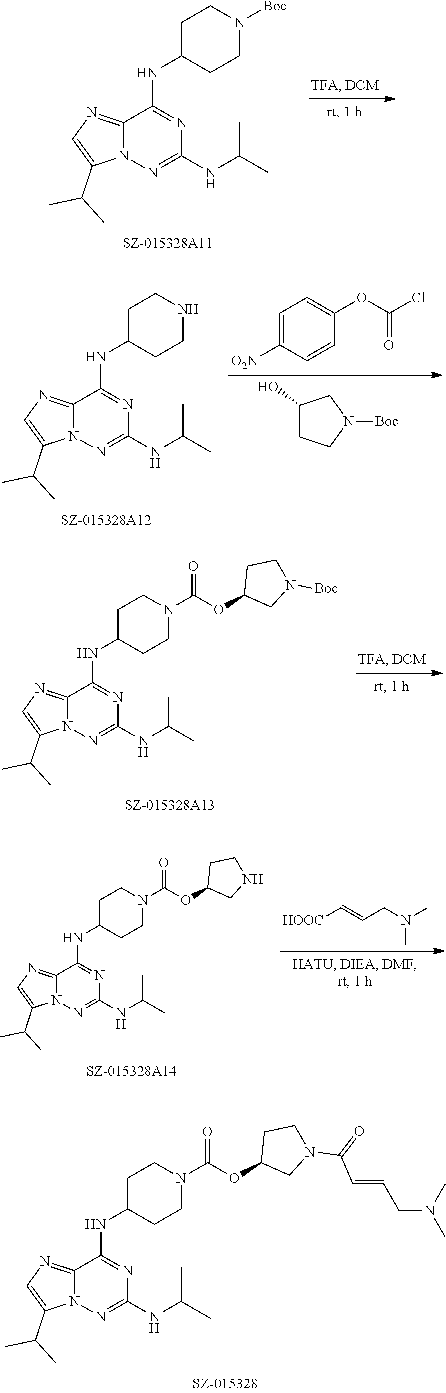

Step 1:

Ethyl imidazole-2-carboxylate (6 g) was dissolved in DMF (400 mL) in a reaction flask, and a solution of potassium tert-butoxide (5.282 g) in DMF (60 mL) was added dropwise to the reaction solution with stirring. The resulting reaction solution was stirred at room temperature for 1 h. O-p-nitrobenzoyl hydroxylamine (7.795 g) was dissolved in DMF (100 mL), and the solution was added dropwise to the reaction solution. The reaction solution changed from dark blue to brown and finally to orange as the dropwise addition proceeded. The reaction solution was stirred at room temperature overnight. After the reaction was completed, the reaction solution was concentrated, saturated sodium bicarbonate solution (400 mL) was added to quench the reaction, and dichloromethane (500 mL×4) was added for extraction. The organic phases were combined, dried over anhydrous sodium sulfate and filtered, and the solvent was removed by rotary evaporation to give a crude product of SZ-015328A1 (4.33 g). LCMS: [M+1]⁺156.05.

Step 2:

SZ-015328A1 (6.7 g) and tetrahydrofuran (50 mL) were added to a reaction flask and stirred when the temperature was reduced to 0° C., and benzoyl isothiocyanate (7.1 g) was added in portions. The reaction solution was stirred at room temperature overnight. After the reaction was completed, the solvent was removed by rotary evaporation to give a crude product of SZ-015328A2, which was directly used in the next step without purification. LCMS: [M+1]⁺319.21.

Step 3:

The crude product SZ-015328A2 in the previous step was added to a reaction flask, and aqueous NaOH solution (2 M, 100 mL) was added. The reaction solution was stirred at 80° C. for 1 h. After the reaction was completed, the reaction solution was adjusted to pH=1 with 2 M aqueous HCl solution, and the precipitate was filtered and washed with DCM to give a crude product of SZ-015328A3 (15 g) containing sodium chloride. LCMS: [M+1]⁺169.02.

Step 4:

SZ-015328A3 (15 g, crude product), NaOH (5 g), H₂O (50 mL) and 1,4-dioxane (200 mL) were added to a reaction flask and stirred at room temperature. Methyl iodide was added in portions. The reaction was monitored by LCMS until it was completed, and then the addition of methyl iodide (4 g) was stopped. After the reaction was completed, the precipitated white solid was filtered out and dried to give a crude product of SZ-015328A4 (4.1 g). LCMS: [M+1]⁺183.01.

Step 5:

SZ-015328A4 (4.1 g, crude product) and POCl₃ (200 mL) were added to a reaction flask and stirred at 105° C. for 24 h. After the reaction was completed, POCl₃ was removed by rotary evaporation, and the residue was purified by silica gel column chromatography (ethyl acetate:petroleum ether=1:3-ethyl acetate:petroleum ether:triethylamine=3:9:1) to give SZ-015328A5 (2.73 g). LCMS: [M+1]⁺200.94.

Step 6:

SZ-015328A5 (2.73 g), 1-Boc-4-aminopiperidine (4.4 g), MeCN (50 mL) and TEA (5 mL) were added to a reaction flask and stirred at 90° C. for 3 h. After the reaction was completed, the reaction solution was concentrated, and the resulting crude product was purified by flash silica gel column chromatography (EA/PE=0%-30%) to give product SZ-015328A6 (2.77 g). LCMS: [M+1]⁺365.11.

Step 7:

SZ-015328A6 (2.77 g) and DCM (30 mL) were added to a reaction flask and stirred at 0° C., and then mCPBA (4.6 g) was added. The reaction solution was warmed to room temperature and stirred for 2 h. Aqueous sodium thiosulfate solution was added to quench the reaction, and saturated potassium carbonate solution (100 mL) and dichloromethane (100 mL×3) were added for extraction. The organic phases were combined and concentrated by rotary evaporation to remove the solvent, and the resulting crude product was purified by flash silica gel column chromatography (EA/DCM=0%-50%) to give product SZ-015328A7 (2.71 g). LCMS: 340.11.

Step 8:

SZ-015328A7 (1.5 g), isopropylamine (4 mL) and NMP (15 mL) were added to an autoclave and stirred at 140° C. for 3 d. After the reaction was completed, Boc anhydride (5 mL) was added, and the reaction solution was stirred for 1 h. After the reaction was completed, the reaction solution was extracted with water (200 mL) and dichloromethane (100 mL×3), and the organic phases were combined and concentrated by rotary evaporation to remove the solvent. The resulting crude product was purified by flash silica gel column chromatography (EA/PE=0%-40%) to give product SZ-015328A8 (869 mg). LCMS: [M+1]+376.24.

Step 9:

SZ-015328A8 (869 mg), NBS (329 mg) and MeCN (50 mL) were added to a reaction flask and stirred at room temperature for 30 min. The reaction solution was concentrated by rotary evaporation under reduced pressure to remove the solvent, and the resulting crude product was purified by flash silica gel column chromatography (EA/PE=0%-20%) to give product SZ-015328A9 (934 mg). LCMS: 455.61, 457.61.

Step 10:

SZ-015328A9 (934 mg), isopropenylboronic acid pinacol ester (672 mg), Pd(dppf)₂Cl₂ (163 mg), potassium carbonate (690 mg), 1,4-dioxane (25 mL) and water (5 mL) were added to a reaction flask and stirred at 80° C. for 3 h under nitrogen atmosphere. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015328A10 (1.02 g). LCMS: [M+1]⁺416.20.

Step 11:

SZ-015328A10 (1.02 g), Pd(OH)₂ (200 mg), ammonium formate (2 g) and methanol (50 mL) were added to a reaction flask and stirred at 80° C. for 1 h. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015328A11 (920 mg). LCMS: [M+1]⁺418.11.

Step 12:

SZ-015328A11 (920 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015328A12, which was directly used in the next step without separation. LCMS: [M+1]+318.10.

Step 13:

(S)-1-N-tert-butoxycarbonyl-3-hydroxypyrrolidine (449 mg), acetonitrile (20 mL), triethylamine (5 mL) and p-nitrophenyl chloroformate (483 mg) were added successively to a reaction flask and stirred at 40° C. for 2 h. The crude product of SZ-015328A12 in the previous step was dissolved in acetonitrile (5 mL), triethylamine was added to adjust the solution to be alkaline, and then the reaction solution was added dropwise to the solution of SZ-015328A12 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-5%) to give SZ-015328A13 (986 mg). LCMS: [M+1]⁺531.13.

Step 14:

SZ-015328A13 (986 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015328A14, which was directly used in the next step without separation. LCMS: [M+1]+431.18.

Step 15:

The crude product of SZ-015328A14 in the previous step, (2E)-4-(dimethylamino)-2-butenoic acid hydrochloride (379 mg), DIEA (3 mL), HATU (798 mg) and DMF (10 mL) were added to a reaction flask and stirred at room temperature for 2 h. After the reaction was completed, the solvent was removed by rotary evaporation, and the residue was separated by a reverse phase column (acetonitrile-water with 0.01% formic acid) to give SZ-015328 (720 mg). LCMS: [M+1]⁺ 542.15.

¹H NMR (400 MHz, DMSO-d₆) δ 8.04 (d, J=8.2 Hz, 1H), 7.11 (s, 1H), 6.70-6.54 (m, 2H), 6.12 (d, J=7.1 Hz, 1H), 5.24-5.17 (m, 1H), 4.28-4.21 (m, 1H), 4.03 (br, 2H), 3.93-3.83 (m, 2H), 3.80-3.75 (m, 1H), 3.68-3.52 (m, 3H), 3.45-3.38 (m, 1H), 3.22-3.16 (m, 1H), 2.88 (br, 2H), 2.55-2.52 (m, 6H), 2.22-2.04 (m, 2H), 1.87-1.84 (m, 2H), 1.63-1.54 (m, 2H), 1.32 (d, J=6.9 Hz, 6H), 1.19 (d, J=6.5 Hz, 6H).

### Example 36 SZ-015338: (S,E)-1-(4-(dimethylamino)but-2-enoyl)pyrrolidin-3-yl 4-((2-cyclopropyl-7-isopropylimidazo[2,1-f][1,2,4]triazin-4-yl)amino)piperidine-1-carboxylate

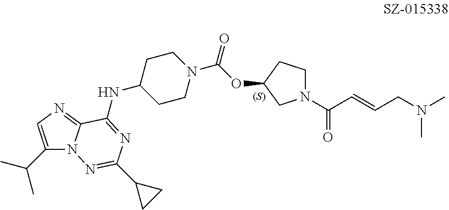

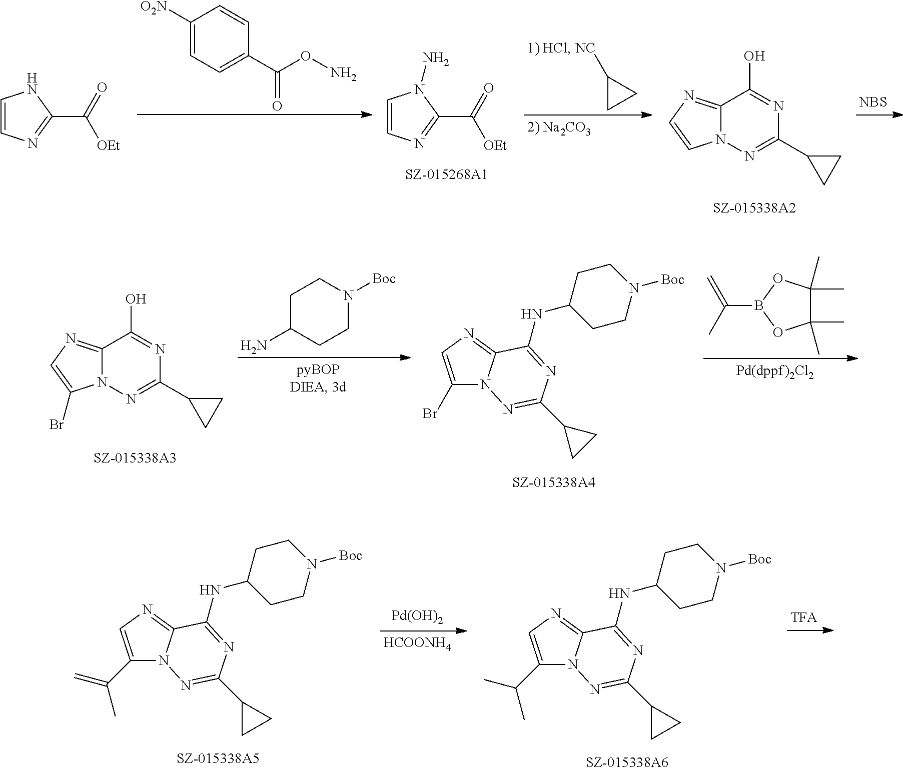

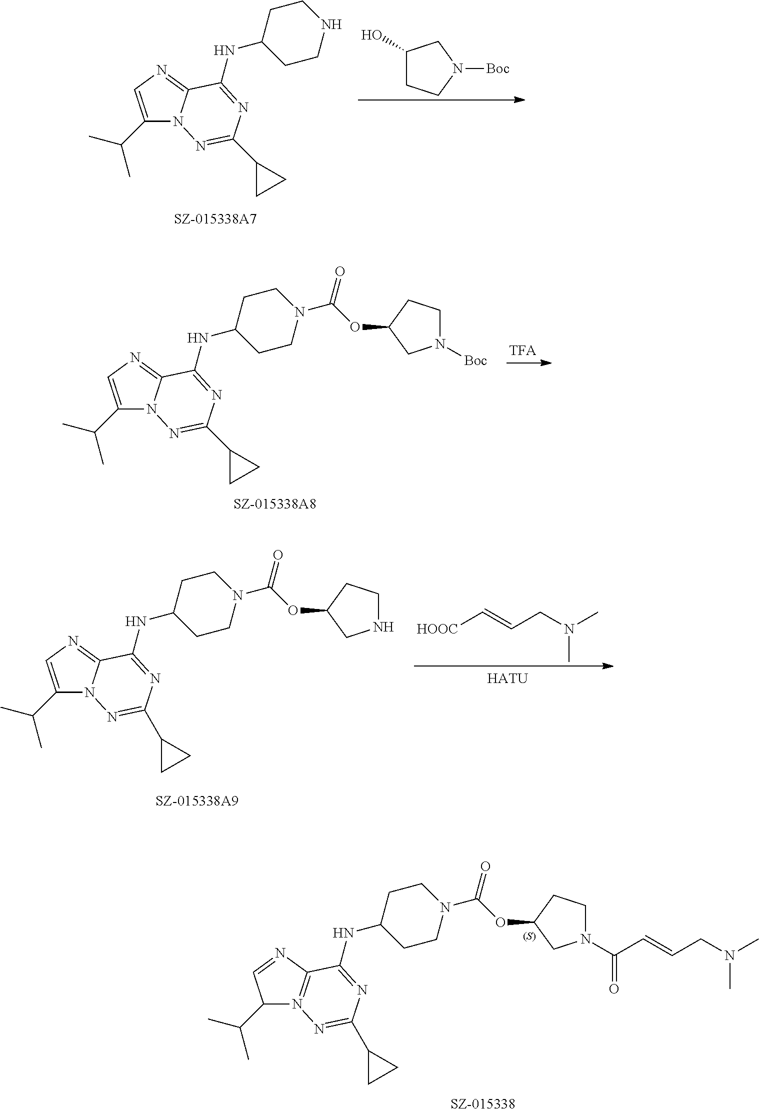

Step 2:

Dry SZ-015268A1 (3.1 g) and anhydrous cyclopropanecarbonitrile (50 mL) were added to a three-necked flask and bubbled for 10 min with nitrogen introduced through a conduit. The reaction solution was cooled to 0° C. and stirred, and then dry hydrogen chloride gas was introduced for 20 min. The reaction solution was then stirred at 80° C. for 1 h. After the reaction was completed, the reaction solution was concentrated, and the remaining solid was slurried with diethyl ether and filtered to give a crude product as an intermediate, which was directly used in the next step without purification. The intermediate, 1,4-dioxane (40 mL), water (30 mL) and sodium bicarbonate (1.6 g) were added to a reaction flask and stirred at 100° C. for 1 h. After the reaction was completed, the solvent was removed under reduced pressure, and the remaining solid was diluted with acetonitrile and filtered to give a crude product of SZ-015338A2 (4 g, a white solid) containing sodium chloride. LCMS: [M+1]+177.07.

Step 3:

SZ-015338A2 (1.5 g, crude product), NBS (1.42 g) and DMF (50 mL) were added to a reaction flask and stirred at 80° C. for 1 h. The reaction solution was concentrated by rotary evaporation under reduced pressure to remove the solvent, and the residue was diluted with dichloromethane and filtered to give a crude product of SZ-015338A3 (1.59 g, a white solid) containing sodium chloride. LCMS: 256.10.

Step 4:

SZ-015338A3 (1.59 g), 1-Boc-4-aminopiperidine (2.6 g), PyBOP (5.2 g), DCE (20 mL) and DIEA (1.5 mL) were added to a reaction flask and stirred at room temperature for 12 h. After the reaction was completed, the reaction solution was filtered and washed with dichloromethane. The organic phase was concentrated by rotary evaporation to remove the solvent, and the resulting crude product was purified by flash silica gel column chromatography (EA/PE=0%-40%) to give product SZ-015338A4 (1.28 g). LCMS: 438.81.

Step 5:

SZ-015338A4 (1.28 g), isopropenylboronic acid pinacol ester (1 g), Pd(dppf)₂Cl₂ (244 mg), potassium carbonate (1.24 g), 1,4-dioxane (36 mL) and water (6 mL) were added to a reaction flask and stirred at 80° C. for 3 h under nitrogen atmosphere. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015338A5 (1.2 g). LCMS: [M+1]⁺399.24.

Step 6:

SZ-015338A5 (1.2 g), Pd(OH)₂ (160 mg), ammonium formate (1.2 g) and methanol (30 mL) were added to a reaction flask and stirred at 70° C. for 2 h. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015338A6 (1.06 g). LCMS: 401.26.

Step 7:

SZ-015338A6 (880 mg), dichloromethane (10 mL) and TFA (2 mL) were added to a reaction flask and stirred at room temperature for 1 h. After the reaction was completed, the reaction solution was concentrated by rotary evaporation to give a crude product of SZ-015338A7, which was directly used in the next step without separation. LCMS: [M+1]+301.21.

Step 8:

(S)-1-N-tert-butoxycarbonyl-3-hydroxypyrrolidine (449 mg), acetonitrile (10 mL), triethylamine (2 mL) and p-nitrophenyl chloroformate (483 mg) were added successively to a reaction flask and stirred at 40° C. for 2 h. The crude product of SZ-015338A7 in the previous step was dissolved in acetonitrile (5 mL), triethylamine was added to adjust the solution to be alkaline, and then the reaction solution was added dropwise to the solution of SZ-015338A7 in acetonitrile. The resulting reaction solution was stirred at room temperature overnight. After the reaction was completed, the solvent was removed by rotary evaporation, and the residue was purified by flash silica gel column chromatography (methanol:dichloromethane=0%-5%) to give SZ-015338A8 (793 mg). LCMS: [M+1]⁺513.25.

Step 9:

SZ-015338A8 (793 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015338A9, which was directly used in the next step without separation. LCMS: [M+1]+414.25.

Step 10:

The crude product of SZ-015338A9 in the previous step, (2E)-4-(dimethylamino)-2-butenoic acid hydrochloride (305 mg), DIEA (1 mL), HATU (703 mg) and DMF (10 mL) were added to a reaction flask and stirred at room temperature for 2 h. After the reaction was completed, the solvent was removed by rotary evaporation, and the residue was separated by a reverse phase column (acetonitrile-water with 0.01% formic acid) to give SZ-015338 (195.6 mg). LCMS: [M+H]⁺ 525.27.

¹H NMR (400 MHz, DMSO-d₆) δ 8.40 (d, J=7.8 Hz, 1H), 7.3 (s, 1H), 6.70-6.57 (m, 2H), 5.21 (d, J=20.3 Hz, 1H), 4.24-4.17 (m, 1H), 4.00 (br, 2H), 3.87-3.75 (m, 2H), 3.68-3.55 (m, 5H), 3.02-2.88 (m, 2H), 2.63 (d, J=5.6 Hz, 6H), 2.23-1.95 (m, 3H), 1.88-1.85 (m, 2H), 1.64-1.53 (m, 2H), 1.34 (d, J=6.9 Hz, 6H), 0.99-0.90 (m, 4H).

### Example 37 SZ-015339: (S,E)-1-(4-(dimethylamino)but-2-enoyl)pyrrolidin-3-yl 4-((2,7-diisopropylimidazo[2,1-f][1,2,4]triazin-4-yl)amino)piperidine-1-carboxylate

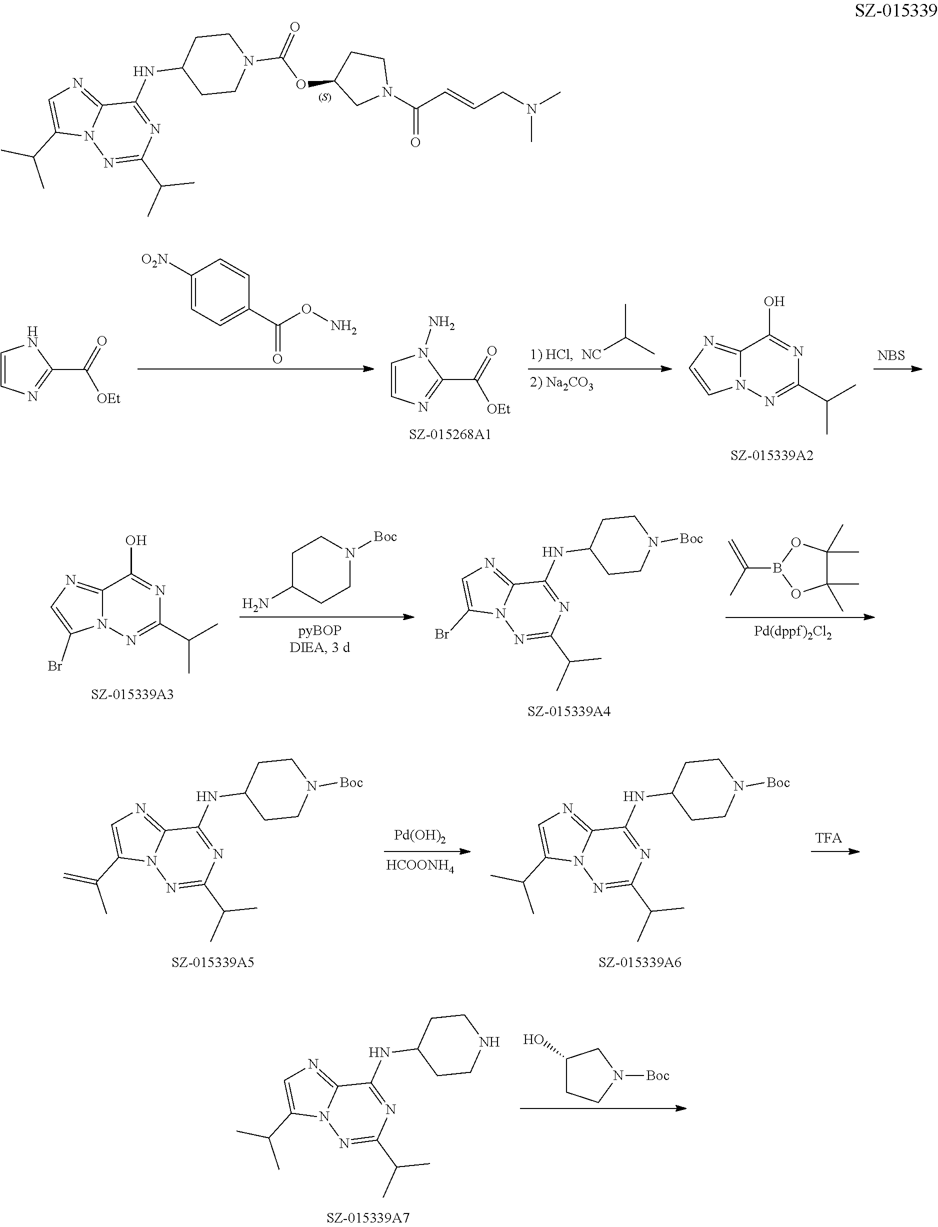

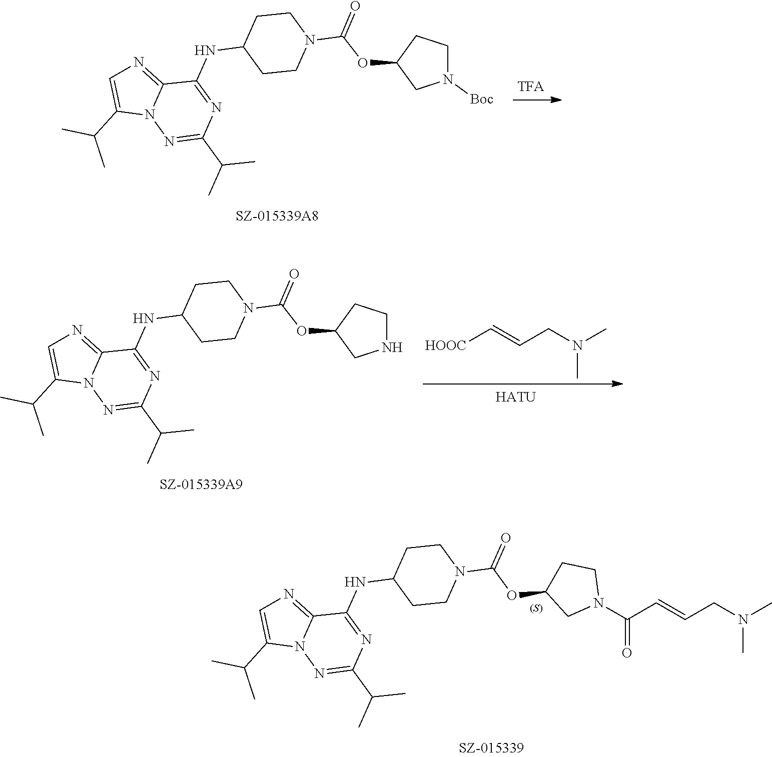

Step 2:

Dry SZ-015339A1 (3.1 g) and anhydrous isobutyronitrile (50 mL) were added to a three-necked flask and bubbled for 10 min with nitrogen introduced through a conduit. The reaction solution was cooled to 0° C. and stirred, and then dry hydrogen chloride gas was introduced for 20 min. The reaction solution was then stirred at 80° C. for 1 h. After the reaction was completed, the reaction solution was concentrated, and the remaining solid was slurried with diethyl ether and filtered to give a crude product as an intermediate, which was directly used in the next step without purification. The intermediate, 1,4-dioxane (40 mL), water (20 mL) and sodium bicarbonate (1.6 g) were added to a reaction flask and stirred at 100° C. for 1 h. After the reaction was completed, the solvent was removed under reduced pressure, and the remaining solid was diluted with acetonitrile and filtered to give a crude product of SZ-015339A2 (4 g, a white solid) containing sodium chloride. LCMS: [M+1]⁺179.09.

Step 3:

SZ-015339A2 (1.5 g), NBS (1.42 g) and DMF (50 mL) were added to a reaction flask and stirred at 80° C. for 1 h. The reaction solution was concentrated by rotary evaporation under reduced pressure to remove the solvent, and the residue was diluted with dichloromethane and filtered to give a crude product of SZ-015339A3 (1.4 g, a white solid) containing sodium chloride. LCMS: 258.42.

Step 4:

SZ-015339A3 (1.43 g), 1-Boc-4-aminopiperidine (2.4 g), PyBOP (4.68 g), DCE (20 mL) and DIEA (1.5 mL) were added to a reaction flask and stirred at room temperature for 12 h. After the reaction was completed, the reaction solution was filtered and washed with dichloromethane. The organic phase was concentrated by rotary evaporation to remove the solvent, and the resulting crude product was purified by flash silica gel column chromatography (EA/PE=0%-40%) to give product SZ-015339A4 (1.43 g). LCMS: 440.60.

Step 5:

SZ-015339A4 (1.27 g), isopropenylboronic acid pinacol ester (1 g), Pd(dppf)₂Cl₂ (244 mg), potassium carbonate (1.24 g), 1,4-dioxane (36 mL) and water (6 mL) were added to a reaction flask and stirred at 80° C. for 3 h under nitrogen atmosphere. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015339A5 (1.29 g). LCMS: [M+1]⁺401.26.

Step 6:

SZ-015339A5 (1.2 g), Pd(OH)₂ (160 mg), ammonium formate (1.2 g) and methanol (30 mL) were added to a reaction flask and stirred at 80° C. for 1 h. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015339A6 (888 mg). LCMS: [M+1]⁺403.27.

Step 7:

SZ-015339A6 (984 mg), dichloromethane (10 mL) and TFA (2 mL) were added to a reaction flask and stirred at room temperature for 1 h. After the reaction was completed, the reaction solution was concentrated by rotary evaporation to give a crude product of SZ-015339A7, which was directly used in the next step without separation. LCMS: [M+1]+303.22.

Step 8:

(S)-1-N-tert-butoxycarbonyl-3-hydroxypyrrolidine (505 mg), acetonitrile (10 mL), triethylamine (2 mL) and p-nitrophenyl chloroformate (544 mg) were added successively to a reaction flask and stirred at 40° C. for 2 h. The crude product of SZ-015339A7 in the previous step was dissolved in acetonitrile (5 mL), triethylamine was added to adjust the solution to be alkaline, and then the reaction solution was added dropwise to the solution of SZ-015339A7 in acetonitrile. The resulting reaction solution was stirred at room temperature overnight. After the reaction was completed, the solvent was removed by rotary evaporation, and the residue was purified by flash silica gel column chromatography (methanol:dichloromethane=0%-5%) to give SZ-015339A8 (987 mg). LCMS: [M+1]⁺516.24.

Step 9:

SZ-015339A8 (987 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015339A9, which was directly used in the next step without separation. LCMS: [M+1]+416.10.

Step 10:

The crude product of SZ-015339A9 in the previous step, (2E)-4-(dimethylamino)-2-butenoic acid hydrochloride (378 mg), DIEA (2 mL), HATU (874 mg) and DMF (10 mL) were added to a reaction flask and stirred at room temperature for 2 h. After the reaction was completed, the solvent was removed by rotary evaporation, and the residue was separated by a reverse phase column (acetonitrile-water with 0.01% formic acid) to give SZ-015339 (440 mg). LCMS: [M+1]⁺ 527.28.

¹H NMR (400 MHz, DMSO-d₆) δ 8.40 (d, J=7.7 Hz, 1H), 7.33 (s, 1H), 6.74-6.60 (m, 2H), 5.21 (d, J=19.3 Hz, 1H), 4.34-4.26 (m, 1H), 4.02 (br, 2H), 3.90-3.76 (m, 3H), 3.69-3.60 (m, 2H), 3.56-3.27 (m, 2H), 3.02-2.86 (m, 3H), 2.79 (s, 6H), 2.28-2.02 (m, 2H), 1.93-1.90 (m, 2H), 1.67-1.54 (m, 2H), 1.34 (d, J=6.9 Hz, 6H), 1.28 (d, J=6.8 Hz, 6H).

### Example 38 SZ-015346: (S,E)-1-(4-(dimethylamino)but-2-enoyl)pyrrolidin-3-yl 4-((2-isopropoxy-7-isopropylimidazo[2,1-f][1,2,4]triazin-4-yl)amino)piperidine-1-carboxylate

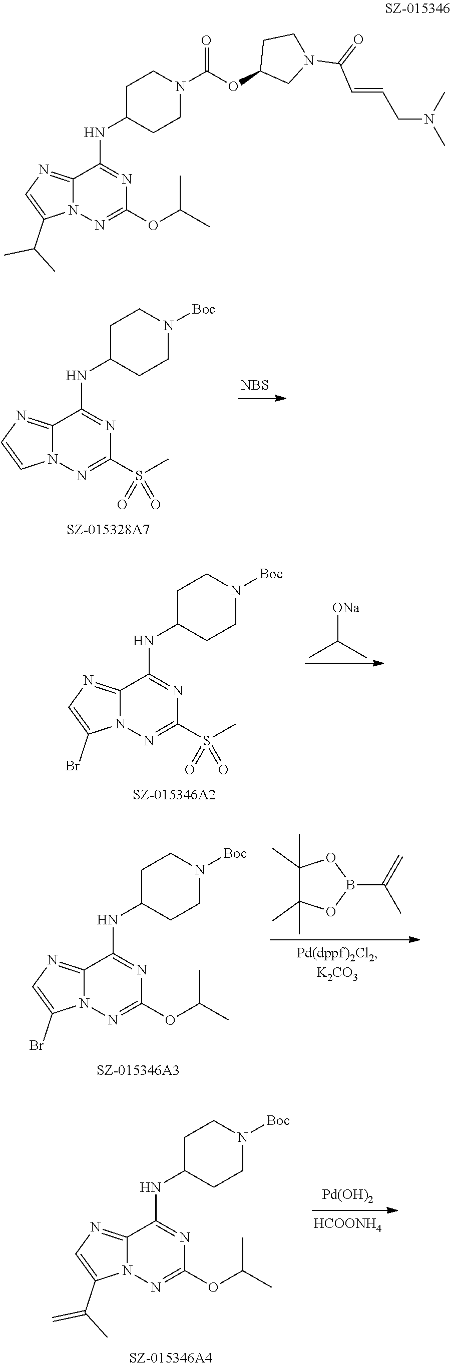

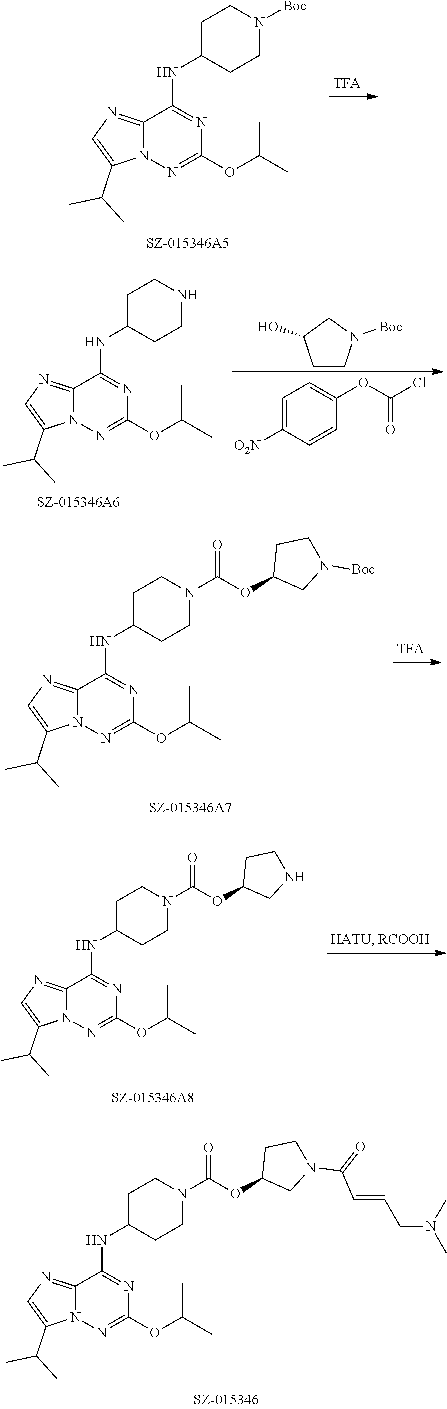

Step 1:

SZ-015328A7 (792 mg), NBS (711 mg) and MeCN (30 mL) were added to a reaction flask and stirred at 80° C. for 1 h. After the reaction was completed, the solvent was removed by rotary evaporation, and the resulting crude product was purified by flash silica gel column chromatography (EA/PE=0%-40%) to give product SZ-015346A2 (1.3 g). LCMS: 374.81, 376.77, 420.80, 418.83.

Step 2:

Isopropanol (100 mL) was added to a reaction flask, and NaH (500 mg) was added with stirring at 0° C. The reaction solution was stirred at room temperature for 30 min, and then SZ-015346A2 (1.3 g) was added. The resulting reaction solution was stirred at 80° C. for 1 h. Water (400 mL) was added to the reaction solution to quench the reaction, and dichloromethane (200 mL×3) was added for extraction. The organic phases were combined and concentrated by rotary evaporation under reduced pressure to remove the solvent, and the resulting crude product was purified by flash silica gel column chromatography (EA/PE=0%-30%) to give product SZ-015346A3 (808 mg). LCMS: 418.81, 420.82

Step 3:

SZ-015346A3 (808 mg), isopropenylboronic acid pinacol ester (504 mg), Pd(dppf)₂Cl₂ (163 mg), potassium carbonate (552 mg), 1,4-dioxane (20 mL) and water (5 mL) were added to a reaction flask and stirred at 80° C. for 3 h under nitrogen atmosphere. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015346A4 (900 mg). LCMS: [M+1]⁺417.11.

Step 4:

SZ-015346A4 (900 mg), Pd(OH)₂ (200 mg), ammonium formate (3 g) and methanol (30 mL) were added to a reaction flask and stirred at 80° C. for 1 h. The solvent was removed by rotary evaporation, and the residue was diluted with ethyl acetate and filtered. The organic phase was concentrated by rotary evaporation to remove the solvent, and the resulting crude product was purified by flash silica gel column chromatography (EA/PE=0%-30%) to give product SZ-015346A5 (438 mg). LCMS: [M+1]⁺419.11.

Step 5:

SZ-015346A5 (438 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015346A6, which was directly used in the next step without separation. LCMS: [M+1]+319.10.

Step 6:

(S)-1-N-tert-butoxycarbonyl-3-hydroxypyrrolidine (280 mg), acetonitrile (20 mL), triethylamine (5 mL) and p-nitrophenyl chloroformate (302 mg) were added successively to a reaction flask and stirred at 40° C. for 2 h. The crude product of SZ-015346A6 in the previous step was dissolved in acetonitrile (5 mL), triethylamine was added to adjust the solution to be alkaline, and then the reaction solution was added dropwise to the solution of SZ-015346A6 in acetonitrile. The resulting reaction solution was stirred at room temperature for 4 h. After the reaction was completed, the solvent was removed by rotary evaporation, and the residue was purified by flash silica gel column chromatography (methanol:dichloromethane=0%-5%) to give SZ-015346A7 (688 mg). LCMS: [M+1]⁺532.24.

Step 7:

SZ-015346A7 (688 mg), dichloromethane (10 mL) and TFA (3 mL) were added to a reaction flask and stirred at room temperature for 1 h. After the reaction was completed, the reaction solution was concentrated by rotary evaporation to give a crude product of SZ-015346A8, which was directly used in the next step without separation. LCMS: [M+1]+432.10.

Step 8:

The crude product of SZ-015346A8 in the previous step, (2E)-4-(dimethylamino)-2-butenoic acid hydrochloride (330 mg), DIEA (3 mL), HATU (760 mg) and DMF (20 mL) were added to a reaction flask and stirred at room temperature for 2 h. After the reaction was completed, the solvent was removed by rotary evaporation, and the residue was separated by a reverse phase column (acetonitrile-water with 0.01% formic acid) to give SZ-015346 (324 mg). LCMS: [M+1]⁺543.35.

¹H NMR (400 MHz, DMSO-d₆) δ 8.55 (d, J=8.1 Hz, 1H), 7.24 (s, 1H), 6.66-6.59 (m, 1H), 6.41-6.32 (m, 1H), 5.21 (d, J=19.3 Hz, 1H), 5.10-5.01 (m, 1H), 4.28-4.20 (m, 1H), 3.98 (br, 2H), 3.82-3.70 (m, 1H), 3.62-3.34 (m, 3H), 3.24-3.17 (m, 1H), 3.05 (d, J=6.1 Hz, 2H), 2.91 (br, 2H), 2.16 (s, 6H), 2.15-1.97 (m, 2H), 1.82-1.80 (m, 2H), 1.61-1.52 (m, 2H), 1.33-1.30 (m, 12H).

### Example 39 SZ-015297: (S,E)-1-(4-(dimethylamino)but-2-enoyl)pyrrolidin-3-yl-4-((2-(hydroxymethyl)-9-isopropyl-9H-purin-6-yl)amino)piperidine-1-carboxylate

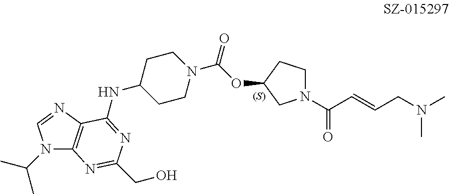

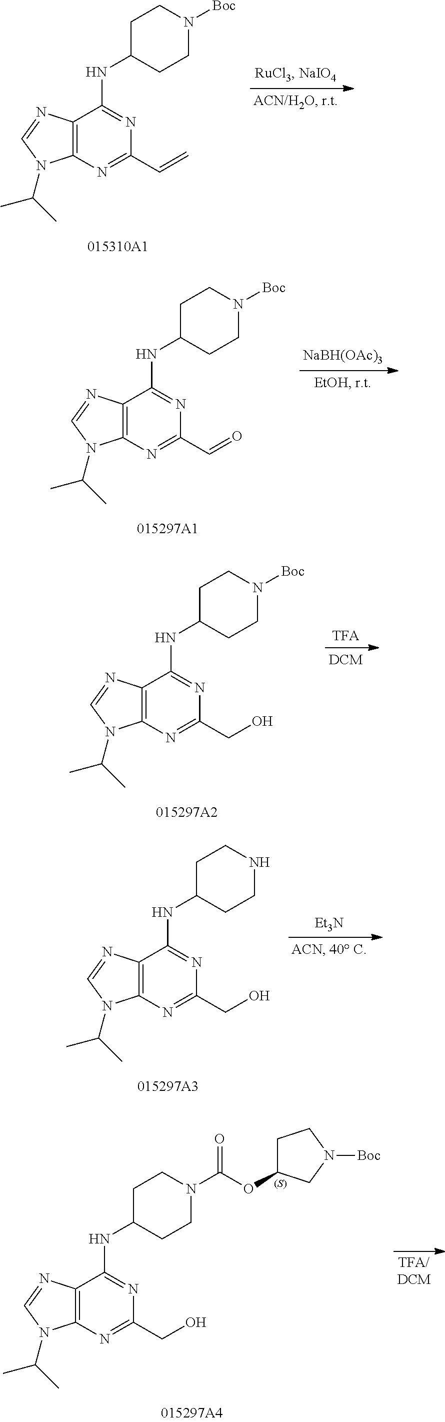

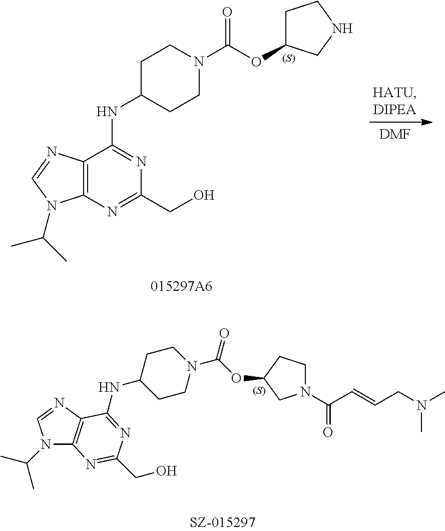

Step 1:

015310A1 (1.9 g, 5 mmol) was dissolved in acetonitrile (50 mL) and water (10 mL), and ruthenium trichloride (82 mg, 5 mol %) and NaIO₄ (3.2 g, 15 mmol) were added. The reaction solution was stirred at room temperature for 12 h, and then water (100 mL) was added and ethyl acetate (30 mL×3) was added for extraction. The organic phase was dried over sodium sulfate and concentrated by rotary evaporation under reduced pressure to give a crude product, which was purified by flash silica gel column chromatography to give 015297A1(1.2 g, a white solid). LCMS: [M+H]⁺ 389.3.

Step 2:

015297A1(1.2 g, 3 mmol) was dissolved in ethanol (40 mL), and NaBH(OAc)₃ (1.27 g, 6 mmol) was added. The reaction solution was stirred at room temperature for 2 h and concentrated by rotary evaporation under reduced pressure to give a crude product, which was purified by flash silica gel column chromatography to give 015297A2 (380 mg, a white solid). LCMS: [M+H]⁺ 391.4.

Step 3:

Compound 015297A2 (340 mg, 0.85 mmol) was dissolved in dichloromethane (9 mL), and trifluoroacetic acid (3 mL) was added. The reaction solution was stirred at room temperature for 1 h and concentrated by rotary evaporation under reduced pressure to give 015297A3 trifluoroacetate (340 mg, 100%, a colorless oily liquid). LCMS: [M+H]⁺ 291.3.

Step 4:

Compound 015297A3 trifluoroacetate (340 mg, 0.85 mmol) was dissolved in acetonitrile (9 mL), and triethylamine (258 mg, 2.55 mmol) and tert-butyl (S)-3-(((4-nitrophenoxy) carbonyl)oxo)pyrrolidine-1-carboxylate (300 mg, 0.85 mmol) were added successively. The reaction solution was stirred at room temperature for 2 h and concentrated by rotary evaporation under reduced pressure to give a crude product of 015297A4, which was purified by flash silica gel column chromatography to give 015297A4 (340 mg, a white solid). LCMS: [M+H]⁺ 504.5.

Step 5:

Compound 015297A4 (340 mg, 0.68 mmol) was dissolved in dichloromethane (6 mL), and trifluoroacetic acid (2 mL) was added. The reaction solution was stirred at room temperature for 0.5 h and concentrated by rotary evaporation under reduced pressure to give 015297A5 trifluoroacetate (340 mg, 100%, a colorless oily liquid). LCMS: [M+H]⁺ 404.4.

Step 6:

Compound 015297A5 trifluoroacetate (340 mg, 0.68 mmol) was dissolved in DMF (5 mL), and DIPEA (260 mg, 2 mmol), (E)-4-(dimethylamino)but-2-enoic acid hydrochloride (112.5 mg, 0.68 mmol) and HATU (310 mg, 0.82 mmol) were added successively. The reaction solution was stirred at room temperature for 0.5 h and concentrated by rotary evaporation under reduced pressure to give a crude product of SZ-015297, which was subjected to reverse phase column chromatography (acetonitrile/water with 0.1% formic acid) and lyophilized to give SZ-015297 (70 mg, a white solid). LCMS: [M+H]⁺ 515.13.

¹H NMR (400 MHz, DMSO-d₆) δ 8.22 (s, 1H), 7.61 (d, J=6.4 Hz, 1H), 6.62 (ddd, J=16.3, 10.5, 6.1 Hz, 1H), 6.50-6.31 (m, 1H), 5.15 (d, J=23.4 Hz, 1H), 4.74 (dt, J=13.2, 6.6 Hz, 1H), 4.41 (s, 3H), 4.04-3.27 (m, 6H), 3.15 (d, J=5.3 Hz, 2H), 2.88 (t, J=26.8 Hz, 3H), 2.34-1.75 (m, 10H), 1.60-1.40 (m, 8H).

### Efficacy Example 1: Activity Assay

IC₅₀ of test compounds inhibiting CDK7 was detected using the mobility shift assay, and effective compounds were evaluated and screened. The initial concentration of compound was 10 μM, and the compound was 3-fold diluted to obtain 10 concentrations. Duplicate wells were set for the assay, and the pre-incubation time of compound and enzyme was 60 min.

1. Preparation of Compounds

Compound powders were dissolved in 100% DMSO to prepare 10 mM stock solution, which was 3-fold diluted with DMSO, with 0.5 mM as the initial concentration, to obtain 10 concentration gradients of compound solutions.

2. Reaction Process of Kinase

(1) Compound at each concentration was 8.3-fold diluted with ddH₂O, and then 2 μL of each compound solution and positive control were added to a 384-well plate.

(2) 5 μL of a kinase working solution (CDK7/Cyclin H/MAT1) was added to the compound wells and positive control wells to make the final concentration of CDK7 be 3 nM.

(3) The plate was pre-incubated at room temperature for 60 min.

(4) 5 μL of substrate ((5-FAM)-YSPTSPSYSPTSPSYSPTSPSKKKK-NH2) solution was added to each well to initiate the reaction; the concentration of ATP is 2 mM, and the concentration of peptide substrate was 2 μM.

(5) The 384-well plate was incubated at 27° C. for 36 min.

(6) 4 μL of 80 mM EDTA was added to terminate the reaction.

(7) The conversion rates were read using a Caliper EZ Reader II.

3. The results are shown in Table 1 below

TABLE 1

| | IC₅₀ |
|---|---|
| Compound | (nM) |
| SZ-015200 (Example 1 | 54.06 |
| in WO2019099298) | |
| SZ-015201 (Example 3 | 134.00 |
| in WO2019099298) | |
| CT7001 | 33.20 |
| SZ-015256 | 54.29 |
| SZ-015095 | 128.30 |
| SZ-015272 | 278.20 |
| SZ-015273 | 44.46 |
| SZ-0015268 | 23.56 |
| SZ-015274 | 197.16 |

### Efficacy Example 2: Inhibition of Compounds Against Cell Proliferation

1. Preparation of Compounds

The compounds were each prepared with 100% DMSO into 10 mM mother liquor.

2. Test Method

(1) Day 0: cell plating

HCC70 cells and OVCAR3 cells were digested and counted. Cells were diluted to an appropriate concentration according to cell density. 100 μL of cells were added to each well of a 96-well plate. Medium wells served as blank control (Min). The cells were incubated overnight in an incubator at 37° C./5% CO₂.

(2) Day 1: treatment of compounds

A 200-fold stock solution of a compound was prepared with DMSO, and the compound was diluted with growth medium to a 3-fold stock solution, i.e., 3 μL of each of 200-fold stock solutions of compound at different concentrations was added to 197 μL of medium. 50 μL of diluted compound was added to each well and the mixture was incubated at 37° C./5% CO₂ for 72 h.

(3) Day 4: detection

The 96-well plate was equilibrated to room temperature before detection. 40 μL of CellTiter-Glo solution was added to each well. The mixture was mixed for 2 min using a horizontal shaker to induce cell lysis. The mixture was incubated at room temperature for 60 min to stabilize fluorescence signals. Fluorescence values were read on ENVISION.

3. Data Analysis

(1) Data was analyzed by GraphPad Prism 5

(2) % inhibition rate=(maximum signal−compound signal)/(maximum signal−minimum signal)×100

(3) Maximum signal: signal of DMSO well

(4) Minimum signal: signal of medium well

4. The results are shown in Table 2 below

TABLE 2

| | HCC70 | OVCAR-3 |
|---|---|---|
| | IC₅₀ | IC₅₀ |
| Compound No. | (nM) | (nM) |
| SZ-015200 | 34.14 | 32.00 |
| (i.e., compound | | |
| of Example 1 in | | |
| WO2019099298A1) | | |
| SZ-015201 | 25.47 | 22.56 |
| (compound | | |
| of Example 3 in | | |
| WO2019099298A1) | | |
| CT7001 | 310.20 | 467.00 |
| SZ-015256 | 174.70 | 75.10 |
| SZ-015095 | 479.30 | 137.50 |
| SZ-015264 | 181.00 | 233.70 |
| SZ-015272 | 947.90 | 460.2 |
| SZ-015273 | 291.20 | 31.85 |
| SZ-015268 | 57.55 | 29.92 |
| SZ-015274 | 186.6 | 150.9 |
| SZ-015282 | 82.36 | 92.14 |
| SZ-015294 | 50.19 | 72.07 |
| SZ-015284 | 97.44 | — |
| SZ-015289 | 116.9 | — |
| SZ-015285 | 1049 | 620 |
| SZ-015286 | 569.6 | 166.9 |
| SZ-015303 | 128.8 | 114.3 |
| SZ-015307 | 77.95 | 138.8 |
| SZ-015308 | 92.06 | — |
| SZ-015295 | 110.3 | 62.81 |
| SZ-015298 | 405.4 | — |
| SZ-015310 | 29.49 | 58.4 |
| SZ-015306 | 205.2 | 280.7 |
| SZ-015311 | 95.01 | 99.6 |
| SZ-015297 | 252.5 | 167.6 |
| SZ-015317 | 237.4 | 428.6 |
| SZ-015319 | 50.53 | 72.46 |
| SZ-015326 | 50.26 | 33.23 |
| SZ-015330 | 60.99 | 43.96 |
| SZ-015331 | 70.10 | 36.73 |
| SZ-015332 | 51.51 | 44.68 |
| SZ-015342 | 418.9 | 204 |
| SZ-015338 | 48.69 | 29.7 |
| SZ-015339 | 54.68 | 64.28 |
| SZ-015328 | 252.3 | 110.4 |
| SZ-015346 | 139.5 | 48.77 |

Note:
“—” represents not tested.

### Efficacy Example 3: Inhibition of Compounds Against Cell Proliferation

The effect of SZ-015200, SZ-015256 and SZ-015268 on cell proliferation after incubation with HCC70, OVCAR-3, HCT116 and HCC1806 cells for 3 days was detected according to the method described in Efficacy Example 2, and the results are shown in Table 3.

TABLE 3
Cell proliferation inhibition rates

| | HCC70 | OVCAR-3 | HCT116 | HCC1806 |
|---|---|---|---|---|
| | IC₅₀ | IC₅₀ | IC₅₀ | IC₅₀ |
| Compound No. | (nM) | (nM) | (nM) | (nM) |
| SZ-015200 | 68.61 | 101.50 | 24.11 | 65.43 |
| (i.e., compound of | | | | |
| Example 1 in | | | | |
| WO2019099298A1) | | | | |
| SZ-015256 | 50.85 | 45.31 | 25.26 | 44.47 |
| SZ-015268 | 33.00 | 80.56 | 12.53 | 61.55 |

### Efficacy Example 4: In Vivo Pharmacokinetic Properties after Intragastric Administration of Compounds in Mice

Male 1CR mice were divided into different groups with 3 mice in each group, and single intragastric administration was conducted for each group. The compounds were each formulated with 10% DMSO/10% Solutol/80% (2000 Captisol) to reach an appropriate concentration and then administered intragastrically at 20 mg/kg. Blood was taken 5 min, 15 min, 30 min, 1 h, 2 h, 4 h, 6 h, 8 h and 24 h after administration, and then subjected to EDTA-K2 anticoagulation. The concentration of the compound in the plasma was quantitatively detected by an LC-MS/MS method. Pharmacokinetic parameters of each compound were calculated by Phoenix WinNonlin 7.0, and the results are summarized in Table 4 below.

TABLE 4
Pharmacokinetic parameters of each group after intragastric
administration of compounds in mice

| | Dosage | T_{1/2} | Tₘₐₓ | Cₘₐₓ | AUC₍₀₋ₜ₎ | AUC_{(0-∞)} |
|---|---|---|---|---|---|---|
| | mg/kg | h | h | ng/mL | h*ng/mL | h*ng/mL |
| SZ-015200 | 20.00 | 1.97 | 0.67 | 1209.14 | 2926.85 | 3121.49 |
| SZ-015256 | 20.00 | 1.48 | 0.50 | 3379.92 | 6258.34 | 6375.00 |
| SZ-015268 | 20.00 | 1.39 | 0.42 | 1544.63 | 3095.46 | 3149.75 |

The results show that compared with the control compound SZ-015200, the Cₘₐₓ and the AUC of SZ-015256 and SZ-015268 in mice after single intragastric administration were not lower than those of SZ-015200, with those of SZ-015256 being significantly higher.

### Efficacy Example 5: In Vivo Pharmacokinetic Properties after Intragastric Administration of Compounds in Rats

Male SD rats were divided into different groups with 3 rats in each group, and single intragastric administration was conducted for each group. The compounds were each formulated with 10% DMSO/10% Solutol/80% (20% Captisol) to reach an appropriate concentration and then administered intragastrically at 40 mg/kg. Blood was taken 5 min, 15 min, 30 min, 1 h, 2 h, 4 h, 6 h, 8 h and 24 h after administration, and then subjected to EDTA-K2 anticoagulation. The concentration of the compound in the plasma was quantitatively detected by an LC-MS/MS method. Pharmacokinetic parameters of each compound were calculated by Phoenix WinNonlin 7.0, and the results are summarized in Table 5 below.

TABLE 5
Pharmacokinetic parameters of each group after intragastric
administration of compounds in rats

| | Dosage | T_{1/2} | Tₘₐₓ | Cₘₐₓ | AUC₍₀₋ₜ₎ | AUC_{(0-∞)} |
|---|---|---|---|---|---|---|
| | mg/kg | h | h | ng/mL | h*ng/mL | h*ng/mL |
| SZ-015200 | 40.00 | 4.11 | 3.33 | 322.98 | 1988.83 | 2554.45 |
| SZ-015256 | 40.00 | 2.99 | 4.67 | 783.01 | 7828.87 | 7879.45 |
| SZ-015268 | 40.00 | 3.92 | 4.00 | 542.53 | 4449.20 | 4513.87 |

The results show that compared with the control compound SZ-015200, the Cₘₐₓ and the AUC of SZ-015256 and SZ-015268 in rats after single intragastric administration were significantly higher, which indicates that SZ-015256 and SZ-015268 have better pharmacokinetic properties.

### Efficacy Example 6: In Vivo Efficacy of Compounds in Subcutaneous Xenograft Tumor Model of Human Colon Cancer Cells HCT116

Test Method

Cell Culturing

HCT116 tumor cells used in this test were purchased from ATCC. HCT116 tumor cells were cultured in McCoy's 5a medium containing inactivated 10% fetal bovine serum, 100 U/mL penicillin, 100 μg/mL streptomycin and 2 mM glutamine in an incubator at 37° C./5% CO₂. The cells were divided and transferred to other bottles every 3 to 4 days for passaging when the culture bottle was full of cells. The tumor cells in the logarithmic growth phase were used for tumor seeding in vivo.

Seeding and Grouping of Tumor Cells

HCT116 tumor cells resuspended in serum-free McCoy's 5a culture solution were seeded subcutaneously into the right costal part of male BALB/c nude mice at 5×10⁶/0.1 mL. When the tumor grew to 101 mm³, animals with uniform tumor volume were selected and divided into groups with 8 animals in each group for administration, and the specific administration regimens are shown in Table 6 below.

TABLE 6
Administration regimens

| | | | | Administration | | |
|---|---|---|---|---|---|---|
| | Cases of | | Dosage | volume | Route of | Course of |
| Group | animals | Treatment | (mg/kg)* | (μL/g) | administration | treatment |
| 1 | 8 | Vehicle | — | 10 | p.o. | QD × 21 |
| | | (control) | | | | days |
| | | group | | | | |
| 2 | 8 | SZ-015200 | 25 | 10 | p.o. | QD × 21 |
| | | | | | | days |
| 3 | 8 | SZ-015256 | 25 | 10 | p.o. | QD × 21 |
| | | | | | | days |
| 4 | 8 | SZ-015256 | 15 | 10 | p.o. | QD × 21 |
| | | | | | | days |
| 5 | 8 | SZ-015268 | 25 | 10 | p.o. | QD × 21 |
| | | | | | | days |

Note:
*: dosage refers to the amount of the pure drug

Test Indexes

Tumor volume: the long and short diameters of the tumor were measured twice every week using a vernier caliper for calculating the tumor volume, and the calculation formula was:

volume=0.5×long diameter×short diameter².

Reaction of animals after the administration: the mice were weighed while measuring tumor volume. The relationship between the change in the body weight of mice and the time of administration was recorded. At the same time, the survival and health of the mice, such as animal activity, feeding and other general states during the administration were observed.

Drug Evaluation Indexes

Relative increase ratio of tumor volume TIC (%)

T/C(0%)=mean value of relative tumor volume of treatment group/mean value of relative tumor volume of control group×100

Tumor growth inhibition rate (TGI, 0%)

Tumor growth inhibition rate(TGI,%)=(1−T/C)×100

Statistical Analysis

Statistical analysis between groups was performed for tumor volume using One-Way ANOVA test, and a significant difference was considered to be present when p<0.05.

Test Results

Change in Body Weight of Animal

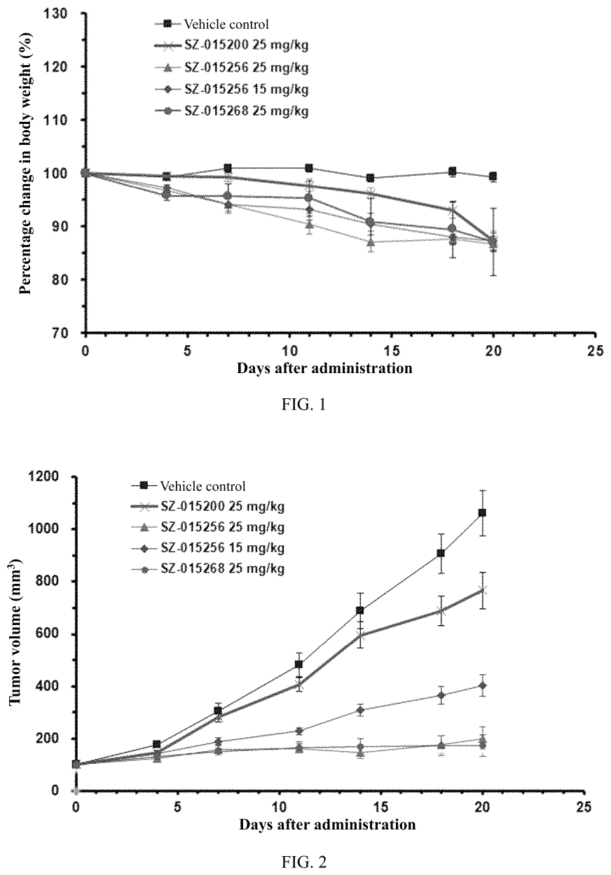

The body weight of the experimental animal was used as a reference index for indirectly measuring the toxicity of the drug. Relative body weight changes are shown in FIG. 1. The results show that the body weight of animals in each administration group was reduced by a certain degree compared with that in vehicle control group, but there were no significant differences in body weight of animals between administration groups. The experimental animals in all groups were in good state in terms of activity, feeding and other general states, and no obvious clinical abnormalities were found. It is indicated that the animal can tolerate an administration dose of 25 mg/kg.

Tumor Growth Curves

The tumor growth curves are shown in FIG. 2.

Calculation of Anti-Tumor Efficacy Evaluation Index TGI

TGI data are summarized in Table 7.

TABLE 7
Inhibition of compounds against tumor growth in xenograft model
of HCT116 human colon cancer

| | | Tumor | % proliferation | | |
|---|---|---|---|---|---|
| | | volume | percentage of | Tumor | |
| | | (mm³, day | tumor volume | inhibition | |
| | | 21 after | (Day 21 after | rate | P value ^{b} |
| Group | Treatment | grouping) ^{a} | grouping) ^{a} | (%) ^{c} | (Dunnett T3) |
| 1 | Solvent control | 1,062 ± 86 | 1,061 ± 65 | — | — |
| 2 | SZ-015200 | 766 ± 71 | 764 ± 58 | 27.9 | 0.210 |
| | 25 mg/kg QD × 21 | | | | |
| 3 | SZ-015256 | 201 ± 43 | 192 ± 38 | 81.9 | <0.001 |
| | 25 mg/kg QD × 21 | | | | |
| 4 | SZ-015256 | 402 ± 42 | 407 ± 40 | 61.6 | 0.001 |
| | 15 mg/kg QD × 21 | | | | |
| 5 | SZ-015268 | 172 ± 41 | 167 ± 39 | 84.3 | <0.001 |
| | 25 mg/kg QD × 21 | | | | |

Note:
^{a} mean value ± standard error,
^{b} vs. solvent control group;
^{c} tumor growth inhibition rate (TGI, %) = (1 − T/C) × 100, where T/C (%) = mean value of relative tumor volume of treatment group/mean value of relative tumor volume of control group × 100

The above results show that: compound SZ-015256 shows significant anti-tumor effect in both 25 mg/kg and 15 mg/kg treatment groups of this compound, compound SZ-015268 shows significant anti-tumor effect in 25 mg/kg treatment group of this compound, and compound SZ-015200 shows slight anti-tumor tendency in 25 mg/kg treatment group of this compound. Compared with the control compound SZ-015200, SZ-015256 and SZ-015268 have stronger effect on inhibiting tumor proliferation and have extremely significant anti-tumor advantage.

1. A compound of formula I: 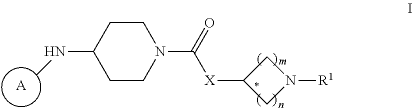
or a tautomer, a stereoisomer or an isotopic derivative thereof, or a pharmaceutically acceptable salt of any of the foregoing, or a crystalline form or a solvate of any of the foregoing;
wherein, ring A is 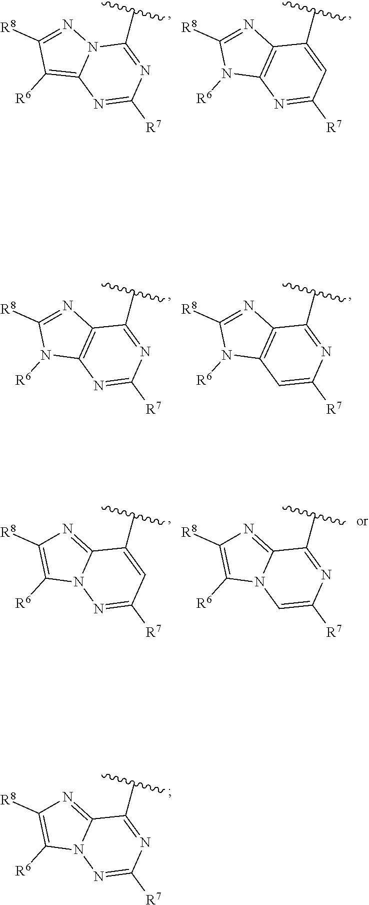
X is O or NR^{a};
R^{a} is hydrogen, methyl or ethyl;
m is 1 or 2;
n is 1, 2 or 3;
R¹ is 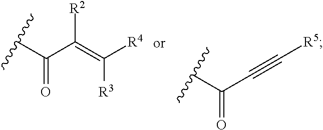
R² is hydrogen or C₁₋₆ alkyl;
R³ is hydrogen or C₁₋₆ alkyl;
R⁴ is hydrogen, halogen, C₁₋₆ alkyl or —CH₂—NR^{4a}R^{4b};
R^{4a} and R^{4b} are each independently hydrogen or C₁₋₆ alkyl; or, R^{4a} and R^{4b}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{b};
each R^{b} is independently halogen, hydroxy or C₁₋₄ alkyl;
R⁵ is hydrogen or C₁₋₆ alkyl;
each R⁶ is independently hydrogen, cyclopropyl or C₁₋₆ alkyl;
each R⁷ is independently hydrogen, a substituted or unsubstituted C₁₋₆ alkyl, a substituted or unsubstituted phenyl, a substituted or unsubstituted 5-6 membered heteroaryl, a substituted or unsubstituted C₃₋₆ cycloalkyl, —OR^{7a} or —NR^{7b}R^{7c}, wherein the substituted C₁₋₆ alkyl, the substituted C₃₋₆ cycloalkyl, the substituted phenyl and the substituted 5-6 membered heteroaryl mean that the C₁₋₆ alkyl, the C₃₋₆ cycloalkyl, the phenyl and the 5-6 membered heteroaryl are each independently substituted with 1, 2, 3 or 4 R^{7d};
each R^{7d} is independently hydroxy, halogen, C₁₋₄ alkyl, —NR^{a1}R^{a2} or C₁₋₄ alkoxy;
R^{7a} is hydrogen, a substituted or unsubstituted C₁₋₆ alkyl, a substituted or unsubstituted C₃₋₆ cycloalkyl, a substituted or unsubstituted 4-6 membered heterocycloalkyl, or a substituted or unsubstituted 5-6 membered heteroaryl, wherein the substituted C₁₋₆ alkyl, the substituted C₃₋₆ cycloalkyl, the substituted 4-6 membered heterocycloalkyl and the substituted 5-6 membered heteroaryl mean that the C₁₋₆ alkyl, the C₃₋₆ cycloalkyl, the 4-6 membered heterocycloalkyl and the 5-6 membered heteroaryl are each independently substituted with 1, 2, 3 or 4 R^{c};
each R^{c} is independently hydroxy, halogen, C₁₋₄ alkyl, —NR^{c1}R^{c2} or C₁₋₄ alkoxy;
R^{7b} and R^{7c} are each independently hydrogen, a substituted or unsubstituted C₁₋₄ alkyl, a substituted or unsubstituted C₃₋₆ cycloalkyl, a substituted or unsubstituted 4-6 membered heterocycloalkyl, or a substituted or unsubstituted 5-6 membered heteroaryl, wherein the substituted C1.4 alkyl, the substituted C₃₋₆ cycloalkyl, the substituted 4-6 membered heterocycloalkyl and the substituted 5-6 membered heteroaryl mean that the C₁₋₄ alkyl, the C₃0.6 cycloalkyl, the 4-6 membered heterocycloalkyl and the 5-6 membered heteroaryl are each independently substituted with 1, 2, 3 or 4 R^{d};
or, R^{7b} and R^{7c}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e};
each R^{d} is independently hydroxy, halogen, C₁₋₄ alkyl, —NR^{d1}R^{d2} or C₁₋₄ alkoxy;
each R^{e} is independently hydroxy, halogen, C₁₋₄ alkyl, —NR^{e1}R^{e2} or C₁₋₄ alkoxy;
each R^{a1}, each R^{a2}, each R^{c1}, each R^{c2}, each R^{d1}, each R^{d2}, each R^{e1} and each R^{e2} are each independently hydrogen or C₁₋₄ alkyl;
each R^{g} is independently hydrogen or C₁₋₄ alkyl;
when a carbon atom marked by * has chirality, the carbon atom is in S configuration, R configuration, or a mixture thereof,
the number of heteroatoms in the 4-6 membered heterocycloalkyl and the number of heteroatoms in the 5-6 membered heteroaryl are independently 1, 2 or 3, and each heteroatom is independently selected from N, O and S.

2. The compound or the tautomer, the stereoisomer or the isotopic derivative thereof, or the pharmaceutically acceptable salt of any of the foregoing, or the crystalline form or the solvate of any of the foregoing according to claim 1, wherein each C₁₋₄ alkyl is independently methyl, ethyl, n-propyl, isopropyl, n-butyl, isobutyl, sec-butyl or tert-butyl;
or, each halogen is independently fluorine, chlorine, bromine or iodine;
or, each C₁₋₆ alkyl is independently methyl, ethyl, n-propyl, isopropyl, n-butyl, isobutyl, sec-butyl or tert-butyl;
or, each C₃₋₆ cycloalkyl is independently cyclopropyl, cyclobutyl, cyclopentyl or cyclohexyl;
or, each C₁₋₄ alkoxy is independently methoxy, ethoxy, n-propoxy, isopropoxy, n-butoxy, isobutoxy, sec-butoxy or tert-butoxy.

3. The compound or the tautomer, the stereoisomer or the isotopic derivative thereof, or the pharmaceutically acceptable salt of any of the foregoing, or the crystalline form or the solvate of any of the foregoing according to claim 1, wherein,
ring A is 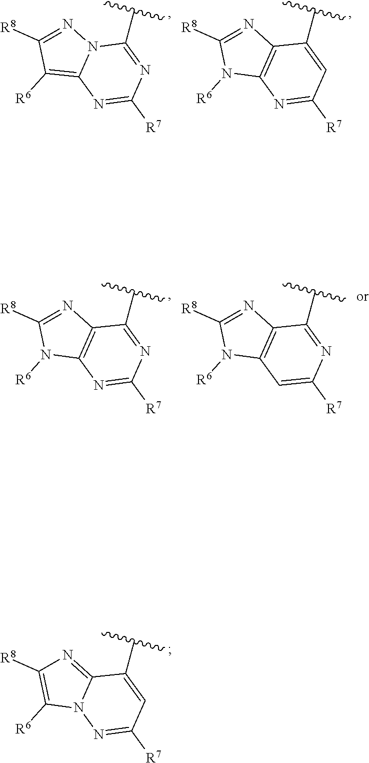
X is O or NR^{a};
R^{a} is hydrogen, methyl or ethyl;
m is 1 or 2;
n is 1, 2 or 3;
R¹ is 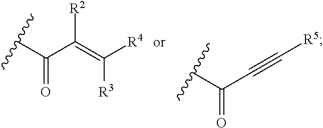
R² is hydrogen or C₁₋₆ alkyl;
R³ is hydrogen or C₁₋₆ alkyl;
R⁴ is hydrogen, halogen, C₁₋₆ alkyl or —CH₂—NR^{4a}R^{4b};
R^{4a} and R^{4b} are each independently hydrogen or C₁₋₆ alkyl; or, R^{4a} and R^{4b}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{b};
each R^{b} is independently halogen, hydroxy or C₁₋₄ alkyl;
R⁵ is hydrogen or C₁₋₆ alkyl;
each R⁶ is independently hydrogen, cyclopropyl or C₁₋₆ alkyl;
each R⁷ is independently hydrogen, C₁₋₆ alkyl, phenyl, 5-6 membered heteroaryl, —OR^{7a} or —NR^{7b}R^{7c}, wherein the number of heteroatoms in the 5-6 membered heteroaryl is 1, 2 or 3, and each heteroatom is independently selected from N, O and S;
R^{7a} is hydrogen or a substituted or unsubstituted C₁₋₆ alkyl, wherein the substituted C₁₋₆ alkyl means that the C₁₋₆ alkyl is substituted with 1, 2, 3 or 4 R^{c};
each R^{c} is independently hydroxy, halogen, C₁₋₄ alkyl or C₁₋₄ alkoxy;
R^{7b} and R^{7c} are each independently hydrogen or a substituted or unsubstituted C₁₋₄ alkyl, wherein the substituted C₁₋₄ alkyl means that the C₁₋₄ alkyl is substituted with 1, 2, 3 or 4 R^{d};
or, R^{7b} and R^{7c}, together with a nitrogen atom attached thereto, form a substituted or unsubstituted 4-6 membered heterocycloalkyl, wherein the 4-6 membered heterocycloalkyl has 0, 1 or 2 additional heteroatoms independently selected from N, O and S, and the substituted 4-6 membered heterocycloalkyl means that the 4-6 membered heterocycloalkyl is substituted with 1, 2, 3 or 4 R^{e};
each R^{d} is independently hydroxy, halogen, C₁₋₄ alkyl or C₁₋₄ alkoxy;
each R^{e} is independently hydroxy, halogen, C₁₋₄ alkyl or C₁₋₄ alkoxy;
each R^{g} is independently hydrogen or C₁₋₄ alkyl;
when a carbon atom marked by * has chirality, the carbon atom is in S configuration, R configuration, or a mixture thereof.

4. The compound or the tautomer, the stereoisomer or the isotopic derivative thereof, or the pharmaceutically acceptable salt of any of the foregoing, or the crystalline form or the solvate of any of the foregoing according to claim 1, wherein, when R^{4a} is C₁₋₆ alkyl, the C₁₋₆ alkyl is methyl;
or when R^{4b} is C₁₋₆ alkyl, the C₁₋₆ alkyl is methyl;
or when R⁵ is C₁₋₆ alkyl, the C₁₋₆ alkyl is methyl;
or when R⁶ is C₁₋₆ alkyl, the C₁₋₆ alkyl is methyl, ethyl, n-propyl or isopropyl;
or, when R⁷ is a substituted or unsubstituted C₁₋₆ alkyl, the C₁₋₆ alkyl is methyl, ethyl or isopropyl;
or, when R⁷ is a substituted or unsubstituted 5-6 membered heteroaryl, the 5-6 membered heteroaryl is pyrazolyl, e.g., 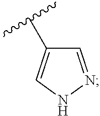 [SMILES: *c1cn[nH]c1]
or, when R⁷ is a substituted or unsubstituted C₃₋₆ cycloalkyl, the C₃₋₆ cycloalkyl is cyclopropyl;
or, when R^{7d} is C₁₋₄ alkoxy, the C₁₋₄ alkoxy is methoxy;
or, when R^{7a} is a substituted or unsubstituted C₁₋₆ alkyl, the C₁₋₆ alkyl is isopropyl;
or, when R^{7a} is a substituted or unsubstituted C₃₋₆ cycloalkyl, the C₃₋₆ cycloalkyl is cyclopropyl;
or, when R^{7b} and R^{7c} are each independently a substituted or unsubstituted C₁₋₄ alkyl, the C₁₋₄ alkyl is ethyl, isopropyl or sec-butyl;
or, when R^{7b} and R^{7c} are each independently a substituted or unsubstituted C₃₋₆ cycloalkyl, the C₃₋₆ cycloalkyl is cyclopropyl or cyclopentyl;
or, when R^{7b} and R^{7c} are each independently a substituted or unsubstituted 5-6 membered heteroaryl, the 5-6 membered heteroaryl is pyrazolyl, e.g., 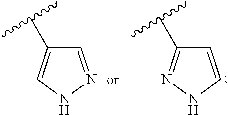 [SMILES: *c1cn[nH]c1 ; *c1cc[nH]n1]
or, when R^{7b} and R^{7c}, together with a nitrogen atom attached thereto, form a substituted or unsubstituted 4-6 membered heterocycloalkyl, the 4-6 membered heterocycloalkyl is azetidinyl;
or, when R^{c} is C₁₋₄ alkoxy, the C₁₋₄ alkoxy is methoxy;
or, when R^{d} is C₁₋₄ alkoxy, the C₁₋₄ alkoxy is methoxy;
or, when R^{e} is C₁₋₄ alkoxy, the C₁₋₄ alkoxy is methoxy;
or, when each R^{a1}, each R^{a2}, each R^{c1}, each R^{c2}, each R^{d1}, each R^{d2}, each R^{e1} and each R^{e2} are each independently C₁₋₄ alkyl, the C₁₋₄ alkyl is methyl;
or, when R^{g} is C₁₋₄ alkyl, the C₁₋₄ alkyl is methyl.

5. The compound or the tautomer, the stereoisomer or the isotopic derivative thereof, or the pharmaceutically acceptable salt of any of the foregoing, or the crystalline form or the solvate of any of the foregoing according to claim 1, wherein, when R^{7a} is a substituted or unsubstituted C₁₋₆ alkyl, the substituted or unsubstituted C₁₋₆ alkyl is isopropyl;
or, when R^{7a} is a substituted or unsubstituted C₃₋₆ cycloalkyl, the substituted or unsubstituted C₃₋₆ cycloalkyl is cyclopropyl;
or, when R^{7b} and R^{7c} are each independently a substituted or unsubstituted C₁₋₄ alkyl, the substituted or unsubstituted C₁₋₄ alkyl is ethyl, isopropyl, 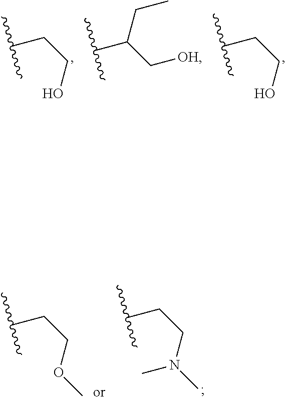
or, when R^{7b} and R^{7c} are each independently a substituted or unsubstituted C₃₋₆ cycloalkyl, the substituted or unsubstituted C₃₋₆ cycloalkyl is cyclopropyl or 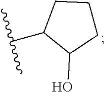 [SMILES: *C1CCCC1O]
or when R^{7b} and R^{7c} are each independently a substituted or unsubstituted 5-6 membered heteroaryl, the substituted or unsubstituted 5-6 membered heteroaryl is 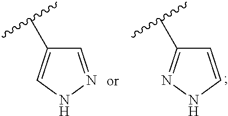 [SMILES: *c1cn[nH]c1 ; *c1cc[nH]n1]
or, when R^{7b} and R^{7c}, together with a nitrogen atom attached thereto, form a substituted or unsubstituted 4-6 membered heterocycloalkyl, the substituted or unsubstituted 4-6 membered heterocycloalkyl is 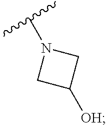 [SMILES: *N1CC(O)C1]
or, when R^{c} is —NR^{c1}R^{c2}, the —NR^{c1}R^{c2} is —N(CH₃)₂;
or, when R^{d} is —NR^{d1}R^{d2}, the —NR^{d1}R^{d2} is —N(CH₃)₂;
or, when R^{e} is —NR^{e1}R^{e2}, the —NR^{e1}R^{e2} is —N(CH₃)₂;
or, when R^{7d} is —NR^{a1}R^{a2}, the —NR^{a1}R^{a2} is —N(CH₃)₂.

6. The compound or the tautomer, the stereoisomer or the isotopic derivative thereof, or the pharmaceutically acceptable salt of any of the foregoing, or the crystalline form or the solvate of any of the foregoing according to claim 1, wherein when R¹ is 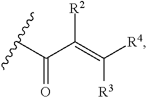
the 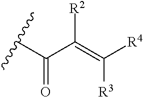
is 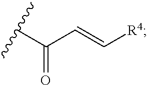
or, when R⁴ is —CH₂—NR^{4a}R^{4b}, the —CH₂—NR^{4a}R^{4b} is —CH₂—N(CH₃)₂;
or, when R⁷ is a substituted or unsubstituted C₁₋₆ alkyl, the substituted or unsubstituted C₁₋₆ alkyl is methyl, ethyl, isopropyl or —CH₂OH;
or, when R⁷ is a substituted or unsubstituted 5-6 membered heteroaryl, the substituted or unsubstituted 5-6 membered heteroaryl is 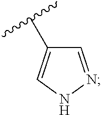 [SMILES: *c1cn[nH]c1]
or, when R⁷ is a substituted or unsubstituted C₃₋₆ cycloalkyl, the substituted or unsubstituted C₃₋₆ cycloalkyl is cyclopropyl;
or, when R⁷ is —OR^{7a}, the —OR^{7a} is 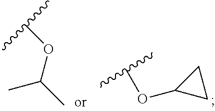
or, when R⁷ is —NR^{7b}R^{7c}, the —NR^{7b}R^{7c} is

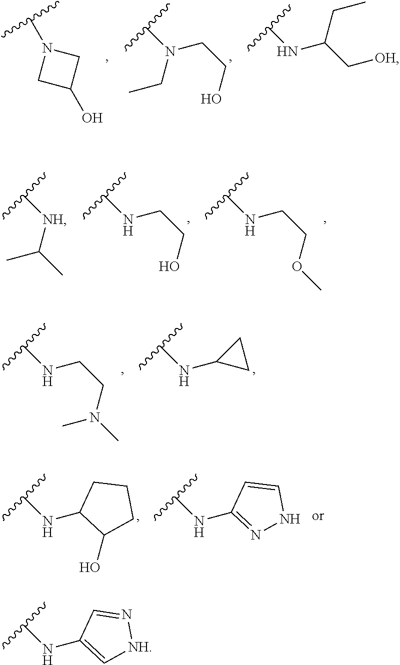

7. The compound or the tautomer, the stereoisomer or the isotopic derivative thereof, or the pharmaceutically acceptable salt of any of the foregoing, or the crystalline form or the solvate of any of the foregoing according to claim 1, wherein R^{a} is hydrogen;
or, n is 1;
or, R² is hydrogen;
or, R³ is hydrogen;
or, R⁴ is —CH₂—NR^{4a}R^{4b};
or, R^{4a} is C₁₋₆ alkyl;
or, R^{4b} is C₁₋₆ alkyl;
or, R⁵ is C₁₋₆ alkyl;
or, each R⁷ is independently hydrogen, a substituted or unsubstituted C₁₋₆ alkyl, a substituted or unsubstituted 5-6 membered heteroaryl, a substituted or unsubstituted C₃₋₆ cycloalkyl, —OR^{7a} or —NR^{7b}R^{7c}, wherein the substituted C₁₋₆ alkyl and the substituted 5-6 membered heteroaryl mean that the C₁₋₆ alkyl, the C₃₋₆ cycloalkyl and the 5-6 membered heteroaryl are each independently substituted with 1, 2, 3 or 4 R^{7d};
or, each R^{7d} is independently hydroxy, —NR^{a1}R^{a2} or C₁₋₄ alkoxy;
or, R^{a1} and R^{a2} are C₁₋₄ alkyl;
or, R^{7a} is hydrogen, a substituted or unsubstituted C₁₋₆ alkyl, or a substituted or unsubstituted C₃₋₆ cycloalkyl, wherein the substituted C₁₋₆ alkyl and the substituted C₃₋₆ cycloalkyl mean that the C₁₋₆ alkyl and the C₃₋₆ cycloalkyl are each independently substituted with 1, 2, 3 or 4 R^{c};
or, R^{c} is hydroxy, —NR^{c1}R^{c2} or C₁₋₄ alkoxy;
or, R^{c1} and R^{c2} are C₁₋₄ alkyl;
or R^{7b} and R^{7c} are each independently hydrogen, a substituted or unsubstituted C₁₋₄ alkyl, a substituted or unsubstituted C₃₋₆ cycloalkyl, or a substituted or unsubstituted 5-6 membered heteroaryl, wherein the substituted C₁0.4 alkyl, the substituted C₃₋₆ cycloalkyl and the substituted 5-6 membered heteroaryl mean that the C₁₋₄ alkyl, the C₃₋₆ cycloalkyl and the 5-6 membered heteroaryl are each independently substituted with 1, 2, 3 or 4 R^{d};
or, R^{d} is hydroxy, —NR^{d1}R^{d2} or C₁₋₄ alkoxy;
or, R^{d1} and R^{d2} are C₁₋₄ alkyl.

8. The compound or the tautomer, the stereoisomer or the isotopic derivative thereof, or the pharmaceutically acceptable salt of any of the foregoing, or the crystalline form or the solvate of any of the foregoing according to claim 1, wherein the compound is selected from any of the following structures: 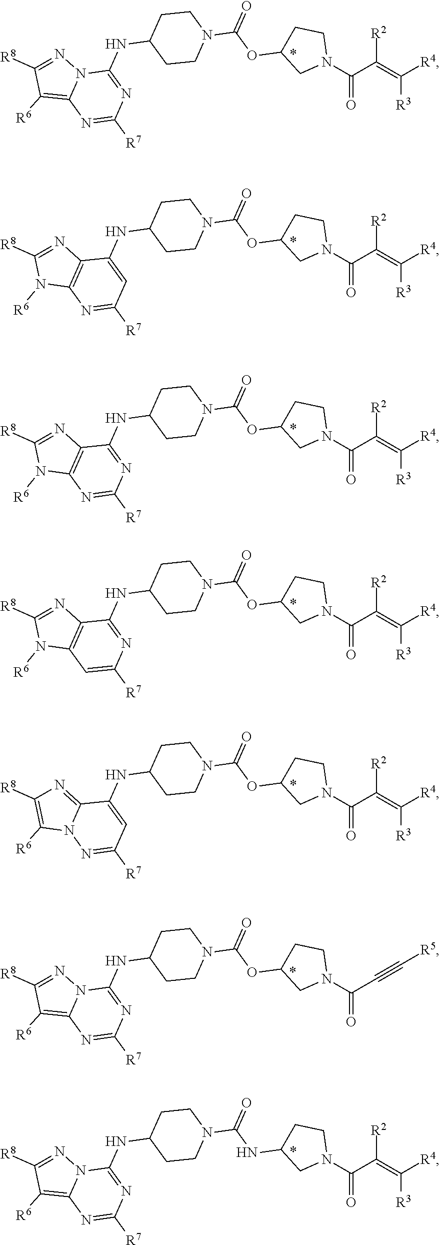 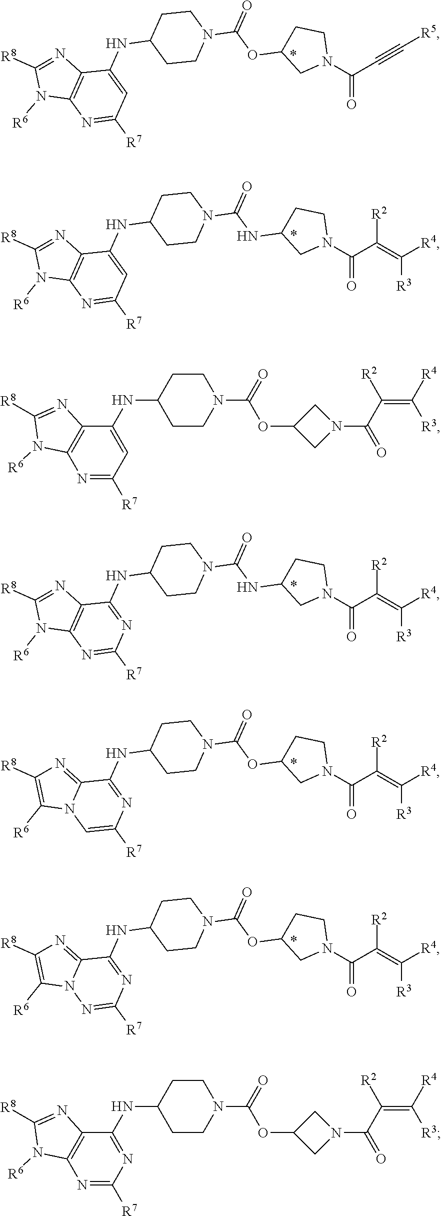
wherein R², R³, R⁴, R⁵, R⁶, R⁷, R⁸ and * are defined as above.

9. The compound of the tautomer, the stereoisomer or the isotopic derivative thereof, or the pharmaceutically acceptable salt of any of the foregoing, or the crystalline form or the solvate of any of the foregoing according to claim 1, wherein R⁶ is C₁₋₆ alkyl;
or, R⁷ is C₁₋₆ alkyl;
or, R⁸ is hydrogen.

10. The compound or the tautomer, the stereoisomer or the isotopic derivative thereof, or the pharmaceutically acceptable salt of any of the foregoing, or the crystalline form or the solvate of any of the foregoing according to claim 1, wherein the compound is any of the following structures:

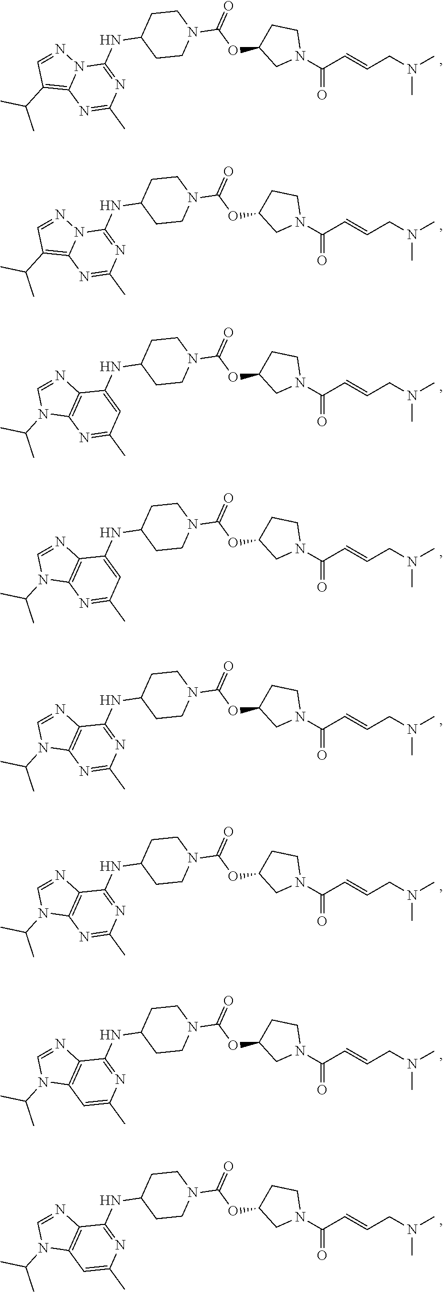

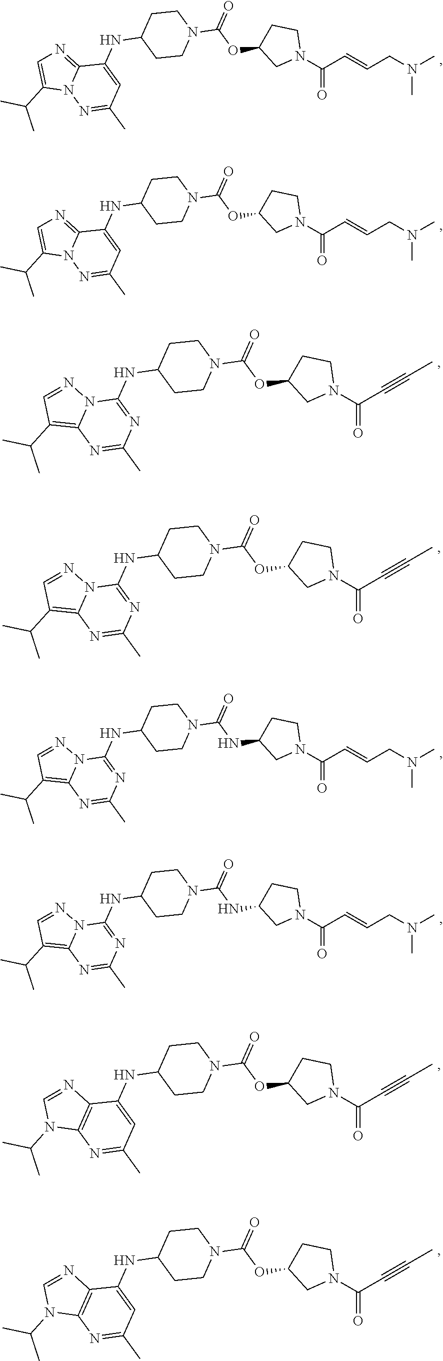

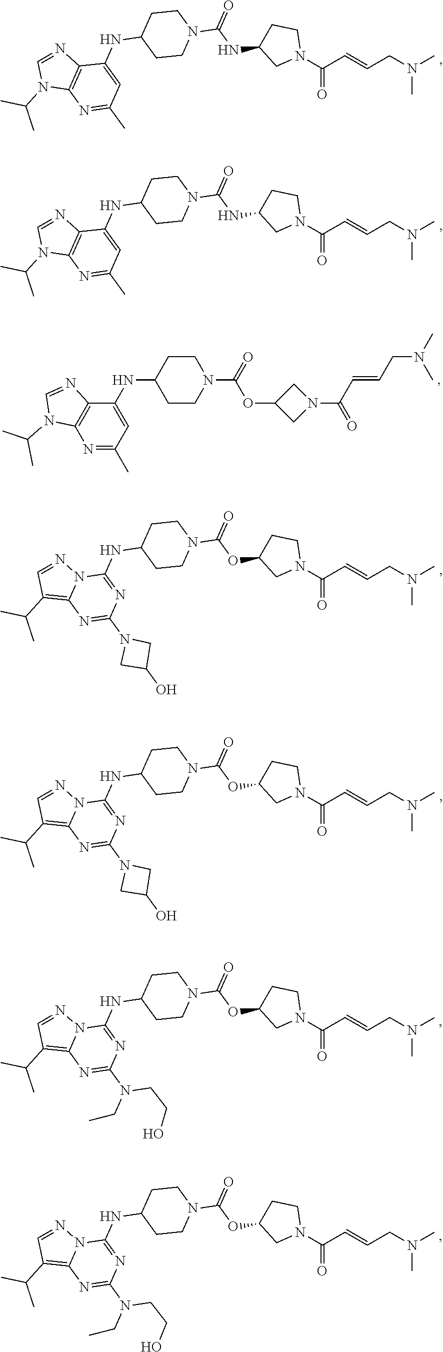

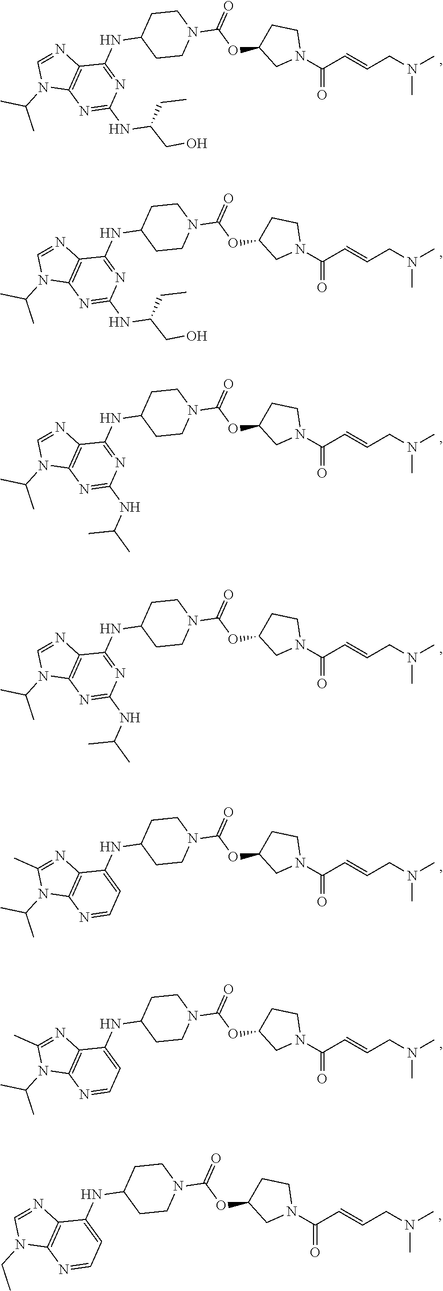

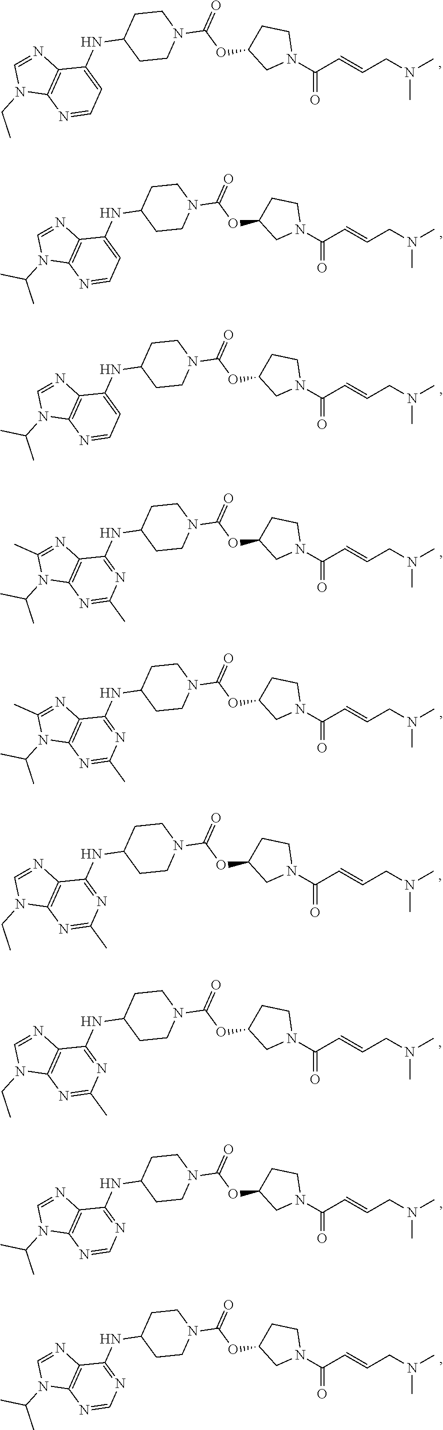

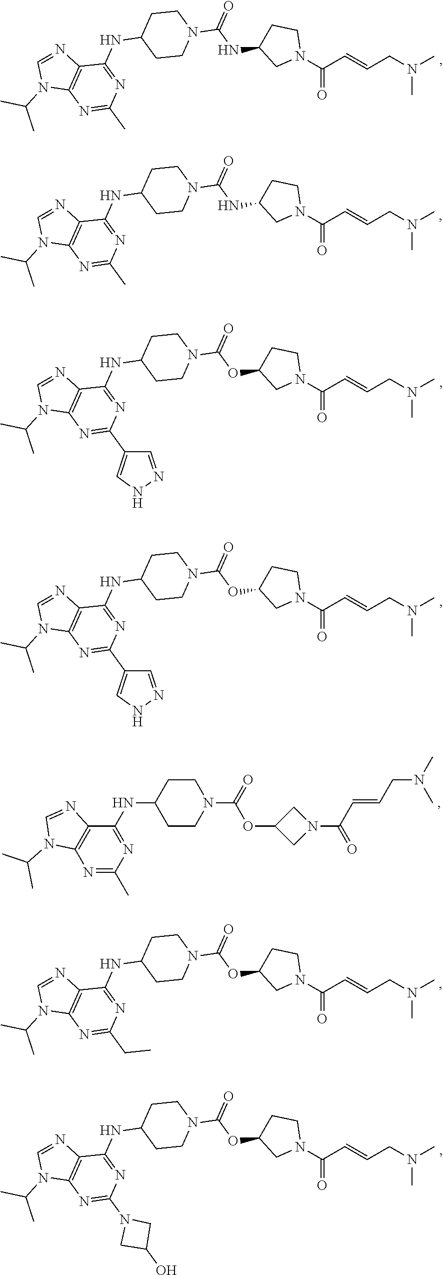

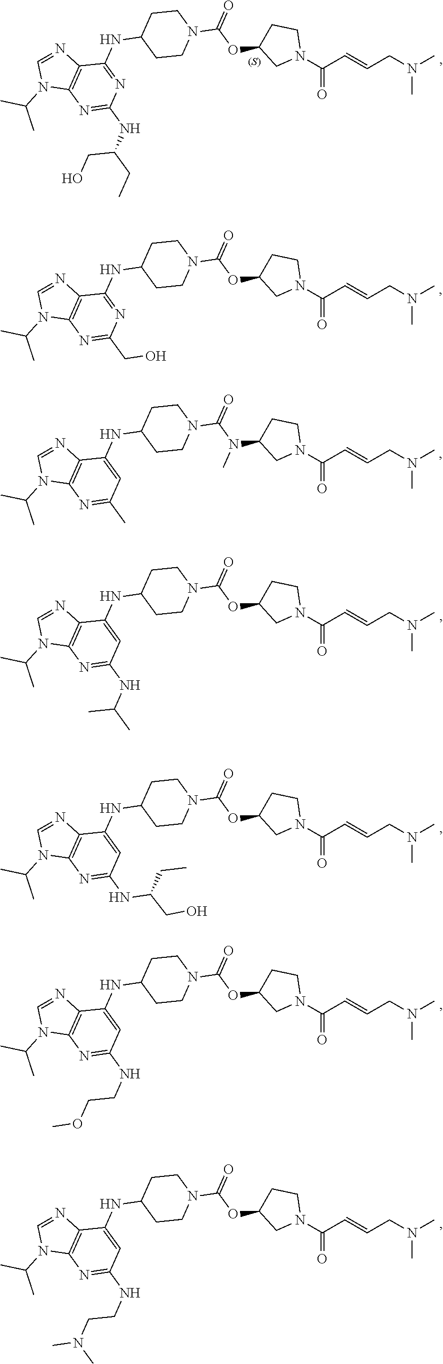

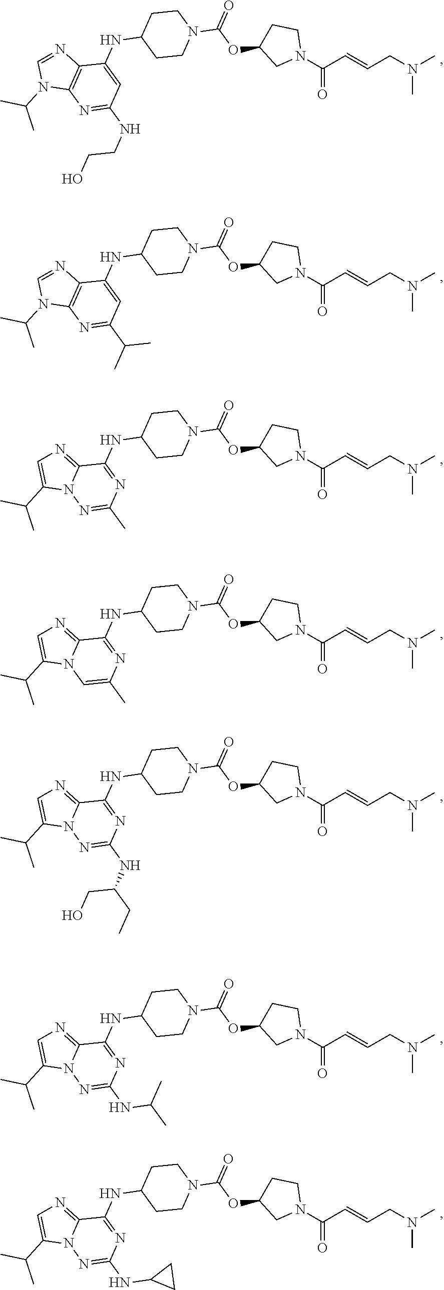

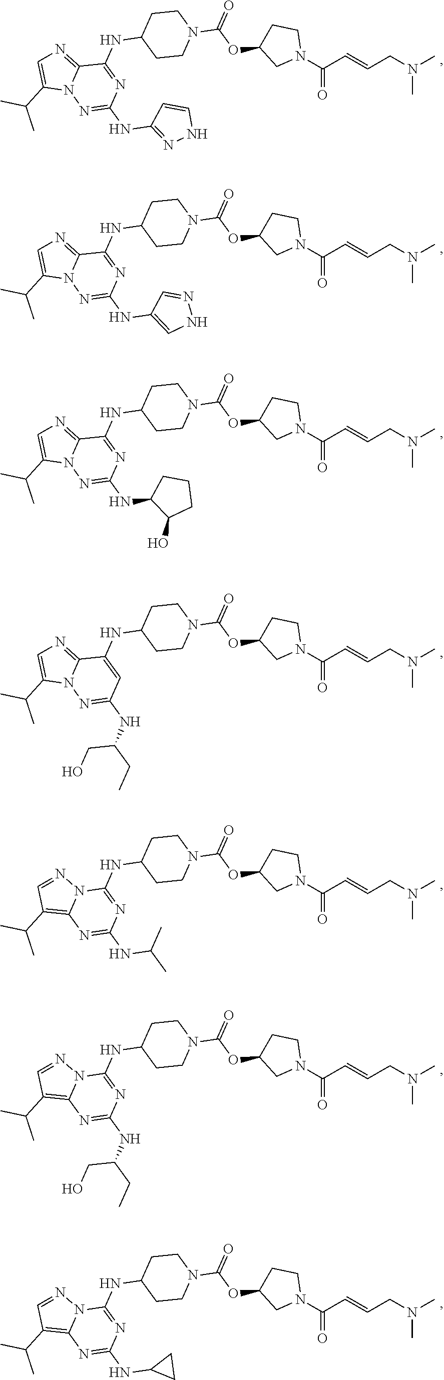

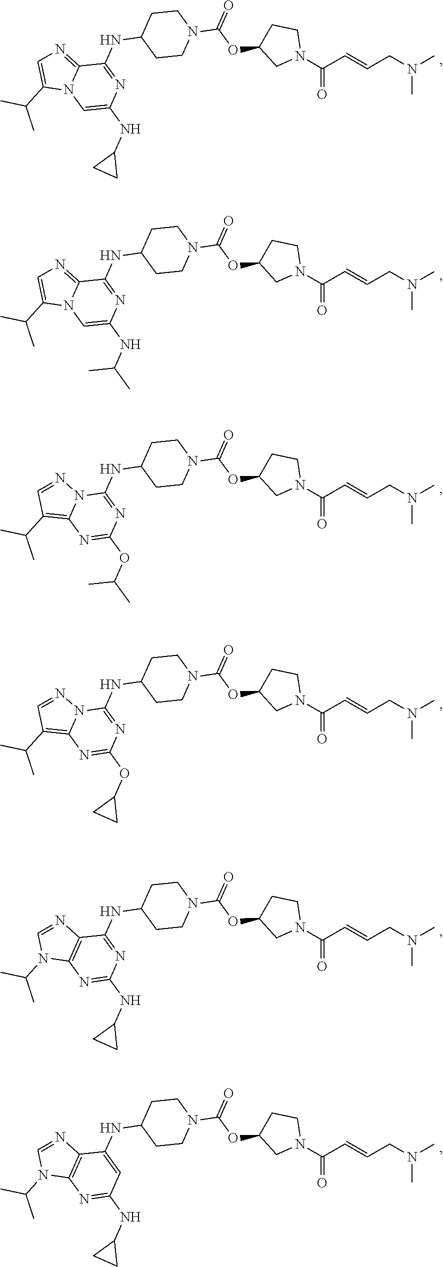

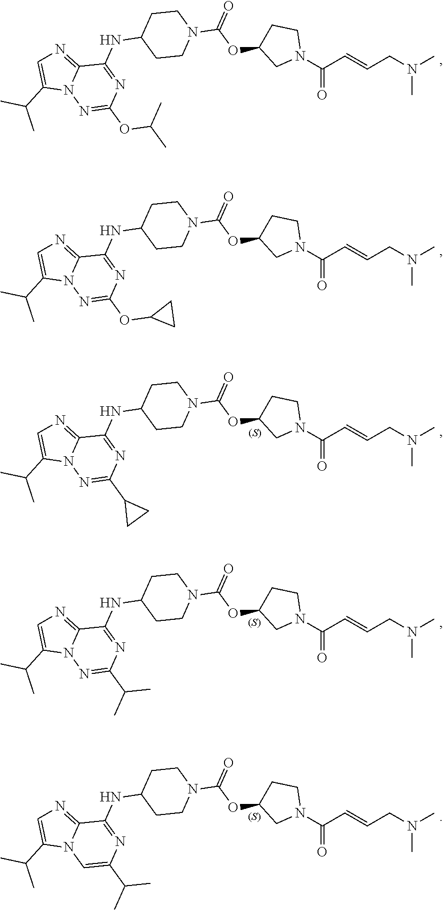

11. A method for preparing the compound of formula I according to claim 1, comprising: subjecting a compound of formula II and 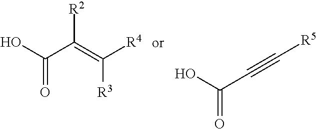
to a condensation reaction shown below in an organic solvent in the presence of a condensing agent and a base to obtain the compound of formula I, wherein ring A, X, m, n, R¹, R², R³, R⁴, R⁵ and * are defined as above;

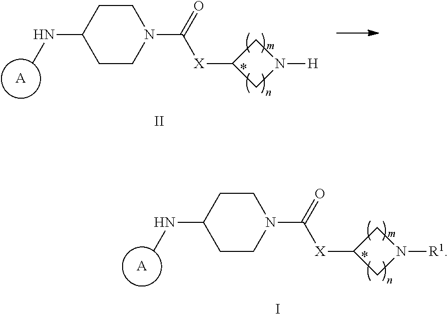

12. A compound or a tautomer, a stereoisomer or an isotopic derivative thereof, or a pharmaceutically acceptable salt of any of the foregoing, or a crystalline form or a solvate of any of the foregoing, wherein the compound is selected from any of the following structures: 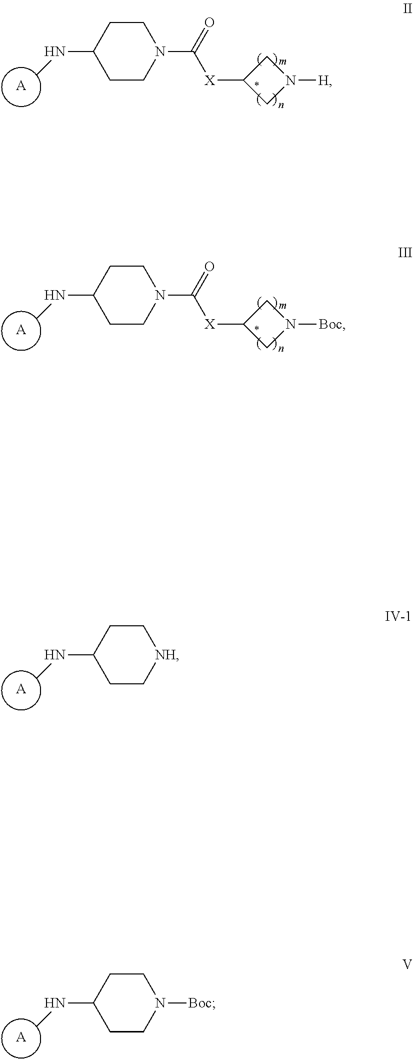
wherein ring A, X, m, n and * are defined as in claim 1.

13. A pharmaceutical composition, comprising:
(i) the compound of formula I or the tautomer, the stereoisomer or the isotopic derivative thereof, or the pharmaceutically acceptable salt of any of the foregoing, or the crystalline form or the solvate of any of the foregoing according to claim 1; and
(ii) at least one pharmaceutical adjuvant.

14. (canceled)

15. (canceled)

16. (canceled)

17. (canceled)

18. A method for preventing or treating a CDK7-mediated disease in a subject in need thereof, comprising administering the compound or the tautomer, the stereoisomer or the isotopic derivative thereof, or the pharmaceutically acceptable salt of any of the foregoing, or the crystalline form or the solvate of any of the foregoing according to claim 1 to the subject.

19. The method according to claim 18, wherein the CDK7-mediated disease is a tumor.

20. The method according to claim 19, wherein the CDK7-mediated disease is breast cancer, ovarian cancer, small cell lung cancer, acute myeloid leukemia, acute lymphocytic leukemia, bladder cancer, colon cancer, prostate cancer, epithelial sarcoma or soft tissue sarcoma.

21. A method for preventing or treating a CDK7-mediated disease in a subject in need thereof, comprising administering the compound or the tautomer, the stereoisomer or the isotopic derivative thereof, or the pharmaceutically acceptable salt of any of the foregoing, or the crystalline form or the solvate of any of the foregoing according to claim 10 to the subject.

22. The method according to claim 21, wherein the CDK7-mediated disease is a tumor.

23. The method according to claim 22, wherein the CDK7-mediated disease is breast cancer, ovarian cancer, small cell lung cancer, acute myeloid leukemia, acute lymphocytic leukemia, bladder cancer, colon cancer, prostate cancer, epithelial sarcoma or soft tissue sarcoma.
